(19) United States
(12) Patent Application Publication
(10) Pub. No.: US 2020/0306346 A1
(43) Pub. Date: Oct. 1, 2020

(54) COMPOSITIONS IN THE FORM OF AN INJECTABLE AQUEOUS SOLUTION COMPRISING HUMAN GLUCAGON AND A CO-POLYAMINO ACID

(71) Applicant: ADOCIA, Lyon (FR)

(72) Inventors: Alexandre GEISSLER, Lyon (FR); Grégory MEIFFREN, Meyzieu (FR)

(73) Assignee: ADOCIA, Lyon (FR)

(21) Appl. No.: 16/902,153

(22) Filed: Jun. 15, 2020

Related U.S. Application Data
(63) Continuation of application No. 16/213,929, filed on Dec. 7, 2018.

(30) Foreign Application Priority Data
Dec. 7, 2017 (FR) 17/61772
Dec. 7, 2017 (FR) 17/61777
Jun. 29, 2018 (FR) 18/56068
Jun. 29, 2018 (FR) 18/56069

(51) Int. Cl. A61K 38/26; A61K 9/00; A61K 9/08; A61K 47/02; A61K 47/12; A61K 47/34; C07K 14/605; A61P 3/10

(52) U.S. Cl. CPC A61K 38/26; A61K 9/0019; A61K 9/08; A61P 3/10; A61K 47/12; A61K 47/34; C07K 14/605; A61K 47/02

(57) ABSTRACT

Physically stable compositions in the form of an injectable aqueous solution, the pH of which is comprised from 6.0 to 8.0, having at least: human glucagon and a co-polyamino acid bearing carboxylate charges and hydrophobic radicals Hy, in one embodiment, the compositions according to the invention further includes a gastrointestinal hormone.

This is a Continuation of application Ser. No. 16/213,929 filed Dec. 7, 2018, which claims the benefit of French Application No. 17/61772 filed Dec. 7, 2017, French Application No. 17/61777 filed Dec. 7, 2017, French Application No. 18/56068 filed Jun. 29, 2018, and French Application No. 18/56069 filed Jun. 29, 2018. The disclosure of the prior applications is hereby incorporated by reference herein in its entirety.

Human glucagon is a short-acting hyperglycemic hormone that increases blood sugar, thereby correcting a hypoglycemic level that may result from excess insulin. It permits the release of glucose by stimulation of hepatic glycogenolysis, and has (hypoglycemic) insulin antagonistic properties. Human glucagon is normally secreted by the alpha cells of Langerhans islets in the pancreas when hypoglycemia is detected.

Human glucagon is used for therapeutic purposes, such as the emergency treatment of severe hypoglycemia, also called “rescue”, as well as within a diagnostic framework during medical exams, for example to inhibit gastrointestinal motility. Human glucagon is also envisaged for other applications, especially its use in a bi-hormonal blood glucose control system also called artificial pancreas and in congenital hyperinsulinism which is a rare disease characterized by very high levels of insulin.

The clinical use of human glucagon has been limited because of some of its poor properties for developing a stable pharmaceutical product for therapeutic purposes. In fact, human glucagon has a very low solubility at physiological pH and a high physical instability because of its propensity to form fibrils over a wide range of pH. It is for this reason that the only commercial products based on human glucagon (Glucagen®, NOVO NORDISK and Glucagon for injection, ELI LILLY) are freeze-dried forms to be reconstituted extemporaneously.

The studies by Onoue et al. (Pharm Res 2004, 21 (7), 1274-83) have shown the potentially dangerous nature of these fibrils: fibrillated human glucagon being cytotoxic in mammalian cells in culture.

In addition to its physical instability, human glucagon undergoes various types of chemical degradation. In aqueous solution, it degrades rapidly to form several degradation products. At least 16 human glucagon degradation products have been identified by Kirsh et al. (International Journal of Pharmaceutics, 2000, 203, 115-125). The chemical degradation of this human glucagon is therefore rapid and complex.

The poor chemical and physical stability of human glucagon in solution has led pharmaceutical companies like NOVO NORDISK, ELI LILLY and more recently FRESENIUS KABI to market this human glucagon in the form of a lyophilizate to be reconstituted at acidic pH (pH<3) just before injection. Human glucagon in freeze-dried form is more stable, and preparing the formulation at acidic pH immediately prior to use provides a clear solution. However, once the product is reconstituted, it must be used rapidly because it undergoes extremely rapid chemical and physical degradation in the acidic reconstitution buffer, with the appearance of human glucagon fibrils within 24 hours of reconstitution, and/or gelation of the composition. This form of the product is nonetheless unsatisfactory because it requires a rapid use of the formulation. This instability not only makes use in pump impossible, but it also has the disadvantage of leading to significant product losses in diagnostic use. This type of composition is actually wasteful since it is no longer usable a few hours after preparation.

Finally, even in its use for emergency treatment of severe hypoglycemic reactions, which may occur during insulin therapy in diabetic patients, the formulation to be reconstituted is not ideal either, because it involves a long and complicated preparation, for example GlucaGen® user guide describes a 5-step process for injecting the recommended dose. Moreover, a study from LOCEMIA indicates that very few people (about 10% of the participants) in need of emergency reconstitution were able to deliver the correct dose. Finally, the acidic pH of human glucagon solutions can cause pain upon injection for the patient.

There is therefore a need for a ready-to-use human glucagon solution. Today, the most clinically advanced solutions for delivering human glucagon circumvent the stability issue with human glucagon in aqueous solution in different ways.

The Locemia COMPANY has developed a freeze-dried human glucagon spray, currently tested in a phase 3 clinical study, which is intended to be administered intranasally. This spray is suitable for “rescue” use, that is, in the case of severe hypoglycemia, because it is ready for use and therefore easy to use, unlike the solutions to be prepared. However, this product is not suitable for pump use or a use requiring an accurate control of the amount of human glucagon delivered.

Meanwhile, XERIS has developed a liquid formulation of human glucagon based on a polar aprotic solvent, such as DMSO, currently tested in clinical studies. However, if the injection of organic solvent solution for “rescue” use is conceivable, it is highly preferable to have a human glucagon aqueous solution for chronic use. Compositions comprising an association with other peptides are envisaged, in particular amylin or GLP-1 RA (glucagon like peptide-1 receptor agonist).

Finally, with increasing difficulties of formulating human glucagon, human glucagon analogues are being developed by major pharmaceutical companies, such as NOVO NORDISK, SANOFI OR ELI LILLY, in order to obtain formulations having a stability compatible with a pharmaceutical use. However, these peptides for which the primary sequence has been modified in relation to human peptide may pose a safety risk for patients.

There is therefore a major interest for a solution that would improve both the chemical and physical solubilization and stability of human glucagon in aqueous solution at a pH close to physiological pH, hence from 6.0 to 8.0. This could lead to a pharmaceutical product easier to use by a patient in an emergency, but also pave the way for new therapeutic uses of human glucagon, such as its use in a bihormonal artificial pancreas.

The prior art proposes solutions to attempt to solve this problem.

Some documents recommend applying abasic pH. For example, US2015291680 recommends the solubilization of human glucagon at 1 mg/ml at a pH ranging from 8.8 to 9.4 and using ferulic acid or tetrahydrocurcumin. However, in addition to being at basic pH, this solution has the disadvantage of leading to a fairly time-limited human glucagon stability. The article by Jackson et al. (Curr Diab, Rep., 2012, 12, 705-710) proposes to formulate human glucagon at basic pH (approximately 10) in order to limit the formation of fibrils. However, this solution does not prevent rapid chemical degradation of human glucagon.

Patent application WO2014096440 (NOVOZYME) on the contrary envisages a slightly acidic pH (about 5.5) in the presence of albumin and polysorbate, in order to improve the stability by reducing the rate of fibrillation. However, this solution exhibits limited improvement of the stability. Most solutions described in the prior art making it possible to obtain a clear solution of human glucagon and to prevent aggregation, gelation or precipitation of human glucagon involve the use of known surfactants, detergents or solubilizing agents.

For example, Matilainen et al. (J. Pharm Sci, 2008, 97, 2720-2729 and Eur J. Pharm Sci., 2009, 36, 412-420) described the use of cyclodextrin to limit the formation rate of human glucagon fibrils. However, the improvement provided seems insufficient to consider a use in pump.

Among the proposed solutions are hydrophilic surfactants:
GB1202607 (NOVO NORDISK) describes the use of anionic or cationic detergents.
U.S. Pat. No. 6,384,016 (NOVO NORDISK) and US2011097386 (BIODEL) use lysophospholipids (or lysolecithins).
WO2015095389 (AEGIS) discloses non-ionic surfactants, like dodecyl maltoside, for improving the bioavailability of therapeutic agents, in the case of delivery by application to the mucous membranes or the epidermis, and especially in the case of ocular, nasal, oral or nasolacrimal delivery. This document outlines that the presence of alkyl glycosides leads to improved absorption of human glucagon in the eye,
WO2012059764 (ARECOR) application discloses cationic surfactants, and more specifically aromatic ammonium chlorides.

The surfactants indicated in the above documents may be too toxic or irritating for chronic subcutaneous use. For example, lysophospholipids (or lysolecithins) are known to lyse red blood cells because of their hemolytic properties. When injected subcutaneously, this can cause local tissue damage and pain at the injection site. In the case of continuous injection by a pump, this can lead to pain and/or irritation at the insertion site of the needle. International application WO2011138802 (Sun Pharma) discloses a ready-to-use solution of human glucagon in micellar aqueous solution at a pH ranging from 5 to 7.5 in the presence of a pegylated lipid (pegylated distearoyl-phosphotidylethanolamine). However, Garay et al. (Expert Opin Drug Deliv (2012) 9, 1319-1323) reveal that polyethylene glycol (PEG) is both immunogenic and antigenic. This can be detrimental to patients with anti-PEG antibodies. Furthermore, Ganson et al. (J. Allergy Clin Immunol (2015) doi: 10.1016/j.jaci.2015.10.034) outline that a clinical study of pegnivacogin coupled with 40 kDa methoxypolyethylene glycol (mPEG) led to inflammatory responses at the first dose of pegnivacogin in 3 out of 640 patients. Of these three patients, two met the criteria for anaphylaxis and one had an isolated dermal reaction, each event was considered serious, and one was even considered to be life-threatening. These adverse events triggered the discontinuation of the clinical trial and raised the problem of adverse effects of pegylated compounds.

WO2013101749 (LATITUDE) discloses nano-emulsions of human glucagon. However, it claims rather modest performance in terms of chemical stability, i.e. the composition comprises at least 75% of the initial concentration after 3-7 days at 37° C.

In addition, it should be noted that to date, to the applicant's knowledge, no pharmaceutical formulation comprising human glucagon in the form of an aqueous solution has been tested in clinical study.

Therefore, there remains a need for a liquid aqueous formulation at a pH close to physiological pH ranging from 6.0 to 8.0 to solubilize and obtain good stability of human glucagon, both in terms of physical stability and chemical stability. More specifically, there is a need for such a formulation that can be used in a bihormonal pump (insulin/human glucagon).

This need is so clear that Tan et al. (Diabetes, 2013, 62, 1131-138) illustrate that combining human glucagon with a GLP-1 RA is an attractive proposition for the treatment of obesity and diabetes. However, the ability to formulate human glucagon in a stable manner in aqueous solution at a pH close to physiological pH ranging from 6.0 to 8.0 makes it possible to be in more favorable conditions for improving the stability of GLP-1 RAs; sensitive to acidic or basic conditions.

The co-polyamino acids bearing carboxylate charges and hydrophobic radicals Hy according to the invention exhibit excellent resistance to hydrolysis. This can especially be viewed under accelerated conditions, for example by hydrolysis tests at basic pH (pH 12).

In addition, forced oxidation tests, for example of the fenton oxidation type, reveal that the co-polyamino acids bearing carboxylate charges and hydrophobic radicals Hy have a good resistance to oxidation.

Moreover, the present invention makes it possible to obtain a faster action and/or a faster absorption of glucagon.

It also aims to improve the efficiency of the co-polyamino acids bearing carboxylate charges and hydrophobic radicals Hy with respect to glucagon.

The invention thus concerns physically stable compositions in the form of an injectable aqueous solution, the pH of which is comprised from 6.0 to 8.0, comprising at least:

a) human glucagon and

b) a co-polyamino acid bearing carboxylate charges and hydrophobic Hy radicals, the said co-polyamino acid consisting of glutamic or aspartic units and said hydrophobic Hy radicals being according to the following formula I:

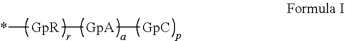

wherein
GpR is a radical according to formulas II, II ‘or II′’:

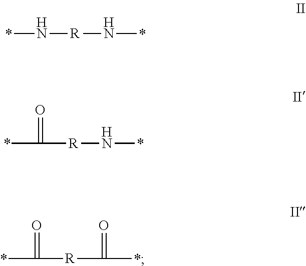

GpA is a radical according to formulas III or III′:

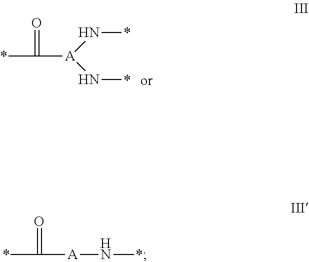

GpC is a radical according to formula IV:

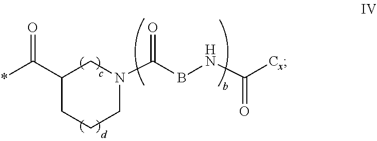

* indicate the attachment sites of the various groups;
a is an integer equal to 0 or 1;
b is an integer equal to 0 or 1;
p is an integer equal to 1 or 2 and
   if p is equal to 1 then a is equal to 0 or 1 and GpA is a radical according to formula III′ and,
   if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
   if r is equal to 0, then the hydrophobic radical according to formula I is bound to the co-polyamino acid by a covalent bond between a carbonyl of the hydrophobic radical and a nitrogen atom in the N-terminal position of the co-polyamino acid, thereby forming an amide function from the reaction of an amine function at the N-terminal position of the precursor of the co-polyamino acid and an acid function borne by the precursor of the hydrophobic radical, and
   if r is equal to 1 or 2, then the hydrophobic radical according to formula I is bound to the co-polyamino acid:
      by a covalent bond between a nitrogen atom of the hydrophobic radial and a carbonyl of the copolyamino acid, thus forming an amide function from the reaction of an amine function of the precursor of the hydrophobic radical and an acid function borne by the precursor of the co-polyamino acid or
      by a covalent bond between a carbonyl of the hydrophobic radical and a nitrogen atom in N-terminal position of the co-polyamino acid, thus forming an amide function from the reaction of an acid function of the precursor of the hydrophobic radical and an amine function in N-terminal position borne by the precursor of the co-polyamino acid;
R is a radical chosen from the group consisting of a linear or branched divalent alkyl radical comprising from 1 to 12 carbon atoms, a divalent linear or branched alkyl radical comprising from 1 to 12 carbon atoms bearing one or more —CONH2 functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or a unsubstituted ether or polyether radical comprising from 4 to 14 carbon atoms and from 1 to 5 oxygen atoms;
more precisely, R is a radical chosen from the group consisting of:
   a divalent alkyl radical, linear or branched, comprising from 2 to 12 carbon atoms if GpR is a radical according to formula II, from 1 to 11 carbon atoms if GpR is a radical according to formula II′ or from 1 to 10 carbon atoms if GpR is a radical according to formula II″;
   a divalent, linear or branched alkyl radical, comprising from 2 to 11 carbon atoms if GpR is a radical according to formula II from 1 to 11 carbon atoms if GpR is a radical according to formula II′, or from 1 to 10 carbon atoms if GpR is a radical according to formula II″, said alkyl radical bearing one or more functions —CONH₂, and
   an unsubstituted ether or polyether radical comprising from 4 to 14 carbon atoms and from 1 to 5 oxygen atoms;
A is a linear or branched alkyl radical comprising from 1 to 8 carbon atoms and optionally substituted by a radical resulting from a saturated, unsaturated or aromatic ring;
B is a radical chosen from the group consisting of an unsubstituted ether or polyether radical comprising from 4 to 14 carbon atoms and from 1 to 5 oxygen atoms or a linear or branched alkyl radical, optionally comprising an aromatic ring, comprising from 1 to 9 carbon atoms;
Cₓ is a linear or branched monovalent alkyl radical, optionally comprising a cyclic part, wherein x indicates the number of carbon atoms and:
   if p is equal to 1, x is comprised from 11 to 25 (11≤x≤25)
   if p is equal to 2, x is comprised from 9 to 15 (9≤x≤15),
the ratio i between the number of hydrophobic radicals and the number of glutamic or aspartic units being comprised between 0<i≤0.5;
when several hydrophobic radicals are borne by a co-polyamino acid they are therefore identical or different,
the degree of polymerization DP of glutamic or aspartic units is comprised from 5 to 250;
the free acid functions being in the form of an alkaline cation salt chosen from the group consisting of Na⁺ and K⁺.

The invention further relates to a method for preparing stable injectable compositions.

“Soluble” is understood to mean capable of producing a clear and particle-free solution at a concentration of less than 100 mg/ml in distilled water at 25° C.

“Solution” is understood to mean a liquid solution which is free of visible particles, using the method according to the European pharmacopoeia 8.0, in point 2.9.20, and the American pharmacopoeia <790>.

“Physically stable composition” is understood to mean compositions which, after a certain storage time at a certain temperature satisfy the criteria of the visual inspection described in the European pharmacopoeia, the American pharmacopoeia and the international pharmacopoeia, meaning compositions which are clear and which contain no visible particles, but also colorless.

“Chemically stable composition” is understood to mean compositions which, after storage for a certain time and at a certain temperature, have a minimum recovery of the active ingredients and which comply with the specifications applicable to pharmaceutical products.

A conventional method for measuring the stability of proteins or peptides is to measure the formation of fibrils using Thioflavin T, also known as ThT. This method makes it possible to measure the latency time before the formation of fibrils by measuring the increase in fluorescence, under temperature and stirring conditions which permit an acceleration of the phenomenon. The compositions according to the invention have a latency time before the formation of fibrils markedly greater than that of glucagon at the pH of interest.

“Injectable aqueous solution” is understood to mean water-based solutions which meet the conditions of the European and American pharmacopoeias and which are liquid enough to be injected.

“Co-polyamino acid consisting of glutamic or aspartic units” is understood to mean non-cyclic linear chains of glutamic acid or aspartic acid units bound together by peptide bonds, said chains having a C-terminal part, corresponding to the carboxylic acid at one end, and an N-terminal part, corresponding to the amine at the other end of the sequence.

“Alkyl radical” is understood to mean a linear or branched carbon chain, which does not include a heteroatom.

Co-polyamino acid is a statistical or block co-polyamino acid.

Co-polyamino acid is a statistical co-polyamino acid bound to glutamic and/or aspartic units.

In the formulas, the * indicate the attachment sites of the various elements represented.

In formulas I, V and VI, the * indicate the attachment sites of the hydrophobic radicals to the co-polyamino acid. The radicals Hy are attached to the co-polyamino acid by amide functions.

In formulas II and II′, the * indicate, from left to right respectively, the GpR attachment sites:
to the co-polyamino acid and
to GpR if r=2 or GpA if a=1 or GPC if a=0.

In formulas II and II′, the * indicate, from left to right respectively, the GpA attachment sites:
to GpR if r=1 or 2 or to the co-polyamino acid if r=0 and
to GpC.

In formula IV, the * indicates the GpC attachment site:
to GpA if a=1, GpR if r=1 or 2 and a=0 or to the co-polyamino acid if r=0 and a=0.

All attachments between the different groups GpR, GpA and GpC are amide functions.

Radicals Hy, GpR, GpA, GpC, and D are each independently identical or different from one monomeric unit to another.

In one embodiment, the composition is characterized in that the pH is comprised from 6.6 to 7.8.

In one embodiment, the composition is characterized in that the pH is comprised from 7.0 to 7.8.

In one embodiment, the composition is characterized in that the pH is comprised from 6.8 to 7.4.

In one embodiment, the composition according to the invention is characterized in that Hy comprises between 15 and 100 carbon atoms.

In one embodiment, the composition according to the invention is characterized in that Hy comprises between 30 and 70 carbon atoms.

In one embodiment, the composition according to the invention is characterized in that Hy comprises between 40 and 60 carbon atoms.

In one embodiment, the composition according to the invention is characterized in that Hy comprises between 20 and 30 carbon atoms

In one embodiment, Hy comprises more than 15 carbon atoms.

In one embodiment, Hy comprises more than 30 carbon atoms.

In one embodiment, the composition is characterized in that the hydrophobic radical according to formula I is a radical wherein R is a divalent linear alkyl radical comprising from 2 to 12 carbon atoms.

In one embodiment, the composition is characterized in that the hydrophobic radical according to formula I is a radical wherein R is a divalent alkyl radical comprising from 2 to 6 carbon atoms.

In one embodiment, the composition is characterized in that the hydrophobic radical according to formula I is a radical wherein R is a divalent linear alkyl radical comprising from 2 to 6 carbon atoms.

In one embodiment, the composition is characterized in that the hydrophobic radical according to formula I is a radical wherein R is a divalent alkyl radical comprising from 2 to 4 carbon atoms.

In one embodiment, the composition is characterized in that the hydrophobic radical according to formula I is a radical wherein R is a divalent linear alkyl radical comprising from 2 to 4 carbon atoms.

In one embodiment, the composition is characterized in that the hydrophobic radical according to formula I is a radical wherein R is a divalent alkyl radical comprising 2 carbon atoms.

In one embodiment, the composition is characterized in that the hydrophobic radical according to formula I is a radical wherein R is a divalent linear alkyl radical comprising from 1 to 11 carbon atoms.

In one embodiment, the composition is characterized in that the hydrophobic radical according to formula I is a radical wherein R is a divalent alkyl radical comprising from 1 to 6 carbon atoms.

In one embodiment, the composition is characterized in that the hydrophobic radical according to formula I is a radical wherein R is a divalent alkyl radical comprising from 1 to 10 carbon atoms.

In one embodiment, the composition is characterized in that the hydrophobic radical according to formula I is a radical wherein R is a divalent alkyl radical comprising from 2 to 5 carbon atoms and bearing one or more amide functions (—CONH2).

In one embodiment, the composition is characterized in that the hydrophobic radical according to formula I is a radical wherein R is a divalent linear alkyl radical comprising from 2 to 5 carbon atoms and bearing one or more amide functions (—CONH2).

In one embodiment, the composition is characterized in that the hydrophobic radical according to formula I is a radical wherein R is a radical chosen from the group consisting of the radicals represented by the formulas below:

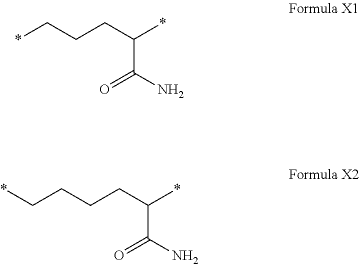 [SMILES: *CCCC(*)C(N)=O ; *CCCCC(*)C(N)=O]

In one embodiment, the composition is characterized in that the hydrophobic radical according to formula VI is a radical wherein R is a radical according to formula X1.

In one embodiment, the composition is characterized in that the hydrophobic radical according to formula I is a radical wherein R is a radical according to formula X2.

In one embodiment, the composition is characterized in that the hydrophobic radical according to formula I is a radical wherein R of GpR if r=1 or of GpR1 if r=2 is bound to the co-polyamino acid by an amide function borne by the carbon in the delta or epsilon position (or position 4 or 5) with respect to the amide function (—CONH2).

In one embodiment, the composition is characterized in that the hydrophobic radical according to formula I is a radical wherein R is an unsubstituted linear ether or polyether radical comprising from 4 to 14 carbon atoms and from 1 to 5 oxygen atoms.

In one embodiment, the composition is characterized in that the hydrophobic radical according to formula I is a radical wherein R is an ether radical.

In one embodiment, the composition is characterized in that the hydrophobic radical according to formula I is a radical wherein R is a divalent alkyl radical comprising from 4 to 6 carbon atoms.

In one embodiment, the composition is characterized in that the hydrophobic radical according to formula I is a radical wherein R is a divalent alkyl radical comprising 6 carbon atoms.

In one embodiment, the composition is characterized in that the hydrophobic radical according to formula I is a radical wherein R is an ether radical represented by the formula

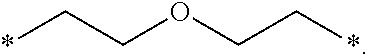

In one embodiment, the composition is characterized in that the hydrophobic radical according to formula I is a radical wherein R is a polyether radical.

In one embodiment, the composition is characterized in that the hydrophobic radical according to formula I is a radical wherein R is a linear polyether radical comprising from 6 to 10 carbon atoms and from 2 to 3 oxygen atoms.

In one embodiment, the composition is characterized in that the hydrophobic radical according to formula I is a radical wherein R is a radical chosen from the group consisting of the radicals represented by the formulas below:

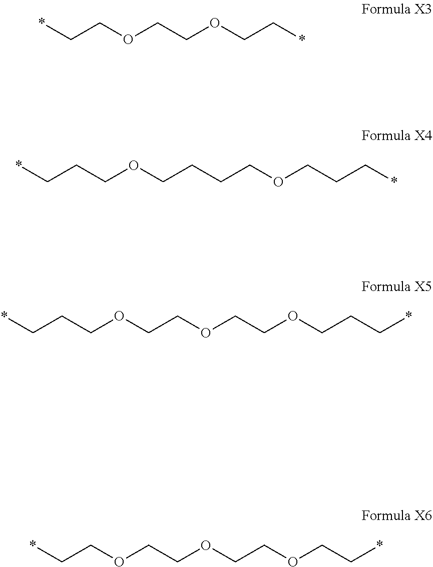 [SMILES: *CCOCCOCC* ; *CCCOCCCCOCCC* ; *CCCOCCOCCOCCC* ; *CCOCCOCCOCC*]

In one embodiment, the composition is characterized in that the hydrophobic radical according to formula I is a radical wherein R is a radical according to formula X3.

In one embodiment, the composition is characterized in that the hydrophobic radical according to formula I is a radical wherein R is a radical according to formula X4.

In one embodiment, the composition is characterized in that the hydrophobic radical according to formula I is a radical wherein R is a polyether radical chosen from the group consisting of the radicals represented by formulas X5 and X6 below:

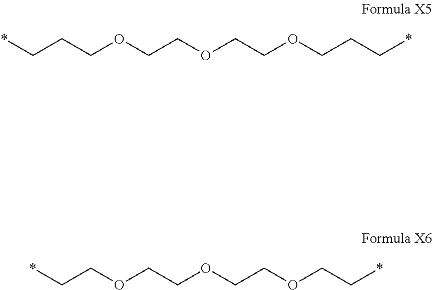 [SMILES: *CCCOCCOCCOCCC* ; *CCOCCOCCOCC*]

In one embodiment, the composition is characterized in that the hydrophobic radical according to formula I is a radical wherein R is a polyether radical according to formula X5.

In one embodiment, the composition is characterized in that the hydrophobic radical according to formula I is a radical wherein R is a polyether radical according to formula X6.

In one embodiment, the at least one hydrophobic radical according to formula I is chosen from the radicals according to formula I wherein, r=1, a=0, p=1, GpR is according to formula II wherein R is —CH₂—CH₂—, GpC is according to formula IVd wherein x=15 and Cₓ is

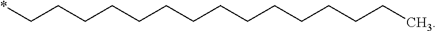 [SMILES: *CCCCCCCCCCCCCCC]

In one embodiment, at least one hydrophobic radical according to formula I is chosen from the radicals according to formula I wherein, r=1, a=0, p=1, GpR is according to formula II wherein R is —CH₂—CH₂—, GpC is according to formula IVd wherein x=16 and Cₓ is

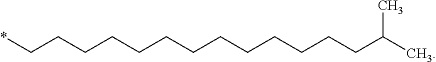 [SMILES: *CCCCCCCCCCCCCC(C)C]

In one embodiment, the at least one hydrophobic radical according to formula I is chosen from the radicals according to formula I wherein, r=1, a=0, p=1, GpR is according to formula II wherein R is

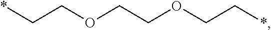

GpC is according to formula IVd wherein x=15 and Cₓ is

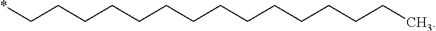 [SMILES: *CCCCCCCCCCCCCCC]

In one embodiment, the at least one hydrophobic radical according to formula I is chosen from the radicals according to formula I wherein, r=1, a=0, p=1, GpR is according to formula II wherein R is

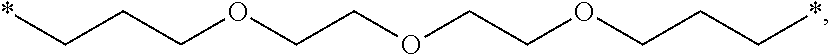

GpC is according to formula IVd wherein x=15 and Cₓ is

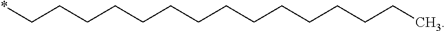 [SMILES: *CCCCCCCCCCCCCCC]

In one embodiment, the at least one hydrophobic radical according to formula I is chosen from the radicals according to formula I wherein, r=1, a=0, p=1, GpR is according to formula II wherein R is, GpC is according to formula IVd wherein x=15 and Cx is.

In one embodiment, the at least one hydrophobic radical according to formula I is chosen from the radicals according to formula I wherein, r=1, a=0, p=1, GpR is according to formula II wherein R is

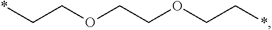

GpC is according to formula IVd wherein x=17 and Cₓ is

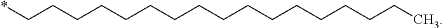 [SMILES: *CCCCCCCCCCCCCCCCC]

In one embodiment, the at least one hydrophobic radical according to formula I is chosen from the radicals according to formula I wherein, r=1, a=0, p=1, GpR is according to formula II wherein R is

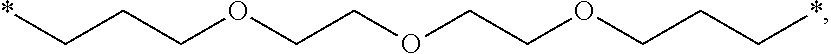

GpC is according to formula IVd wherein x=19 and Cₓ is

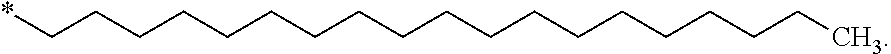 [SMILES: *CCCCCCCCCCCCCCCCCCC]

In one embodiment, the at least one hydrophobic radical according to formula I is chosen from the radicals according to formula I wherein, r=1, a=0, p=1, GpR is according to formula II wherein R is —CH₂—CH₂—, GpC is according to formula IVa wherein b=1, B is

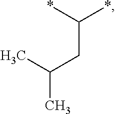

x=15 and Cₓ is

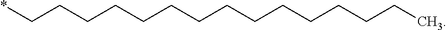 [SMILES: *CCCCCCCCCCCCCCC]

In one embodiment, the at least one hydrophobic radical according to formula I is chosen from the radicals according to formula I wherein, r=1, a=0, p=1, GpR is according to formula II wherein R is —CH₂—CH₂—, GpC is according to formula IVa wherein b=1, B is

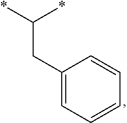 [SMILES: *C(*)Cc1ccccc1]

x=11 and Cₓ is

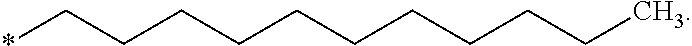 [SMILES: *CCCCCCCCCCC]

In one embodiment, the at least one hydrophobic radical according to formula I is chosen from the radicals according to formula I wherein, r=1, a=0, p=1, GpR is according to formula II wherein R is

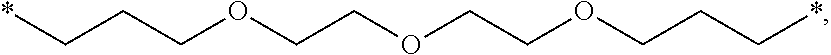

GpC is according to formula IVf wherein x=19 and Cₓ is

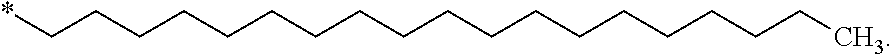 [SMILES: *CCCCCCCCCCCCCCCCCCC]

In one embodiment, at least one hydrophobic radical according to formula I is chosen from the radicals according to formula I wherein, r=1, a=0, p=1, GpR is according to formula II wherein R is —CH₂—CH₂—, GpC is according to formula IVd wherein x=13 and Cₓ is

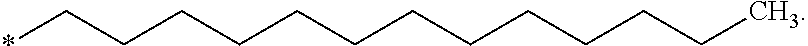 [SMILES: *CCCCCCCCCCCCC]

In one embodiment, the at least one hydrophobic radical according to formula I is chosen from the radicals according to formula I wherein, r=1, a=1, p=2, GpR corresponds to formula II wherein R is —CH₂—CH₂—, GpA corresponds to formula IIIb, GpC is according to formula IVd wherein x=9 and Cₓ is

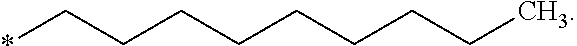 [SMILES: *CCCCCCCCC]

In one embodiment, at least one hydrophobic radical according to formula I is chosen from the radicals according to formula I wherein, r=1, a=1, p=2, GpR corresponds to formula II wherein R is —CH₂CH₂—, GpA corresponds to formula IIIb, GpC corresponds to formula IVd wherein x=11 and Cₓ is

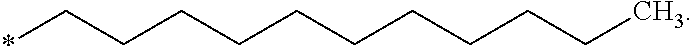 [SMILES: *CCCCCCCCCCC]

In one embodiment, at least one hydrophobic radical according to formula I is chosen from the radicals according to formula I wherein, r=1, a=1, p=2, GpR corresponds to formula II wherein R is —CH₂CH₂—, GpA corresponds to formula IIIb, GpC corresponds to formula IVd wherein x=13 and Cₓ is

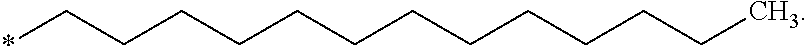 [SMILES: *CCCCCCCCCCCCC]

In one embodiment, the at least one hydrophobic radical according to formula I is chosen from radicals according to formula I wherein, r=1, a=1, p=2, GpR corresponds to formula II wherein R is

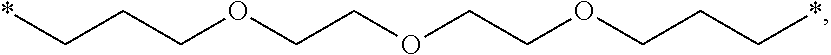

GpA corresponds to formula IIIb, GpC is according to formula IVd wherein x=13 and Cₓ is

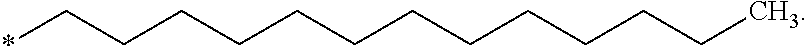 [SMILES: *CCCCCCCCCCCCC]

In one embodiment, at least one hydrophobic radical according to formula I is chosen from the radicals according to formula I wherein, r=1, a=1, p=2, GpR corresponds to formula II wherein R is —CH₂CH₂—, GpA corresponds to formula IIIb, GpC corresponds to formula IVd wherein x=15 and Cₓ is

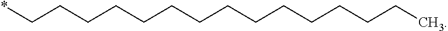 [SMILES: *CCCCCCCCCCCCCCC]

In one embodiment, the at least one hydrophobic radical according to formula I is chosen from the radicals according to formula I wherein, r=1, a=1, p=2, GpR corresponds to formula II wherein R is —CH₂—CH₂—, GpA corresponds to formula IIIb, GpC is according to formula IVd wherein x=13 and Cₓ is

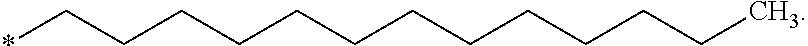 [SMILES: *CCCCCCCCCCCCC]

In one embodiment the at least one hydrophobic radical according to formula I is chosen from the radicals according to formula I wherein, r=1, a=1, p=2, GpR corresponds to formula II wherein R is —(CH₂)₆—, GpA corresponds to formula IIIb, GpC is according to formula IVd wherein x=15 and Cₓ is

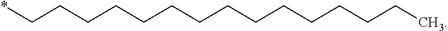 [SMILES: *CCCCCCCCCCCCCCC]

In one embodiment, the composition is characterized in that the said hydrophobic radicals are chosen from the hydrophobic radicals according to formula I wherein if p is equal to 1 and if x is less than or equal to 14 (x≤14) then r=0 or r=1.

In one embodiment, the composition is characterized in that said hydrophobic radicals are chosen from hydrophobic radicals according to formula I wherein, if p is equal to 1 and if x is comprised from 15 to 16 (15≤x≤16), then r=1.

In one embodiment, the composition is characterized in that the said hydrophobic radicals are chosen from the hydrophobic radicals according to formula I wherein if p is equal to 1 and if x is greater than 17 (17≤x) then r=1 and R is an ether or polyether radical.

In one embodiment, the composition is characterized in that the said hydrophobic radicals are chosen from the hydrophobic radicals according to formula I wherein, if p is equal to 1, then x is comprised from 17 to 25 (17≤x≤25).

In one embodiment, the composition is characterized in that the hydrophobic radical is a radical according to formula I wherein a is equal to 0 (a=0) and r is equal to 0 (r=0).

In one embodiment, the composition is characterized in that the hydrophobic radical is a radical according to formula I wherein a is equal to 1 (a=1) and the radical GpA according to formula III′ is chosen from the group consisting of radicals represented by the formulas below:

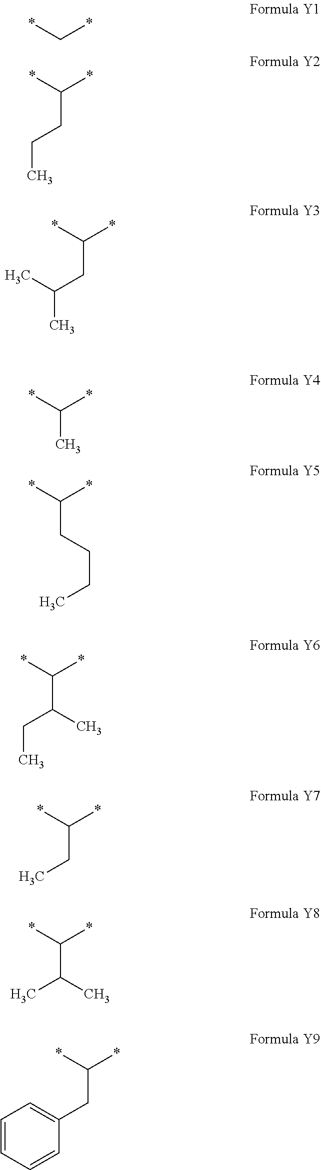

In one embodiment, the composition is characterized in that the hydrophobic radical is a radical according to formula I wherein a is equal to 1 (a=1) and the radical GpA according to formula III′ is a radical according to formula Y1.

In one embodiment, the composition is characterized in that the hydrophobic radical is a radical according to formula I wherein a is equal to 1 (a=1) and the radical GpA according to formula III′ is a radical according to formula Y2.

In one embodiment, the composition is characterized in that the hydrophobic radical is a radical according to formula I wherein a is equal to 1 (a=1) and the radical GpA according to formula III′ is a radical according to formula Y3.

In one embodiment, the composition is characterized in that the hydrophobic radical is a radical according to formula I wherein a is equal to 1 (a=1) and the radical GpA according to formula III′ is a radical according to formula Y4.

In one embodiment, the composition is characterized in that the hydrophobic radical is a radical according to formula I wherein a is equal to 1 (a=1) and the radical GpA according to formula III′ is a radical according to formula Y5.

In one embodiment, the composition is characterized in that the hydrophobic radical is a radical according to formula I wherein a is equal to 1 (a=1) and the radical GpA according to formula III′ is a radical according to formula Y6.

In one embodiment, the composition is characterized in that the hydrophobic radical is a radical according to formula I wherein a is equal to 1 (a=1) and the radical GpA according to formula III′ is a radical according to formula Y7.

In one embodiment, the composition is characterized in that the hydrophobic radical is a radical according to formula I wherein a is equal to 1 (a=1) and the radical GpA according to formula III′ is a radical according to formula Y8.

In one embodiment, the composition is characterized in that the hydrophobic radical is a radical according to formula I wherein a is equal to 1 (a=1) and the radical GpA according to formula III′ is a radical according to formula Y9.

In one embodiment, the composition is characterized in that the hydrophobic radical is a radical according to formula I wherein the radical GpC according to formula IV is chosen from the group consisting of the radicals according to formulas IVa, IVb or IVc hereinafter represented:

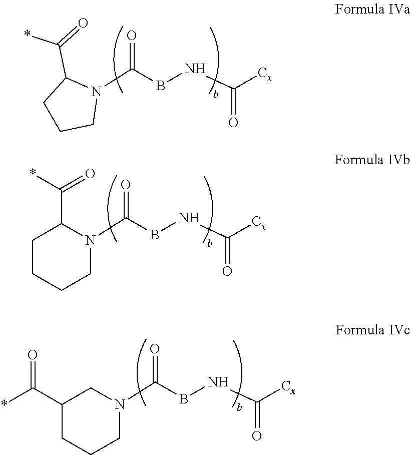

In one embodiment, the composition is characterized in that the hydrophobic radical is a radical according to formula I wherein the radical GpC is according to formula IVa.

In one embodiment, the composition is characterized in that the hydrophobic radical is a radical according to formula I wherein the radical GpC according to formula IV is chosen from the group consisting of the radicals according to formulas IVa, IVb or IVc wherein b is equal to 0, respectively corresponding to formulas IVd, IVe, and IVf hereinafter represented:

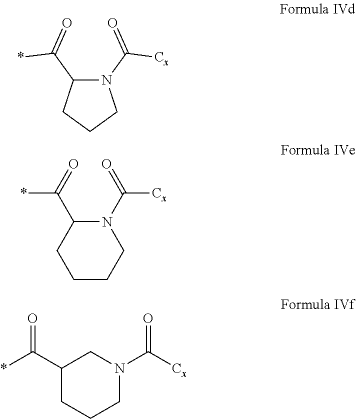

In one embodiment, the composition is characterized in that the hydrophobic radical is a radical according to formula I wherein the radical GpC corresponds to formula IV or IVa wherein b=0, and corresponds to formula IVd.

In one embodiment, the composition is characterized in that the hydrophobic radical is a radical according to formula I wherein the radical GpC according to formula IV wherein b=1 is chosen from the group consisting of radicals wherein B is a residue of amino acid chosen from the group consisting of the radicals represented by the formulas below:

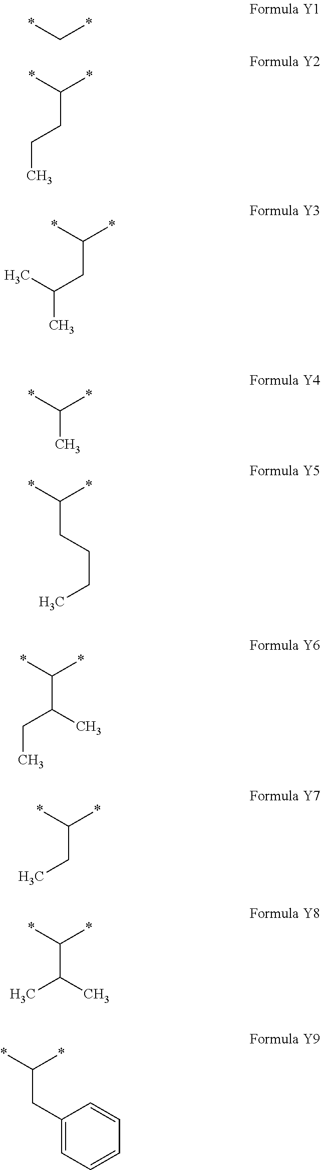

In one embodiment, the composition is characterized in that the hydrophobic radical is a radical according to formula I wherein the radical GpC according to formula IV or IVa wherein b=1 is chosen from the group consisting of radicals wherein B is an amino acid residue chosen from the group consisting of the radicals represented by the formulas below:

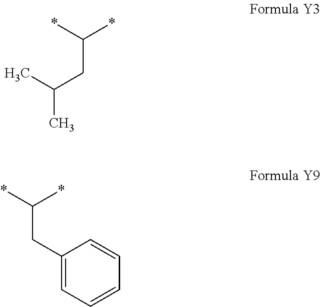

In one embodiment, the composition is characterized in that the hydrophobic radical is a radical according to formula I wherein the radical GpC according to formula IV is chosen from the group consisting of radicals wherein Cₓ is chosen from the group consisting of linear alkyl radicals.

In one embodiment, the composition is characterized in that the hydrophobic radical is a radical according to formula I wherein the radical GpC according to formula IV is chosen from the group consisting of radicals wherein Cₓ is chosen from the group consisting of branched alkyl radicals.

In one embodiment, the composition is characterized in that the hydrophobic radical is a radical according to formula I wherein the radical GpC according to formula IV is chosen from the group consisting of radicals wherein Cₓ is chosen from the group consisting of alkyl radicals comprising from 11 to 14 carbon atoms.

In one embodiment, the composition is characterized in that the hydrophobic radical is a radical according to formula I wherein the radical GpC according to formula IV is chosen from the group consisting of radicals wherein Cₓ is chosen from the group consisting of the radicals represented by the formulas below:

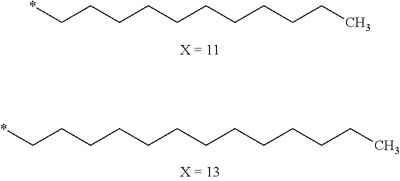 [SMILES: *CCCCCCCCCCC ; *CCCCCCCCCCCCC]

In one embodiment, the composition is characterized in that the hydrophobic radical is a radical according to formula I wherein the radical GpC according to formula IV is chosen from the group consisting of radicals wherein Cₓ is chosen from the group consisting of alkyl radicals comprising from 15 to 16 carbon atoms.

In one embodiment, the composition is characterized in that the hydrophobic radical is a radical according to formula I wherein the radical GpC according to formula IV is chosen from the group consisting of radicals wherein Cₓ is chosen from the group consisting of radicals represented by the formulas below:

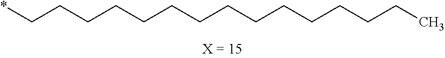 [SMILES: *CCCCCCCCCCCCCCC]

In one embodiment, the composition is characterized in that the hydrophobic radical is a radical according to formula I wherein the radical GpC according to formula IV is chosen from the group consisting of radicals wherein Cₓ is chosen from the group consisting of radicals represented by the formulas below:

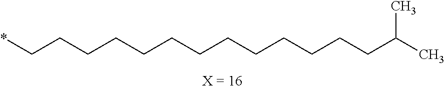 [SMILES: *CCCCCCCCCCCCCC(C)C]

In one embodiment, the composition is characterized in that the hydrophobic radical is a radical according to formula I wherein the radical GpC according to formula IV is chosen from the group consisting of radicals wherein Cₓ is chosen from the group consisting of alkyl radicals comprising from 17 to 25 carbon atoms.

In one embodiment, the composition is characterized in that the hydrophobic radical is a radical according to formula I wherein the radical GpC according to formula IV is chosen from the group consisting of radicals wherein Cₓ is chosen from the group consisting of alkyl radicals comprising from 17 to 18 carbon atoms.

In one embodiment, the composition is characterized in that the hydrophobic radical is a radical according to formula I wherein the radical GpC according to formula IV is chosen from the group consisting of radicals wherein Cₓ is chosen from the group consisting of alkyl radicals represented by the formulas below:

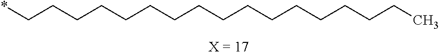 [SMILES: *CCCCCCCCCCCCCCCCC]

In one embodiment, the composition is characterized in that the hydrophobic radical is a radical according to formula I wherein the radical GpC according to formula IV is chosen from the group consisting of radicals wherein Cₓ is chosen from the group consisting of alkyl radicals comprising from 18 to 25 carbon atoms.

In one embodiment, the composition is characterized in that the hydrophobic radical is a radical according to formula I wherein the radical GpC according to formula IV is chosen from the group consisting of radicals wherein Cx is chosen from the group consisting of alkyl radicals represented by the formulas below:

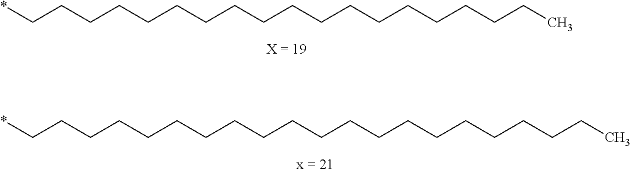 [SMILES: *CCCCCCCCCCCCCCCCCCC ; *CCCCCCCCCCCCCCCCCCCCC]

In one embodiment, the composition is characterized in that the hydrophobic radical is a radical according to formula I wherein r=1, a=1, p=1, GpR is according to formula II, GpA is according to formula III′ wherein A corresponds to formula Y9, GpC corresponds to formula IVd.

In one embodiment, the composition is characterized in that the hydrophobic radical is a radical according to formula I wherein r=1, a=1, p=1, GpR is according to formula II wherein R is a divalent linear alkyl, GpA corresponds to formula III′ wherein A is according to formula Y9, GpC corresponds to formula IVd.

In one embodiment, the composition is characterized in that the hydrophobic radical is a radical according to formula I wherein r=1, a=1, p=1, GpR corresponds to formula II wherein R is —CH2-CH2-, GpA is according to formula III′ wherein A is according to formula Y9, GpC is according to formula IVd

In one embodiment, the composition is characterized in that the hydrophobic radical is a radical according to formula I wherein r=1, a=1, p=1, GpR is according to formula II wherein R is —CH2-CH2-, GpA is according to formula III′ wherein A is according to formula Y9, GpC is according to formula IVd wherein x=13 and Cx is

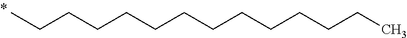

In one embodiment, when r=2, then the GpR group bound to PLG is chosen from GpR according to formula II.

In one embodiment, when r=2 then the GpR group bound to PLG is chosen from GpR according to formula II and the second GpR is chosen from GpR according to formula II″.

In one embodiment, when r=2 then the GpR group bound to PLG is chosen from GpR according to formula II″.

In one embodiment, when r=2 then the GpR group bound to PLG is chosen from the GpR according to formula II″ and the second GpR is chosen from the GpR according to formula II.

In one embodiment, GpR is a radical according to formula II:

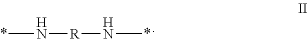

In one embodiment, at least one hydrophobic radical -Hy is chosen from the radicals according to formula I wherein r=2 according to formula Xc′, as defined below:

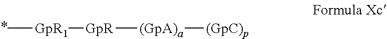

wherein GpR₁ is a radical according to formula II.

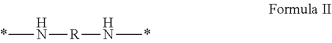

wherein GpR, GpA, GpC, R, a, and p have the definitions given above.

In one embodiment, at least one hydrophobic radical -Hy is chosen from the radicals according to formula I wherein r=2 according to formula Xc′, as defined below:

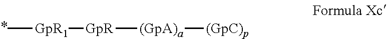

wherein GpR₁ is a radical according to formula II″.

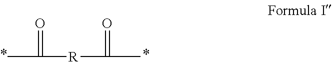

wherein GpR, GpA, GpC, R, a, and p have the definitions given above.

In one embodiment, the composition is characterized in that the said hydrophobic radicals are chosen from the hydrophobic radicals according to formula I wherein p=1, represented by formula V below:

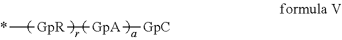

GpR, GpA, GpC, r and a have the definitions given above.

In one embodiment, the composition is characterized in that the hydrophobic radical is a radical according to formula V wherein: r is equal to 1 (r=1) and a is equal to 0 (a=0).

In one embodiment, the composition is characterized in that the hydrophobic radical is a radical according to formula V wherein: r is equal to 1 (r=1) and a is equal to 1 (a=1).

In one embodiment, the composition is characterized in that the hydrophobic radical according to formula V is a radical wherein R is a divalent linear alkyl radical comprising from 2 to 12 carbon atoms.

In one embodiment, the composition is characterized in that the hydrophobic radical according to formula V is a radical wherein R is a divalent alkyl radical comprising from 2 to 6 carbon atoms.

In one embodiment, the composition is characterized in that the hydrophobic radical according to formula V is a radical wherein R is a divalent linear alkyl radical comprising from 2 to 6 carbon atoms.

In one embodiment, the composition is characterized in that the hydrophobic radical according to formula V is a radical wherein R is a divalent alkyl radical comprising from 2 to 4 carbon atoms.

In one embodiment, the composition is characterized in that the hydrophobic radical according to formula V is a radical wherein R is a divalent linear alkyl radical comprising from 2 to 4 carbon atoms.

In one embodiment, the composition is characterized in that the hydrophobic radical according to formula V is a radical wherein R is a divalent alkyl radical comprising 2 carbon atoms.

In one embodiment, the composition is characterized in that the hydrophobic radical according to formula V is a radical wherein R is a divalent linear alkyl radical comprising from 1 to 11 carbon atoms.

In one embodiment, the composition is characterized in that the hydrophobic radical according to formula V is a radical wherein R is a divalent alkyl radical comprising from 1 to 6 carbon atoms.

In one embodiment, the composition is characterized in that the hydrophobic radical according to formula V is a radical wherein R is a divalent alkyl radical comprising from 1 to 10 carbon atoms.

In one embodiment, the composition is characterized in that the hydrophobic radical according to formula V is a radical wherein R is a divalent alkyl radical comprising from 2 to 5 carbon atoms and bearing one or more amide functions (—CONH₂).

In one embodiment, the composition is characterized in that the hydrophobic radical according to formula V is a radical wherein R is a divalent linear alkyl radical comprising from 2 to 5 carbon atoms and bearing one or more amide functions (—CONH₂).

In one embodiment, the composition is characterized in that the hydrophobic radical according to formula V is a radical wherein R is a radical chosen from the group consisting of the radicals represented by the formulas below:

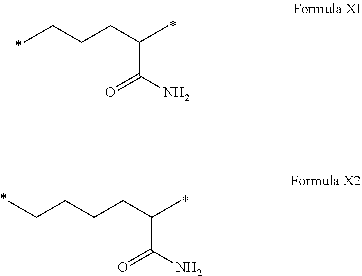

In one embodiment, the composition is characterized in that the hydrophobic radical according to formula V is a radical wherein R is a radical according to formula X1.

In one embodiment, the composition is characterized in that the hydrophobic radical according to formula V is a radical wherein R is a radical according to formula X2.

In one embodiment, the composition is characterized in that the hydrophobic radical according to formula V is a radical wherein R is bound to the co-polyamino acid via an amide function borne by the carbon in the delta or epsilon position (or in position 4 or 5) with respect to the amide function (—CONH₂).

In one embodiment, the composition is characterized in that the hydrophobic radical according to formula V is a radical wherein R is an unsubstituted linear ether or polyether radical comprising from 4 to 14 carbon atoms and from 1 to 5 oxygen atoms.

In one embodiment, the composition is characterized in that the hydrophobic radical according to formula V is a radical wherein R is an ether radical.

In one embodiment, the composition is characterized in that the hydrophobic radical according to formula V is a radical wherein R is a divalent alkyl radical comprising from 4 to 6 carbon atoms.

In one embodiment, the composition is characterized in that the hydrophobic radical according to formula V is a radical wherein R is a divalent alkyl radical comprising 6 carbon atoms.

In one embodiment, the composition is characterized in that the hydrophobic radical according to formula V is a radical wherein R is an ether radical represented by the formula

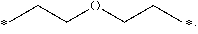

In one embodiment, the composition is characterized in that the hydrophobic radical according to formula V is a radical wherein R is a polyether radical.

In one embodiment, the composition is characterized in that the hydrophobic radical according to formula V is a radical wherein R is a linear polyether radical comprising from 6 to 10 carbon atoms and from 2 to 3 oxygen atoms.

In one embodiment, the composition is characterized in that the hydrophobic radical according to formula V is a radical wherein R is a polyether radical chosen from the group consisting of the radicals represented by the formulas below:

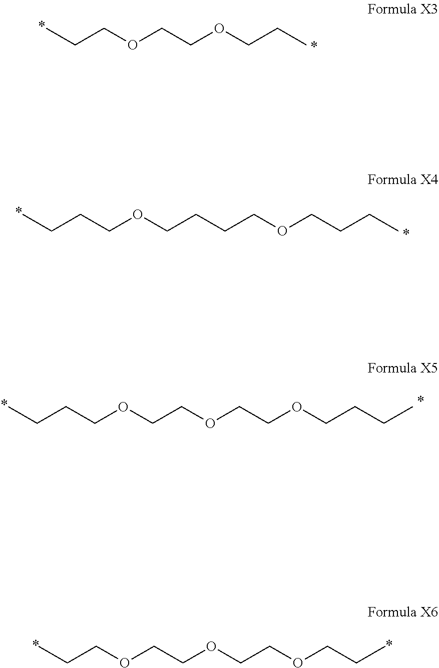

In one embodiment, the composition is characterized in that the hydrophobic radical according to formula V is a radical wherein R is a radical according to formula X3.

In one embodiment, the composition is characterized in that the hydrophobic radical according to formula V is a radical wherein R is a radical according to formula X4.

In one embodiment, the composition is characterized in that the hydrophobic radical according to formula V is a radical wherein R is a polyether radical chosen from the group consisting of the radicals represented by formulas X5 and X6 below:

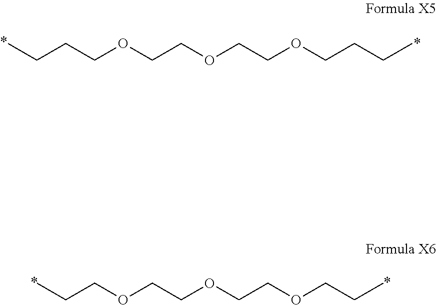

In one embodiment, the composition is characterized in that the hydrophobic radical according to formula V is a radical wherein R is a polyether radical according to formula X5.

In one embodiment, the composition is characterized in that the hydrophobic radical according to formula V is a radical wherein R is a polyether radical according to formula X6.

In one embodiment, the composition is characterized in that the hydrophobic radical is a radical according to formula V wherein GpR is a radical according to formula II.

In one embodiment, the composition is characterized in that the hydrophobic radical is a radical according to formula V wherein GpR is a radical according to formula II wherein R is a divalent alkyl radical comprising from 2 to 12 carbon atoms.

In one embodiment, the composition is characterized in that the hydrophobic radical is a radical according to formula V wherein GpR is a radical according to formula II wherein R is a divalent alkyl radical comprising from 2 to 6 carbon atoms.

In one embodiment, the composition is characterized in that the hydrophobic radical is a radical according to formula V wherein GpR is a radical according to formula II wherein R is a divalent linear alkyl radical comprising from 2 to 6 carbon atoms.

In one embodiment, the composition is characterized in that the hydrophobic radical is a radical according to formula V wherein GpR is a radical according to formula II wherein R is a divalent alkyl radical comprising from 2 to 4 carbon atoms.

In one embodiment, the composition is characterized in that the hydrophobic radical is a radical according to formula V wherein GpR is a radical according to formula II wherein R is a divalent linear alkyl radical comprising from 2 to 4 carbon atoms.

In one embodiment, the composition is characterized in that the hydrophobic radical is a radical according to formula V wherein GpR is a radical according to formula II wherein R is a divalent alkyl radical comprising 2 carbon atoms.

In one embodiment, the composition is characterized in that the hydrophobic radical is a radical according to formula V wherein GpR is a radical according to formula II′.

In one embodiment, the composition is characterized in that the hydrophobic radical is a radical according to formula V wherein GpR is a radical according to formula II′ wherein R is a divalent linear alkyl radical comprising from 1 to 11 carbon atoms.

In one embodiment, the composition is characterized in that the hydrophobic radical is a radical according to formula V wherein GpR is a radical according to formula II′ wherein R is a divalent alkyl radical comprising from 1 to 6 carbon atoms.

In one embodiment, the composition is characterized in that the hydrophobic radical is a radical according to formula V wherein GpR is a radical according to formula II″ wherein R is a divalent alkyl radical comprising from 1 to 10 carbon atoms.

In one embodiment, the composition is characterized in that the hydrophobic radical is a radical according to formula V wherein GpR is a radical according to formula II, II′ or II″, wherein R is a divalent linear alkyl radical, comprising of 2 to 5 carbon atoms and bearing one or more amide functions (—CONH₂).

In one embodiment, the composition is characterized in that the hydrophobic radical is a radical according to formula V wherein GpR is a radical according to formula II, II ‘or II′’, wherein R is a divalent linear alkyl radical, comprising from 2 to 5 carbon atoms and bearing one or more amide functions (—CONH₂).

In one embodiment, the composition is characterized in that the hydrophobic radical is a radical according to formula V wherein GpR is a radical according to formula II, II′ or II″ wherein R is a radical chosen from the group consisting of the radicals represented by the formulas below:

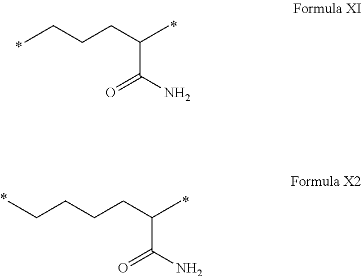

In one embodiment, the composition is characterized in that the hydrophobic radical according to formula V wherein GpR is a radical according to formula II, II ‘or II′’, wherein R is a radical according to formula X1.

In one embodiment, the composition is characterized in that the hydrophobic radical according to formula V wherein GpR is a radical according to formula II, II ‘or II′’, wherein R is a radical according to formula X2.

In one embodiment, the composition is characterized in that the R radical of GpR₁ is bound to the co-polyamino acid via an amide function borne by the carbon in the delta or epsilon position (or in position 4 or 5) relative to the amide function (—CONH₂).

In one embodiment, the composition is characterized in that the hydrophobic radical is a radical according to formula V wherein GpR is a radical according to formula II, II′ or II″, wherein R is a linear ether or non-substituted polyether radical comprising from 4 to 14 carbon atoms and 1 to 5 oxygen atoms.

In one embodiment, the composition is characterized in that the hydrophobic radical is a radical according to formula V wherein GpR is a radical according to formula II, II′ or II″, wherein R is an ether radical.

In one embodiment, the composition is characterized in that the hydrophobic radical is a radical according to formula V wherein GpR is a radical according to formula II, II′ or II″, wherein R is an ether radical comprising from 4 to 6 carbon atoms.

In one embodiment, the composition according to the invention is characterized in that the hydrophobic radical is a radical according to formula V wherein GpR is a radical according to formula II wherein R is a divalent alkyl radical comprising 6 carbon atoms.

In one embodiment, the composition is characterized in that the hydrophobic radical is a radical according to formula V wherein GpR is a radical according to formula II, II′ or II″ wherein R is an ether radical represented by formula

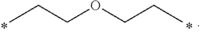

In one embodiment, the composition is characterized in that the hydrophobic radical is a radical according to formula V wherein GpR is a radical according to formula II, II′ or II″, wherein R is a polyether radical.

In one embodiment, the composition is characterized in that the hydrophobic radical is a radical according to formula V wherein GpR is a radical according to formula II, II′ or II″, wherein R is a linear polyether radical comprising from 6 to 10 carbon atoms and 2 to 3 oxygen atoms.

In one embodiment, the composition is characterized in that the hydrophobic radical is a radical according to formula V wherein GpR is a radical according to formula II, II′ or II″ wherein R is a polyether radical chosen from the group consisting of the radicals represented by the formulas below:

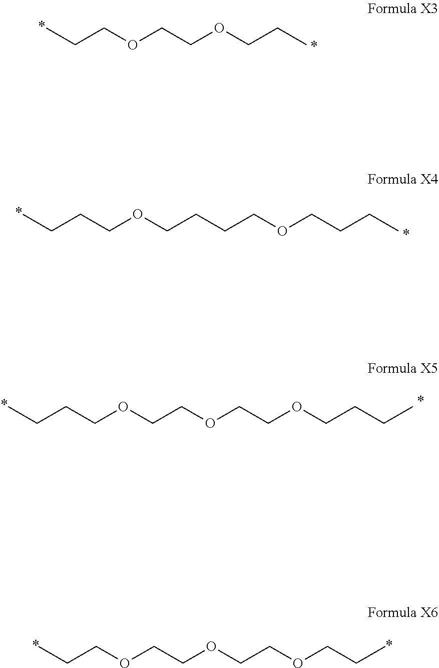

In one embodiment, the composition is characterized in that the hydrophobic radical according to formula V wherein GpR is a radical according to formula II, II ‘or II′’, wherein R is a radical according to formula X3.

In one embodiment, the composition is characterized in that the hydrophobic radical according to formula V wherein GpR is a radical according to formula II, II ‘or II′’, wherein R is a radical according to formula X4.

In one embodiment, the composition is characterized in that the hydrophobic radical according to formula V wherein GpR is a radical according to formula II, II ‘or II′’, wherein R is a radical according to formula X5.

In one embodiment, the composition is characterized in that the hydrophobic radical according to formula V wherein GpR is a radical according to formula II, II ‘or II′’, wherein R is a radical according to formula X6.

In an embodiment, the composition is characterized in that the hydrophobic radical according to formula V wherein GpR is a radical according to formula II wherein R is a polyether radical chosen from the group consisting of the radicals represented by formulas X5 and X6 below:

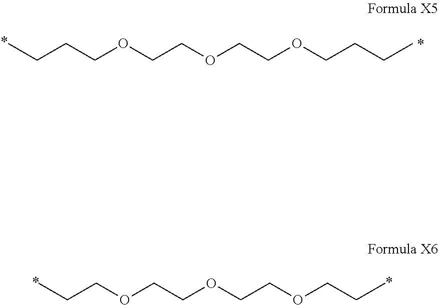

In one embodiment, the composition is characterized in that the hydrophobic radical according to formula V wherein GpR is a radical according to formula II wherein R is a polyether radical according to formula X5.

In one embodiment, the composition is characterized in that the hydrophobic radical according to formula V wherein GpR is a radical according to formula II wherein R is a polyether radical according to formula X6.

In one embodiment, the composition is characterized in that the hydrophobic radical is a radical according to formula V wherein a is equal to 0 (a=0) and r is equal to 0 (r=0).

In one embodiment, the composition is characterized in that the hydrophobic radical is a radical according to formula V wherein a is equal to 1 (a=1) and the radical GpA according to formula III′ is chosen from the group consisting of the radicals represented by the formulas below:

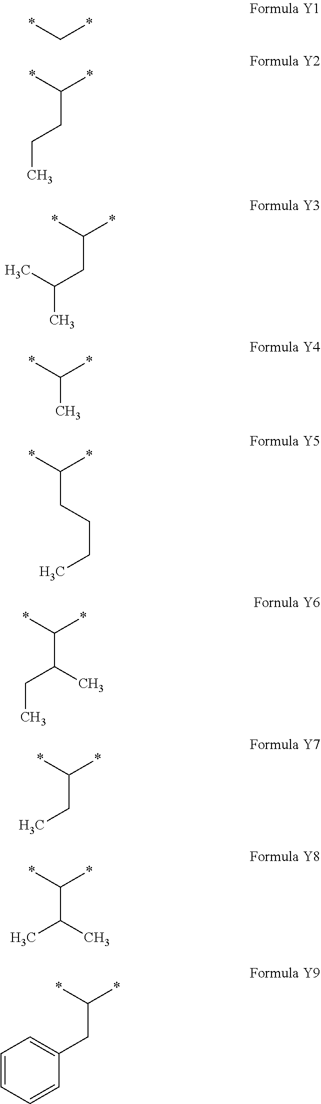

In one embodiment, the composition is characterized in that the hydrophobic radical is a radical according to formula V wherein a is equal to 1 (a=1) and the radical GpA according to formula III′ is a radical according to formula Y1.

In one embodiment, the composition is characterized in that the hydrophobic radical is a radical according to formula V wherein a is equal to 1 (a=1) and the radical GpA according to formula III′ is a radical according to formula Y2.

In one embodiment, the composition is characterized in that the hydrophobic radical is a radical according to formula V wherein a is equal to 1 (a=1) and the radical GpA according to formula III′ is a radical according to formula Y3.

In one embodiment, the composition is characterized in that the hydrophobic radical is a radical according to formula V wherein a is equal to 1 (a=1) and the radical GpA according to formula III′ is a radical according to formula Y4.

In one embodiment, the composition is characterized in that the hydrophobic radical is a radical according to formula V wherein a is equal to 1 (a=1) and the radical GpA according to formula III′ is a radical according to formula Y5.

In one embodiment, the composition is characterized in that the hydrophobic radical is a radical according to formula V wherein a is equal to 1 (a=1) and the radical GpA according to formula III′ is a radical according to formula Y6.

In one embodiment, the composition is characterized in that the hydrophobic radical is a radical according to formula V wherein a is equal to 1 (a=1) and the radical GpA according to formula III′ is a radical according to formula Y7.

In one embodiment, the composition is characterized in that the hydrophobic radical is a radical according to formula V wherein a is equal to 1 (a=1) and the radical GpA according to formula III′ is a radical according to formula Y8.

In one embodiment, the composition is characterized in that the hydrophobic radical is a radical according to formula V wherein a is equal to 1 (a=1) and the radical GpA according to formula III′ is a radical according to formula Y9.

In one embodiment, the composition is characterized in that the hydrophobic radical is a radical according to formula V wherein the radical GpC according to formula IV is chosen from the group consisting of the radicals according to formulas IVa, IVb or IVc hereinafter represented:

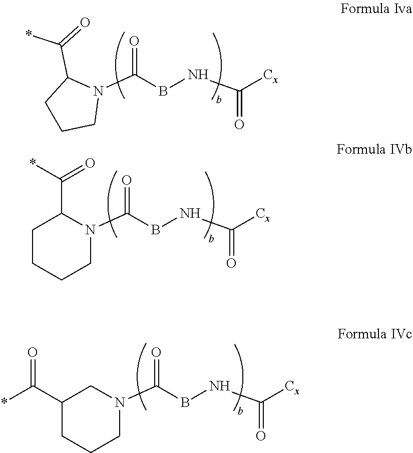

In one embodiment, the composition is characterized in that the hydrophobic radical is a radical according to formula V wherein the radical GpC is according to formula IVa.

In one embodiment, the composition is characterized in that the hydrophobic radical is a radical according to formula I wherein the radical GpC according to formula IV is chosen from the group consisting of the radicals according to formulas IVa, IVb or IVc wherein b is equal to 0, respectively corresponding to formulas IVd, IVe, and IVf hereinafter represented:

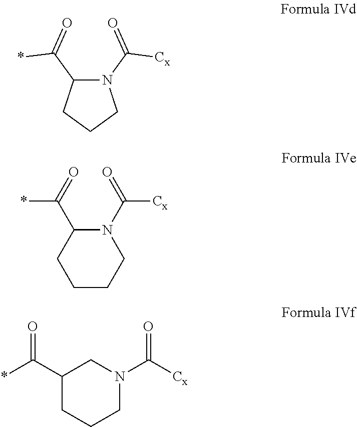

In one embodiment, the composition is characterized in that the hydrophobic radical is a radical according to formula V wherein the radical GpC corresponds to formula IV or IVa wherein b=0, and corresponds to formula IVd.

In one embodiment, the composition is characterized in that the hydrophobic radical is a radical according to formula V wherein the radical GpC according to formula IV wherein b=1 is chosen from the group consisting of radicals wherein B is a residue of amino acid chosen from the group consisting of the radicals represented by the formulas below:

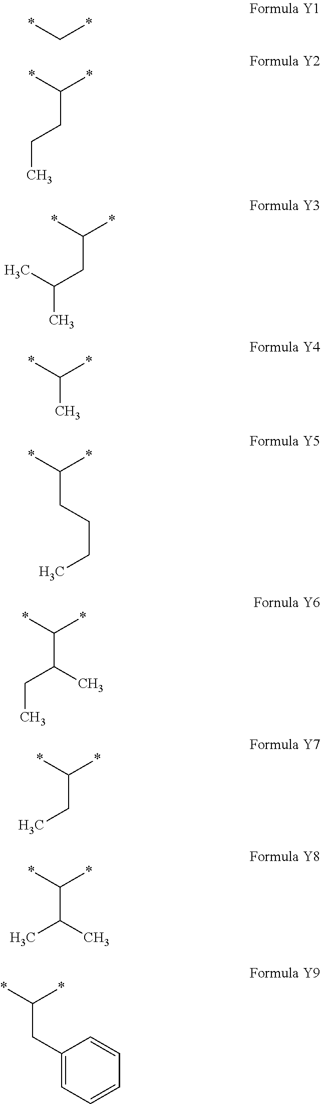

In one embodiment, the composition is characterized in that the hydrophobic radical is a radical according to formula V wherein the radical GpC according to formula IV or IVa wherein b=1 is chosen from the group consisting of radicals wherein B is an amino acid residue chosen from the group consisting of the radicals represented by the formulas below:

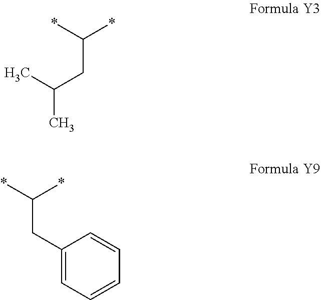

In one embodiment, the composition is characterized in that the hydrophobic radical is a radical according to formula V wherein the radical GpC according to formula IV is chosen from the group consisting of radicals wherein Cₓ is chosen from the group consisting of linear alkyl radicals.

In one embodiment, the composition is characterized in that the hydrophobic radical is a radical according to formula V wherein the radical GpC according to formula IV is chosen from the group consisting of radicals wherein Cₓ is chosen from the group consisting of branched alkyl radicals.

In one embodiment, the composition is characterized in that the hydrophobic radical is a radical according to formula V wherein the radical GpC according to formula IV is chosen from the group consisting of radicals wherein Cₓ is chosen from the group consisting of alkyl radicals comprising from 11 to 14 carbon atoms.

In one embodiment, the composition is characterized in that the hydrophobic radical is a radical according to formula V wherein the radical GpC according to formula IV is chosen from the group consisting of radicals wherein Cₓ is chosen from the group consisting of the radicals represented by the formulas below:

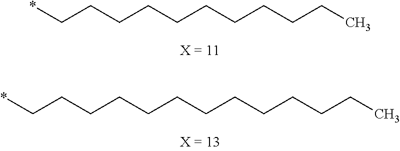 [SMILES: *CCCCCCCCCCC ; *CCCCCCCCCCCCC]

In one embodiment, the composition is characterized in that the hydrophobic radical is a radical according to formula V wherein the radical GpC according to formula IV is chosen from the group consisting of radicals wherein Cₓ is chosen from the group consisting of alkyl radicals comprising from 15 to 16 carbon atoms.

In one embodiment, the composition is characterized in that the hydrophobic radical is a radical according to formula V wherein the radical GpC according to formula IV is chosen from the group consisting of radicals wherein Cₓ is chosen from the group consisting of the radicals represented by the formulas below:

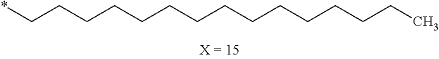 [SMILES: *CCCCCCCCCCCCCCC]

In one embodiment, the composition is characterized in that the hydrophobic radical is a radical according to formula V wherein the radical GpC according to formula IV is chosen from the group consisting of radicals wherein Cx is chosen from the group consisting of the radicals represented by the formulas below:

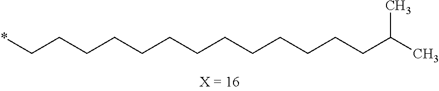 [SMILES: *CCCCCCCCCCCCCC(C)C]

In one embodiment, the composition is characterized in that the hydrophobic radical is a radical according to formula V wherein the radical GpC according to formula IV is chosen from the group consisting of radicals wherein Cx is chosen from the group consisting of alkyl radicals comprising from 17 to 25 carbon atoms.

In one embodiment, the composition is characterized in that the hydrophobic radical is a radical according to formula V wherein the radical GpC according to formula IV is chosen from the group consisting of radicals wherein Cₓ is chosen from the group consisting of alkyl radicals comprising from 17 to 18 carbon atoms.

In one embodiment, the composition is characterized in that the hydrophobic radical is a radical according to formula V wherein the radical GpC according to formula IV is chosen from the group consisting of radicals wherein Cₓ is chosen from the group consisting of linear alkyl radicals represented by the formulas below:

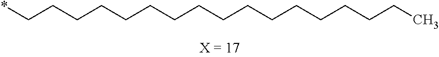 [SMILES: *CCCCCCCCCCCCCCCCC]

In one embodiment, the composition is characterized in that the hydrophobic radical is a radical according to formula V wherein the radical GpC according to formula IV is chosen from the group consisting of radicals wherein Cx is chosen from the group consisting of alkyl radicals comprising from 18 to 25 carbon atoms.

In one embodiment, the composition is characterized in that the hydrophobic radical is a radical according to formula V wherein the radical GpC according to formula IV is chosen from the group consisting of radicals wherein Cx is chosen from the group consisting of linear alkyl radicals represented by the formulas below:

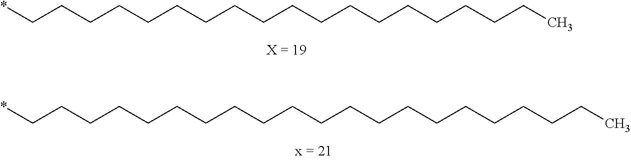 [SMILES: *CCCCCCCCCCCCCCCCCCC ; *CCCCCCCCCCCCCCCCCCCCC]

In one embodiment, the composition is characterized in that the hydrophobic radical is a radical according to formula V wherein r=1, a=1, GpR corresponds to formula II, GpA corresponds to formula III′ wherein A corresponds to formula Y9, GpC corresponds to formula IVd.

In one embodiment, the composition is characterized in that the hydrophobic radical is a radical according to formula V wherein r=1, a=1, GpR corresponds to formula II wherein R is a divalent linear alkyl, GpA corresponds to formula III′ wherein A corresponds to formula Y9, GpC corresponds to formula IVd.

In one embodiment, the composition is characterized in that the hydrophobic radical is a radical according to formula V wherein r=1, a=1, GpR corresponds to formula II wherein R is —CH2-CH2-, GpA corresponds to formula III′ wherein A corresponds to formula Y9, GpC corresponds to formula IVd

In one embodiment, the composition is characterized in that the hydrophobic radical is a radical according to formula V wherein r=1, a=1, GpR corresponds to formula II wherein R is —CH2-CH2-, GpA corresponds to formula III′ wherein A corresponds to formula Y9, GpC corresponds to formula IVd wherein x=13 and Cx is

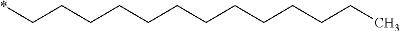 [SMILES: *CCCCCCCCCCCCC]

In one embodiment, the composition is characterized in that the said hydrophobic radicals are chosen from the hydrophobic radicals according to formula I wherein a=1 wherein p=2, represented by formula VI below:

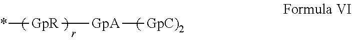

wherein

GpR, GpA, GpC, r and a have the definitions given above.

In one embodiment, the composition is characterized in that the hydrophobic radical according to formula V is a radical wherein R is a divalent linear alkyl radical comprising from 2 to 12 carbon atoms.

In one embodiment, the composition is characterized in that the hydrophobic radical according to formula VI is a radical wherein R is a divalent alkyl radical comprising from 2 to 6 carbon atoms.

In one embodiment, the composition is characterized in that the hydrophobic radical according to formula VI is a radical wherein R is a divalent linear alkyl radical comprising from 2 to 6 carbon atoms.

In one embodiment, the composition is characterized in that the hydrophobic radical according to formula VI is a radical wherein R is a divalent alkyl radical comprising from 2 to 4 carbon atoms.

In one embodiment, the composition is characterized in that the hydrophobic radical according to formula VI is a radical wherein R is a divalent linear alkyl radical comprising from 2 to 4 carbon atoms.

In one embodiment, the composition is characterized in that the hydrophobic radical according to formula VI is a radical wherein R is a divalent alkyl radical comprising 2 carbon atoms.

In one embodiment, the composition is characterized in that the hydrophobic radical according to formula VI is a radical wherein R is a divalent linear alkyl radical comprising from 1 to 11 carbon atoms.

In one embodiment, the composition is characterized in that the hydrophobic radical according to formula VI is a radical wherein R is a divalent alkyl radical comprising from 1 to 6 carbon atoms.

In one embodiment, the composition is characterized in that the hydrophobic radical according to formula VI is a radical wherein R is a divalent alkyl radical comprising from 1 to 10 carbon atoms.

In one embodiment, the composition is characterized in that the hydrophobic radical according to formula V is a radical wherein R is a divalent alkyl radical comprising from 2 to 5 carbon atoms and bearing one or more amide functions (—CONH₂).

In one embodiment, the composition is characterized in that the hydrophobic radical according to formula VI is a radical wherein R of GpR if r=1 or GpR1 if r=2 is a divalent linear alkyl radical comprising from 2 to 5 atoms of carbon and bearing one or more amide functions (—CONH₂).

In one embodiment, the composition is characterized in that the hydrophobic radical according to formula VI is a radical wherein R is a radical chosen from the group consisting of the radicals represented by the formulas below:

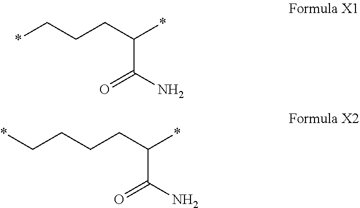

In one embodiment, the composition is characterized in that the hydrophobic radical according to formula VI is a radical wherein R is a radical according to formula X1.

In one embodiment, the composition is characterized in that the hydrophobic radical according to formula VI is a radical wherein R is a radical according to formula X2.

In one embodiment, the composition is characterized in that the hydrophobic radical according to formula I is a radical wherein R of GpR if r=1 or of GpR1 if r=2 is bound to the co-polyamino acid via an amide function borne by carbon in the delta or epsilon position (or position 4 or 5) relative to the amide function (—CONH₂).

In one embodiment, the composition is characterized in that the hydrophobic radical according to formula VI is a radical wherein R is an unsubstituted linear ether or polyether radical comprising from 4 to 14 carbon atoms and from 1 to 5 oxygen atoms.

In one embodiment, the composition is characterized in that the hydrophobic radical according to formula VI is a radical wherein R is an ether radical.

In one embodiment, the composition is characterized in that the hydrophobic radical according to formula VI is a radical wherein R is a divalent alkyl radical comprising from 4 to 6 carbon atoms.

In one embodiment, the composition is characterized in that the hydrophobic radical according to formula VI is a radical wherein R is a divalent alkyl radical comprising 6 carbon atoms.

In one embodiment, the composition is characterized in that the hydrophobic radical according to formula VI is a radical wherein R is an ether radical represented by the formula

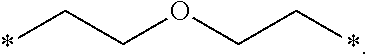

In one embodiment, the composition is characterized in that the hydrophobic radical according to formula VI is a radical wherein R is a polyether radical.

In one embodiment, the composition is characterized in that the hydrophobic radical according to formula VI is a radical wherein R is an unsubstituted linear ether or polyether radical comprising from 6 to 10 carbon atoms and from 2 to 3 oxygen atoms.

In one embodiment, the composition is characterized in that the hydrophobic radical according to formula VI is a polyether radical wherein R is a radical chosen from the group consisting of the radicals represented by the formulas below:

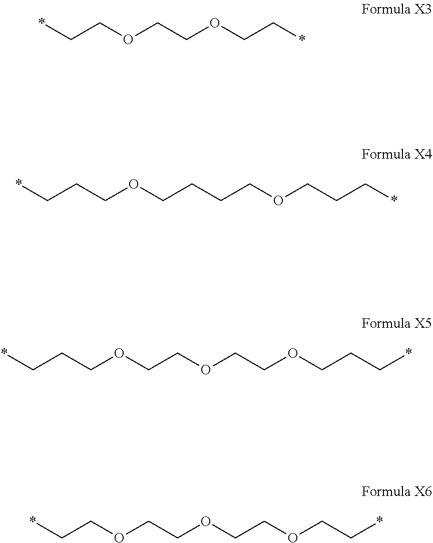 [SMILES: *CCOCCOCC* ; *CCCOCCCCOCCC* ; *CCCOCCOCCOCCC* ; *CCOCCOCCOCC*]

In one embodiment, the composition is characterized in that the hydrophobic radical according to formula VI is a radical wherein R is a radical according to formula X3.

In one embodiment, the composition is characterized in that the hydrophobic radical according to formula VI is a radical wherein R is a radical according to formula X4.

In one embodiment, the composition is characterized in that the hydrophobic radical according to formula VI is a radical wherein R is a polyether radical chosen from the group consisting of the radicals represented by formulas X5 and X6 below:

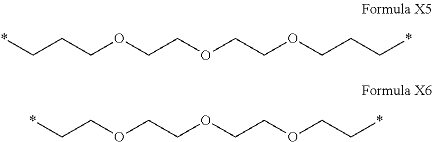 [SMILES: *CCCOCCOCCOCCC* ; *CCOCCOCCOCC*]

In one embodiment, the composition is characterized in that the hydrophobic radical according to formula VI is a radical wherein R is a polyether radical according to formula X5.

In one embodiment, the composition is characterized in that the hydrophobic radical according to formula VI is a radical wherein R is a polyether radical according to formula X6.

In one embodiment, the composition is characterized in that the hydrophobic radical is a radical according to formula VI wherein GpR is a radical according to formula II.

In one embodiment, the composition is characterized in that the hydrophobic radical is a radical according to formula VI wherein GpR is a radical according to formula II wherein R is a divalent linear alkyl radical comprising from 2 to 12 carbon atoms.

In one embodiment, the composition is characterized in that the hydrophobic radical is a radical according to formula VI wherein GpR is a radical according to formula II wherein R is a divalent alkyl radical comprising from 2 to 6 carbon atoms.

In one embodiment, the composition is characterized in that the hydrophobic radical is a radical according to formula VI wherein GpR is a radical according to formula II wherein R is a divalent linear alkyl radical comprising from 2 to 6 carbon atoms.

In one embodiment, the composition is characterized in that the hydrophobic radical is a radical according to formula VI wherein GpR is a radical according to formula II wherein R is an alkyl radical comprising from 2 to 4 carbon atoms.

In one embodiment, the composition is characterized in that the hydrophobic radical is a radical according to formula VI wherein GpR is a radical according to formula II wherein R is a divalent linear alkyl radical comprising from 2 to 4 carbon atoms.

In one embodiment, the composition is characterized in that the hydrophobic radical is a radical according to formula VI wherein GpR is a radical according to formula II wherein R is a divalent linear alkyl radical comprising 2 carbon atoms.

In one embodiment, the composition is characterized in that the hydrophobic radical is a radical according to formula VI wherein GpR is a radical according to formula II′.

In one embodiment, the composition is characterized in that the hydrophobic radical is a radical according to formula VI wherein GpR is a radical according to formula II′ wherein R is a divalent linear alkyl radical comprising from 1 to 11 carbon atoms.

In one embodiment, the composition is characterized in that the hydrophobic radical is a radical according to formula VI wherein GpR is a radical according to formula II′ wherein R is a divalent alkyl radical comprising from 1 to 6 carbon atoms.

In an embodiment, the composition is characterized in that the hydrophobic radical is a radical according to formula VI wherein GpR is a radical according to formula II′.

In one embodiment, the composition is characterized in that the hydrophobic radical is a radical according to formula VI wherein GpR is a radical according to formula II′ wherein R is a divalent linear alkyl radical comprising from 1 to 10 carbon atoms.

In one embodiment, the composition is characterized in that the hydrophobic radical is a radical according to formula VI wherein GpR is a radical according to formula II, II′ or II″, wherein R is a divalent linear alkyl radical, comprising from 2 to 5 carbon atoms and bearing one or more amide functions (—CONH2).

In one embodiment, the composition is characterized in that the hydrophobic radical is a radical according to formula VI wherein GpR is a radical according to formula II, II′ or II″, wherein R is a divalent linear alkyl radical, comprising from 2 to 5 carbon atoms and bearing one or more amide functions (—CONH2).

In one embodiment, the composition is characterized in that the hydrophobic radical is a radical according to formula VI wherein GpR is a radical according to formula II, II′ or II″ wherein R is a radical chosen from the group consisting of the radicals represented by the formulas below:

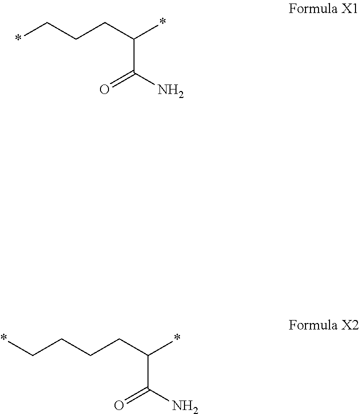 [SMILES: *CCCC(*)C(N)=O ; *CCCCC(*)C(N)=O]

In one embodiment, the composition is characterized in that the hydrophobic radical is a radical according to formula VI wherein the amine function of the radical GpR involved in the formation of the amide function which binds said radical GpR to the co-polyamino acid is borne by a carbon in delta or epsilon position (or in position 4 or 5) with respect to the amide function (—CONH2).

In one embodiment, the composition is characterized in that the hydrophobic radical is a radical according to formula VI wherein GpR is a radical according to formula II, II′ or II″, wherein R is a linear ether or non-substituted polyether radical with 4 to 14 carbon atoms and 1 to 5 oxygen atoms.

In one embodiment, the composition is characterized in that the hydrophobic radical is a radical according to formula VI wherein GpR is a radical according to formula II, II′ or II″, wherein R is an ether radical.

In one embodiment, the composition is characterized in that the ether radical R is a radical comprising from 4 to 6 carbon atoms.

In one embodiment, the composition is characterized in that the ether radical is

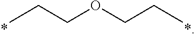

In one embodiment, the composition is characterized in that the hydrophobic radical is a radical according to formula VI wherein GpR is a radical according to formula II, II′ or II″, wherein R is a polyether radical.

In one embodiment, the composition is characterized in that the hydrophobic radical is a radical according to formula VI wherein GpR is a radical according to formula II, II′ or II″, wherein R is a linear polyether radical comprising from 6 to 10 carbon atoms and 2 to 3 oxygen atoms.

In one embodiment, the composition is characterized in that the hydrophobic radical is a radical according to formula VI wherein GpR is a radical according to formula II, II′ or II″ wherein R is a linear polyether radical chosen from the group consisting of the radicals represented by the formulas below:

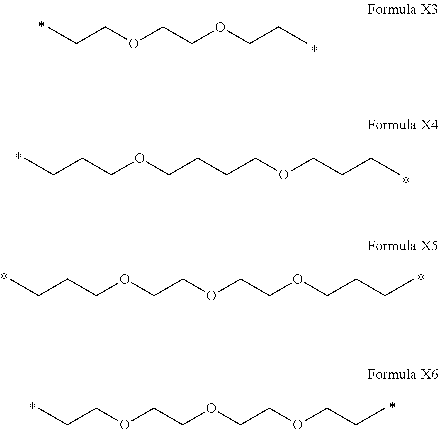

In one embodiment, the composition is characterized in that the hydrophobic radical according to formula VI wherein GpR is a radical according to formula II, II′ or II″, wherein R is a linear polyether radical according to formula X3.

In one embodiment, the composition is characterized in that the hydrophobic radical according to formula VI wherein GpR is a radical according to formula II, II′ or II″, wherein R is a linear polyether radical according to formula X4.

In one embodiment, the composition is characterized in that the hydrophobic radical according to formula VI wherein GpR is a radical according to formula II, II′ or II″, wherein R is a linear polyether radical according to formula X5.

In one embodiment, the composition is characterized in that the hydrophobic radical according to formula VI wherein GpR is a radical according to formula II, II′ or II″, wherein R is a linear polyether radical according to formula X6.

In one embodiment, the composition is characterized in that the hydrophobic radical is a radical according to formula VI wherein the radical GpA according to formula III is chosen from the group consisting of the radicals according to formulas IIIa, IIIb and IIIc represented below:

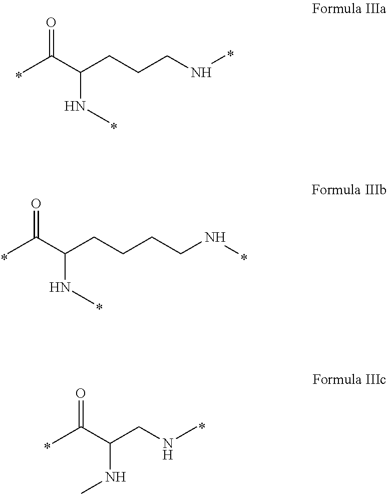

In one embodiment, the composition is characterized in that the hydrophobic radical is a radical according to formula VI wherein the radical GpA according to formula III is a radical according to formula IIIb represented hereafter:

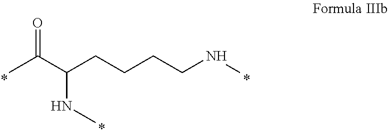

In one embodiment, the composition according to the invention is characterized in that the hydrophobic radical is a radical according to formula VI wherein the radical GpA according to formula III is a radical according to formula IIIc.

In one embodiment, the composition is characterized in that the hydrophobic radical is a radical according to formula VI wherein the radical GpC according to formula IV is chosen from the group consisting of the radicals according to formulas IVa, IVb and IVc represented hereinafter:

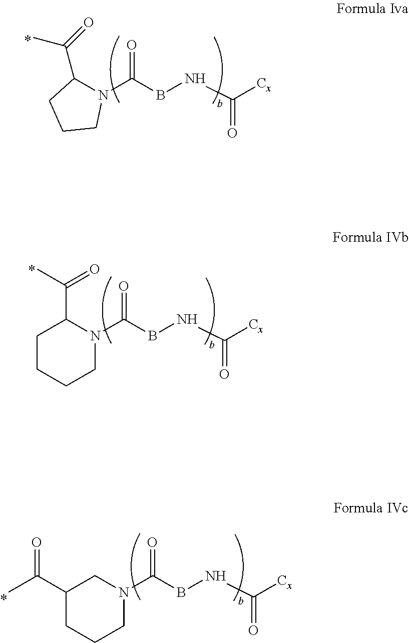

In one embodiment, the composition is characterized in that the hydrophobic radical is a radical according to formula VI wherein the radical GpC is according to formula IVa.

In one embodiment, the composition is characterized in that the hydrophobic radical is a radical according to formula I wherein the radical GpC according to formula IV is chosen from the group consisting of the radicals according to formulas IVa, IVb or IVc wherein b is equal to 0, respectively corresponding to formulas IVd, IVe, and IVf hereinafter represented:

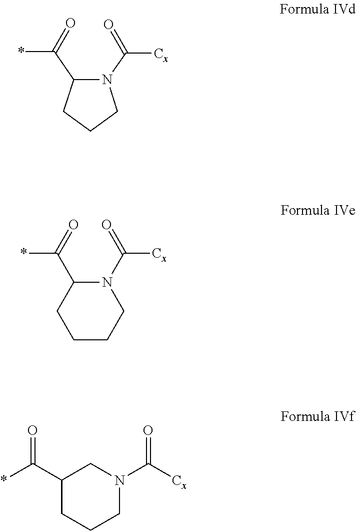

In one embodiment, the composition is characterized in that the hydrophobic radical is a radical according to formula VI wherein the radical GpC corresponds to formula IV or IVa wherein b=0, and corresponds to formula IVd.

In one embodiment, the composition is characterized in that the hydrophobic radical is a radical according to formula VI wherein the radical GpC according to formula IV is chosen from the group consisting of radicals wherein Cₓ is chosen from the group consisting of linear alkyl radicals comprising from 9 to 15 carbon atoms.

In one embodiment, the composition is characterized in that the hydrophobic radical is a radical according to formula VI wherein the radical GpC according to formula IV is chosen from the group consisting of radicals wherein Cₓ is chosen from the group consisting of alkyl radicals comprising from 9 to 15 carbon atoms.

In one embodiment, the composition is characterized in that the hydrophobic radical is a radical according to formula VI in which the radical GpC according to formula IV is chosen from the group consisting of radicals in which Cₓ is chosen from the group consisting of alkyl radicals comprising from 9 or 10 carbon atoms.

In one embodiment, the composition is characterized in that the hydrophobic radical is a radical according to formula VI wherein the radical GpC according to formula IV is chosen from the group consisting of radicals wherein Cx is chosen from the group consisting of alkyl radicals comprising from 11 to 15 carbon atoms.

In one embodiment, the composition is characterized in that the hydrophobic radical is a radical according to formula VI wherein the radical GpC according to formula IV is chosen from the group consisting of radicals wherein Cx is chosen from the group consisting of alkyl radicals comprising from 11 to 13 carbon atoms.

In one embodiment, the composition is characterized in that the hydrophobic radical is a radical according to formula VI wherein the radical GpC according to formula IV is chosen from the group consisting of radicals wherein Cₓ is chosen from the group consisting of radicals represented by the formulas below:

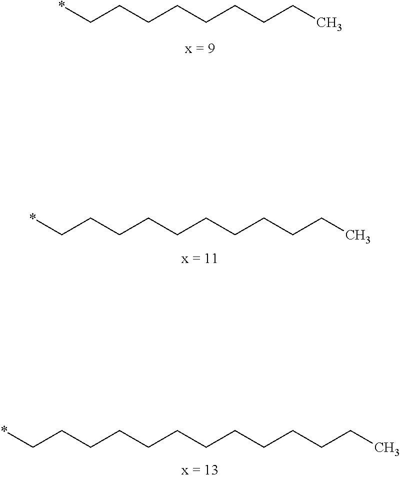

In one embodiment, the composition is characterized in that the hydrophobic radical is a radical according to formula VI in which the radical GpC according to formula IV is chosen from the group consisting of radicals in which Cx is chosen from the group consisting of alkyl radicals comprising from 14 or 15 carbon atoms.

In one embodiment, the composition is characterized in that the hydrophobic radical is a radical according to formula VI wherein the radical GpC according to formula IV is chosen from the group consisting of radicals wherein Cₓ is chosen from the group consisting of linear alkyl radicals:

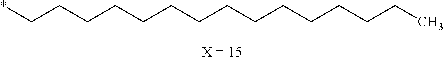 [SMILES: *CCCCCCCCCCCCCCC]

The values of the degree of polymerization DP and the ratio i are estimated by NMR ¹H in D₂O comparing the integration of the signals from the hydrophobic groups to that of the signals from the main chain of the co-polyamino acid.

In one embodiment, the composition is characterized in that the ratio i between the number of hydrophobic radicals and the number of glutamic or aspartic units is comprised from 0.007 to 0.3.

In one embodiment, the composition is characterized in that the ratio i between the number of hydrophobic radicals and the number of glutamic or aspartic units is comprised from 0.01 to 0.3.

In one embodiment, the composition is characterized in that the ratio i between the number of hydrophobic radicals and the number of glutamic or aspartic units is comprised from 0.03 to 0.3.

In one embodiment, the composition is characterized in that the ratio i between the number of hydrophobic radicals and the number of glutamic or aspartic units is comprised from 0.02 to 0.2.

In one embodiment, the composition is characterized in that the hydrophobic radical corresponds to formula VI and the ratio i between the number of hydrophobic radicals and the number of glutamic or aspartic units is comprised from 0.007 to 0.3.

In one embodiment, the composition is characterized in that the hydrophobic radical corresponds to formula VI and the ratio i between the number of hydrophobic radicals and the number of glutamic or aspartic units is comprised from 0.03 to 0.3.

In one embodiment, the composition is characterized in that the hydrophobic radical corresponds to formula VI and the ratio i between the number of hydrophobic radicals and the number of glutamic or aspartic units is comprised from 0.05 to 0.2.

In one embodiment, the composition is characterized in that the hydrophobic radical corresponds to formula VI and the ratio i between the number of hydrophobic radicals and the number of glutamic or aspartic units is comprised from 0.007 to 0.15.

In one embodiment, the composition is characterized in that the hydrophobic radical corresponds to formula VI and the ratio i between the number of hydrophobic radicals and the number of glutamic or aspartic units is comprised from 0.01 to 0.1.

In one embodiment, the composition is characterized in that the hydrophobic radical corresponds to formula VI and the ratio i between the number of hydrophobic radicals and the number of glutamic or aspartic units is comprised from 0.02 to 0.08.

In an embodiment, the composition is characterized in that the hydrophobic radical corresponds to formula VI wherein the radical Cx comprises from 9 to 10 carbon atoms and the ratio i between the number of hydrophobic radicals and the number of glutamic or aspartic units is comprised from 0.05 to 0.3.

In an embodiment, the composition is characterized in that the hydrophobic radical corresponds to formula VI wherein the radical Cx comprises from 9 to 10 carbon atoms and the ratio i between the number of hydrophobic radicals and the number of glutamic or aspartic units is comprised from 0.03 to 0.15.

In an embodiment, the composition is characterized in that the hydrophobic radical corresponds to formula VI wherein the radical Cx comprises from 11 to 12 carbon atoms and the ratio i between the number of hydrophobic radicals and the number of glutamic or aspartic units is comprised from 0.05 to 0.2.

In an embodiment, the composition is characterized in that the hydrophobic radical corresponds to formula VI wherein the radical Cx comprises from 11 to 12 carbon atoms and the ratio i between the number of hydrophobic radicals and the number of glutamic or aspartic units is comprised from 0.03 to 0.2.

In an embodiment, the composition is characterized in that the hydrophobic radical corresponds to formula VI wherein the radical Cx comprises from 11 to 12 carbon atoms and the ratio i between the number of hydrophobic radicals and the number of glutamic or aspartic units is comprised from 0.015 to 0.1.

In an embodiment, the composition is characterized in that the hydrophobic radical corresponds to formula VI wherein the radical Cx comprises from 11 to 12 carbon atoms and the ratio i between the number of hydrophobic radicals and the number of glutamic or aspartic units is comprised from 0.02 to 0.08.

In an embodiment, the composition is characterized in that the hydrophobic radical corresponds to formula VI wherein the radical Cx comprises from 13 to 15 carbon atoms and the ratio i between the number of hydrophobic radicals and the number of glutamic or aspartic units is comprised from 0.03 to 0.15.

In an embodiment, the composition is characterized in that the hydrophobic radical corresponds to formula VI wherein the radical Cx comprises from 13 to 15 carbon atoms and the ratio i between the number of hydrophobic radicals and the number of glutamic or aspartic units is comprised from 0.01 to 0.1.

In an embodiment, the composition is characterized in that the hydrophobic radical corresponds to formula VI wherein the radical Cx comprises from 13 to 15 carbon atoms and the ratio i between the number of hydrophobic radicals and the number of glutamic or aspartic units is comprised from 0.01 to 0.06.

In one embodiment, the composition is characterized in that the hydrophobic radical corresponds to formula V and the ratio i between the number of hydrophobic radicals and the number of glutamic or aspartic units is comprised from 0.007 to 0.3.

In one embodiment, the composition is characterized in that the hydrophobic radical corresponds to formula V and the ratio i between the number of hydrophobic radicals and the number of glutamic or aspartic units is comprised from 0.01 to 0.3.

In one embodiment, the composition is characterized in that the hydrophobic radical corresponds to formula V and the ratio i between the number of hydrophobic radicals and the number of glutamic or aspartic units is comprised from 0.05 to 0.3.

In one embodiment, the composition is characterized in that the hydrophobic radical corresponds to formula V and the ratio i between the number of hydrophobic radicals and the number of glutamic or aspartic units is comprised from 0.1 to 0.3.

In one embodiment, the composition is characterized in that the hydrophobic radical corresponds to formula V and the ratio i between the number of hydrophobic radicals and the number of glutamic or aspartic units is comprised from 0.015 to 0.2.

In an embodiment, the composition is characterized in that the hydrophobic radical corresponds to formula V wherein the radical Cₓ comprises from 11 to 14 carbon atoms and the ratio i between the number of hydrophobic radicals and the number of glutamic or aspartic units is comprised from 0.1 to 0.3.

In an embodiment, the composition is characterized in that the hydrophobic radical corresponds to formula V wherein the radical Cx comprises from 11 to 14 carbon atoms and the ratio i between the number of hydrophobic radicals and the number of glutamic or aspartic units is comprised from 0.1 to 0.2.

In an embodiment, the composition is characterized in that the hydrophobic radical corresponds to formula V wherein the radical Cx comprises from 15 to 16 carbon atoms and the ratio i between the number of hydrophobic radicals and the number of glutamic or aspartic units is comprised from 0.04 to 0.2.

In an embodiment, the composition is characterized in that the hydrophobic radical corresponds to formula V wherein the radical Cₓ comprises from 15 to 16 carbon atoms and the ratio i between the number of hydrophobic radicals and the number of glutamic or aspartic units is comprised from 0.06 to 0.2.

In an embodiment, the composition is characterized in that the hydrophobic radical corresponds to formula V wherein the radical Cx comprises from 15 to 16 carbon atoms and the ratio i between the number of hydrophobic radicals and the number of glutamic or aspartic units is comprised from 0.04 to 0.15.

In an embodiment, the composition is characterized in that the hydrophobic radical corresponds to formula V wherein the radical Cx comprises from 17 to 18 carbon atoms and the ratio i between the number of hydrophobic radicals and the number of glutamic or aspartic units is comprised from 0.02 to 0.2.

In an embodiment, the composition is characterized in that the hydrophobic radical corresponds to formula V wherein the radical Cx comprises from 17 to 18 carbon atoms and the ratio i between the number of hydrophobic radicals and the number of glutamic or aspartic units is comprised from 0.02 to 0.15.

In an embodiment, the composition is characterized in that the hydrophobic radical corresponds to formula V wherein the radical Cx comprises from 17 to 18 carbon atoms and the ratio i between the number of hydrophobic radicals and the number of glutamic or aspartic units is comprised from 0.02 to 0.06.

In an embodiment, the composition is characterized in that the hydrophobic radical corresponds to formula V wherein the radical Cx comprises from 19 to 25 carbon atoms and the ratio i between the number of hydrophobic radicals and the number of glutamic or aspartic units is comprised from 0.01 to 0.1.

In an embodiment, the composition is characterized in that the hydrophobic radical corresponds to formula V wherein the radical Cx comprises from 19 to 25 carbon atoms and the ratio i between the number of hydrophobic radicals and the number of glutamic or aspartic units is comprised from 0.01 to 0.06.

In an embodiment, the composition is characterized in that the hydrophobic radical corresponds to formula V wherein the radical Cx comprises from 19 to 25 carbon atoms and the ratio i between the number of hydrophobic radicals and the number of glutamic or aspartic units is comprised from 0.01 to 0.05.

In one embodiment, the composition is characterized in that the co-polyamino acid bearing carboxylate charges and hydrophobic radicals is chosen from the co-polyamino acids according to formula VII below:

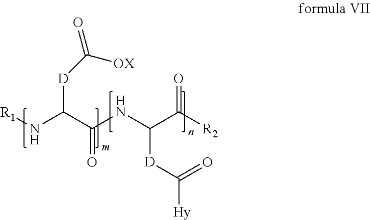

wherein,
D represents, independently, either a —CH₂— group (aspartic unit) or a —CH₂—CH₂-group (glutamic unit),
Hy is a hydrophobic radical chosen from hydrophobic radicals according to formula I, V or VI,
R₁ is a hydrophobic radical chosen from the hydrophobic radicals of according to formula I, V or VI, or a radical chosen from the group consisting of a H, a C₂ at C₁₀ linear acyl group, a C₃ to C₁₀ branched acyl group, a benzyl, a terminal “amino acid” unit and a pyroglutamate,
R₂ is a hydrophobic radical chosen from hydrophobic radicals according to formula I, V or VI, or a radical —NR′R″, R′ and R″ identical or different chosen from the group consisting of H, linear or branched alkyls or cyclical in C₂ to C₁₀, benzyl and said R′ and R″ alkyls may form together one or more saturated carbon rings, unsaturated and/or aromatic and/or may contain heteroatoms, chosen from the group consisting of O, N and S,
X represents a H or a cationic entity chosen from the group consisting of metal cations;
n+m represents the degree of polymerization DP of the co-polyamino acid, namely the average number of monomeric units per co-polyamino acid chain and 5≤n+m≤250.

In one embodiment, the composition is characterized in that the co-polyamino acid bearing carboxylate charges and hydrophobic radicals is chosen from the co-polyamino acids according to formula VII below:

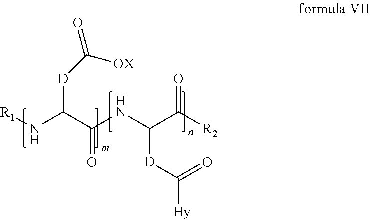

wherein,
D represents, independently, either a —CH₂— group (aspartic unit) or a —CH₂—CH₂-group (glutamic unit),
Hy is a hydrophobic radical chosen from hydrophobic radicals according to formula I, V or VI,
R₁ is a hydrophobic radical chosen from the hydrophobic radicals of according to formula I, V or VI, or a radical chosen from the group consisting of a H, a C₂ at C₁₀ linear acyl group, a C₃ to C₁₀ branched acyl group, a benzyl, a terminal “amino acid” unit and a pyroglutamate,
R₂ is a hydrophobic radical chosen from hydrophobic radicals according to formulas I, V or VI wherein r=1 or 2 and GpR is a radical according to Formula II, or a radical —NR′R″, R′ and R″ identical or different being chosen from the group consisting of H, linear or branched or cyclic alkyls in C₂ at C₁₀, benzyl and said alkyl R′ and R″ may form together one or more saturated carbon rings, unsaturated and/or aromatic and/or may contain heteroatoms, chosen from the group consisting of O, N and S,
at least one of the R₁ or R₂ is a hydrophobic radical as defined above,
X represents a H or a cationic entity chosen from the group consisting of metal cations;
n+m represents the degree of polymerization DP of the co-polyamino acid, namely the average number of monomeric units per co-polyamino acid chain and 5≤n+m≤250.

In one embodiment, the composition according to the invention is characterized in that the co-polyamino acid bearing carboxylate charges and hydrophobic radicals is chosen from the co-polyamino acids according to formula VII wherein at least one of the R₁ or R₂ is a hydrophobic radical according to formula I, V or VI.

In one embodiment, the composition according to the invention is characterized in that the co-polyamino acid bearing carboxylate charges and hydrophobic radicals is chosen from the co-polyamino acids according to formula VII wherein at least one of the R₁ or R₂ is a hydrophobic radical according to formula VI.

In one embodiment, the composition according to the invention is characterized in that the co-polyamino acid bearing carboxylate charges and hydrophobic radicals is chosen from the co-polyamino acids according to formula VII wherein at least one of the R₁ or R₂ is a hydrophobic radical according to formula VI, and Hy is a radical according to formula VI, wherein r=0.

In one embodiment, the composition according to the invention is characterized in that the co-polyamino acid bearing carboxylate charges and hydrophobic radicals is chosen from the co-polyamino acids according to formula VII wherein at least one of the R₁ or R₂ is a hydrophobic radical according to formula VI, and Hy is a radical according to formula VI, wherein r=1.

In one embodiment, the composition according to the invention is characterized in that the co-polyamino acid bearing carboxylate charges and hydrophobic radicals is chosen from the co-polyamino acids according to formula VII wherein at least one of the R₁ or R₂ is a hydrophobic radical according to formula VI, and Hy is a radical according to formula VI, wherein r=2.

In one embodiment, the composition according to the invention is characterized in that the co-polyamino acid bearing carboxylate charges and hydrophobic radicals is chosen from the co-polyamino acids according to formula VII wherein at least one of the R₁ or R₂ is a hydrophobic radical according to formula VI wherein r=1, and for GpC, b=0.

In one embodiment, the composition according to the invention is characterized in that the co-polyamino acid bearing carboxylate charges and hydrophobic radicals is chosen from the co-polyamino acids according to formula VII wherein at least one of the R₁ or R₂ is a hydrophobic radical according to formula VI wherein r=2, and for GpC, b=0.

In one embodiment, the composition according to the invention is characterized in that the co-polyamino acid bearing carboxylate charges and hydrophobic radicals is chosen from the co-polyamino acids according to formula VII wherein R₁ is a hydrophobic radical according to formula VI.

In one embodiment, the composition according to the invention is characterized in that the co-polyamino acid bearing carboxylate charges and hydrophobic radicals is chosen from the co-polyamino acids according to formula VII wherein R₁ is a hydrophobic radical according to formula VI and R₂ is a radical —NR′R″, R′ and R″ being as defined above.

In one embodiment, the composition according to the invention is characterized in that the co-polyamino acid bearing carboxylate charges and hydrophobic radicals is chosen from the co-polyamino acids according to formula VII wherein R₁ is a hydrophobic radical according to formula VI and R₂ is a radical —NR′R″, R′ and R″ being as defined above, and Hy is a radical according to formula VI, wherein r=1.

In one embodiment, the composition according to the invention is characterized in that the co-polyamino acid bearing carboxylate charges and hydrophobic radicals is chosen from the co-polyamino acids according to formulas VII. In one embodiment, the invention is characterized in that the co-polyamino acid bearing carboxylate charges and hydrophobic radicals is chosen from co-polyamino acids according to formula VII wherein R₁ is a hydrophobic radical according to formula VI and R₂ is a radical —NR′R″, R′ and R″ being as defined above, and Hy is a radical according to formula VI, wherein r=1, and for GpC, b=0.

In one embodiment, the composition according to the invention is characterized in that the co-polyamino acid bearing carboxylate charges and hydrophobic radicals is chosen from the co-polyamino acids according to formula VII wherein R₁ is a hydrophobic radical according to formula VI and R₂ is a radical —NR′R″, R′, and R″ being as defined above, and Hy is a radical according to formula VI, wherein r=2.

In one embodiment, the composition according to the invention is characterized in that the co-polyamino acid bearing carboxylate charges and hydrophobic radicals is chosen from the co-polyamino acids according to formulas VII. In one embodiment, the invention is characterized in that the co-polyamino acid bearing carboxylate charges and hydrophobic radicals is chosen from the co-polyamino acids according to formula VII wherein R₁ is a hydrophobic radical according to formula VI and R₂ is a radical —NR′R″, R′ and R″ being as defined above, and Hy is a radical according to formula VI, wherein r=2, and for GpC, b=0.

In one embodiment, the composition according to the invention is characterized in that the co-polyamino acid bearing carboxylate charges and hydrophobic radicals is chosen from the co-polyamino acids according to formula VII wherein R₂ is a hydrophobic radical according to formula I, V or VI.

In one embodiment, the composition according to the invention is characterized in that the co-polyamino acid bearing carboxylate charges and hydrophobic radicals is chosen from the co-polyamino acids according to formula VII wherein R₂ is a hydrophobic radical according to formula VI.

In one embodiment, the composition according to the invention is characterized in that the co-polyamino acid bearing carboxylate charges and hydrophobic radicals is chosen from the co-polyamino acids according to formula VII wherein R2 is a hydrophobic radical according to formula VI and R1 is a radical —NR′R″, R′ and R″ being as defined above.

In one embodiment, the composition according to the invention is characterized in that the co-polyamino acid bearing carboxylate charges and hydrophobic radicals is chosen from the co-polyamino acids according to formula VII wherein R2 is a hydrophobic radical according to formula VI and R1 is a radical —NR′R″, R′, and R″ being as defined above, and Hy is a radical according to formula VI, wherein r=1.

In one embodiment, the composition according to the invention is characterized in that the co-polyamino acid bearing carboxylate charges and hydrophobic radicals is chosen from the co-polyamino acids according to formula VII wherein R₂ is a hydrophobic radical according to formula VI and R₁ is a radical —NR′R″, R′, and R″ being as defined above, and Hy is a radical according to formula VI, wherein r=1 and for GpC, b=0.

In one embodiment, the composition according to the invention is characterized in that the co-polyamino acid bearing carboxylate charges and hydrophobic radicals is chosen from the co-polyamino acids according to formula VII wherein R₁ et R₂ is a hydrophobic radical according to formula I, V or VI.

In one embodiment, the composition according to the invention is characterized in that the co-polyamino acid bearing carboxylate charges and hydrophobic radicals is chosen from the co-polyamino acids according to formula VII wherein R₁ et R₂ is a hydrophobic radical according to formula VI.

In one embodiment, the composition according to the invention is characterized in that the co-polyamino acid bearing carboxylate charges and hydrophobic radicals is chosen from the co-polyamino acids according to formula VII wherein at least one of the R₁ or R₂ is a hydrophobic radical according to formula VI, and Hy is a radical according to formula VI, wherein r=1.

In one embodiment, the composition according to the invention is characterized in that the co-polyamino acid bearing carboxylate charges and hydrophobic radicals is chosen from the co-polyamino acids according to formula VII wherein R₁ and R₂ is a hydrophobic radical according to formula VI, and Hy is a radical according to formula VI, where r=1, and for GpC, b=0.

In one embodiment, the composition according to the invention is characterized in that the co-polyamino acid bearing carboxylate charges and hydrophobic radicals is chosen from the co-polyamino acids according to formula VII wherein R₂ is a hydrophobic radical according to formula I, V or VI wherein r=1 and GpR is according to Formula II.

In one embodiment, the composition according to the invention is characterized in that the co-polyamino acid bearing carboxylate charges and hydrophobic radicals is chosen from the co-polyamino acids according to formula VII wherein R₂ is a hydrophobic radical according to formula VI wherein r=1 and GpR is according to Formula II.

In one embodiment, the composition according to the invention is characterized in that the co-polyamino acid bearing carboxylate charges and hydrophobic radicals is chosen from the co-polyamino acids according to formula VII wherein R₂ is a hydrophobic radical according to formula VI wherein r=1, GpR is according to Formula II and GpC is according to formula IV wherein b=0, c=0 and d=1.

In one embodiment, the composition according to the invention is characterized in that the co-polyamino acid bearing carboxylate charges and hydrophobic radicals is chosen from the co-polyamino acids according to formula VII wherein R₂ is a hydrophobic radical according to formula VI wherein r=1, GpR is according to Formula II and GpC is according to formula IV wherein b=0, c=0 and d=1 and x=13.

In one embodiment, the composition according to the invention is characterized in that when the co-polyamino acid comprises aspartate units, then the co-polyamino acid may further comprise monomeric units according to Formula VIII and/or VIII′:

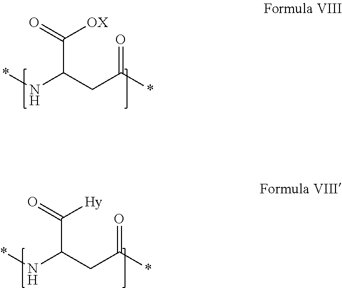

The co-polyamino acid bearing carboxylate charges and at least one hydrophobic radical according to formula I can also be referred to as “co-polyamino acid” in the present description.

In one embodiment, the composition according to the invention is characterized in that the co-polyamino acid bearing carboxylate charges and hydrophobic radicals is chosen from the co-polyamino acids according to formula VII wherein n≥1 and at least one of the R₁ or R₂ is a hydrophobic radical according to formula I, V or VI.

In one embodiment, the composition according to the invention is characterized in that the co-polyamino acid bearing carboxylate charges and hydrophobic radicals is chosen from the co-polyamino acids according to formula VII wherein n≥1 and R1 is a hydrophobic radical according to formula I, V or VI.

In one embodiment, the composition according to the invention is characterized in that the co-polyamino acid bearing carboxylate charges and hydrophobic radicals is chosen from the co-polyamino acids according to formula VII wherein n≥1 and R₂ is a hydrophobic radical according to formula I, V or VI.

In one embodiment, the composition according to the invention is characterized in that the co-polyamino acid bearing carboxylate charges and hydrophobic radicals is chosen from the co-polyamino acids according to formula VII wherein n≥1, R₁ is a hydrophobic radical according to formula I, V or VI wherein r=0.

In one embodiment, the composition according to the invention is characterized in that the co-polyamino acid bearing carboxylate charges and hydrophobic radicals is chosen from the co-polyamino acids according to formula VII wherein n≥1, R₂ is a hydrophobic radical according to formula I, V or VI wherein r=1 or 2 and GpR is according to Formula II.

In one embodiment, the composition according to the invention is characterized in that the co-polyamino acid bearing carboxylate charges and hydrophobic radicals is chosen from the co-polyamino acids according to formula VII wherein ≥1, R₂ is a hydrophobic radical according to formula VI hydrophobic radical wherein r=1 or 2 and GpR is according to Formula II.

In one embodiment, the composition according to the invention is characterized in that the co-polyamino acid bearing carboxylate charges and hydrophobic radicals is chosen from the co-polyamino acids according to formula VII wherein n≥1, R₂ is a hydrophobic radical according to formula VI wherein r=1 or 2, GpR is according to Formula II and GpC is according to formula IV.

In one embodiment, the composition according to the invention is characterized in that the co-polyamino acid bearing carboxylate charges and hydrophobic radicals is chosen from the co-polyamino acids according to formula VII wherein n≥1, R₂ is a hydrophobic radical according to formula VI wherein r=1 or 2, GpR is according to Formula II and GpC is according to formula IV wherein b=0, c=0 and d=1.

In one embodiment, the composition according to the invention is characterized in that the co-polyamino acid bearing carboxylate charges and hydrophobic radicals is chosen from the co-polyamino acids according to formula VII wherein n≥1, R₂ is a hydrophobic radical according to formula VI wherein r=1 or 2, GpR is according to Formula II and GpC is according to formula IV wherein b=0, c=0 and d=1 and x=13.

In one embodiment, the composition according to the invention is characterized in that the co-polyamino acid bearing carboxylate charges and hydrophobic radicals is chosen from the co-polyamino acids according to formula VII wherein R₂ is a hydrophobic radical according to formula VI and R₁ is a radical chosen from the group consisting of H, a linear C₂ to C₁₀, acyl group, a branched C₃ to C₁₀, acyl group, a benzyl, a terminal “amino acid” unit and a pyroglutamate.

In one embodiment, the composition according to the invention is characterized in that the co-polyamino acid bearing carboxylate charges and hydrophobic radicals is chosen from the co-polyamino acids according to formula VII wherein R₁ is a hydrophobic radical according to formula VI wherein r=1 or 2, and for GpC, b=0 and R₂ is a —NR′R″, R′ and R′ radical as defined above.

In one embodiment, the composition according to the invention is characterized in that the co-polyamino acid bearing carboxylate charges and hydrophobic radicals is chosen from the co-polyamino acids according to formula VII wherein R₁ and R₂ are hydrophobic radicals according to formula I, V or VI.

In one embodiment, the composition according to the invention is characterized in that the co-polyamino acid bearing carboxylate charges and hydrophobic radicals is chosen from the co-polyamino acids according to formula VII wherein R₁ and R₂ are hydrophobic radicals according to formula VI.

In one embodiment, the composition according to the invention is characterized in that the co-polyamino acid bearing carboxylate charges and hydrophobic radicals is chosen from the co-polyamino acids according to formula VII wherein R₁ and R₂ are hydrophobic radicals according to formula VI, wherein r=1 or 2 and GpR according to formula II for R₂ and r=0 for R₁.

In one embodiment, the composition according to the invention is characterized in that the co-polyamino acid bearing carboxylate charges and hydrophobic radicals is chosen from the co-polyamino acids according to formula VII wherein R₁ and R₂ are hydrophobic radicals according to formula VI, GpA=0 and b=0, and wherein r=1 or 2 and GpR according to formula II for R₂ and r=0 for R₁.

In one embodiment, the composition is characterized in that the co-polyamino acid bearing carboxylate charges and hydrophobic radicals is chosen from the co-polyamino acids according to formula VII wherein R₁=R′₁ and R₂=R′₂, according to formula VIIa below:

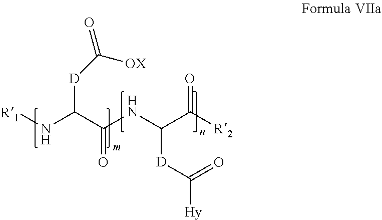

wherein,
D represents, independently, either a —CH₂— group (aspartic unit) or a —CH₂—CH₂-group (glutamic unit),
Hy is a hydrophobic radical chosen from hydrophobic radicals according to formula I, V or VI, wherein r=1 and GpR is a radical according to Formula II,
X represents a H or a cationic entity chosen from the group consisting of metal cations;
n+m represents the degree of polymerization DP of the co-polyamino acid, namely the average number of monomeric units per co-polyamino acid chain and 5≤n+m≤250;
R′ is a radical chosen from the group consisting of H, linear C₂ to C₁₀ acyl group, branched C₄ to C₁₀ acyl group, benzyl, terminal amino acid unit and pyroglutamate,
R′₂ is an identical or different radical —NR′R″, R′ and R″ chosen from the group consisting of H, linear or branched or cyclic C2 to C10 alkyls, benzyl and said R′ and R″ alkyls which can form together one or more saturated, unsaturated and/or aromatic carbon rings and/or which may contain heteroatoms chosen from the group consisting of O, N and S.

In one embodiment, the composition according to the invention is characterized in that the co-polyamino acid bearing carboxylate charges and hydrophobic radicals is chosen from the co-polyamino acids according to formula VIIa wherein Hy is a radical according to formula V or formula VI, wherein r=1.

In one embodiment, the composition according to the invention is characterized in that the co-polyamino acid bearing carboxylate charges and hydrophobic radicals is chosen from the co-polyamino acids according to formula VIIa wherein Hy is a radical according to formula V or formula VI, with GpR according to formula II.

In one embodiment, the composition according to the invention is characterized in that the co-polyamino acid bearing carboxylate charges and hydrophobic radicals is chosen from the co-polyamino acids according to formula VIIa, wherein Hy is a radical according to formula V.

In one embodiment, the composition according to the invention is characterized in that the co-polyamino acid bearing carboxylate charges and hydrophobic radicals is chosen from the co-polyamino acids according to formula VIIa, wherein Hy is according to formula V and GpC is a radical according to formula IVd.

In one embodiment, the composition according to the invention is characterized in that the co-polyamino acid bearing carboxylate charges and hydrophobic radicals is chosen from the co-polyamino acids according to formula VIIa, wherein Hy is according to formula V and GpC is a radical according to formula IVd wherein x=13.

In one embodiment, the composition according to the invention is characterized in that the co-polyamino acid bearing carboxylate charges and hydrophobic radicals is chosen from the co-polyamino acids according to formula VIIa, wherein Hy is according to formula V and GpC is a radical according to formula IVd wherein x=15.

In one embodiment, the composition according to the invention is characterized in that the co-polyamino acid bearing carboxylate charges and hydrophobic radicals is chosen from the co-polyamino acids according to formula VIIa, wherein Hy is according to formula V and GpC is a radical according to formula IVd wherein x=17.

In one embodiment, the composition according to the invention is characterized in that the co-polyamino acid bearing carboxylate charges and hydrophobic radicals is chosen from the co-polyamino acids according to formula VIIa, wherein Hy is according to formula V and GpC is a radical according to formula IVd wherein x=19.

In one embodiment, the composition according to the invention is characterized in that the co-polyamino acid bearing carboxylate charges and hydrophobic radicals is chosen from the co-polyamino acids according to formula VIIa, wherein Hy is a radical according to formula VI.

In one embodiment, the composition according to the invention is characterized in that the co-polyamino acid bearing carboxylate charges and hydrophobic radicals is chosen from the co-polyamino acids according to formula VIIa, wherein Hy is a radical according to formula VI wherein r=1 and GpR according to formula II.

In one embodiment, the composition according to the invention is characterized in that the co-polyamino acid bearing carboxylate charges and hydrophobic radicals is chosen from the co-polyamino acids according to formula VIIa, wherein Hy is a radical according to formula VI wherein r=1, GpR according to formula II and for GpC, b=0.

In one embodiment, the composition according to the invention is characterized in that the co-polyamino acid bearing carboxylate charges and hydrophobic radicals is chosen from the co-polyamino acids according to formulas VIIa, wherein Hy is according to formula VI, GpR according to formula II and GpC is a radical according to formula IVd.

In one embodiment, the composition according to the invention is characterized in that the co-polyamino acid bearing carboxylate charges and hydrophobic radicals is chosen from the co-polyamino acids according to formulas VIIa, wherein Hy is according to formula VI, GpR according to formula II and GpC is a radical according to formula IVd and r=1.

In one embodiment, the composition according to the invention is characterized in that the co-polyamino acid bearing carboxylate charges and hydrophobic radicals is chosen from the co-polyamino acids according to formulas VIIa, wherein Hy is according to formula VI, GpR according to formula II and GpC is a radical according to formula IVd wherein x is comprised from 11 to 15.

In one embodiment, the composition is characterized in that the co-polyamino acid bearing carboxylate charges and hydrophobic radicals is chosen from the co-polyamino acids according to formulas VIIa wherein Hyd is according to formula V, GpR is according to formula II, GpA is according to formula III′ wherein A is Y9 and GpC corresponds to formula IVd.

In one embodiment, the composition is characterized in that the co-polyamino acid bearing carboxylate charges and hydrophobic radicals is chosen from the co-polyamino acids according to formulas VIIa wherein Hyd is according to formula V, GpR is according to formula II wherein R is —CH2-CH2-, GpA is according to formula III′ wherein A is Y9 and GpC corresponds to formula IVd.

In one embodiment, the composition is characterized in that the co-polyamino acid bearing carboxylate charges and hydrophobic radicals is chosen from the co-polyamino acids according to formulas VIIa wherein Hyd is according to formula V.

In one embodiment, the composition is characterized in that the co-polyamino acid bearing carboxylate charges and hydrophobic radicals is chosen from the co-polyamino acids according to formulas VIIa wherein Hyd is according to formula V wherein r=1 and a=0.

In one embodiment, the composition is characterized in that the co-polyamino acid bearing carboxylate charges and hydrophobic radicals is chosen from the co-polyamino acids according to formulas VIIa wherein Hyd is according to formula V wherein r=1 and a=1.

In one embodiment, the composition is characterized in that the co-polyamino acid bearing carboxylate charges and hydrophobic radicals is chosen from the co-polyamino acids according to formulas VIIa wherein Hyd is according to formula V wherein r=1 and GpR corresponds to formula II.

In one embodiment, the composition is characterized in that the co-polyamino acid bearing carboxylate charges and hydrophobic radicals is chosen from the co-polyamino acids according to formulas VIIa wherein Hyd is according to formula V wherein r=1 and GpC is according to formula IVd.

In one embodiment, the composition is characterized in that the co-polyamino acid bearing carboxylate charges and hydrophobic radicals is chosen from the co-polyamino acids according to formulas VIIa wherein Hyd is according to formula VI.

In one embodiment, the composition is characterized in that the co-polyamino acid bearing carboxylate charges and hydrophobic radicals is chosen from the co-polyamino acids according to formulas VIIa wherein Hyd is according to formula VI wherein r=1 GpR is according to formula II.

In one embodiment, the composition is characterized in that the co-polyamino acid bearing carboxylate charges and hydrophobic radicals is chosen from the co-polyamino acids according to formulas VIIa wherein Hyd is according to formula VI wherein GpC is according to formula IVd.

In one embodiment, the composition is characterized in that the co-polyamino acid bearing carboxylate charges and hydrophobic radicals is chosen from the co-polyamino acids according to formulas VIIa wherein Hyd is according to formula VI wherein r=1 and GpC is according to formula IVd.

In one embodiment, the composition is characterized in that the co-polyamino acid bearing carboxylate charges and hydrophobic radicals is chosen from the co-polyamino acids according to formula VIIa wherein the group D is a —CH group —CH₂— (aspartic unit).

In one embodiment, the composition is characterized in that the co-polyamino acid bearing carboxylate charges and hydrophobic radicals is chosen from the co-polyamino acids according to formula VIIa wherein the group D is a —CH₂—CH₂— (glutamic unit) group.

A “defined co-polyamino acid” refers to a co-polyamino acid bearing carboxylate charges and at least one hydrophobic radical, a co-polyamino acid according to formula VIIb.

In one embodiment, the composition is characterized in that the co-polyamino acid bearing carboxylate charges and hydrophobic radicals is chosen from the co-polyamino acids according to formula VII wherein n=0 according to formula VIIb below:

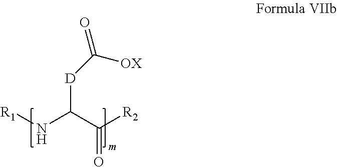

wherein m, X, D, R₁ and R₂ have the definitions given above and at least R₁ or R₂ is a hydrophobic radical according to formula I, V or VI.

In one embodiment, the composition is characterized in that the co-polyamino acid bearing carboxylate charges and hydrophobic radicals is chosen from the co-polyamino acids of according to formula VII wherein n=0 according to formula VIIb and R₁ or R₂ is a hydrophobic radical according to formula I, V or VI.

In one embodiment, the composition according to the invention is characterized in that the co-polyamino acid bearing carboxylate charges and hydrophobic radicals is chosen from the co-polyamino acids according to formula VII wherein at least one of the R₁ or R₂ is a hydrophobic radical according to formula VI.

In one embodiment, the composition according to the invention is characterized in that the co-polyamino acid bearing carboxylate charges and hydrophobic radicals is chosen from the co-polyamino acids according to formula VIIb wherein R₁ is a hydrophobic radical according to formula VI.

In one embodiment, the composition according to the invention is characterized in that the co-polyamino acid bearing carboxylate charges and hydrophobic radicals is chosen from the co-polyamino acids according to formula VIIb wherein R₁ is a hydrophobic radical according to formula VI and R₂ is a radical —NR′R″, R′ and R″ being as defined above.

In one embodiment, the composition according to the invention is characterized in that the co-polyamino acid bearing carboxylate charges and hydrophobic radicals is chosen from the co-polyamino acids according to formula VIIb wherein R₂ is a hydrophobic radical according to formula I, V or VI.

In one embodiment, the composition according to the invention is characterized in that the co-polyamino acid bearing carboxylate charges and hydrophobic radicals is chosen from the co-polyamino acids according to formula VIIb wherein R2 is a hydrophobic radical according to formula VI.

In one embodiment, the composition according to the invention is characterized in that the co-polyamino acid bearing carboxylate charges and hydrophobic radicals is chosen from the co-polyamino acids according to formula VIIb wherein R₂ is a hydrophobic radical according to formula VI wherein r=0 and R₁ is a radical chosen from the group consisting of H, a linear C₂ to C₁₀, acyl group, a branched C₃ to C₁₀, acyl group, a benzyl, a terminal “amino acid” unit and a pyroglutamate.

In one embodiment, the composition is characterized in that the co-polyamino acid bearing carboxylate charges and hydrophobic radicals is chosen from the co-polyamino acids according to formula VIIb wherein R₁ is a hydrophobic radical according to formula VI wherein r=0.

In one embodiment, the composition is characterized in that the co-polyamino acid bearing carboxylate charges and hydrophobic radicals is chosen from the co-polyamino acids according to formula VIIb wherein R₁ is a hydrophobic radical according to formula VI wherein r=0, and GpC is according to formula IV.

In one embodiment, the composition is characterized in that the co-polyamino acid bearing carboxylate charges and hydrophobic radicals is chosen from the co-polyamino acids according to formula VIIb wherein R₁ is a hydrophobic radical according to formula VI wherein r=0, and GpC is according to formula IV with b=0, c=0 and d=1.

In one embodiment, the composition is characterized in that the co-polyamino acid bearing carboxylate charges and hydrophobic radicals is chosen from the co-polyamino acids according to formula VIIb wherein R₁ is a hydrophobic radical according to formula VI wherein r=0, and GpC is according to formula IV with b=0, c=0, d=1 and x=13.

In one embodiment, the composition according to the invention is characterized in that the co-polyamino acid bearing carboxylate charges and hydrophobic radicals is chosen from the co-polyamino acids according to formula VIIb wherein R₂ and Hy are hydrophobic radicals according to formula VI wherein r=0 and for GpC, b=0 and R₁ is a radical chosen from the group consisting of H, a linear C₂ to C₁₀ acyl group, a branched C₃ to C₁₀, acyl group, a benzyl, a terminal “amino acid” unit and a pyroglutamate.

In one embodiment, the composition is characterized in that the co-polyamino acid bearing carboxylate charges and hydrophobic radicals is chosen from the co-polyamino acids according to formula VIIb wherein R₁ is a hydrophobic radical according to formula I, V or VI wherein r=0 or r=1 or r=2 and GpR is according to Formula II′ or II″.

In one embodiment, the composition is characterized in that the co-polyamino acid bearing carboxylate charges and hydrophobic radicals is chosen from the co-polyamino acids according to formulas VIIb wherein R₂ is a hydrophobic radical according to formula I, V or VI wherein r=1 or II and GpR is according to Formula II.

In one embodiment, the composition is characterized in that the co-polyamino acid bearing carboxylate charges and hydrophobic radicals is chosen from the co-polyamino acids according to formula VII wherein R₂ is a hydrophobic radical, particularly with n≥1, or VIIb wherein R₁ is a radical chosen from the group consisting of a linear C₂ to C₁₀ acryl group, a branched C₃ to C₁₀ acyl group, a benzyl, a terminal “amino acid” unit and a pyroglutamate.

In one embodiment, the composition is characterized in that the co-polyamino acid bearing carboxylate charges and hydrophobic radicals is chosen from the co-polyamino acids according to formulas VIIb wherein Hyd is according to formula V.

In one embodiment, the composition is characterized in that the co-polyamino acid bearing carboxylate charges and hydrophobic radicals is chosen from the co-polyamino acids according to formulas VIIb wherein Hyd is according to formula V wherein r=1 and a=0.

In one embodiment, the composition is characterized in that the co-polyamino acid bearing carboxylate charges and hydrophobic radicals is chosen from the co-polyamino acids according to formulas VIIb wherein Hyd is according to formula V wherein r=1 and a=1.

In one embodiment, the composition is characterized in that the co-polyamino acid bearing carboxylate charges and hydrophobic radicals is chosen from the co-polyamino acids according to formulas VIIb wherein Hyd is according to formula V wherein r=1 and GpR is according to formula II.

In one embodiment, the composition is characterized in that the co-polyamino acid bearing carboxylate charges and hydrophobic radicals is chosen from the co-polyamino acids according to formulas VIIb wherein Hyd is according to formula V wherein r=1 and GpC is according to formula IVd.

In one embodiment, the composition is characterized in that the co-polyamino acid bearing carboxylate charges and hydrophobic radicals is chosen from the co-polyamino acids according to formulas VIIb wherein Hyd is according to formula VI.

In one embodiment, the composition is characterized in that the co-polyamino acid bearing carboxylate charges and hydrophobic radicals is chosen from the co-polyamino acids according to formulas VIIb wherein Hyd is according to formula VI wherein r=0.

In one embodiment, the composition is characterized in that the co-polyamino acid bearing carboxylate charges and hydrophobic radicals is chosen from the co-polyamino acids according to formulas VIIb wherein Hyd is according to formula VI wherein r=1 and GpR corresponds to formula II.

In one embodiment, the composition is characterized in that the co-polyamino acid bearing carboxylate charges and hydrophobic radicals is chosen from the co-polyamino acids according to formulas VIIb wherein Hyd is according to formula VI wherein GpC is according to formula IVd.

In one embodiment, the composition is characterized in that the co-polyamino acid bearing carboxylate charges and hydrophobic radicals is chosen from the co-polyamino acids according to formulas VIIb wherein Hyd is according to formula VI wherein r=1 and GpC is according to formula IVd.

### Group D

In one embodiment, the composition is characterized in that the co-polyamino acid bearing carboxylate charges and hydrophobic radicals is chosen from the co-polyamino acids according to formula VII, VIIa or VIIb wherein the group D is a —CH₂— (aspartic unit) group.

In one embodiment, the composition is characterized in that the co-polyamino acid bearing carboxylate charges and hydrophobic radicals is chosen from the co-polyamino acids according to formula VII, VIIa or VIIb wherein the group D is a —CH₂—CH₂— (glutamic unit) group.

When the co-polyamino acid comprises one or more aspartic unit (s), the latter can undergo structural rearrangements.

In one embodiment, the composition according to the invention is characterized in that the co-polyamino acids may further comprise monomeric units according to formula VIII and/or VIII′:

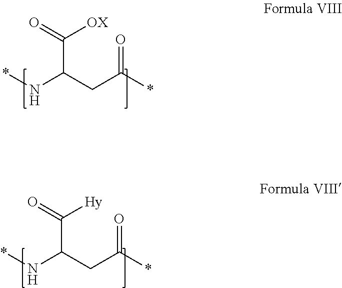

In one embodiment, the composition according to the invention is characterized in that n+m is comprised from 10 to 250.

In one embodiment, the composition according to the invention is characterized in that n+m is comprised from 10 to 200.

In one embodiment, the composition according to the invention is characterized in that n+m is comprised from 10 to 100.

In one embodiment, the composition according to the invention is characterized in that n+m is comprised from 10 to 50.

In one embodiment, the composition according to the invention is characterized in that n+m is comprised from 15 to 150.

In one embodiment, the composition according to the invention is characterized in that n+m is comprised from 15 to 100.

In one embodiment, the composition according to the invention is characterized in that n+m is comprised from 15 to 80.

In one embodiment, the composition according to the invention is characterized in that n+m is comprised from 15 to 65.

In one embodiment, the composition according to the invention is characterized in that n+m is comprised from 20 to 60.

In one embodiment, the composition according to the invention is characterized in that n+m is comprised from 20 to 50.

In one embodiment, the composition according to the invention is characterized in that n+m is comprised from 20 to 40.

In one embodiment, the concentration of co-polyamino acid bearing carboxylate charges and hydrophobic radicals is at most 40 mg/mL.

In one embodiment, the concentration of co-polyamino acid bearing carboxylate charges and hydrophobic radicals is at most 30 mg/mL.

In one embodiment, the concentration of co-polyamino acid bearing carboxylate charges and hydrophobic radicals is at most 20 mg/mL.

In one embodiment, the concentration of co-polyamino acid bearing carboxylate charges and hydrophobic radicals is at most 10 mg/mL.

In one embodiment, the concentration of co-polyamino acid bearing carboxylate charges and hydrophobic radicals is at most 5 mg/mL.

In one embodiment, the concentration of co-polyamino acid bearing carboxylate charges and hydrophobic radicals is at most 2.5 mg/mL.

In one embodiment, the concentration of co-polyamino acid bearing carboxylate charges and hydrophobic radicals is at most 1 mg/mL.

In one embodiment, the concentration of co-polyamino acid bearing carboxylate charges and hydrophobic radicals is at most 0.5 mg/mL.

The values of the degree of polymerization DP and the ratio i are estimated by ¹H NMR in D₂O by comparing the integration of the signals from the hydrophobic groups to that of the signals from the main chain of the co-polyamino acid.

In one embodiment, the co-polyamino acid bearing carboxylate charges and hydrophobic radicals is a co-polyamino acid according to formula VII or VIIa, wherein DP=23+/−5, i=0.05+/−0.02 and the at least one hydrophobic radical according to formula I is chosen from radicals according to formula I wherein, r=1, a=0, p=1, GpR corresponds to formula II wherein R is —CH2-CH2, GpC corresponds to formula IVd wherein x=15 and Cx is

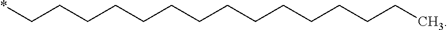 [SMILES: *CCCCCCCCCCCCCCC]

In one embodiment, the co-polyamino acid bearing carboxylate charges and hydrophobic radicals is a co-polyamino acid according to formula VII or VIIa, wherein DP=35+/−5, i=0.05+/−0.02 and the at least one hydrophobic radical according to formula I is chosen from radicals according to formula I wherein, r=1, a=0, p=1, GpR corresponds to formula II wherein R is —CH₂—CH₂, GpC corresponds to formula IVd wherein x=15 and Cx is

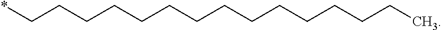 [SMILES: *CCCCCCCCCCCCCCC]

In one embodiment, the co-polyamino acid bearing carboxylate charges and hydrophobic radicals is a co-polyamino acid according to formula VII or VIIa, wherein DP=35+/−5, i=0.10+/−0.02 and the at least one hydrophobic radical according to formula I is chosen from radicals according to formula I wherein, r=1, a=0, p=1, GpR corresponds to formula II wherein R is —CH2-CH2, GpC corresponds to formula IVd wherein x=15 and Cx is

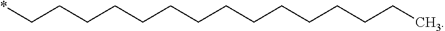 [SMILES: *CCCCCCCCCCCCCCC]

In one embodiment, the co-polyamino acid bearing carboxylate charges and hydrophobic radicals is a co-polyamino acid according to formula VII or VIIa, wherein DP=35+/−5, i=0.05+/−0.02 and the at least one hydrophobic radical according to formula I is chosen from the radicals according to formula I wherein, r=1, a=0, p=1, GpR corresponds to formula II wherein R is —CH₂CH₂—, GpC corresponds to formula IVd wherein x=16 and Cx is

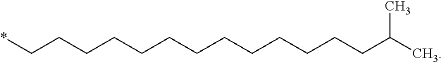 [SMILES: *CCCCCCCCCCCCCC(C)C]

In one embodiment, the co-polyamino acid bearing carboxylate charges and hydrophobic radicals is a co-polyamino acid according to formula VII or VIIa, wherein DP=23+/−5, i=0.05+/−0.02 and the at least one hydrophobic radical according to formula I is chosen from radicals according to formula I wherein, r=1, a=0, p=1, GpR corresponds to formula II wherein R is

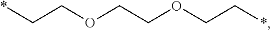

GpC corresponds to formula IVd wherein x=17 and Cx is

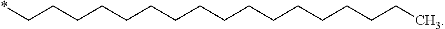 [SMILES: *CCCCCCCCCCCCCCCCC]

In one embodiment, the co-polyamino acid bearing carboxylate charges and hydrophobic radicals is a co-polyamino acid according to formula VII or VIIa, wherein DP=22+/−5, i=0.05+/−0.02 and the at least one hydrophobic radical according to formula I is chosen from radicals according to formula I wherein, r=1, a=0, p=1, GpR corresponds to formula II wherein R is

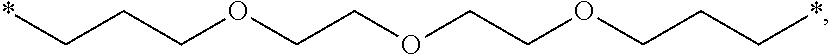

GpC corresponds to formula IVd wherein x=19 and Cx is

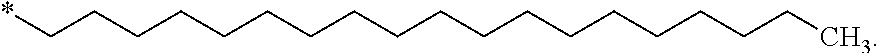 [SMILES: *CCCCCCCCCCCCCCCCCCC]

In one embodiment, the co-polyamino acid bearing carboxylate charges and hydrophobic radicals is a co-polyamino acid according to formula VII or VIIa, wherein DP=30+/−5, i=0.10+/−0.03 and the at least one hydrophobic radical according to formula I is chosen from radicals according to formula I wherein, r=1, a=0, p=1, GpR corresponds to formula II wherein R is

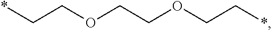

GpC corresponds to formula IVd wherein x=15 and Cx is

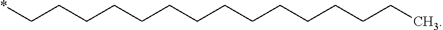 [SMILES: *CCCCCCCCCCCCCCC]

In one embodiment, the co-polyamino acid bearing carboxylate charges and hydrophobic radicals is a co-polyamino acid according to formula VII or VIIa, wherein DP=23+/−5, i=0.07+/−0.02 and the at least one hydrophobic radical according to formula I is chosen from radicals according to formula I wherein, r=1, a=0, p=1, GpR corresponds to formula II wherein R is

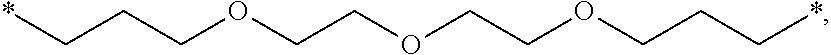

GpC corresponds to formula IVd wherein x=15 and Cx is

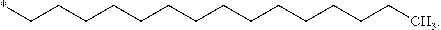 [SMILES: *CCCCCCCCCCCCCCC]

In one embodiment, the co-polyamino acid bearing carboxylate charges and hydrophobic radicals is a co-polyamino acid according to formula VII or VIIa, wherein DP=23+/−5, i=0.05+/−0.02 and the at least one hydrophobic radical according to formula I is chosen from radicals according to formula I wherein, r=1, a=0, p=1, GpR corresponds to formula II wherein R is, GpC corresponds to formula IVd wherein x=15 and Cx is.

In one embodiment, the co-polyamino acid bearing carboxylate charges and hydrophobic radicals is a co-polyamino acid according to formula VII or VIIa, wherein DP=26+/−5, i=0.04+/−0.02 and the at least one hydrophobic radical according to formula I is chosen from radicals according to formula I wherein, r=1, a=0, p=1, GpR corresponds to formula II wherein R is —CH₂—CH₂, GpC corresponds to formula IVa wherein b=1, B is

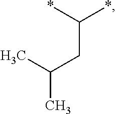

x=15 and Cx is

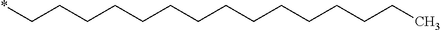 [SMILES: *CCCCCCCCCCCCCCC]

In one embodiment, the co-polyamino acid bearing carboxylate charges and hydrophobic radicals is a co-polyamino acid according to formula VII or VIIa, wherein DP=35+/−5, i=0.13+/−0.04 and the at least one hydrophobic radical according to formula I is chosen from the radicals according to formula I wherein, r=1, a=0, p=1, GpR corresponds to formula II wherein R is —CH₂CH₂—, GpC corresponds to formula IVa wherein b=1, B is

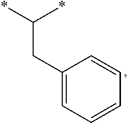 [SMILES: *C(*)Cc1ccccc1]

x=11 and Cx is

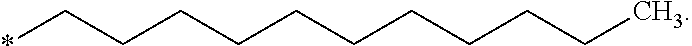 [SMILES: *CCCCCCCCCCC]

In one embodiment, the co-polyamino acid bearing carboxylate charges and hydrophobic radicals is a co-polyamino acid according to formula VII or VIIa, wherein DP=23+/−5, i=0.05+/−0.02 and the at least one hydrophobic radical according to formula I is chosen from radicals according to formula I wherein, r=1, a=0, p=1, GpR corresponds to formula II wherein R is

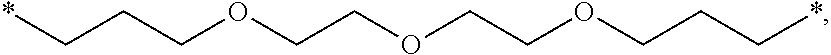

GpC corresponds to formula IVd in which x=19 and Cx is

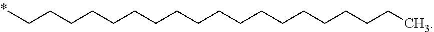 [SMILES: CCCCCCCCCCCCCCCCCCCC]

In one embodiment, the co-polyamino acid bearing carboxylate charges and hydrophobic radicals is a co-polyamino acid according to formula VII or VIIa, wherein DP=22+/−5, i=0.05+/−0.02 and the at least one hydrophobic radical according to formula I is chosen from radicals according to formula I wherein, r=1, a=1, p=2, GpR corresponds to formula II wherein R is —CH₂CH₂—, GpA corresponds to formula IIIb, GpC corresponds to formula IVd wherein x=11 and Cx is

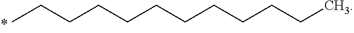

In one embodiment, the co-polyamino acid bearing carboxylate charges and hydrophobic radicals is a co-polyamino acid according to formula VII or VIIa, wherein DP=35+/−5, i=0.05+/−0.02 and the at least one hydrophobic radical according to formula I is chosen from radicals according to formula I wherein, r=1, a=1, p=2, GpR corresponds to formula II and wherein R is —CH₂CH₂—, GpA corresponds to formula IIIb, GpC corresponds to formula IVd wherein x=11 and Cx is

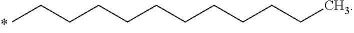

In one embodiment, the co-polyamino acid bearing carboxylate charges and hydrophobic radicals is a co-polyamino acid according to formula VII or VIIa, wherein DP=65+/−5, i=0.05+/−0.02 and the at least one hydrophobic radical according to formula I is chosen from radicals according to formula I wherein, r=1, a=1, p=2, GpR corresponds to formula II wherein R is —CH2CH2-, GpA corresponds to formula IIIb, GpC corresponds to formula IVd wherein x=11 and Cx is

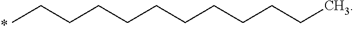

In one embodiment, the co-polyamino acid bearing carboxylate charges and hydrophobic radicals is a co-polyamino acid according to formula VII or VIIa, wherein DP=22+/−5, i=0.04+/−0.02 and the at least one hydrophobic radical according to formula I is chosen from radicals according to formula I wherein, r=1, a=1, p=2, GpR corresponds to formula II wherein R is —CH2CH2-, GpA corresponds to formula IIIb, GpC corresponds to formula IVd wherein x=13 and Cx is

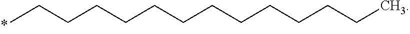

In one embodiment, the co-polyamino acid bearing carboxylate charges and hydrophobic radicals is a co-polyamino acid according to formula VII or VIIa, wherein DP=22+/−5, 0.03+/−0.01 and the at least one hydrophobic radical according to formula I is chosen from radicals according to formula I wherein, r=1, a=1, p=2, GpR corresponds to formula II wherein R is

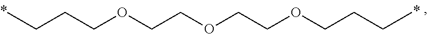

GpA corresponds to formula IIIb, GpC corresponds to formula IVd wherein x=13 and Cx is

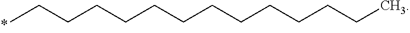

In one embodiment, the co-polyamino acid bearing carboxylate charges and hydrophobic radicals is a co-polyamino acid according to formula VII or VIIa, wherein DP=22+/−5, 0.07+/−0.02 and the at least one hydrophobic radical according to formula I is chosen from radicals according to formula I wherein, r=1, a=1, p=2, GpR corresponds to formula II wherein R is

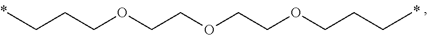

GpA corresponds to formula IIIb, GpC corresponds to formula IVd wherein x=9 and Cx is

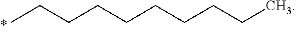

In one embodiment, the co-polyamino acid bearing carboxylate charges and hydrophobic radicals is a co-polyamino acid according to formula VII or VIIb, wherein DP=25+/−5, 0.033≤i≤0.05 and the hydrophobic radical according to formula I is chosen from the radicals according to formula I wherein, r=1, a=0, p=1, GpR corresponds to formula II wherein R is —CH₂—CH₂—, GpC corresponds to formula IVd wherein x=15 and Cₓ is

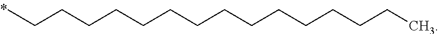 [SMILES: CCCCCCCCCCCCCCCC]

In one embodiment, the co-polyamino acid bearing carboxylate charges and hydrophobic radicals is a co-polyamino acid according to formula VII or VIIb, wherein DP=30+/−5, 0.028≤i≤0.04 and the at least one hydrophobic radical according to formula I is chosen from radicals according to formula I wherein, r=1, a=0, p=1, GpR corresponds to formula II wherein R is

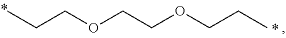

GpC corresponds to formula IVd wherein x=17 and Cₓ is

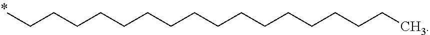 [SMILES: CCCCCCCCCCCCCCCCCC]

In one embodiment, the co-polyamino acid bearing carboxylate charges and hydrophobic radicals is a co-polyamino acid according to formula VII or VIIb, wherein DP=45+/−10, 0.018≤i≤0.028 and the at least one hydrophobic radical according to formula I is chosen from radicals according to formula I wherein, r=1, a=0, p=1, GpR corresponds to formula II wherein R is, GpC corresponds to formula IVd wherein x=17 and Cx is

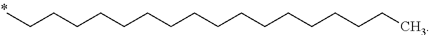 [SMILES: CCCCCCCCCCCCCCCCCC]

In one embodiment, the co-polyamino acid bearing carboxylate charges and hydrophobic radicals is a co-polyamino acid according to formula VII or VIIb, wherein DP=60+/−10, 0.014≤i≤0.02 and the at least one hydrophobic radical according to formula I is chosen from radicals according to formula I wherein, r=1, a=0, p=1, GpR corresponds to formula II wherein R is

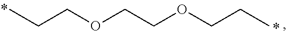

GpC corresponds to formula IVd wherein x=17 and Cₓ is

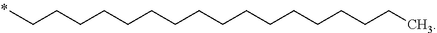 [SMILES: CCCCCCCCCCCCCCCCCC]

In one embodiment, the co-polyamino acid bearing carboxylate charges and hydrophobic radicals is a co-polyamino acid according to formula VII or VIIb, wherein DP=25+/−5, 0.033≤i≤0.05 and hydrophobic radical according to formula I is chosen from radicals according to formula I wherein, r=1, a=0, p=1, GpR corresponds to formula II wherein R is

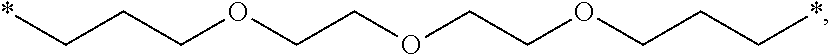

GpC corresponds to formula IVd wherein x=19 and Cₓ is

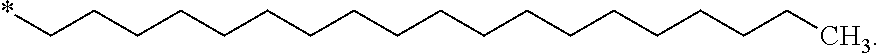 [SMILES: *CCCCCCCCCCCCCCCCCCC]

In one embodiment, the co-polyamino acid bearing carboxylate charges and hydrophobic radicals is a co-polyamino acid according to formula VII or VIIb, wherein DP=25+/−5, 0.025≤i≤0.07 and the at least one hydrophobic radical according to formula I is chosen from radicals according to formula I wherein, r=1, a=0, p=1, GpR corresponds to formula II wherein R is, GpC corresponds to formula IVd wherein x=19 and Cx is

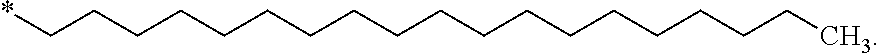 [SMILES: *CCCCCCCCCCCCCCCCCCC]

In one embodiment, the co-polyamino acid bearing carboxylate charges and hydrophobic radicals is a co-polyamino acid according to formula VII or VIIb, wherein DP=27+/−5, 0.031≤i≤0.045 and the at least one hydrophobic radical according to formula I is chosen from radicals according to formula I wherein, r=1, a=1, p=2, GpR corresponds to formula II wherein R is —CH₂CH₂—, GpA corresponds to formula IIIb, GpC corresponds to formula IVd wherein x=11 and Cₓ is

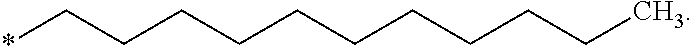 [SMILES: *CCCCCCCCCCC]

In one embodiment, the co-polyamino acid bearing carboxylate charges and hydrophobic radicals is a co-polyamino acid according to formula VII or VIIb, wherein DP=22+/−5, 0.037≤i≤0.055 and the at least one hydrophobic radical according to formula I is chosen from radicals according to formula I wherein, r=1, a=1, p=2, GpR corresponds to formula II wherein R is —CH₂CH₂—, GpA corresponds to formula IIIb, GpC corresponds to formula IVd wherein x=13 and Cₓ is

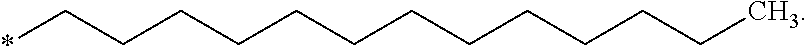 [SMILES: *CCCCCCCCCCCCC]

In one embodiment, the co-polyamino acid bearing carboxylate charges and hydrophobic radicals is a co-polyamino acid according to formula VII or VIIb, wherein DP=22+/−5, 0.037≤i≤0.055 and the at least one radical hydrophobic compound according to formula I is chosen from the radicals according to formula I wherein, r=1, a=1, p=2, GpR corresponds to formula II wherein R is

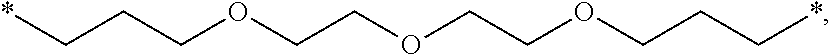

GpA corresponds to formula IIIb, GpC corresponds to formula IVd wherein x=13 and Cₓ is

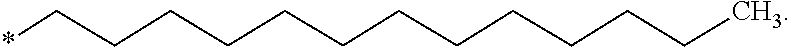 [SMILES: *CCCCCCCCCCCCC]

In one embodiment, the co-polyamino acid bearing carboxylate charges and hydrophobic radicals is a co-polyamino acid according to formula VII or VIIb, wherein DP=60+/−10, 0.014≤i≤0.02 and the at least one hydrophobic radical according to formula I is chosen from radicals according to formula I wherein, r=1, a=1, p=2, GpR corresponds to formula II wherein R is —CH2CH2-, GpA corresponds to formula IIIb, GpC corresponds to formula IVd wherein x=13 and Cx is

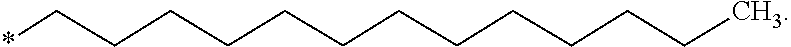 [SMILES: *CCCCCCCCCCCCC]

In one embodiment, the co-polyamino acid bearing carboxylate charges and hydrophobic radicals is a co-polyamino acid according to formula VII or VIIb, wherein DP=40+/−5, 0.022≤i≤0.029 and the at least one hydrophobic radical according to formula I is chosen from radicals according to formula I wherein, r=1, a=1, p=2, GpR corresponds to formula II wherein R is —CH2CH2-, GpA corresponds to formula IIIb, GpC corresponds to formula IVd wherein x=13 and Cx is

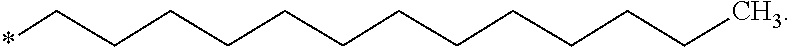 [SMILES: *CCCCCCCCCCCCC]

In one embodiment, the co-polyamino acid bearing carboxylate charges and hydrophobic radicals is a co-polyamino acid according to formula VII or VIIb, wherein DP=25+/−5, 0.02≤i≤0.06 and the at least one hydrophobic radical according to formula I is chosen from radicals according to formula I wherein, r=1, a=1, p=2, GpR corresponds to formula II wherein R is —CH2CH2-, GpA corresponds to formula IIIb, GpC corresponds to formula IVd wherein x=13 and Cx is

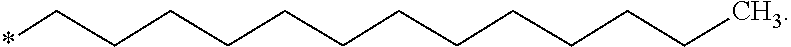 [SMILES: *CCCCCCCCCCCCC]

In one embodiment, the co-polyamino acid bearing carboxylate charges and hydrophobic radicals is a co-polyamino acid according to formula VII or VIIb, wherein DP=17+/−4, 0.04≤i≤0.1 and the at least one hydrophobic radical according to formula I is chosen from radicals according to formula I wherein, r=1, a=1, p=2, GpR corresponds to formula II wherein R is —CH2CH2-, GpA corresponds to formula IIIb, GpC corresponds to formula IVd wherein x=13 and Cx is

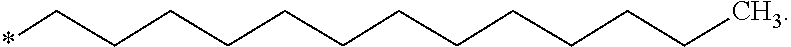 [SMILES: *CCCCCCCCCCCCC]

In one embodiment, the co-polyamino acid bearing carboxylate charges and hydrophobic radicals is a co-polyamino acid according to formula VII or VIIb, wherein DP=9+/−2, 0.09≤i≤0.2 and the at least one hydrophobic radical according to formula I is chosen from radicals according to formula I wherein, r=1, a=1, p=2, GpR corresponds to formula II wherein R is —CH2CH2-, GpA corresponds to formula IIIb, GpC corresponds to formula IVd wherein x=13 and Cx is

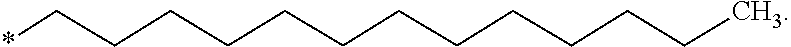 [SMILES: *CCCCCCCCCCCCC]

In one embodiment, the co-polyamino acid bearing carboxylate charges and hydrophobic radicals is a co-polyamino acid according to formula VII or VIIb, wherein DP=20+/−5, 0.04≤i≤0.08 and the at least one hydrophobic radical according to formula I is chosen from radicals according to formula I wherein, r=1, a=1, p=2, GpR corresponds to formula II wherein R is —CH2CH2-, GpA corresponds to formula IIIb, GpC corresponds to formula IVd wherein x=13 and Cx is

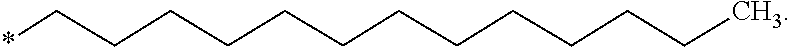 [SMILES: *CCCCCCCCCCCCC]

In one embodiment, the co-polyamino acid bearing carboxylate charges and hydrophobic radicals is a co-polyamino acid according to formula VII or VIIb, wherein DP=23+/−5, 0.035≤i≤0.08 and the at least one hydrophobic radical according to formula I is chosen from radicals according to formula I wherein, r=1, a=1, p=2, GpR corresponds to formula II wherein R is —CH2CH2-, GpA corresponds to formula IIIb, GpC corresponds to formula IVd wherein x=15 and Cx is

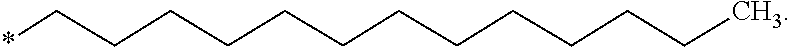 [SMILES: *CCCCCCCCCCCCC]

In one embodiment, the co-polyamino acid bearing carboxylate charges and hydrophobic radicals is a co-polyamino acid according to formula VII or VIIb, wherein DP=20+/−5, 0.04≤i≤0.08 and the at least one hydrophobic radical according to formula I is chosen from radicals according to formula I wherein, r=1, a=1, p=2, GpR corresponds to formula II wherein R is —CH₂CH₂—, GpA corresponds to formula IIIc, GpC corresponds to formula IVd wherein x=13 and Cₓ is

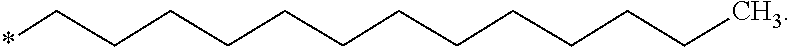 [SMILES: *CCCCCCCCCCCCC]

In one embodiment, the co-polyamino acid bearing carboxylate charges and hydrophobic radicals is a co-polyamino acid according to formula VII or VIIb, wherein DP=20+/−5, 0.04≤i≤0.08 and Hy, as well as R₁ and/or R₂ is a hydrophobic radical according to formula I is chosen from radicals according to formula I wherein, r=1, a=1, p=2, GpR corresponds to formula II wherein R is —CH₂—CH₂—, GpA corresponds to formula IIIb, GpC corresponds to formula IVd wherein x=13 and Cₓ is

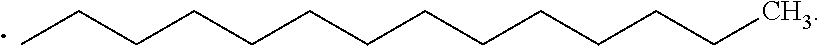

In one embodiment, the co-polyamino acid bearing carboxylate charges and hydrophobic radicals is a co-polyamino acid according to formula VII or VIIb, wherein DP=20+/−5, 0.04≤i≤0.08 and the at least one hydrophobic radical according to formula I is chosen from radicals according to formula I wherein, r=0, a=1, p=2, GpR corresponds to formula II wherein R is —CH₂—CH₂—, GpA corresponds to formula IIIb, GpC corresponds to formula IVd wherein x=13 and Cₓ is

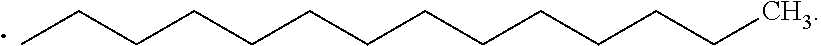

In one embodiment, the co-polyamino acid bearing carboxylate charges and hydrophobic radicals is a co-polyamino acid according to formula VII or VIIb, wherein DP=20+/−5, 0.08≤i≤0.20 and R₁ is a hydrophobic radical according to formula I chosen from radicals according to formula I wherein, r=0, a=1, p=2, GpR corresponds to formula II wherein R is —CH₂—CH₂—, GpA corresponds to formula IIIb, GpC satisfies formula IVd wherein x=13 and Cₓ is

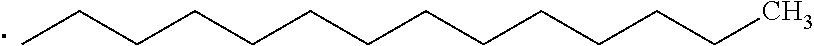

and R₂ a hydrophobic radical according to formula I is chosen from radicals according to formula I wherein, r=1, a=1, p=2, GpR corresponds to formula II wherein R is —CH₂CH₂—, GpA corresponds to formula IIIb, GpC corresponds to formula IVd wherein x=13 and Cₓ is

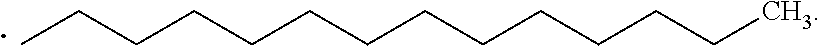

In one embodiment, the co-polyamino acid bearing carboxylate charges and hydrophobic radicals is a co-polyamino acid according to formula VII or VIIb, in which DP=25+/−5, 0.035≤i≤0.08 and the at least one hydrophobic radical according to formula I is chosen from radicals according to formula I in which, r=1, a=1, p=2, GpR corresponds to formula II in which R is —(CH₂)₆—, GpA corresponds to Formula IIIb, GpC corresponds to formula IVd in which x=14 and Cₓ is

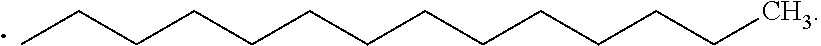

The invention also relates to said co-polyamino acids bearing carboxylate charges and hydrophobic radicals according to formula I and the precursors of said hydrophobic radicals.

The co-polyamino acids bearing carboxylate charges and hydrophobic radicals according to formula I are soluble in distilled water at a pH ranging from 6.0 to 8.0, at a temperature of 25° C. and at a concentration of less than 100 mg/ml.

In one embodiment, the invention also relates to the precursors of said hydrophobic radicals according to formula I′, V′ and VI′:

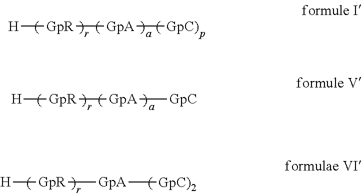

GpR, GpA, GpC, r, a, p have the above definitions.

In one embodiment, the composition according to the invention is characterized in that the co-polyamino acid is obtained from a polyamino acid obtained by polymerization.

In one embodiment, the composition according to the invention is characterized in that the co-polyamino acid is obtained from a polyamino acid obtained by ring opening polymerization of a glutamic acid N-carboxyanhydride derivative or an aspartic acid N-carboxyanhydride derivative.

In one embodiment, the composition according to the invention is characterized in that the co-polyamino acid is obtained from a polyamino acid obtained by polymerization of a glutamic acid N-carboxyanhydride derivative or an aspartic acid N-carboxyanhydride derivative as described in Adv. Polym. Sci. 2006, 202, 1-18 (Deming, T J).

In one embodiment, the composition according to the invention is characterized in that the co-polyamino acid is obtained from a polyamino acid obtained by polymerization of a glutamic acid N-carboxyanhydride derivative.

In one embodiment, the composition according to the invention is characterized in that the co-polyamino acid is obtained from a polyamino acid obtained by polymerization of a glutamic acid N-carboxyanhydride derivative chosen from the group consisting ofN-carboxyanhydride methyl glutamate (GluOMe-NCA), benzyl N-carboxyanhydride glutamate (GluOBzl-NCA) and t-butyl N-carboxyanhydride glutamate (GluOtBu-NCA).

In one embodiment, the glutamic acid N-carboxyanhydride derivative is methyl N-carboxyanhydride L-glutamate (L-GluOMe-NCA).

In one embodiment, the glutamic acid N-carboxyanhydride derivative is benzyl N-carboxyanhydride L-glutamate (L-GluOBzl-NCA).

In one embodiment, the composition according to the invention is characterized in that the co-polyamino acid is obtained from a polyamino acid obtained by polymerization of a glutamic acid N-carboxyanhydride derivative or an aspartic acid N-carboxyanhydride derivative using an organometallic complex of a transition metal as initiator, as described in Nature 1997, 390, 386-389 (Deming, T J).

In one embodiment, the composition according to the invention is characterized in that the co-polyamino acid is obtained from a polyamino acid obtained by polymerization of a glutamic acid N-carboxyanhydride derivative or an aspartic acid N-carboxyanhydride derivative using as initiator ammonia or a primary amine as described in FR 2,801,226 (Touraud, F. et al.) and references cited therein.

In one embodiment, the composition according to the invention is characterized in that the co-polyamino acid is obtained from a polyamino acid obtained by polymerization of a glutamic acid N-carboxyanhydride derivative or an aspartic acid N-carboxyanhydride derivative using as initiator hexamethyldisilazane as described in J. Am. Chem. Soc. 2007, 129, 14114-14115 (Lu H., et al.) or a silylated amine as described in J. Am. Chem. Soc. 2008, 130, 12562-12563 (Lu H., et al.).

In one embodiment, the composition according to the invention is characterized in that the process for synthesizing the polyamino acid obtained by polymerization of a glutamic acid N-carboxyanhydride derivative or an aspartic acid N-carboxyanhydride derivative acid from which the co-polyamino acid is obtained, comprises a ester function hydrolysis step.

In one embodiment, this ester hydrolysis step may consist of hydrolysis in an acidic medium or hydrolysis in a basic medium or may be carried out by hydrogenation.

In one embodiment, this ester group hydrolysis step is a hydrolysis in an acidic medium.

In one embodiment, this ester group hydrolysis step is carried out by hydrogenation.

In one embodiment, the composition according to the invention is characterized in that the co-polyamino acid is obtained from a polyamino acid obtained by depolymerization of a polyamino acid of higher molecular weight.

In one embodiment, the composition according to the invention is characterized in that the co-polyamino acid is obtained from a polyamino acid obtained by enzymatic depolymerization of a polyamino acid of higher molecular weight.

In one embodiment, the composition according to the invention is characterized in that the co-polyamino acid is obtained from a polyamino acid obtained by chemical depolymerization of a polyamino acid of higher molecular weight.

In one embodiment, the composition according to the invention is characterized in that the co-polyamino acid is obtained from a polyamino acid obtained by enzymatic and chemical depolymerization of a polyamino acid of higher molecular weight.

In one embodiment, the composition according to the invention is characterized in that the co-polyamino acid is obtained from a polyamino acid obtained by depolymerization of a polyamino acid of higher molecular weight chosen from the group consisting of sodium polyglutamate and sodium polyaspartate.

In one embodiment, the composition according to the invention is characterized in that the co-polyamino acid is obtained from a polyamino acid obtained by depolymerization of a sodium polyglutamate of higher molecular weight.

In one embodiment, the composition according to the invention is characterized in that co-polyamino acid is obtained from a polyamino acid obtained by depolymerization of a sodium polyaspartate of higher molecular weight.

In one embodiment, the composition according to the invention is characterized in that co-polyamino acid is obtained by grafting a hydrophobic group onto an acid poly-L-glutamic acid or poly-L-aspartic acid using amide bond formation processes well known to people skilled in the art.

In one embodiment, the composition according to the invention is characterized in that co-polyamino acid is obtained by grafting a hydrophobic group onto a poly-L-glutamic acid or poly-L-aspartic acid using amide bond formation processes used for peptide synthesis.

In one embodiment, the composition according to the invention is characterized in that co-polyamino acid is obtained by grafting a hydrophobic group on a poly-L-glutamic acid or poly-L-aspartic acid as described in patent FR 2,840,614 (Chan, Y P et al.).

Human glucagon is a highly conserved polypeptide comprising a single chain of 29 amino acid residues having the following sequence H-His-Ser-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Asn-Thr-OH.

It can be obtained in various ways, by peptide synthesis or by recombination.

Human glucagon is available via many sources. For example, it may be human glucagon produced by Bachem via peptide synthesis, in particular under reference 407473.

In one embodiment, the mass ratio of co-polyamino acid bearing carboxylate charges and hydrophobic radicals on glucagon is comprised from 1.5 to 25.

In one embodiment, the mass ratio of co-polyamino acid bearing carboxylate charges and hydrophobic radicals on glucagon is comprised from 2 to 20.

In one embodiment, the mass ratio of co-polyamino acid bearing carboxylate charges and hydrophobic radicals on glucagon is comprised from 2.5 to 15.

In one embodiment, the mass ratio of co-polyamino acid bearing carboxylate charges and hydrophobic radicals on glucagon is comprised from 2 to 10.

In one embodiment, the mass ratio of co-polyamino acid bearing carboxylate charges and hydrophobic radicals on glucagon is comprised from 2 to 7.

Human glucagon is used in dosages that vary depending on the application.

In emergency treatment of hypoglycemia the recommended dose is 1 mg intramuscularly or intravenously (0.5 mg if body weight is below 25 kg). This administration is carried out with a solution of human glucagon at a concentration of 1 mg/ml.

In pumps, the envisaged daily dose is about 0.5 mg; the solutions can thus comprise from 0.25 mg/ml to 5 mg/ml of human glucagon.

According to one embodiment, the solutions may comprise from 0.5 mg/ml to 3 mg/ml of human glucagon.

In the treatment of obesity the envisaged daily dose is about 0.5 mg; the solutions can thus comprise from 0.25 mg/ml to 5 mg/ml of human glucagon.

In one embodiment, the concentration of human glucagon is comprised from 0.25 to 5 mg/mL.

In one embodiment, the concentration of human glucagon is comprised from 0.5 to 4 mg/mL.

In one embodiment, the concentration of human glucagon is comprised from 0.75 to 3 mg/mL.

In one embodiment, the concentration of human glucagon is comprised from 0.75 to 2.5 mg/mL.

In one embodiment, the concentration of human glucagon is comprised from 0.75 to 2 mg/mL.

In one embodiment, the concentration of human glucagon is comprised from 1 to 2 mg/mL.

In one embodiment, the molar ratio [hydrophobic radical]/[human glucagon] is less than 20.

In one embodiment, the molar ratio [hydrophobic radical]/[human glucagon] is less than 15.

In one embodiment, the molar ratio [hydrophobic radical]/[human glucagon] is less than 10.

In one embodiment, the molar ratio [hydrophobic radical]/[human glucagon] is less than 5.

In one embodiment, the molar ratio [hydrophobic radical]/[human glucagon] is less than 2.5.

In one embodiment, the molar ratio [hydrophobic radical]/[human glucagon] is less than 1.5.

In one embodiment, the molar ratio [co-polyamino acid bearing carboxylate charges and hydrophobic radicals Hy]/[human glucagon] is less than 20.

In one embodiment, the molar ratio [co-polyamino acid bearing carboxylate charges and hydrophobic radicals Hy]/[human glucagon] is less than 15.

In one embodiment, the molar ratio [co-polyamino acid bearing carboxylate charges and hydrophobic radicals Hy]/[human glucagon] is less than 10.

In one embodiment, the molar ratio [co-polyamino acid bearing carboxylate charges and hydrophobic radicals Hy]/[human glucagon] is less than 5.

In one embodiment, the molar ratio [co-polyamino acid bearing carboxylate charges and hydrophobic radicals Hy]/[human glucagon] is less than 2.5.

In one embodiment, the molar ratio [co-polyamino acid bearing carboxylate charges and hydrophobic radicals Hy]/[human glucagon] is less than 1.5.

In one embodiment, the composition further comprises a nicotinic compound or one of its derivatives.

In one embodiment, the composition comprises nicotinamide.

In one embodiment, the concentration of nicotinamide ranges from 10 to 160 mM.

In one embodiment, the concentration of nicotinamide ranges from 20 to 150 mM.

In one embodiment, the concentration of nicotinamide ranges from 40 to 120 mM.

In one embodiment, the concentration of nicotinamide ranges from 60 to 100 mM.

In one embodiment, the composition further comprises a polyanionic compound.

In one embodiment, the polyanionic compound is chosen from the group consisting of polycarboxylic acids and their Na⁺, K⁺, Ca²⁺ or Mg²⁺ salts.

In one embodiment, polycarboxylic acid is chosen from the group consisting of citric acid, tartaric acid, and their Na⁺, K⁺, Ca²⁺ or Mg²⁺ salts.

In one embodiment, the polyanionic compound is chosen from the group consisting of polycarboxylic acids and their Na⁺, K⁺, Ca²⁺ or Mg²⁺ salts.

In one embodiment, the polyphosphoric acid is triphosphate and its N⁺, K⁺, Ca²⁺ or Mg²⁺ salts.

In one embodiment, the polyanionic compound is citric acid and its Na⁺, K⁺, Ca²⁺ or Mg²⁺ salts.

In one embodiment, the polyanionic compound is tartaric acid and its Na⁺, K⁺, Ca²⁺ or Mg²⁺ salts.

In one embodiment, the polyanionic compound is triphosphoric acid and its Na⁺, K⁺, Ca²⁺ or Mg²⁺ obtained salts.

In one embodiment, the concentration of polyanionic compound is comprised from 1 to 20 mM.

In one embodiment, the concentration of polyanionic compound is comprised from 2 to 15 mM.

In one embodiment, the concentration of polyanionic compound is comprised from 3 to 12 mM.

In one embodiment, the concentration of polyanionic compound is 10 mM.

In one embodiment, the concentration of polyanionic compound is 5 mM.

In one embodiment, the concentration of polyanionic compound is 10 mM for glucagon concentrations ranging between 0.5 mg/ml and 3 mg/ml.

In one embodiment, the concentration of polyanionic compound is 10 mM for glucagon concentrations ranging between 0.5 mg/ml and 2 mg/ml.

In one embodiment, the concentration of polyanionic compound is 10 mM for glucagon concentrations ranging between 1 mg/ml and 2 mg/ml.

In one embodiment, the concentration of polyanionic compound is 5 mM for glucagon concentrations ranging between 0.5 mg/ml and 3 mg/ml.

In one embodiment, the concentration of polyanionic compound is 5 mM for glucagon concentrations ranging between 0.5 mg/ml and 2 mg/ml.

In one embodiment, the concentration of polyanionic compound is 5 mM for glucagon concentrations ranging between 1 mg/ml and 2 mg/ml.

In one embodiment, the concentration of citric acid and its Na⁺, K⁺, Ca²⁺ or Mg²⁺ salts is comprised from 1 to 20 mM.

In one embodiment, the concentration of citric acid and its Na⁺, K⁺, Ca²⁺ or Mg²⁺ salts is comprised from 2 to 15 mM.

In one embodiment, the concentration of citric acid and its Na⁺, K⁺, Ca²⁺ or Mg²⁺ salts is comprised from 3 to 12 mM.

In one embodiment, the concentration of citric acid and its Na⁺, K⁺, Ca²⁺ or Mg²⁺ salts is 10 mM.

In one embodiment, the concentration of citric acid and its Na⁺, K⁺, Ca²⁺ or Mg²⁺ salts is 5 mM.

In one embodiment, the concentration of citric acid and its Na⁺, K⁺, Ca²⁺ or Mg²⁺ salts is 10 mM for glucagon concentrations between 0.5 mg/ml and 3 mg/ml.

In one embodiment, the concentration of citric acid and its Na⁺, K⁺, Ca²⁺ or Mg²⁺ salts is 10 mM for glucagon concentrations between 0.5 mg/ml and 2 mg/ml.

In one embodiment, the concentration of citric acid and its Na⁺, K⁺, Ca²⁺ or Mg²⁺ salts is 10 mM for glucagon concentrations between 1 mg/ml and 2 mg/ml.

In one embodiment, the concentration of citric acid and its Na⁺, K⁺, Ca²⁺ or Mg²⁺ salts is 5 mM for glucagon concentrations between 0.5 mg/ml and 3 mg/ml.

In one embodiment, the concentration of citric acid and its Na⁺, K⁺, Ca²⁺ or Mg²⁺ salts is 5 mM for glucagon concentrations between 0.5 mg/ml and 2 mg/ml.

In one embodiment, the concentration of citric acid and its Na⁺, K⁺, Ca²⁺ or Mg²⁺ salts is 5 mM for glucagon concentrations between 1 mg/ml and 2 mg/ml.

In one embodiment, the pharmaceutical composition further comprises at least one absorption promoter chosen from absorption promoters, diffusion promoters or vasodilator agents, individually or in combination.

Absorption promoters include, but are not limited to, surfactants, for example, bile salts, fatty acid salts, or phospholipids; nicotinic agents, such as nicotinamides, nicotinic acids, niacin, niacin amide, vitamin B3 and their salts; inhibitors of pancreatic trypsin; magnesium salts; polyunsaturated fatty acids; phosphatidylcholine didecanoyl; aminopolycarboxylates; tolmetin; sodium caprate; salicylic acid; oleic acid; linoleic acid; eicosapentaenoic acid (EPA); docosahexaenoic acid (DHA); benzyl acid; donors of nitric oxide, for example, 3-(2-Hydroxy-1-(1-methylethyl)-2-nitrosohydrazino)-1-propanamine, N-ethyl-2-(1-ethyl-hydroxy 2-1-nitrosohydrazino)-ethanamine, or S-nitroso-N-acetylpenicillamine; bile acids, glycine in its bile acid conjugated form; sodium ascorbate, potassium ascorbate; sodium salicylate, potassium salicylate, acetylsalicylic acid, salicylosalicylic acid, aluminum acetylsalicylate, choline salicylate, salicylamide, lysine acetylsalicylate; exalamide; the diflunisal; ethenzamide; EDTA; alone or in combination.

In one embodiment, the pharmaceutical composition further comprises at least one diffusion promoter. Examples of diffusion promoters include, but are not limited to, glycosaminoglycanases, for example, hyaluronidase.

In one embodiment, the pharmaceutical composition further comprises at least one vasodilator.

In one embodiment, the pharmaceutical composition further comprises at least one vasodilator causing hyperpolarization by blocking calcium ion channels.

In one embodiment, the vasodilator agent causing hyperpolarization by blocking the ion channels of calcium is adenosine, a hyperpolarizing agent obtained from endothelium, a phosphodiesterase type 5 (PDE5) inhibitor, a potassium channel opening agent or any combination of these agents.

In one embodiment, the pharmaceutical composition further comprises at least one cAMP mediated vasodilator.

In one embodiment, the pharmaceutical composition further comprises at least one cGMP-mediated vasodilator.

In one embodiment, the pharmaceutical composition further comprises at least one vasodilating agent chosen from the group consisting of vasodilator agents that react by causing hyperpolarization by blocking calcium ion channels, cAMP-mediated vasodilator agents, and cGMP-mediated vasodilators agents.

At least one vasodilator is chosen from the group consisting of nitrogen monoxide donors, for example, nitroglycerin, isosorbide dinitrate, isosorbide mononitrate, amyl nitrate, erythrityl, tetranitrate, and nitroprusside); prostacyclin and its analogues, for example epoprostenol sodium, iloprost, epoprostenol, treprostinil or selexipag; histamine, 2-methylhistamine, 4-methylhistamine; 2-(2-pyridyl) ethylamine, 2-(2-thiazolyl) ethylamine; papaverine, papaverine hydrochloride; minoxidil; dipyridamole; hydralazine; adenosine, adenosine triphosphate; uridine trisphosphate; the GPLC; L-carnitine; arginine; prostaglandin D2; potassium salts; and in some cases, α1 and α2 receptor antagonists, e.g., prazosin, phenoxybenzamine, phentolamine, dibenamine, moxisylyte hydrochloride and tolazoline), betazole, dimaprit; J32 receptor agonists, for example, isoproterenol, dobutamine, albuterol, terbutaline, aminophylline, theophylline, caffeine; alprostadil, ambrisentan; the cabergoline; diazoxide; dihydralazine mesilate; diltiazem hydrochloride; enoximone; flunarizine hydrochloride; Ginkgo biloba extract; levosimendan; molsidomine; the acidic oxalate of naftidrofuryl, nicorandil, pentoxifylline; phenoxybenzamine chloride; the piribedil base; the piribedil mesilate; regadenoson monohydrate; riociguat; sildenafil citrate, tadalafil, vardenafil hydrochloride trihydrate; trimetazidine hydrochloride; trinitrine; verapamil hydrochloride; endothelin receptor antagonists, for example avanafil and bosentran monohydrate; and calcium channel blockers, for example, amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nimodipine, nisoldipine, nitrendipine, prandipine; alone or in combination.

According to one embodiment, the vasodilator agent is treprostinil.

In one embodiment, the composition comprises in combination, a polyanionic compound and an absorption promoter.

In one embodiment, the composition comprises in combination, citric acid and/or its Na⁺, K⁺, Ca²⁺ or Mg²⁺ salts and an absorption promoter.

In one embodiment, the polyanionic compound is citric acid and its Na⁺, K⁺, Ca²⁺ or Mg²⁺ salts.

In one embodiment, the composition comprises a combination of a polyanionic compound, an absorption promoter and optionally NaCl.

In one embodiment, the composition comprises a combination of citric acid and/or its Na⁺, K⁺, Ca²⁺ or Mg²⁺ salts, nicotinamide or treprostinil and optionally NaCl.

In one embodiment, the composition comprises in combination, citric acid and/or its Na⁺, K⁺, Ca²⁺ or Mg²⁺ salts, nicotinamide or treprostinil and NaCl, and is intended to be administered intramuscularly.

In one embodiment, the composition comprises in combination citric acid and/or its Na⁺, K⁺, Ca²⁺ or Mg²⁺ salts, nicotinamide and optionally NaCl and is intended to be administered intramuscularly.

In one embodiment, the composition comprises in combination citric acid and/or its Na⁺, K⁺, Ca²⁺ or Mg²⁺ salts, treprostinil and optionally NaCl and is intended to be administered intramuscularly.

In one embodiment, the composition comprises in combination, citric acid and/or its Na⁺, K⁺, Ca²⁺ or Mg²⁺ salts, nicotinamide or treprostinil and optionally NaCl, and is intended to be administered intramuscularly.

In one embodiment, the composition comprises in combination citric acid and/or its Na⁺, K⁺, Ca²⁺ or Mg²⁺ salts, nicotinamide and optionally NaCl and is intended to be administered subcutaneously.

In one embodiment, the composition comprises in combination citric acid and/or its Na salts⁺, K⁺, Ca²⁺ or Mg²⁺ salts, treprostinil and optionally NaCl and is intended to be administered subcutaneously.

In one embodiment, the compositions according to the invention further comprise a gastrointestinal hormone.

“Gastrointestinal hormones” is understood to mean hormones chosen from the group consisting of GLP-1 RAs for human Glucagon-Like Peptide-1 (Glucagon like peptide-1 receptor agonist) Glucagon-like and GIP (Glucose-dependent insulinotropic peptide) agonists, dependent insulinotropic peptide), oxyntomodulin (a derivative of human proglucagon), peptide YY, amylin, cholecystokinin, pancreatic polypeptide (PP), ghrelin and enterostatin, their analogues or derivatives and/or their pharmaceutically acceptable salts.

In one embodiment, gastrointestinal hormones are analogues or derivatives of GLP-1 RA (glucagon-like peptide-1 receptor agonist) chosen from the group consisting of exenatide or Byetta® (ASTRA-ZENECA), liraglutide or Victoza® (NOVO NORDISK), Lixisenatide or Lyxumia® (SANOFI), albiglutide or Tanzeum® (GSK) or dulaglutide or Trulicity® (ELI LILLY & CO), their analogues or derivatives and their pharmaceutically acceptable salts.

In one embodiment, the gastrointestinal hormone is pramlintide or Symlin® (ASTRA-ZENECA).

In one embodiment, the gastrointestinal hormone is exenatide or Byetta® its analogues or derivatives and their pharmaceutically acceptable salts.

In one embodiment, the gastrointestinal hormone is liraglutide or Victoza® its analogues or derivatives and their pharmaceutically acceptable salts.

In one embodiment, the gastrointestinal hormone is lixisenatide or Lyxumia® its analogues or derivatives and their pharmaceutically acceptable salts.

In one embodiment, the gastrointestinal hormone is albiglutide or Tanzeum® its analogues or derivatives and their pharmaceutically acceptable salts.

In one embodiment, the gastrointestinal hormone is dulaglutide or Trulicity® its analogues or derivatives and their pharmaceutically acceptable salts.

In one embodiment, the gastrointestinal hormone is pramlintide or Symlin®, its analogues or derivatives and their pharmaceutically acceptable salts.

“Analogue”, when it is used in reference to a peptide or a protein, is understood to mean a peptide or a protein, wherein one or more constituent amino acid residues have been substituted by other amino acid residues and/or wherein one or more constituent amino acid residues have been deleted and/or wherein one or more constituent amino acid residues have been added. The percentage of homology allowed for the current definition of an analogue is 50%.

“Derivative”, when used in reference to a peptide or a protein, is understood to mean a peptide or protein or an analogue chemically modified with a substituent that is not present in the peptide or protein or reference analogue, in other words, a peptide or protein that has been modified by creating covalent bond to introduce substituents.

In one embodiment, the substituent is chosen from the group consisting of fatty chains.

In one embodiment, the concentration of gastrointestinal hormone is comprised from 0.01 to 10 mg/mL.

In one embodiment, the concentration of exenatide, its analogues or derivatives and their pharmaceutically acceptable salts is within a range of 0.04 to 0.5 mg/mL.

In one embodiment, the concentration of liraglutide, its analogues or derivatives and their pharmaceutically acceptable salts is within a range of 1 to 10 mg/mL.

In one embodiment, the concentration of lixisenatide, its analogues or derivatives and their pharmaceutically acceptable salts is within a range of 0.01 to 1 mg/mL.

In one embodiment, the concentration of pramlintide, its analogues or derivatives and their pharmaceutically acceptable salts is comprised from 0.1 to 5 mg/mL.

In one embodiment, the compositions according to the invention are produced by mixing human glucagon solutions obtained by reconstituting lyophilisate and GLP-1 RA (Glucagon like peptide-1 receptor agonist) GLP-1 RA solutions, analogue or GLP-1 RA derivative, said GLP-1 RA solutions being commercial or reconstituted from lyophilizate.

In one embodiment, the compositions according to the invention further comprise buffers.

In one embodiment, the compositions according to the invention comprise buffers at concentrations ranging from 0 to 100 mM.

In one embodiment, the compositions according to the invention comprise buffers at concentrations ranging from 15 to 50 mM.

In one embodiment, the compositions according to the invention comprise a buffer chosen from the group consisting of a phosphate buffer or Tris (trishydroxymethylaminomethane).

In one embodiment, the buffer is sodium phosphate.

In one embodiment, the buffer is Tris (trishydroxymethylaminomethane).

The invention also relates to compositions which further comprise ionic species, the said ionic species improve the stability of the compositions.

In one embodiment, the ionic species comprise less than 10 carbon atoms.

The invention also relates to the use of ionic species chosen from the group of anions, cations and/or zwitterions to improve the physicochemical stability of the compositions.

The said ionic species are chosen from the group of anions, cations and/or zwitterions. Zwitterion refers to a species bearing at least one positive charge and at least one negative charge on two non-adjacent atoms.

The said ionic species are used alone or as a mixture and preferably as a mixture.

In one embodiment, the anions are chosen from organic anions.

In one embodiment the organic anions comprise less than 10 carbon atoms.

In one embodiment, the organic anions are chosen from the group consisting of acetate, citrate and succinate

In one embodiment, the anions are chosen from anions of mineral origin.

In one embodiment, the anions of mineral origin are chosen from the group consisting of sulfates, phosphates and halides, in particular chlorides.

In one embodiment, the cations are chosen from organic cations.

In one embodiment, the organic cations comprise less than 10 carbon atoms.

In one embodiment, the organic cations are chosen from the group consisting of ammoniums, like 2-Amino-2-(hydroxymethyl) propane-1,3-diol wherein the amine is in ammonium form.

In one embodiment, cations are chosen from cations of mineral origin.

In one embodiment, the cations of mineral origin are chosen from the group consisting of zinc, in particular Zn2+ and alkali metals, especially Na+ and K+.

In one embodiment, the zwitterions are chosen from zwitterions of organic origin.

In one embodiment, zwitterions of organic origin are chosen from amino acids.

In one embodiment the amino acids are chosen from aliphatic amino acids in the group consisting of glycine, alanine, valine, isoleucine and leucine.

In one embodiment, the amino acids are chosen from cyclic amino acids in the group consisting of proline.

In one embodiment the amino acids are chosen from hydroxylated or sulfur amino acids in the group consisting of cysteine, serine, threonine, and methionine.

In one embodiment, the amino acids are chosen from aromatic amino acids in the group consisting of phenylalanine, tyrosine and tryptophan.

In one embodiment, the amino acids are chosen from amino acids whose carboxyl function of the side chain is amidified in the group consisting of asparagine and glutamine.

In one embodiment, zwitterions of organic origin are chosen from the group consisting of amino acids having an uncharged side chain.

In one embodiment, zwitterions of organic origin are chosen from the group consisting of amino diacids or acidic amino acids.

In one embodiment, the amino diacids are chosen from the group consisting of glutamic acid and aspartic acid, optionally in the form of salts.

In one embodiment, zwitterions of organic origin are chosen from the group consisting of basic or so-called “cationic” amino acids.

In one embodiment, “cationic” amino acids are chosen from arginine, histidine and lysine, especially arginine and lysine.

In particular, zwitterions comprise as many negative charges as positive charges and therefore a nil overall charge at the isoelectric point and/or at a pH ranging from 6 to 8.

Said ionic species are introduced into the compositions in the form of salts. The introduction of these can be in solid form before dissolution in the compositions, or in the form of a solution, in particular of concentrated solution.

For example, mineral-based cations are provided in the form of salts chosen from sodium chloride, zinc chloride, sodium phosphate, sodium sulfate, etc.

For example, anions of organic origin are provided in the form of salts chosen from sodium or potassium citrate, sodium acetate.

For example, amino acids are added in the form of salts chosen from arginine hydrochloride, histidine hydrochloride or in non-salified form, for example histidine or arginine.

In one embodiment, the total molar concentration of ionic species in the composition is greater than or equal to 10 mM.

In one embodiment, the total molar concentration of ionic species in the composition is greater than or equal to 20 mM.

In one embodiment, the total molar concentration of ionic species in the composition is greater than or equal to 30 mM.

In one embodiment, the total molar concentration of ionic species in the composition is greater than or equal to 50 mM.

In one embodiment, the total molar concentration of ionic species in the composition is greater than or equal to 75 mM.

In one embodiment, the total molar concentration of ionic species in the composition is greater than or equal to 100 mM.

In one embodiment, the total molar concentration of ionic species in the composition is greater than or equal to 200 mM.

In one embodiment, the total molar concentration of ionic species in the composition is greater than or equal to 300 mM.

In one embodiment, the total molar concentration of ionic species in the composition is greater than or equal to 500 mM.

In one embodiment, the total molar concentration of ionic species in the composition is greater than or equal to 600 mM.

In one embodiment, the total molar concentration of ionic species in the composition is greater than or equal to 700 mM.

In one embodiment, the total molar concentration of ionic species in the composition is greater than or equal to 800 mM.

In one embodiment, the total molar concentration of ionic species in the composition is greater than or equal to 900 mM.

In one embodiment, the total molar concentration of ionic species in the composition is less than or equal to 1000 mM.

In one embodiment, the total molar concentration of ionic species in the composition is less than or equal to 1500 mM.

In one embodiment, the total molar concentration of ionic species in the composition is less than or equal to 1200 mM.

In one embodiment, the total molar concentration of ionic species in the composition is less than or equal to 1000 mM.

In one embodiment, the total molar concentration of ionic species in the composition is less than or equal to 900 mM.

In one embodiment, the total molar concentration of ionic species in the composition is less than or equal to 800 mM.

In one embodiment, the total molar concentration of ionic species in the composition is less than or equal to 700 mM.

In one embodiment, the total molar concentration of ionic species in the composition is less than or equal to 600 mM.

In one embodiment, the total molar concentration of ionic species in the composition is less than or equal to 500 mM.

In one embodiment, the total molar concentration of ionic species in the composition is less than or equal to 400 mM.

In one embodiment, the total molar concentration of ionic species in the composition is less than or equal to 300 mM.

In one embodiment, the total molar concentration of ionic species in the composition is less than or equal to 200 mM.

In one embodiment, the total molar concentration of ionic species in the composition is less than or equal to 100 mM.

In one embodiment, the total molar concentration of ionic species in the composition is comprised from 10 to 1000 mM.

In one embodiment, the total molar concentration of ionic species in the composition is comprised from 20 to 1000 mM.

In one embodiment, the total molar concentration of ionic species in the composition is comprised from 30 to 1000 mM.

In one embodiment, the total molar concentration of ionic species in the composition is comprised from 50 to 1000 mM.

In one embodiment, the total molar concentration of ionic species in the composition is comprised from 75 to 1000 mM.

In one embodiment, the total molar concentration of ionic species in the composition is comprised from 100 to 1000 mM.

In one embodiment, the total molar concentration of ionic species in the composition is comprised from 200 to 1000 mM.

In one embodiment, the total molar concentration of ionic species in the composition is comprised from 300 to 1000 mM.

In one embodiment, the total molar concentration of ionic species in the composition is comprised from 400 to 1000 mM.

In one embodiment, the total molar concentration of ionic species in the composition is comprised from 500 to 1000 mM.

In one embodiment, the total molar concentration of ionic species in the composition is comprised from 600 to 1000 mM.

In one embodiment, the total molar concentration of ionic species in the composition is comprised from 10 to 900 mM.

In one embodiment, the total molar concentration of ionic species in the composition is comprised from 20 to 900 mM.

In one embodiment, the total molar concentration of ionic species in the composition is comprised from 30 to 900 mM.

In one embodiment, the total molar concentration of ionic species in the composition is comprised from 50 to 900 mM.

In one embodiment, the total molar concentration of ionic species in the composition is comprised from 75 to 900 mM.

In one embodiment, the total molar concentration of ionic species in the composition is comprised from 100 to 900 mM.

In one embodiment, the total molar concentration of ionic species in the composition is comprised from 200 to 900 mM.

In one embodiment, the total molar concentration of ionic species in the composition is comprised from 300 to 900 mM.

In one embodiment, the total molar concentration of ionic species in the composition is comprised from 400 to 900 mM.

In one embodiment, the total molar concentration of ionic species in the composition is comprised from 500 to 900 mM.

In one embodiment, the total molar concentration of ionic species in the composition is comprised from 600 to 900 mM.

In one embodiment, the total molar concentration of ionic species in the composition is comprised from 10 to 800 mM.

In one embodiment, the total molar concentration of ionic species in the composition is comprised from 20 to 800 mM.

In one embodiment, the total molar concentration of ionic species in the composition is comprised from 30 to 800 mM.

In one embodiment, the total molar concentration of ionic species in the composition is comprised from 50 to 800 mM.

In one embodiment, the total molar concentration of ionic species in the composition is comprised from 75 to 800 mM.

In one embodiment, the total molar concentration of ionic species in the composition is comprised from 100 to 800 mM.

In one embodiment, the total molar concentration of ionic species in the composition is comprised from 200 to 800 mM.

In one embodiment, the total molar concentration of ionic species in the composition is comprised from 300 to 800 mM.

In one embodiment, the total molar concentration of ionic species in the composition is comprised from 400 to 800 mM.

In one embodiment, the total molar concentration of ionic species in the composition is comprised from 500 to 800 mM.

In one embodiment, the total molar concentration of ionic species in the composition is comprised from 600 to 800 mM.

In one embodiment, the total molar concentration of ionic species in the composition is comprised from 10 to 700 mM.

In one embodiment, the total molar concentration of ionic species in the composition is comprised from 20 to 700 mM.

In one embodiment, the total molar concentration of ionic species in the composition is comprised from 30 to 700 mM.

In one embodiment, the total molar concentration of ionic species in the composition is comprised from 50 to 700 mM.

In one embodiment, the total molar concentration of ionic species in the composition is comprised from 75 to 700 mM.

In one embodiment, the total molar concentration of ionic species in the composition is comprised from 100 to 700 mM.

In one embodiment, the total molar concentration of ionic species in the composition is comprised from 200 to 700 mM.

In one embodiment, the total molar concentration of ionic species in the composition is comprised from 300 to 700 mM.

In one embodiment, the total molar concentration of ionic species in the composition is comprised from 400 to 700 mM.

In one embodiment, the total molar concentration of ionic species in the composition is comprised from 500 to 700 mM.

In one embodiment, the total molar concentration of ionic species in the composition is comprised from 600 to 700 mM.

In one embodiment, the total molar concentration of ionic species in the composition is comprised from 10 to 600 mM.

In one embodiment, the total molar concentration of ionic species in the composition is comprised from 20 to 600 mM.

In one embodiment, the total molar concentration of ionic species in the composition is comprised from 30 to 600 mM.

In one embodiment, the total molar concentration of ionic species in the composition is comprised from 50 to 600 mM.

In one embodiment, the total molar concentration of ionic species in the composition is comprised from 75 to 600 mM.

In one embodiment, the total molar concentration of ionic species in the composition is comprised from 100 to 600 mM.

In one embodiment, the total molar concentration of ionic species in the composition is comprised from 200 to 600 mM.

In one embodiment, the total molar concentration of ionic species in the composition is comprised from 300 to 600 mM.

In one embodiment, the total molar concentration of ionic species in the composition is comprised from 400 to 600 mM.

In one embodiment, the total molar concentration of ionic species in the composition is comprised from 500 to 600 mM.

In one embodiment, the total molar concentration of ionic species in the composition is comprised from 10 to 500 mM.

In one embodiment, the total molar concentration of ionic species in the composition is comprised from 20 to 500 mM.

In one embodiment, the total molar concentration of ionic species in the composition is comprised from 30 to 500 mM.

In one embodiment, the total molar concentration of ionic species in the composition is comprised from 50 to 500 mM.

In one embodiment, the total molar concentration of ionic species in the composition is comprised from 75 to 500 mM.

In one embodiment, the total molar concentration of ionic species in the composition is comprised from 100 to 500 mM.

In one embodiment, the total molar concentration of ionic species in the composition is comprised from 200 to 500 mM.

In one embodiment, the total molar concentration of ionic species in the composition is comprised from 300 to 500 mM.

In one embodiment, the total molar concentration of ionic species in the composition is comprised from 400 to 500 mM.

In one embodiment, the total molar concentration of ionic species in the composition is comprised from 10 to 400 mM.

In one embodiment, the total molar concentration of ionic species in the composition is comprised from 20 to 400 mM.

In one embodiment, the total molar concentration of ionic species in the composition is comprised from 30 to 400 mM.

In one embodiment, the total molar concentration of ionic species in the composition is comprised from 50 to 400 mM.

In one embodiment, the total molar concentration of ionic species in the composition is comprised from 75 to 400 mM.

In one embodiment, the total molar concentration of ionic species in the composition is comprised from 100 to 400 mM.

In one embodiment, the total molar concentration of ionic species in the composition is comprised from 200 to 400 mM.

In one embodiment, the total molar concentration of ionic species in the composition is comprised from 300 to 400 mM.

In one embodiment, the total molar concentration of ionic species in the composition is comprised from 10 to 300 mM.

In one embodiment, the total molar concentration of ionic species in the composition is comprised from 20 to 300 mM.

In one embodiment, the total molar concentration of ionic species in the composition is comprised from 30 to 300 mM.

In one embodiment, the total molar concentration of ionic species in the composition is comprised from 50 to 300 mM.

In one embodiment, the total molar concentration of ionic species in the composition is comprised from 75 to 300 mM.

In one embodiment, the total molar concentration of ionic species in the composition is comprised from 100 to 300 mM.

In one embodiment, the total molar concentration of ionic species in the composition is comprised from 200 to 300 mM.

In one embodiment, the total molar concentration of ionic species in the composition is comprised from 10 to 200 mM.

In one embodiment, the total molar concentration of ionic species in the composition is comprised from 20 to 200 mM.

In one embodiment, the total molar concentration of ionic species in the composition is comprised from 30 to 200 mM.

In one embodiment, the total molar concentration of ionic species in the composition is comprised from 50 to 200 mM.

In one embodiment, the total molar concentration of ionic species in the composition is comprised from 75 to 200 mM.

In one embodiment, the total molar concentration of ionic species in the composition is comprised from 100 to 200 mM.

In one embodiment, the total molar concentration of ionic species in the composition is comprised from 10 to 100 mM.

In one embodiment, the total molar concentration of ionic species in the composition is comprised from 20 to 100 mM.

In one embodiment, the total molar concentration of ionic species in the composition is comprised from 30 to 100 mM.

In one embodiment, the total molar concentration of ionic species in the composition is comprised from 50 to 100 mM.

In one embodiment, the total molar concentration of ionic species in the composition is comprised from 75 to 100 mM.

In one embodiment, the total molar concentration of ionic species in the composition is comprised from 10 to 75 mM.

In one embodiment, the total molar concentration of ionic species in the composition is comprised from 20 to 75 mM.

In one embodiment, the total molar concentration of ionic species in the composition is comprised from 30 to 75 mM.

In one embodiment, the total molar concentration of ionic species in the composition is comprised from 50 to 75 mM.

In one embodiment, the total molar concentration of ionic species in the composition is comprised from 10 to 50 mM.

In one embodiment, the total molar concentration of ionic species in the composition is comprised from 20 to 50 mM.

In one embodiment, the total molar concentration of ionic species in the composition is comprised from 30 to 50 mM.

In one embodiment, said ionic species are present in a concentration ranging from 5 to 400 mM.

In one embodiment, said ionic species are present in a concentration ranging from 5 to 300 mM.

In one embodiment, said ionic species are present in a concentration ranging from 5 to 200 mM.

In one embodiment, said ionic species are present in a concentration ranging from 5 to 100 mM.

In one embodiment, said ionic species are present in a concentration ranging from 5 to 75 mM.

In one embodiment, said ionic species are present in a concentration ranging from 5 to 50 mM.

In one embodiment, said ionic species are present in a concentration ranging from 5 to 25 mM.

In one embodiment, said ionic species are present in a concentration ranging from 5 to 20 mM.

In one embodiment, said ionic species are present in a concentration ranging from 5 to 10 mM.

In one embodiment, said ionic species are present in a concentration ranging from 10 to 400 mM.

In one embodiment, said ionic species are present in a concentration ranging from 10 to 300 mM.

In one embodiment, said ionic species are present in a concentration ranging from 10 to 200 mM.

In one embodiment, said ionic species are present in a concentration ranging from 10 to 100 mM.

In one embodiment, said ionic species are present in a concentration ranging from 10 to 75 mM.

In one embodiment, said ionic species are present in a concentration ranging from 10 to 50 mM.

In one embodiment, said ionic species are present in a concentration ranging from 10 to 25 mM.

In one embodiment, said ionic species are present in a concentration ranging from 10 to 20 mM.

In one embodiment, said ionic species are present in a concentration ranging from 20 to 300 mM.

In one embodiment, said ionic species are present in a concentration ranging from 20 to 200 mM.

In one embodiment, said ionic species are present in a concentration ranging from 20 to 100 mM.

In one embodiment, said ionic species are present in a concentration ranging from 20 to 75 mM.

In one embodiment, said ionic species are present in a concentration ranging from 20 to 50 mM.

In one embodiment, said ionic species are present in a concentration ranging from 20 to 25 mM.

In one embodiment, said ionic species are present in a concentration ranging from 50 to 300 mM.

In one embodiment, said ionic species are present in a concentration ranging from 50 to 200 mM.

In one embodiment, said ionic species are present in a concentration ranging from 50 to 100 mM.

In one embodiment, said ionic species are present in a concentration ranging from 50 to 75 mM.

In the case of cations of mineral origin and in particular Zn2+, its molar concentration in the composition may comprise from 0.25 to 20 mM, in particular from 0.25 to 10 mM or from 0.25 to 5 mM.

In one embodiment, the ionic species is NaCl.

In one embodiment NaCl is present in a concentration ranging from 5 to 250 mM.

In one embodiment NaCl is present in a concentration ranging from 10 to 150 mM.

In one embodiment NaCl is present in a concentration ranging from 20 to 100 mM.

In one embodiment, the ionic species is citric acid and/or its salts

In one embodiment, citric acid and/or its salts is present in a concentration ranging from 5 to 40 mM.

In one embodiment, citric acid and/or its salts is present in a concentration ranging from 7 to 30 mM.

In one embodiment, citric acid and/or its salts is present in a concentration ranging from 8 to 20 mM.

In one embodiment, citric acid and/or its salts is present in a concentration ranging from 10 to 15 mM.

In one embodiment, the composition further comprises a zinc salt, especially zinc chloride.

In one embodiment, the concentration of zinc salt is comprised from 50 to 5000 μM.

In one embodiment, the concentration of zinc salt is comprised from 100 to 2000 μM.

In one embodiment, the concentration of zinc salt is comprised from 200 to 1500 μM.

In one embodiment, the concentration of zinc salt is comprised from 200 to 1000 μM.

In one embodiment, the zinc concentration is such that the molar ratio [zinc]/[glucagon] is comprised from 0.1 to 2.5.

In one embodiment, the zinc concentration is such that the molar ratio [zinc]/[glucagon] is comprised from 0.2 to 2.

In one embodiment, the zinc concentration is such that the molar ratio [zinc]/[glucagon] is comprised from 0.5 to 1.5.

In one embodiment, the zinc concentration is such that the [zinc]/[glucagon] molar ratio is 1.

In one embodiment, the compositions according to the invention further comprise preservatives.

In an embodiment, the preservatives are chosen from the group consisting of m-cresol and phenol, alone or in a mixture.

In one embodiment, the compositions according to the invention further comprise antioxidants.

In one embodiment, the antioxidants are chosen from methionine.

In one embodiment, the concentration of preservatives is comprised from 10 to 50 mM.

In one embodiment, the concentration of preservatives is comprised from 10 to 40 mM.

In one embodiment, the compositions according to the invention further comprise a surfactant.

In one embodiment, the surfactant is chosen from the group consisting of propylene glycol or polysorbate.

The compositions according to the invention may further comprise additives such as tonicity agents.

In one embodiment, the tonicity agents are chosen from the group consisting of sodium chloride, mannitol, sucrose, sobitol, and glycerol.

The compositions according to the invention may also comprise all excipients compatible with pharmacopoeias and compatible with human glucagon and gastrointestinal hormones, especially GLP-1 RAs, used at usage concentrations.

The invention also relates to a pharmaceutical formulation according to the invention, characterized in that it is obtained by drying and/or lyophilization.

In the case of local and systemic releases, the proposed modes of administration are intravenous, subcutaneous, intradermal or intramuscular.

In one embodiment, the mode of administration is subcutaneous.

In one embodiment, the mode of administration is intramuscular.

Transdermal, oral, nasal, vaginal, ocular, oral, and pulmonary routes of administration are also envisaged.

The invention also relates to single dose formulations at a pH ranging from 6.6 to 7.8 comprising human glucagon.

The invention also relates to single dose formulations at a pH ranging from 6.6 to 7.8 comprising human glucagon and a gastrointestinal hormone, as defined above.

In one embodiment, the single-dose formulations further comprise a substituted co-polyamino acid as defined above.

In one embodiment, the formulations are in the form of an injectable solution. In one embodiment, GLP-1 RA, analogue or obtained from GLP-1 RA is chosen from the group consisting of exenatide (Byetta®), liraglutide (Victoza®), lixisenatide (Lyxumia®), albiglutide (Tanzeum®), dulaglutide (Trulicity®) or a derivative thereof.

In one embodiment, the gastrointestinal hormone is exenatide.

In one embodiment, the gastrointestinal hormone is liraglutide.

In one embodiment, the gastrointestinal hormone is lixisenatide.

In one embodiment, the gastrointestinal hormone is albiglutide.

In one embodiment, the gastrointestinal hormone is dulaglutide.

Moreover, equally importantly, the applicant was able to verify that human glucagon in the presence of a co-polyamino acid bearing carboxylate charges and at least one hydrophobic radical according to the invention, retains its action whether alone or in combination with a gastrointestinal hormone.

The preparation of a composition according to the invention has the advantage of being able to be realized by a simple mixing of a solution of human glucagon, a solution of GLP-1 RA, an analogue or a derivative of GLP-1 RA, and a co-polyamino acid bearing carboxylate charges and at least one hydrophobic radical according to the invention, in aqueous solution or in freeze-dried form. If necessary, the pH of the preparation is adjusted to pH 7.

In one embodiment the mixture of human glucagon and substituted co-polyamino acid is concentrated by ultrafiltration prior to mixing with GLP-1 RA, an analogue or derivative of GLP-1 RA in aqueous solution or in freeze-dried form.

If necessary, the composition of the mixture is adjusted to excipients such as glycerol, m-cresol, and polysorbate (Tween®) by adding concentrated solutions of these excipients within the mixture. If necessary, the pH of the preparation is adjusted to 7.

### DESCRIPTION OF FIGURES

FIG. 1:

This figure is a graphic representation of the determination of the latency time (LT) by monitoring the fluorescence of Thioflavin T, on a curve showing the fluorescence value (in ua arbitrary units) on the y-axis and the time in minutes on the horizontal axis.

FIG. 2:

The median pharmacodynamic glucose curves expressed by the difference in glucose versus baseline level are shown in FIG. 2. This figure represents on the x-axis, the post-injection time and on the y-axis the percentage of glucose level.

This figure illustrates the pharmacodynamic results of glucagon obtained with the compositions described in examples CR0-1-I (solid triangles) and CR0-5—(solid squares).

FIG. 3:

The pharmacokinetic curves are illustrated in FIG. 3. This figure represents on the x-axis, post-injection time and on the y-axis, the glucose level (pmol/L) versus baseline.

This figure illustrates the pharmacokinetic results of glucagon obtained with the compositions described in examples CR0-1-I (solid triangles) and CR0-5-1 (solid squares).

FIG. 4:

The median pharmacodynamic glucose curves expressed by the difference in glucose versus baseline level are shown in FIG. 4. This figure represents on the x-axis, the post-injection time and on the y-axis the percentage of glucose level.

This figure illustrates the pharmacodynamic results of glucagon obtained with the compositions described in examples CR0-1-I (solid triangles) and CV0-14-1 (solid squares).

FIG. 5:

The pharmacokinetic curves are illustrated in FIG. 5. This figure represents on the x-axis, post-injection time and on the y-axis, the glucose level (pmol/L) versus baseline.

This figure illustrates the pharmacokinetic results of glucagon obtained with the compositions described in examples CR0-1-I (solid triangles) and CV0-14-I (solid squares).

FIG. 6:

The median pharmacodynamic glucose curves expressed by the difference in glucose versus baseline level are shown in FIG. 6. This figure represents on the x-axis, the post-injection time and on the y-axis the percentage of glucose level.

This figure shows the pharmacodynamic results of glucagon obtained with the compositions described in examples CV0-14-I (full triangles) and CV0-15-I (full squares).

The following examples illustrate the present application.

### AA: Synthesis of Intermediate Hydrophobic Compounds Hv to Obtain the Radicals −Hv Wherein p=1

The hydrophobic intermediate compounds are represented in the following table by the corresponding hydrophobic molecule before co-polyamino acid grafting.

TABLE 1A
list and structures of the hydrophobic intermediate compounds synthesized according to the invention.

| No | HYDROPHOBIC INTERMEDIATE COMPOUNDS |
|---|---|
| AA1 | 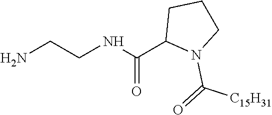 |
| AA2 | 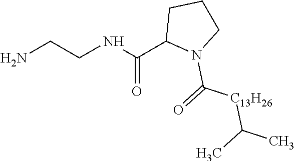 |
| AA3 | 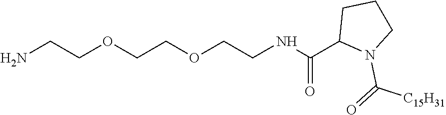 |
| AA4 | 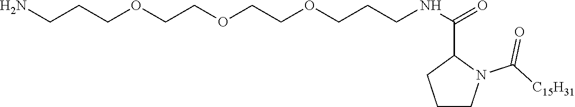 |
| AA5 | 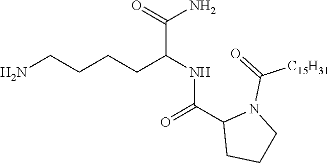 |
| AA6 | 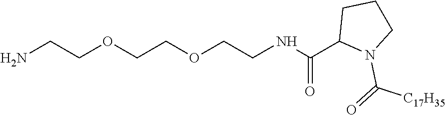 |
| AA7 | 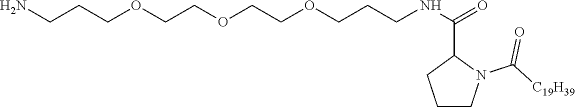 |
| AA8 | 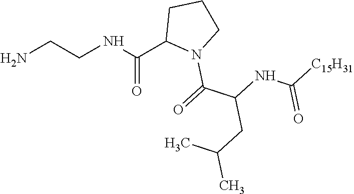 |
| AA9 | 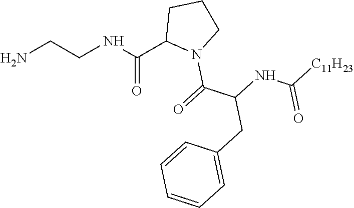 |
| AA10 | 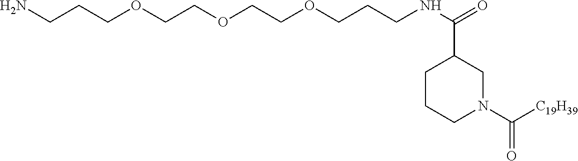 |
| AA11-1 | 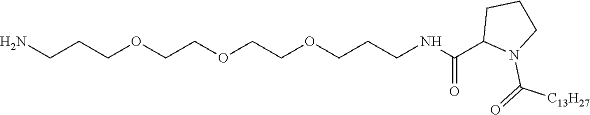 |
| AA12 | 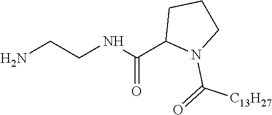 |
| AA12-1 | 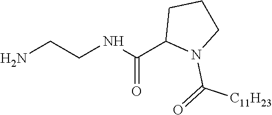 |
| AA13-1 | 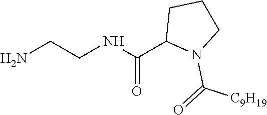 |
| AA14 | 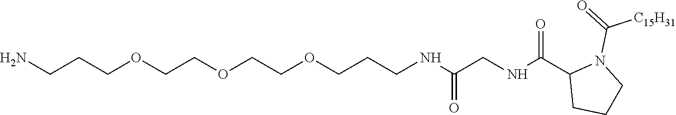 |
| AA14-1 | 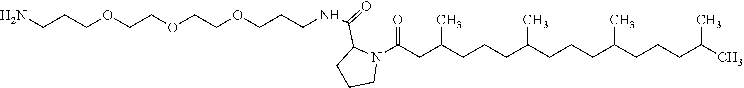 [SMILES: CC(C)CCCC(C)CCCC(C)CCCC(C)CC(=O)N1CCCC1C(=O)NCCCOCCOCCOCCCN] |
| AA15 | 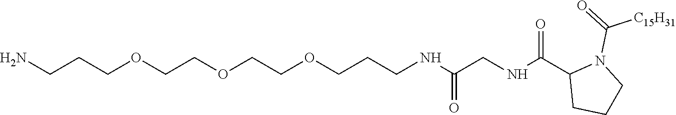 |
| AA16 | 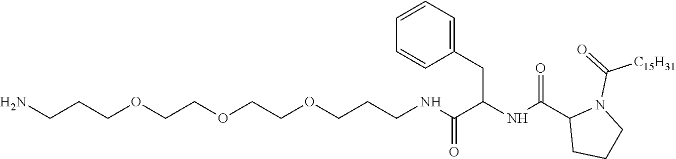 |
| AA17 | 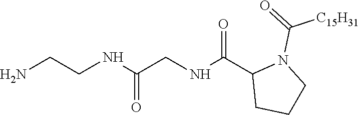 |

### EXAMPLE AA1: MOLECULE AA1

### Molecule A1: Product Obtained by the Reaction Between Palmitoyl Chloride and L-Proline

To a solution of L-proline (10.6 g, 92.1 mmol) in 1N aqueous sodium hydroxide (230 mL, 230 mmol) is added dropwise for 90 minutes a solution of palmitoyl chloride (23.0 g, 83.7 mmol) in acetone (167 mL). After stirring for 14 hours at room temperature, the heterogeneous mixture is cooled to 0° C., then filtered through a sintered filter to give a white solid which is washed with water (2×100 mL), then diisopropyl ether (100 mL). The solid is dried under reduced pressure. The solid is then dissolved under reflux in 200 mL of water, then 8 mL of a 37% hydrochloric acid solution are added to obtain a pH=1. The opalescent reaction medium is then cooled to 0° C. The precipitate obtained is filtered through a sintered filter, then washed with water (5×50 mL) until filtrates of physiological pH ranging from 6.0 to 8.0 are obtained, then dried in an oven at 50° C. under vacuum overnight. The product is purified by recrystallization in diisopropyl ether. A white solid is obtained.

Yield: 22.7 g (77%).

NMR ¹H (CDCl₃, ppm): 0.88 (3H); 1.19-1.45 (24H); 1.58-1.74 (2H); 1.88-2.14 (3H); 2.15-2.54 (3H); 3.47 (1H); 3.58 (1H); 4.41 (0.1H); 4.61 (0.9H); 6.60-8.60 (1H).

### A2 Molecule: Product Obtained by Reaction Between Molecule A1 and Boc-Ethylenediamine

N,N-diisopropylethylamine (DIPEA) (68.8 g, 532.3 mmol), 1-hydroxybenzotriazole (HOBt) (37.1 g, 274.6 mmol), then N(3-dimethylaminopropyl)-N′-ethylcarbodiimide (EDC) (53.1 g, 277.0 mmol) are successively added at room temperature to a solution of molecule A1 (75.1 g, 212.4 mmol) in 1500 mL of chloroform. After stirring for 15 minutes at room temperature, a solution of Boc-ethylenediamine (Boc-ethylenediamine) (37.6 g, 234.7 mmol) in 35 mL of chloroform is added. After stirring for 18 hours at room temperature, a solution of 0.1 N HCl (2.1 L), then a saturated solution of NaCl (1 L) are added. The phases are separated then the organic phase is washed successively with a solution of 0.1 N HCl/saturated NaCl (2.1 L/1 L), a saturated solution of NaCl (2 L), a saturated NaHCO₃ (2 L) solution, then a saturated NaCl (2 L) solution. The organic phase is dried over anhydrous sodium sulphate, filtered, then concentrated under reduced pressure. The obtained solid is purified by trituration in diisopropyl ether (3×400 mL), to yield a solid after drying under vacuum at 40° C.

Yield: 90.4 g (86%).

NMR ¹H (CDCl₃, ppm): 0.88 (3H); 1.20-1.37 (24H); 1.44 (9H); 1.54-1.70 (2H); 1.79-1.92 (1H); 1.92-2.04 (1H); 2.03-2.17 (1H); 2.17-2.44 (3H); 3.14-3.36 (4H); 3.43 (1H); 3.56 (1H); 4.29 (0.1H); 4.51 (0.9H); 4.82 (0.1H); 5.02 (0.9H); 6.84 (0.1H); 7.22 (0.9H).

### Molecule AA1

A solution of 4 M hydrochloric acid in dioxane (100 mL, 400 mmol) is added dropwise and at 0° C. to a solution of molecule A2 (20.1 g, 40.5 mmol) in 330 mL of dichloromethane. After stirring for 3 h30 at room temperature, the solution is concentrated under reduced pressure. The residue is purified by flash chromatography (methanol, dichloromethane) to yield a white solid of molecule AA1 in the form of a hydrochloride salt.

Yield: 16.3 g (93%).

NMR ¹H (CDCl₃, ppm): 0.88 (3H); 1.07-1.40 (24H); 1.49-1.63 (2H); 1.77-2.18 (4H); 2.18-2.45 (2H); 3.14-3.32 (2H); 3.42-3.63 (2H); 3.63-3.84 (2H); 4.37 (0.1H); 4.48 (0.9H); 6.81-8.81 (4H).

LC/MS (ESI): 396.5; (calculated ([M+H]+): 396.4).

### EXAMPLE AA2: MOLECULE AA2

### Molecule A3: 15-methylhexadecan-1-ol

Magnesium in chips (9.46 g, 389 mmol) is introduced into a three-neck flask under argon. The magnesium is covered with anhydrous THF (40 mL), and a few drops of 1-bromo-3-methylbutane are added at room temperature to initiate the reaction. After the observation of an exotherm and a slight turbidity of the medium, the rest of the 1-bromo-3-methylbutane (53.87 g, 357 mmol) is added dropwise in 90 minutes while the temperature of the medium remains stable between 50 and 60° C. The reaction medium is then heated at 70° C. for 2 hours.

In a three-necked flask under argon, a solution of 12-bromo-1-dodecanol (43 g, 162.1 mmol) in THF (60 mL) is added dropwise at 0° C. to a solution of CuCl (482 mg, 4.86 mmol) dissolved in NMP (62 mL). To this solution is then added dropwise, the hot organomagnesium solution, freshly prepared in order to maintain the temperature of the medium below 20° C. The mixture is then stirred at room temperature for 16 hours. The medium is cooled to 0° C. and the reaction is stopped by addition of a 1N HCl aqueous solution until pH 1 and the medium is extracted with ethyl acetate. After washing the organic phase with saturated NaCl solution and drying over Na₂SO₄, the solution is filtered and concentrated under vacuum to produce an oil. After purification by DCVC on silica gel (cyclohexane, ethyl acetate), an oil which crystallizes at room temperature is obtained.

Yield: 32.8 g (74%)

NMR ¹H (CDCl₃, ppm): 0.87 (6H); 1.14 (2H); 1.20-1.35 (22H); 1.50-1.55 (3H); 3.64 (2H).

### Molecule A4: 15-Methylhexadecanoic Acid

To a solution of molecule A3 (20.65 g, 80.5 mmol) and tetrabutylammonium bromide (14.02 g, 42.5 mmol) in a mixture of acetic acid/dichloroethane/water (124/400/320 mL) at room temperature is added in small portions potassium permanganate (38.2 g, 241.5 mmol). After stirring at reflux for 5 hours and returning to room temperature, the medium is acidified until pH 1 by progressive addition of 5N HCl. Na₂SO₃ (44.6 g, 354.3 mmol) is then gradually added until the decoloration of the medium. The aqueous phase is extracted with dichloromethane, and the combined organic phases are dried over Na₂SO₄, filtered and concentrated under vacuum. After purification by chromatography on silica gel (cyclohexane, ethyl acetate, acetic acid), a white solid is obtained.

Yield: 19.1 g (quantitative)

NMR ¹H (CDCl₃, ppm): 0.87 (6H); 1.14 (2H); 1.22-1.38 (20H); 1.51 (1H); 1.63 (2H); 2.35 (2H).

### Molecule A5: Product Obtained by Reaction Between the A4 Molecule and L-Proline

Dicyclohexyl carbodiimide (DCC) (8.01 g, 38.8 mmol) and N-hydroxysuccinimide (NHS) (4.47 g, 38.8 mmol) are successively added to a solution of molecule A4 (10 g, 37 mmol) in THF (360 mL) at 0° C. After stirring for 17 hours at room temperature, the medium is cooled to 0° C. for 20 minutes, filtered through a sintered filter. L-proline (4 g, 37.7 mmol), triethylamine (34 mL) and water (30 mL) are added to the filtrate. After stirring for 20 h at room temperature, the medium is treated with a 1N HCl aqueous solution until pH 1. The aqueous phase is extracted with dichloromethane (2×125 mL). The combined organic phases are washed with an aqueous solution of 1N HCl (2×100 mL), water (100 mL), then a saturated aqueous solution of NaCl (100 mL). After drying over Na₂SO₄, the organic phase is filtered, concentrated under vacuum, and the residue is purified by chromatography on silica gel (cyclohexane, ethyl acetate, acetic acid)

Yield: 9.2 g (72%)

NMR ¹H (CDCl₃, ppm): 0.86 (6H); 1.14 (2H); 1.22-1.38 (20H); 1.50 (1H); 1.67 (2H); 1.95-2.10 (3H); 2.34 (2H); 2.49 (1H); 3.47 (1H); 3.56 (1H); 4.61 (1H).

LC/MS (ESI): 368.3; (calculated ([M+H]+): 368.6).

### Molecule A6: Product Obtained by Reaction Between the Molecule A5 and Boc-Ethylenediamine

Triethylamine (TEA) (5.23 mL) and 2-(1H-benzotriazol-1-yl)-1,1,3,3-tetramethyluronium tetrafluoroborate (TBTU) are added to a solution of molecule A5 (9.22 g, 25.08 mmol) in a THF/DMF mixture (200/50 mL) at room temperature. After 10 minutes of stirring, Boc-ethylenediamine (4.42 g, 27.6 mmol) is added. After stirring at room temperature for 17 h, the mixture is diluted with water (300 mL) at 0° C. and stirred cold for 20 minutes. The precipitate formed is filtered through a sintered filter and the filtrate is extracted with ethyl acetate. The combined organic phases are washed with saturated NaHCO₃, solution, dried on Na₂SO₄, filtered, concentrated under vacuum and the residue purified by flash chromatography (ethyl acetate, methanol).

Yield: 6.9 g (54%)

NMR ¹H (CDCl₃, ppm): 0.86 (6H); 1.15 (2H); 1.22-1.38 (20H); 1.43 (9H); 1.50 (1H); 1.64 (4H); 1.85 (1H); 1.95 (1H); 2.10 (1H); 2.31 (2H); 3.20-3.35 (3H); 3.45 (1H); 3.56 (1H); 4.51 (1H); 5.05 (1H); 7.24 (1H).

LC/MS (ESI): 510.6; (calculated ([M+H]+): 510.8).

### Molecule AA2

A 4 N HCl solution in dioxane (13 mL) is added to a molecule A6 (5.3 g, 10.40 mmol) solution in dichloromethane (50 mL) at 0° C. After stirring for 5 hours at 0° C., the medium is concentrated under vacuum, returned to water and freeze-dried to give a white solid of molecule AA2 in the form of hydrochloride salt.

Yield: 4.6 g (99%)

NMR ¹H (D₂O, ppm): 0.91 (6H); 1.22 (2H); 1.22-1.50 (20H); 1.63 (3H); 1.98 (1H); 2.10 (2H); 2.26 (1H); 2.39 (1H); 2.43 (1H); 3.22 (2H); 3.45-3.60 (3H); 3.78 (1H); 4.42 (1H).

LC/MS (ESI): 410.4; (calculated ([M+H]+): 410.7).

### EXAMPLE AA3: MOLECULE AA3

### Molecule A7: Product Obtained by the Reaction Between Molecule A1 and Boc-Tri (Ethylene Glycol) Diamine

By a process similar to that used in the preparation of molecule A2 applied to molecule A1 (4.0 g, 11.3 mmol) and Boc-tri (ethylene glycol) diamine (3.1 g, 12.4 mmol), a colorless oil is obtained after purification by flash chromatography (methanol, toluene).

Yield: 5.5 g (84%).

NMR ¹H (CDCl₃, ppm): 0.88 (3H); 1.09-1.39 (24H); 1.44 (9H); 1.64 (2H); 1.79-2.01 (2H); 2.06-2.43 (4H); 3.23-3.68 (14H); 4.33 (0.2H); 4.56 (0.8H); 5.25 (1H); 6.49 (0.2H); 7.13-7.50 (0.8H).

### Molecule AA3

By a process similar to that used in the preparation of molecule AA1 applied to molecule A7 (5.5 g, 9.4 mmol), a white solid of molecule AA3 in the form of a hydrochloride salt is obtained after purification by flash chromatography (methanol, dichloromethane).

Yield: 4.3 g (92%).

NMR ¹H (DMSO-d6, ppm): 0.85 (3H); 1.08-1.40 (24H); 1.40-1.52 (2H); 1.71-2.02 (4H); 2.02-2.31 (2H); 2.90-2.98 (2H); 3.15-3.47 (5H); 3.50-3.66 (7H); 4.24 (0.6H); 4.32 (0.4H); 7.83 (0.6H); 7.95 (3H); 8.17 (0.4H).

LC/MS (ESI): 484.6; (calculated ([M+H]+): 484.4).

### EXAMPLE AA4: MOLECULE AA4

### Molecule A8: Product Obtained by the Reaction Between Molecule A1 and Boc-1-Amino-4,7,10-Trioxa-13-Tridecanamine

By a process similar to that used in the preparation of molecule A2 applied to molecule A1 (4.5 g, 12.7 mmol) and to Boc-1-amino-4,7,10-trioxa-13-tridecane amine (4.5 g, 14.0 mmol), a yellow oil is obtained after purification by flash chromatography (methanol, dichloromethane).

Yield: 7.7 g (92%).

NMR ¹H (CDCl₃, ppm): 0.88 (3H); 1.22-1.37 (24H); 1.44 (9H); 1.59-1.67 (2H); 1.67-2.00 (6H); 2.06-2.45 (4H); 3.18-3.76 (18H); 4.28 (0.2H); 4.52 (0.8H); 4.69-5.04 (1H); 6.77 (0.2H); 7.20 (0.8H).

### Molecule AA4

By a process similar to that used in the preparation of molecule AA1 applied to molecule A8 (7.7 g, 11.8 mmol), a yellow oil is obtained after purification by flash chromatography (methanol, dichloromethane). Co-evaporation with diisopropyl ether makes it possible to obtain the molecule AA4 in the form of a hydrochloride salt in the form of a white solid which is dried under vacuum at 50° C.

Yield: 5.4 g (76%).

NMR ¹H (CDCl₃, ppm): 0.88 (3H); 1.08-1.40 (24H); 1.49-1.65 (2H); 1.76-2.39 (10H); 3.07-3.28 (3H); 3.34-3.80 (15H); 4.34 (0.05H); 4.64 (0.95H); 7.35 (0.05H); 7.66-8.58 (3.95H).

LC/MS (ESI): 556.7; (calculated ([M+H]+): 556.5).

### EXAMPLE AA5: MOLECULE AA5

### Molecule A9: Product Obtained by Reaction Between Molecule A1 and Methyl Ester of NOT-Boc-L-Lysine

By a process similar to that used for the preparation of molecule A2 applied to molecule A1 (4 g, 11.3 mmol) and methyl ester of N-Boc-L-lysine (3.2 g, 12.4 mmol), a colorless oil is obtained after purification by flash chromatography (methanol, dichloromethane).

Yield: 4.9 g (73%).

NMR ¹H (CDCl₃, ppm): 0.88 (3H); 0.99-1.54 (37H); 1.54-1.75 (3H); 1.75-2.04 (3H); 2.04-2.41 (4H); 2.94-3.19 (2H); 3.19-3.81 (5H); 4.28-4.64 (2H); 4.94 (1H); 6.45 (0.1H); 7.36 (0.9H).

LC/MS (ESI): 596.7; (calculated ([M+H]+): 596.5).

### Molecule A10: Product Obtained by Treatment of Molecule A9 with Ammonia

320 mL of a 7 N ammonia solution in methanol are added to a suspension of molecule A9 (4.9 g, 8.2 mmol) in 10 ml of methanol. After stirring for 19 hours at room temperature in a closed atmosphere, an additional 100 mL of ammonia solution are added. After stirring for 24 hours at room temperature in a closed atmosphere, the reaction medium is concentrated under reduced pressure. The residue is purified by trituration in refluxing diisopropyl ether (100 mL) to give a white solid which is dried under vacuum at 50° C.

Yield: 4.1 g (85%).

NMR ¹H (CDCl₃, ppm): 0.88 (3H); 1.06-1.57 (37H); 1.57-1.79 (3H); 1.88-2.41 (7H); 3.09 (2H); 3.49 (1H); 3.62 (1H); 4.34 (1H); 4.51 (1H); 4.69-4.81 (1H); 5.43 (0.95H); 5.57 (0.05H); 6.25 (0.05H); 6.52 (0.95H); 6.83 (0.05H); 7.11 (0.95H).

### Molecule AA5

By a process similar to that used in the preparation of molecule AA1 applied to molecule A10 (388 mg, 0.67 mmol), a white solid of molecule AA5 in the form of a hydrochloride salt is obtained after purification by trituration in diisopropyl ether.

Yield: 292 mg (85%).

NMR ¹H (DMSO-d6, ppm): 0.85 (3H); 1.06-2.34 (38H); 2.61-2.81 (2H); 3.29-3.68 (2H); 4.05-4.17 (1.7H); 4.42 (0.3H); 7.00 (1H); 7.16 (0.7H); 7.43 (0.3H); 7.73-8.04 (3.7H); 8.16 (0.3H).

LC/MS (ESI): 481.6; (calculated ([M+H]+): 481.4).

### EXAMPLE AA6: MOLECULE AA6

### Molecule A11: Product Obtained by the Reaction Between Stearoyl Chloride and L-Proline

By a process similar to that used in the preparation of molecule A1 applied to L-proline (5.0 g, 43.4 mmol) and to stearoyl chloride (12.0 g, 39.6 mmol), a white solid is obtained after purification by flash chromatography (methanol, dichloromethane).

Yield: 5.37 g (36%)

NMR ¹H (CDCl₃, ppm): 0.88 (3H); 1.26-1.37 (28H); 1.64-1.70 (2H); 1.88-2.10 (3H); 2.36 (2H); 2.54-2.58 (1H); 3.46 (1H); 3.56 (1H); 4.62 (1H).

LC/MS (ESI): 382.6; (calculated ([M+H]+): 382.3).

### Molecule A12: Product Obtained by Reaction Between Molecule all and Boc-Tri (Ethylene Glycol) Diamine

By a process similar to that used in the preparation of molecule A6 applied to molecule A11 (33.81 g, 88.6 mmol) and Boc-tri (ethylene glycol) diamine (26.4 g, 106.3 mmol) in THF using DIPEA instead of TEA, a white solid is obtained after purification by flash chromatography (ethyl acetate, methanol).

Yield: 43.3 g (80%)

NMR ¹H (CDCl₃, ppm): 0.87 (3H); 1.24 (30H); 1.43 (9H); 1.61 (2H); 1.82 (1H); 1.96 (1H); 2.25-2.45 (2H); 3.25-3.65 (14H); 4.30 (0.15H); 4.53 (0.85H); 5.25 (1H); 6.43 (0.15H); 7.25 (0.85H).

LC/MS (ESI): 612.6; (calculated ([M+H]+): 612.9).

### Molecule AA6

By a process similar to that used in the preparation of molecule AA2 applied to molecule A12 (43 g, 70.3 mmol), the residue obtained after concentration under vacuum is triturated in acetonitrile. The suspension is filtered, and the solid is washed with acetonitrile then with acetone. After drying under vacuum, a white solid of molecule AA6 in the form of a hydrochloride salt is obtained.

Yield: 31.2 g (81%)

NMR ¹H (DMSO-d₆, ppm): 0.85 (3H); 1.23 (28H); 1.45 (2H); 1.70-2.05 (4H); 2.13 (1H); 2.24 (1H); 2.95 (2H); 3.10-3.25 (2H); 3.30-3.65 (10H); 4.20-4.45 (1H); 7.85-8.25 (4H).

LC/MS (ESI): 512.4; (calculated ([M+H]+): 512.8).

### EXAMPLE AA7: MOLECULE AA7

### Molecule A13: Product Obtained by Reaction Between Arachidic Acid and L-Proline

By a process similar to that used in the preparation of A5 molecule applied to arachidic acid (15.51 g, 49.63 mmol) and L-proline (6 g, 52.11 mmol) using DIPEA in place of TEA, a white solid is obtained after purification by chromatographic column on silica gel (cyclohexane, ethyl acetate, acetic acid).

Yield: 12.9 g (63%)

NMR ¹H (CDCl₃, ppm): 0.88 (3H); 1.28 (34H); 1.66 (2H); 1.95-2.15 (2H); 2.34 (2H); 2.45 (1H); 3.47 (1H); 3.56 (1H); 4.60 (1H).

LC/MS (ESI): 410.4; (calculated ([M+H]+): 410.6).

### Molecule A14: Product Obtained by the Reaction Between Molecule A13 and Boc-1-Amino-4,7,10-Trioxa-13-Tridecane

By a process similar to that used in the preparation of molecule A12 applied to molecule A13 (10.96 g, 26.75 mmol) and Boc-1-amino-4,7,10-trioxa-13-tridecane (10.29 g, 32.11 mmol), a solid is obtained after purification by chromatographic column on silica gel (cyclohexane, ethyl acetate, methanol).

Yield: 14.2 g (75%)

NMR ¹H (CDCl₃, ppm): 0.88 (3H); 1.24 (32H); 1.43 (9H); 1.57-2.00 (8H); 2.10-2.45 (4H); 3.20-3.75 (18H); 4.30 (0.20H); 4.55 (0.80H); 5.03 (1H); 6.75 (0.20H); 7.20 (0.80H).

LC/MS (ESI): 712.8; (calculated ([M+H]+): 713.1).

### Molecule AA7

Following a process similar to that used for the preparation of molecule AA2 applied to molecule A14 (14.25 g, 20.01 mmol), the residue obtained after concentration of the reaction medium under vacuum is dissolved in methanol and evaporated under reduced pressure; the process being repeated 4 times to produce a white solid of molecule AA7 in the form of hydrochloride salt.

Yield: 12.7 g (98%)

NMR ¹H (DMSO-d₆, ppm): 0.85 (3H); 1.23 (32H); 1.45 (2H); 1.64 (2H); 1.70-2.05 (6H); 2.10-2.30 (2H); 2.82 (2H); 3.08 (2H); 3.30-3.60 (14H); 4.15-4.30 (1H); 7.73-8.13 (4H).

LC/MS (ESI): 612.7; (calculated ([M+H]+): 612.9).

### EXAMPLE AA8: MOLECULE AA8

### Molecule A15: Product Obtained by the Reaction Between L-Leucine and Palmitoyl Chloride

By a process similar to that used in the preparation of molecule A1 applied to L-leucine (15.0 g, 114.4 mmol) and to palmitoyl chloride (34.5 g, 125 mmol), a white solid is obtained by trituration in diisopropyl ether.

Yield: 13.0 g (31%)

NMR ¹H (CDCl₃, ppm): 0.88 (3H); 0.96 (6H); 1.16-1.35 (24H); 1.55-1.77 (5H); 2.23 (2H); 4.55-4.60 (1H); 5.88 (1H).

### Molecule A16: Product Obtained by the Reaction Between Molecule A15 and the Methyl Ester of L-Proline

By a process similar to that used for the preparation of molecule A2 applied to molecule A15 (6.00 g, 16.2 mmol) and to the methyl ester of L-proline (3.23 g, 19.5 mmol), a slightly yellow oil is obtained after purification by flash chromatography (methanol, dichloromethane).

Yield: 5.8 g (74%)

NMR ¹H (CDCl₃, ppm): 0.83-1.00 (9H); 1.18-1.32 (24H); 1.40-1.73 (5H); 1.84-2.33 (6H); 3.47-3.89 (2H); 3.70 (1.14H); 3.71 (1.21H); 3.74 (0.53H); 3.76 (0.12H); 4.40-4.56 (1H); 4.63-4.67 (0.04H); 4.84 (0.38); 4.90 (0.40); 5.06 (0.18); 5.99 (0.18H); 6.08-6.21 (0.82).

LC/MS (ESI): 481.6; (calculated ([M+H]+): 481.4).

### Molecule A17: Product Obtained by the Saponification of the Methyl Ester of Molecule A16

To a solution of molecule A16 (5.8 g, 12.06 mmol) in 30 mL of methanol is added 1N sodium hydroxide (13.5 mL, 13.5 mmol). After stirring for 20 h at room temperature, the solution is diluted with water, then acidified with 20 mL of 1N hydrochloric acid at 0° C. The precipitate is filtered, then rinsed with water (50 ml) before being solubilized in 50 ml of dichloromethane. The organic phase is dried over Na₂SO₄, filtered, then concentrated under reduced pressure to yield a colorless oil.

Yield: 4.5 g (80%)

NMR ¹H (CDCl₃, ppm): 0.85-0.99 (9H); 1.14-1.41 (24H); 1.43-1.72 (5H); 1.87-2.47 (7H); 3.48-3.55 (0.6H); 3.56-3.62 (0.4H); 3.83-3.90 (0.4H); 3.90-3.96 (0.6H); 4.52-4.56 (0.6H); 4.56-4.59 (0.4H); 4.80-4.86 (0.4H); 4.86-4.91 (0.6H); 6.05 (0.4H); 6.11 (0.6H).

LC/MS (ESI): 467.6; (calculated ([M+H]+): 467.4).

### Molecule A18: Product Obtained by the Reaction Between Boc-Ethylenediamine and Molecule A17

By a process similar to that used for the preparation of molecule A2 applied to the molecule A17 (4.5 g, 9.64 mmol) and at N-Boc-ethylenediamine (1.70 g, 10.61 mmol), a colorless oil is obtained after purification by flash chromatography (methanol, dichloromethane).

Yield: 2.0 g (34%)

NMR ¹H (CDCl₃, ppm): 0.83-0.99 (9H); 1.19-1.32 (24H); 1.44 (9H); 1.48-2.37 (14H); 3.09-3.99 (4H); 4.28-5.01 (2H); 5.64-6.04 (1H); 6.87-7.06 (1H).

LC/MS (ESI): 609.7; (calculated ([M+H]+): 609.5).

### Molecule AA8

By a process similar to that used in the preparation of molecule AA1 applied to molecule A18 (2 g, 3.28 mmol), a white solid of molecule AA8 in the form of a hydrochloride salt is obtained after purification by flash chromatography (methanol, dichloromethane).

Yield: 1.5 g (90%)

NMR ¹H (CDCl₃, ppm): 0.83-1.00 (9H); 1.18-1.32 (24H); 1.37-1.77 (5H); 1.93-2.41 (6H); 3.07-3.97 (6H); 4.44-4.77 (2H); 7.66-8.21 (2H).

LC/MS (ESI): 509.6; (calculated ([M+H]+): 509.4).

### EXAMPLE AA9: MOLECULE AA9

### Molecule A19: Product Obtained by the Reaction Between Lauric Acid and L-Phenylalanine

By a process similar to that used for the preparation of A5 molecule applied to lauric acid (8.10 g, 40.45 mmol) and L-phenylalanine (7 g, 42.38 mmol), a white solid is obtained.

Yield: 12.7 g (98%)

NMR ¹H (DMSO-d₆, ppm): 0.86 (3H); 1.10-1.30 (16H); 1.36 (2H); 2.02 (2H); 2.82 (1H); 3.05 (1H); 4.42 (1H); 7.15-7.30 (5H); 8.05 (1H); 12.61 (1H).

LC/MS (ESI): 348.2; (calculated ([M+H]+): 348.5).

### Molecule A20: Product Obtained by the Reaction Between Molecule A19 and Hydrochloride Salt of the Methyl Ester of L-Proline

By a process similar to that used in the preparation of molecule A6 applied to molecule A19 (9.98 g, 28.72 mmol) and to L-proline methyl ester hydrochloride salt (5.23 g, 31.59 mmol), a colorless oil is obtained after purification by chromatographic column on silica gel (cyclohexane, ethyl acetate).

Yield: 5.75 g (44%)

NMR ¹H (CDCl₃, ppm): 0.88 (3H); 1.10-1.30 (16H); 1.50-1.75 (3H); 1.80-2.02 (3H); 2.17 (2H); 2.65 (0.5H); 2.95 (1H); 3.05-3.20 (1.5H); 3.50-3.65 (1H); 3.75 (3H); 4.29 (0.5H); 4.46 (0.5H); 4.70 (0.1H); 4.95 (0.9H); 6.20-6.30 (1H); 7.15-7.30 (5H).

LC/MS (ESI): 459.2; (calculated ([M+H]+): 459.6).

### Molecule A21: Product Obtained by Saponification of Molecule A20

Lithium hydroxide (LiOH) (600.49 mg, 25.07 mmol) is added to a solution of molecule A20 (5.75 g, 12.54 mmol) in a THF/methanol/water mixture (40/40/40 mL) at 0° C., then the mixture is stirred at room temperature for 20 hours. After evaporation of the organic solvents under vacuum, the aqueous solution is diluted in water, acidified with an 1N HCl aqueous solution until a pH of 1. The product is then extracted with ethyl acetate. The combined organic phases are washed with a saturated aqueous NaCl solution, dried over Na₂SO₄, filtered and concentrated under reduced pressure to yield a colorless oil.

Yield: 5.7 g (quantitative)

NMR ¹H (CDCl₃, ppm): 0.88 (3H); 1.10-1.30 (16H); 1.50-1.80 (3H); 1.67-2.02 (2H); 2.20 (2H); 2.25 (0.4H); 2.60 (0.6H); 2.85-3.10 (2.6H); 3.55-3.65 (1.4H); 4.35 (0.6H); 4.55 (0.4H); 4.94 (1H); 6.28 (0.4H); 6.38 (0.6H); 7.20-7.30 (5H).

LC/MS (ESI): 445.2; (calculated ([M+H]+): 445.6).

### Molecule A22: Product Obtained by Reaction Between N-Boc-Ethylenediamine and Molecule A21

By a process similar to that used in the preparation of molecule A6 applied to molecule A21 (5.67 g, 12.75 mmol) and N-Boc-ethylenediamine (2.25 g, 14.03 mmol), a colorless oil is obtained after purification by chromatography column on silica gel (dichloromethane, methanol).

Yield: 5.7 g (76%)

NMR ¹H (CDCl₃, ppm): 0.88 (3H); 1.25 (16H); 1.43 (9H); 1.58 (2.6H); 1.75-1.95 (1.4H); 2.15-2.30 (3H); 2.64 (0.5H); 2.95-3.10 (2.5H); 3.20-3.40 (4H); 3.45 (0.5H); 3.55 (0.2H); 3.66 (1H); 4.44 (1H); 4.50 (0.2H); 4.60 (0.6H); 4.99 (0.7H); 5.54 (0.5H); 5.95 (0.2H); 6.17 (1H); 6.60 (0.5H); 7.07 (0.5H); 7.20-7.40 (5H). LC/MS (ESI): 587.4; (calculated ([M+H]+): 587.8).

### Molecule AA9

Following a process similar to that used for the preparation of molecule AA2 applied to molecule A22 (5.66 g, 9.65 mmol), the residue obtained after concentration of the reaction medium under vacuum is dissolved in methanol and evaporated under reduced pressure; the process being repeated 4 times to produce a white foam of molecule AA9 in the form of hydrochloride salt.

Yield: 4.9 g (97%)

NMR ¹H (DMSO-d₆, 120° C., ppm): 0.89 (3H); 1.26 (16H); 1.43 (2H); 1.68 (0.6H); 1.75-2.00 (3H); 2.05-2.25 (2.4H); 2.82-3.05 (5H); 3.38 (2H); 3.50-3.70 (1.4H); 4.25 (0.6H); 4.63 (0.4H); 4.77 (0.6H); 7.25-7.50 (5H); 7.55-8.20 (4H).

LC/MS (ESI): 487.4; (calculated ([M+H]+): 487.7).

### EXAMPLE AA10: MOLECULE AA10

### Molecule A23: Product Obtained by the Reaction Between Nipecotic Acid and Arachidic Acid

By a process similar to that used in the preparation of molecule A5 applied to arachidic acid (2.30 g, 7.37 mmol) and nipecotic acid (1.00 g, 7.74 mmol), a white solid is obtained after filtering the acidified aqueous phase to pH 1 and washing the solid with water, then with dichloromethane.

Yield: 1.65 g (53%)

NMR 1H (CDCl₃, ppm): 0.88 (3H); 1.07-1.88 (37H); 2.10 (1H); 2.28-2.45 (2H); 2.52 (1H); 2.91-3.17 (1.5H); 3.42 (0.5H); 3.72 (0.5H); 3.84 (0.5H); 4.08 (0.5H); 4.56 (0.5H).

LC/MS (ESI): 424.4; 848.0; (calculated ([M+H]+): 424.4; ([2M+H]+): 847.8).

### Molecule A24: Product Obtained by the Reaction Between Molecule A23 and Boc-1-Amino-4,7,10-Trioxa-13-Tridecanamine

DIPEA (1.01 g, 7.79 mmol) and TBTU (1.31 g, 4.09 mmol), at room temperature, are successively added to a suspension of molecule A23 (1.65 g, 3.89 mmol) in 20 mL of THF. After stirring for 30 minutes, Boc-1-amino-4,7,10-trioxa-13-tridecane amine (1.37 g, 4.28 mmol) is added and the reaction medium is stirred at room temperature for 18 hours. After evaporation of the solvent under reduced pressure, the residue is diluted with ethyl acetate (100 mL), the organic phase is washed successively with a saturated NaHCO₃, aqueous solution, an aqueous solution of 1N HCl, and a saturated aqueous solution of NaCl, dried over Na₂SO₄, filtered and concentrated under reduced pressure. A white solid is obtained after purification by flash chromatography (cyclohexane, ethyl acetate, methanol).

Yield: 1.97 g (70%)

NMR ¹H (CDCl₃, ppm): 0.88 (3H); 1.15-2.70 (54H); 3.10-3.46 (6H); 3.46-3.71 (12.6H); 3.92 (0.4H); 4.17 (0.6H); 4.49 (0.4H); 4.80-5.16 (1H); 6.35-6.76 (1H).

LC/MS (ESI): 726.8; (calculated ([M+H]+): 726.6).

### Molecule AA10

By a process similar to that used in the preparation of molecule AA1 applied to molecule A24 (1.97 g, 2.71 mmol), a white solid of molecule AA10 is obtained after evaporation of the solvent, trituration in acetone, filtration and washing with acetone and drying under reduced pressure at 50° C.

Yield: 1.66 g (92%)

NMR ¹H (DMSO-d₆, ppm): 0.86 (3H); 1.09-1.90 (42H); 2.05-2.68 (5H); 2.45-2.68 (1H); 2.78-3.19 (6H); 3.36-3.44 (2H); 3.44-3.60 (10H); 3.69-3.87 (1H); 4.20 (0.4H); 4.35 (0.6H).

LC/MS (ESI): 626.7; (calculated ([M+H]+): 626.5).

### EXAMPLE AA11-1: MOLECULE AA11-1

### Molecule A24-1: Product Obtained by Reaction Between Molecule A26 and Boc-1-Amino-4,7,10-Trioxa-13-Tridecane Amine

By a process similar to that used in the preparation of molecule A27 applied to molecule A26 (24.00 g, 73.73 mmol) and Boc-1-amino-4,7,10-trioxa-13-tridecane amine (28.35 g, 88.48 mmol), an orange oil of molecule A24-1 is obtained.

Yield: 44.50 g (96%)

1H NMR (CDCl3, ppm): 0.87 (3H); 1.08-1.56 (20H); 1.43 (9H); 1.58-1.67 (2H); 1.70-2.00 (6H); 2.04-2.41 (4H); 3.16-3.77 (18H); 4.26-4.29 (0.2H); 4.50-4.54 (0.8H); 4.68-5.10 (1H); 6.74 (0.2H); 7.19 (0.8H).

LC/MS (ESI): 628.4; (calculated ([M+H]+): 628.5).

### Molecule AA11-1

Following a process similar to that used in the preparation of AA1 molecule applied to A24-1 molecule (43.40 g, 69.12 mmol), a white solid of molecule AA11-1 in the form of hydrochloride salt is obtained after trituration 3 times in diethyl ether, solubilization of the residue in water and lyophilization.

Yield: 38.70 g (98%)

1H NMR (DMSO, ppm): 0.85 (3H); 1.07-1.38 (20H); 1.41-1.52 (2H); 1.55-1.66 (2H); 1.70-2.02 (6H); 2.08-2.30 (2H); 2.78-2.87 (2H); 3.00-3.16 (2H); 3.29-3.66 (14H); 4.16-4.22 (0.65H); 4.25-4.30 (0.35H); 7.74 (0.65H); 7.86 (3H); 8.10 (0.35H).

### EXAMPLE AA12: MOLECULE AA12

### Molecule A26: Product Obtained by the Reaction Between Myristoyl Chloride and L-Proline

Myristoyl chloride (322 g, 1.30 mol) dissolved in dichloromethane (1.63 L) is slowly added over 1 h, to a L-proline (300.40 g, 2.61 mol) solution in 2 N aqueous sodium hydroxide (1.63 L) solution at 0° C. At the end of the addition, the reaction medium is raised to 20° C. in 2 hours, then stirred for 2 more hours. The mixture is cooled to 0° C., then a solution of 37% HCl (215 ml) is added within 15 minutes. The reaction mixture is stirred for 10 minutes at 0° C., then 1 h at 0° C. to 20° C. The organic phase is separated, washed with 10% HCl solution (3×430 mL), saturated aqueous NaCl solution (430 mL), dried over Na₂SO₄, filtered through cotton, then concentrated under reduced pressure. The residue is solubilized in heptane (315 ml), then pentane (1.6 L) is added with mechanical stirring. A white solid is obtained after filtration on a sintered filter and drying under reduced pressure.

Yield: 410.6 g (97%)

NMR ¹H (CDCl₃, ppm): 0.88 (3H); 1.28 (20H); 1.70 (2H); 1.90-2.10 (3H); 2.36 (2H); 2.51 (1H); 3.47 (1H); 3.56 (1H); 4.61 (1H).

LC/MS (ESI): 326.4; 651.7; (calculated ([M+H]+): 326.3; ([2M+H]⁺): 651.6).

### Molecule A27: Product Obtained by the Reaction Between Molecule A26 and Boc-Ethylenediamine

To a solution of molecule A26 (3.00 g, 9.21 mmol) at room temperature in methyl-THF (50 mL), are successively added HOBt (1.83 g, 11.98 mmol), then Boc-ethylenediamine (1.62 g, 10.14 mmol) and the medium is cooled to 0° C. EDC (2.29 g, 11.98 mmol) is added and the mixture is stirred for 17 hours between 0° C. and at room temperature. The reaction mixture is then washed with a saturated aqueous solution of NH₄Cl (50 mL), saturated aqueous NaHCO₃ (50 mL) then a saturated aqueous solution of NaCl (50 mL), dried over Na₂SO₄, filtered and concentrated under reduced pressure. A white solid is obtained after recrystallization from methanol.

Yield: 2.34 g (49%).

NMR ¹H (CDCl₃, ppm): 0.88 (3H); 1.16-1.38 (20H); 1.44 (9H); 1.56-1.71 (2H); 1.78-2.45 (6H); 3.11-3.72 (6H); 4.30 (0.1H); 4.51 (0.9H); 4.87 (0.1H); 5.04 (0.9H); 6.87 (0.1H); 7.23 (0.9H).

LC/MS (ESI): 468.0; (calculated ([M+H]+): 468.4).

### Molecule AA12

By a process similar to that used in the preparation of molecule AA1 applied to the molecule A27 (2.34 g, 5.00 mmol), a white solid of molecule AA12 is obtained after evaporation of the solvent and triturations in diisopropyl ether.

Yield: 1.5 g (74%) ¹H NMR (MeOD-d4, ppm): 0.90 (3H); 1.21-1.43 (20H); 1.54-1.66 (2H); 1.85-2.28 (4H); 2.39 (2H); 3.00-3.17 (2H); 3.30-3.40 (1H); 3.43-3.71 (3H); 4.29 (0.94H); 4.48 (0.06H).

LC/MS (ESI): 368.2; (calculated ([M+H]+): 368.3).

### EXAMPLE AA12-1: MOLECULE AA12-1

### Molecule A25-1: Product Obtained by the Reaction Between Molecule B4 and N-Boc-Ethylenediamine

By a process similar to that used in the preparation of molecule A27 applied to molecule B4 (12.00 g, 40.35 mmol) and to Boc-ethylenediamine (7.76 g, 48.42 mmol), a colorless oil is obtained and used without further purification.

Yield: 17.40 g (94%)

1H NMR (CDCl3, ppm): 0.86 (3H); 1.11-1.68 (18H); 1.41 (9H); 1.80-2.38 (6H); 3.06-3.35 (4H); 3.37-3.49 (1H); 3.51-3.73 (1H); 4.26-4.31 (0.1H); 4.45-4.52 (0.9H); 4.91-5.19 (1H); 6.97 (0.1H); 7.23 (0.9H). LC/MS (ESI): 440.4 (calculated ([M+H]+): 440.3).

### Molecule AA12-1

Following a process similar to that used in the preparation of molecule AA1 applied to molecule A25-1 (8.85 g, 20.13 mmol), a white solid of molecule AA12-1 is obtained after basic washing, concentration under reduced pressure, then recrystallization in acetonitrile.

Yield: 6.53 g (96%)

1H NMR (DMSO, ppm): 0.85 (3H); 1.07-1.56 (20H); 1.68-2.03 (4H); 2.09-2.29 (2H); 2.50-2.58 (2H); 2.96-3.11 (2H); 3.21-3.59 (2H); 4.17-4.21 (0.65H); 4.25-4.29 (0.35H); 7.68 (0.65H); 8.00 (0.35H)

LC/MS (ESI): 340.3; (calculated ([M+H]+): 340.3).

### EXAMPLE AA13-1: MOLECULE AA13-1

### Molecule A26-1: Product Obtained by Coupling Between Molecule B1 and the N-Boc-Ethylenediamine

By a process similar to that used in the preparation of molecule A27 applied to molecule B1 (30.00 g, 111.36 mmol) and BocEDA (21.41 g, 133.64 mmol), a white solid is obtained after recrystallization in acetonitrile.

Yield: 34.90 g (76%)

1H NMR (CDCl3, ppm): 0.88 (3H); 1.10-1.70 (14H); 1.43 (9H); 1.80-1.91 (1H); 1.92-2.01 (1H); 2.04-2.42 (4H); 3.13-3.70 (6H); 4.27-4.31 (0.15H); 4.47-4.53 (0.85H); 4.83 (0.15H); 5.02 (0.85H); 6.85 (0.15H); 7.21 (0.85H).

LC/MS (ESI): 412.2; (calculated ([M+H]+): 412.3).

### Molecule AA13-1

Following a process similar to that used in the preparation of molecule AA1 applied to molecule A26-1 (34.90 g, 84.79 mmol), a white solid of molecule AA13-1 in the form of hydrochloride salt is obtained after solubilization in a DCM/acetonitrile mixture and concentration under reduced pressure.

Yield: 29.50 g (99%)

1H NMR (DMSO, ppm): 0.85 (3H); 1.07-1.61 (14H); 1.70-2.06 (4H); 2.10-2.35 (2H); 2.76-2.87 (2H); 3.24-3.47 (3.25H); 3.56-3.64 (0.75H); 4.13-4.19 (0.75H); 4.31-4.36 (0.25H); 8.05-8.36 (3.75H); 8.50 (0.25H).

LC/MS (ESI): 312.2; (calculated ([M+H]+): 312.3).

### EXAMPLE AA14: MOLECULE AA14

### Resin AA14-1: Product Obtained by the Reaction Between 4,7,10-Trioxa-1,13-Tridecanediamine and the 2-Cl-Trityl Chloride Resin

DIPEA (8.64 mL, 49.60 mmol) is added at room temperature to a solution of 4,7,10-trioxa-1,13-tridecanediamine (10.87 mL, 49.60 mmol) in dichloromethane (50 mL). This solution is poured on 2-Cl-trityl chloride resin previously washed with dichloromethane (100-200 mesh, 1% DVB, 1.24 mmol/g) (4.00 g, 4.96 mmol) in a peptide synthesis reactor on a solid support. After stirring for 2 hours at room temperature, HPLC grade methanol (0.8 mL/g resin, 3.2 mL) is added and the mixture is stirred at room temperature for 15 minutes. The resin is filtered, washed successively with dichloromethane (3×50 mL), DMF (2×50 mL), dichloromethane (2×50 mL), isopropanol (1×50 mL) and dichloromethane (3×50 mL).

### Resin AA14-2: Product Obtained by Reaction Between Resin AA14-1 and Fmoc-Glycine

DIPEA (5.18 mL, 29.76 mmol) is added to a suspension of Fmoc-glycine (4.42 g, 14.88 mmol) and 1-[bis (dimethylamino) methylene]-1H-1,2,3-triazolo [4,5-b]pyridinium 3-oxide hexafluorophosphate (HATU, 5.66 g, 14.88 mmol) in 1:1 DMF/dichloromethane (60 mL). After complete solubilization, the solution obtained is poured onto the resin AA14-1. After stirring for 2 hours at room temperature, the resin is filtered, washed successively with DMF (3×60 mL), isopropanol (1×60 mL) and dichloromethane (3×60 mL).

resin AA14-3: Product obtained by reaction between AA14-2 resin and an 80:20 DMF/piperidine mixture. AA14-2 resin is treated with an 80:20 DMF/piperidine mixture (50 mL). After stirring for 30 h at room temperature, the resin is filtered, washed successively with DMF (3×50 mL), isopropanol (1×50 mL) and dichloromethane (3×50 mL).

### Resin AA14-4: Product Obtained by Reaction Between Resin AA14-3 and Fmoc-Proline

By a process similar to that used for resin AA14-2 applied to resin AA14-3 and Fmoc-proline (5.02 g, 14.88 mmol) in DMF (50 mL), resin AA14-4 is obtained.

### Resin AA14-5: Product Obtained by Reaction Between Resin AA14-4 and an 80:20 DMF/Piperidine Mixture

By a process similar to that used for AA14-3 resin applied to AA14-4 resin and a 80:20 DMF/piperidine mixture (50 mL), resin AA14-5 is obtained.

### Resin AA14-6: Product Obtained by Reaction Between Resin AA14-5 and Palmitic Acid

By a process similar to that used in the preparation of AA14-4 resin applied to AA14-5 resin and palmitic acid (3.82 g, 14.88 mmol), resin AA14-6 is obtained.

### Molecule AA14

Resin AA14-6 is treated with a 1:1 TFA/dichloromethane mixture (50 mL). After stirring for 30 minutes at room temperature, the resin is filtered and washed with dichloromethane (3×50 mL). The solvents are evaporated under vacuum. Two co-evaporations are then borne out on the residue with dichloromethane (50 ml), then diisopropyl ether (50 ml). The residue is solubilized in dichloromethane (50 mL) and the organic phase is washed with an aqueous solution of 1N NaOH (1×50 mL), then a saturated solution of NaCl (2×50 mL). After drying on Na₂SO₄, the organic phase is filtered, concentrated in vacuum and the residue is purified by chromatography on silica gel (dichloromethane, methanol, NH₄OH)

Yield: 1.65 g (54% overall over 7 steps)

NMR ¹H (CDCl₃, ppm): 0.88 (3H); 1.18-2.39 (38H); 2.79 (2H); 3.23-3.44 (2H); 3.47-3.69 (14H); 3.76 (0.92H); 3.82 (0.08H); 3.98 (0.08H); 4.03 (0.92H); 4.34 (0.08H); 4.39 (0.92H); 7.00-7.40 (2H).

LC/MS (ESI): 613.7; (calculated ([M+H]+): 613.5).

### EXAMPLE AA14-1: MOLECULE AA14-1

### Molecule A27-1: Product Obtained by Hydrogenation of Phytol

Platinum oxide (PtO₂, 1.15 g, 6.61 mmol) is added to a solution of phytol (30.00 g, 101.20 mmol) in THF (450 mL) under argon and the medium is placed under 1 bar of dihydrogen, then stirred for 4 hours at room temperature. After filtration through celite by rinsing with THF, a black oil of molecule A27-1 is obtained after concentration under reduced pressure.

Yield: 29.00 g (96%)

NMR ¹H (CDCl₃, ppm): 0.84 (6H); 0.86 (6H); 0.89 (3H); 1.00-1.46 (22H); 1.46-1.68 (3H); 3.61-3.73 (2H).

### Molecule A28: Product Obtained by Oxidation of Molecule A27-1

Tetrabutylammonium bromide (16.90 g, 52.45 mmol) acetic acetic acid (150 mL, 2.62 mol) followed by KMnO₄ (46.05 g, 291.40 mmol) are successively added to a solution of molecule A27-1 (29.0 g, 97.13 mmol) in a dichloroethane/water mixture (485 mL/388 mL) in small portions keeping the temperature between 16 and 19° C. The reaction medium is then stirred for 4 h30 at reflux, cooled to 10° C., then acidified to pH 1 with a solution of 6 N HCl (20 mL). Na₂SO₃ (53.90 g) is then added gradually while maintaining the temperature at 10° C., and the mixture is stirred until complete decolorization. Water (200 mL) is added, the phases are separated, and the aqueous phase is extracted with DCM (2×400 mL). The combined organic phases are washed with 10% HCl aqueous solution (20 mmL), water (2×200 mL), a saturated aqueous solution of NaCl (200 mL), dried over Na₂SO₄, filtered and concentrated under reduced pressure. A yellow oil of molecule A28 is obtained after purification by flash chromatography (eluent: cyclohexane, AcOEt).

Yield: 28.70 g (94%)

NMR ¹H (CDCl₃, ppm): 0.84 (6H); 0.86 (6H); 0.97 (3H); 1.00-1.41 (20H); 1.52 (1H); 1.96 (1H); 2.14 (1H); 2.35 (1H); 11.31 (1H).

LC/MS (ESI): 311.1 (calculated ([M−H]⁻): 311.3).

### Molecule A29: Product Obtained by Coupling Between Molecule A28 and Methyl L-Prolinate

By a process similar to that used in the preparation of molecule A2 applied to molecule A28 (18.00 g, 57.59 mmol) and methyl L-prolinate hydrochloride (14.31 g, 86.39 mmol) a yellow oil of molecule A29 is obtained after washing the organic phase with a NaHCO₃ (2×150 mL) saturated aqueous solution, a 10% aqueous solution of HCl (2×150 mL), a saturated aqueous solution of NaCl (2×150 mL), then drying on Na₂SO₄, filtration and concentration under reduced pressure.

Yield: 23.20 g (95%)

NMR ¹H (DMSO-d₆, ppm): 0.78-0.89 (15H); 0.97-1.43 (20H); 1.43-1.56 (1H); 1.70-1.96 (4H); 1.96-2.32 (3H); 3.33-3.56 (2H); 3.59 (0.6H); 3.67 (2.4H); 4.27 (0.8H); 4.57 (0.2H).

LC/MS (ESI): 424.4 (calculated ([M+H]+): 424.4).

### Molecule A30: Product obtained by the saponification of molecule A29

By a process similar to that used in the preparation of molecule A21 applied to molecule A29 (21.05 g, 49.68 mmol), a yellow oil of molecule A30 is obtained.

Yield: 20.40 g (99%)

NMR ¹H (DMSO-d₆, ppm): 0.77-0.91 (15H); 0.97-1.43 (20H); 1.43-1.56 (1H); 1.67-1.96 (4H); 1.96-2.29 (3H); 3.26-3.56 (2H); 4.20 (0.8H); 4.41 (0.2H).

LC/MS (ESI): 410.3 (calculated ([M+H]+): 410.4).

### Molecule A31: Product Obtained by Reaction Between Molecule A30 and Boc-1-Amino-4,7,10-Trioxa-13-Tridecane Amine

By a process similar to that used in the preparation of molecule A27 applied to molecule A30 (8.95 g, 21.85 mmol) and Boc-1-amino-4,7,10-trioxa-13-tridecane amine (8.40 g, 26.21 mmol), a colorless oil of molecule A31 is obtained after purification by flash chromatography (eluent: DCM, AcOEt, methanol).

Yield: 10.08 g (65%)

NMR ¹H (DMSO-d₆, ppm): 0.78-0.89 (15H); 0.97-1.43 (29H); 1.43-1.55 (1H); 1.55-1.66 (4H); 1.71-2.30 (7H); 2.95 (2H); 3.00-3.19 (2H); 3.34-3.58 (14H); 4.17-4.29 (1H); 6.30-6.79 (1H); 7.67 (0.65H); 8.00 (0.35H).

LC/MS (ESI): 712.6 (calculated ([M+H]+): 712.6).

### Molecule AA14-1

Following a process similar to that used in the preparation of molecule AA1 applied to molecule A31 (10.08 g, 14.16 mmol), the residue obtained after concentration under reduced pressure is solubilized in DCM (200 mL). The organic phase is washed with an aqueous solution of 2N NaOH (2×100 mL), dried over Na₂SO₄, filtered and concentrated under reduced pressure. A colorless oil of molecule AA14-1 in neutral amine form is obtained.

Yield: 8.23 g (95%)

NMR ¹H (DMSO-d₆, ppm): 0.78-0.89 (15H); 0.97-1.43 (20H); 1.43-1.69 (6H); 1.69-2.30 (8H); 2.56 (2H); 2.99-3.19 (2H); 3.31-3.58 (14H); 4.15-4.29 (1H); 7.70 (0.65H); 8.04 (0.35H).

LC/MS (ESI): 612.5 (calculated ([M+H]+): 612.5).

### EXAMPLE AA15: MOLECULE AA15

Molecule AA15 is obtained by the conventional method of solid phase peptide synthesis (SPPS) on 2-chlorotrityl resin.

DIPEA (8.64 mL, 49.60 mmol) is added to a solution of 4,7,10-trioxa-1,13-tridecanediamine (TOTA, 10.87 mL, 49.60 mmol) in DCM (50 mL). This solution is then poured onto 2-chlorotrityl resin (4.00 g, 1.24 mmol/g) previously washed with DCM in a reactor adapted to SPPS. After stirring for 2 hours at room temperature, methanol (0.8 mL/g, 3.2 mL) is added and the medium is stirred for 15 minutes. The resin is filtered, washed successively with DCM (3×50 mL), DMF (2×50 mL), DCM (2×50 mL), isopropanol (1×50 mL) and DCM (3×50 mL). Protected amino acids N-Fmoc-L-glycine and N-Fmoc-L-proline, then palmitic acid (3 equivalents) are coupled successively using 1-[bis (dimethylamino) methylene]-1H-1,2,3-triazolo [4.5-b] pyridinium 3-oxide hexafluorophosphate (HATU, 3 equivalents) as coupling agent in the presence of DIPEA (6 equivalents) in a 1:1 DCM/DMF mixture. A solution of 20% piperidine in DMF is used for the cleavage steps of the Fmoc protecting group. The resin is washed with DCM, DMF and isopropanol after each coupling and deprotection step. Cleavage of the resin product is carried out using a 1:1 TFA/DCM mixture. The solvents are evaporated under reduced pressure, the residue is solubilized in DCM (50 mL) and the organic phase is washed with a 1N aqueous solution of NaOH (1×50 mL), then a saturated solution of NaCl (2×50 mL). After drying on Na₂SO₄, the organic phase is filtered, concentrated under reduced pressure and the residue is purified by chromatography on silica gel (dichloromethane, methanol, NH₄OH).

Yield: 1.65 g (54% overall over 7 steps).

NMR ¹H (CDCl₃, ppm): 0.88 (3H); 1.18-2.39 (38H); 2.79 (2H); 3.23-3.44 (2H); 3.47-3.69 (14H); 3.76 (0.92H); 3.82 (0.08H); 3.98 (0.08H); 4.03 (0.92H); 4.34 (0.08H); 4.39 (0.92H); 7.00-7.40 (2H).

LC/MS (ESI): 613.7; (calculated ([M+H]+): 613.5).

### EXAMPLE AA16: MOLECULE AA16

By a SPPS process similar to that used in the preparation of molecule AA15 and using the N-Fmoc-L-phenylalanine (3 equivalents) instead of N-Fmoc-L-glycine, molecule AA16 is obtained in the form of a yellow oil.

Yield: 14.07 g (69%)

NMR ¹H (CDCl₃, ppm): 0.88 (3H); 1.19-1.34 (24H); 1.41-1.61 (2H); 1.68-2.28 (12H); 2.84 (2H); 3.14 (2H); 3.23-3.67 (16H); 4.19-4.25 (0.1H); 4.38-4.45 (0.9H); 4.59-4.69 (1H); 6.86 (1H); 7.03 (1H); 7.12-7.30 (5H).

LC/MS (ESI): 703.5; (calculated ([M+H]+): 703.5).

### EXAMPLE AA17: MOLECULE AA17

Molecule AA17 is obtained in the form of a white solid by a SPPS process similar to that used in the preparation of AA15 molecule and using EDA (30.48 mL, 0.456 mol) instead of TOTA.

Yield: 9.19 g (89%)

¹H NMR (MeOD-d4, ppm): 0.90 (3H); 1.22-1.43 (24H); 1.55-1.67 (2H); 1.91-2.04 (2H); 2.04-2.15 (1H); 2.17-2.29 (1H); 2.39 (2H); 2.69-2.82 (2H); 3.25-3.36 (2H); 3.58-3.70 (2H); 3.70-3.97 (2H); 4.25-4.34 (0.9H); 4.44-4.50 (0.1H).

LC/MS (ESI): 453.3; (calculated ([M+H]+): 453.4).

### AB: Co-Polyamino Acids Defined According to Formula VII, VIIa or VIIb

TABLE 1c
list of co-polyamino acids synthesized according to the invention.

| | CO-POLYAMINOACIDES BEARING CARBOXYLATE LOADS AND |
|---|---|
| No | HYDROPHOBIC RADICALS |
| AB1 | 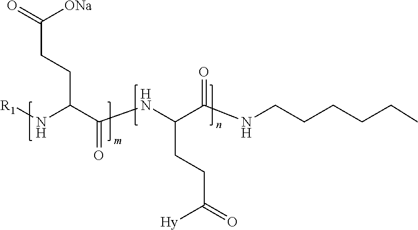 |
| | I = 0.05, DP (m + n) = 23 |
| | Hy = |
| | 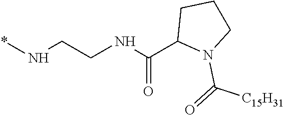 |
| | R₁ = H or pyroglutamate |
| AB2 | 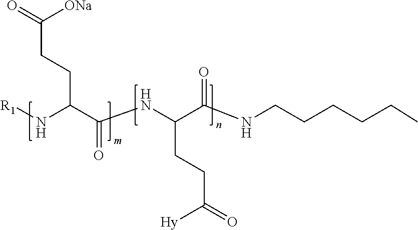 |
| | i = 0.05, DP (m + n) = 35 |
| | Hy = |
| | 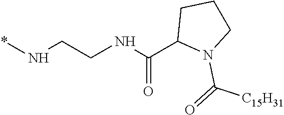 |
| | R₁ = H or pyroglutamate |
| AB3 | 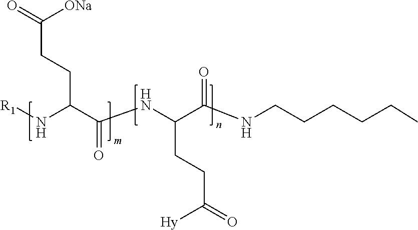 |
| | i = 0.10, DP (m + n) = 35 |
| | Hy = |
| | 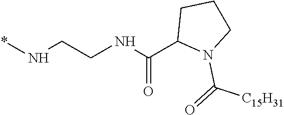 |
| | R₁ = H or pyroglutamate |
| AB4 | 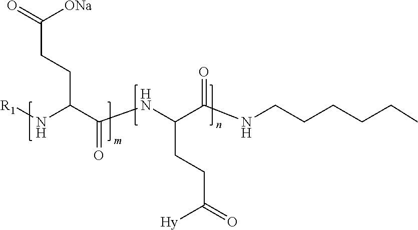 |
| | i = 0.052, DP (m + n) = 35 |
| | Hy = |
| | 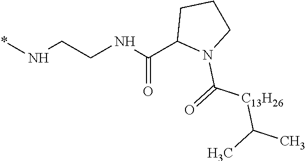 |
| | R₁ = H or pyroglutamate |
| AB5 | 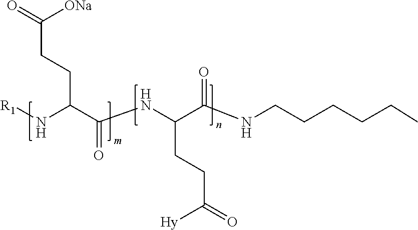 |
| | i = 0.05, DP (m + n) = 23 |
| | Hy = |
| | 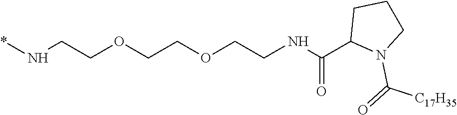 |
| | R₁ = H or pyroglutamate |
| AB6 | 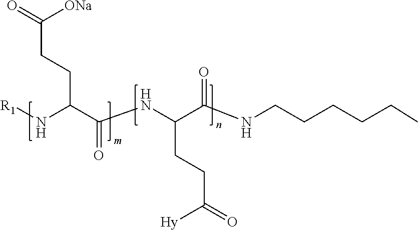 |
| | i = 0.025, DP (m + n) = 20 |
| | Hy = |
| | 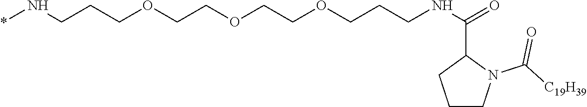 |
| | R₁ = H or pyroglutamate |
| AB7 | 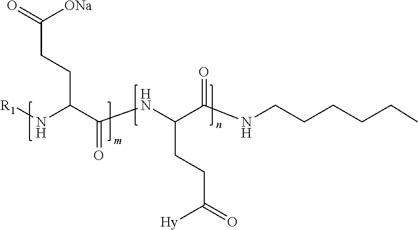 |
| | i = 0.03, DP (m + n) = 21 |
| | Hy = |
| | 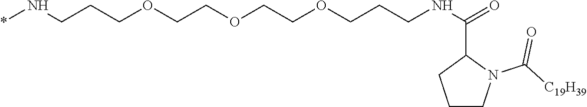 |
| | R₁ = CH₃—CO—, H or pyroglutamate |
| AB8 | 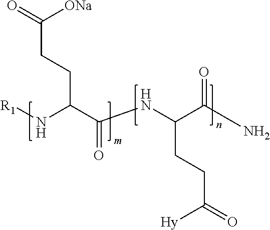 |
| | i = 0.03, DP (m + n) = 24 |
| | Hy = |
| | 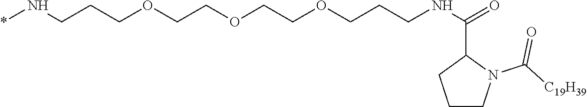 |
| | R₁ = H or pyroglutamate |
| AB9 | 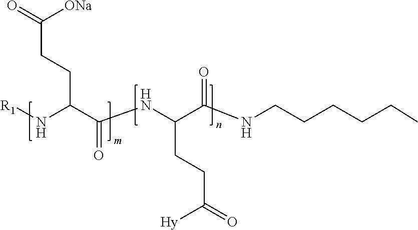 |
| | i = 0.12, DP (m + n) = 30 |
| | Hy = |
| | 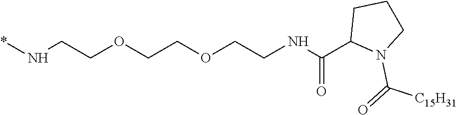 |
| | R₁ = H or pyroglutamate |
| AB10 | 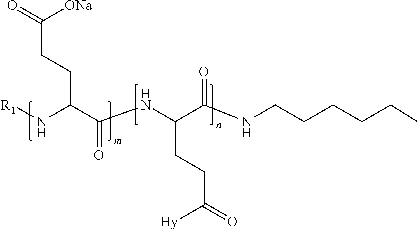 |
| | i = 0.08, DP (m + n) = 25 |
| | Hy = |
| | 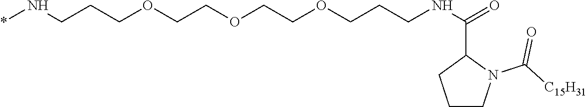 |
| | R₁ = H or pyroglutamate |
| AB11 | 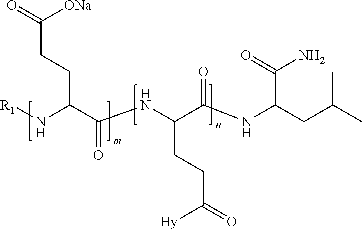 |
| | i = 0.05, DP (m + n) = 23 |
| | Hy = |
| | 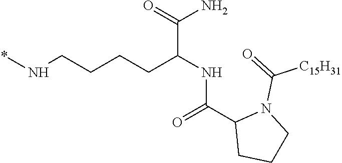 |
| | R₁ = H or pyroglutamate |
| AB12 | 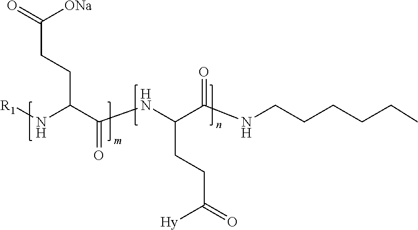 |
| | i = 0.04, DP (m + n) = 26 |
| | Hy = |
| | 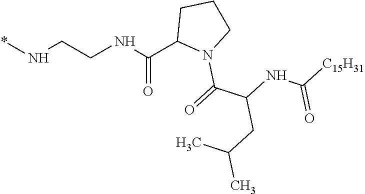 |
| | R₁ = H or pyroglutamate |
| AB13 | 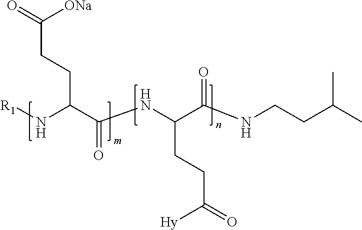 |
| | i = 0.12, DP (m + n) = 35 |
| | Hy = |
| | 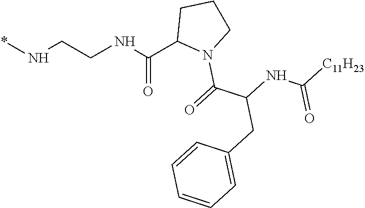 |
| | R₁ = H or pyroglutamate |
| AB14 | 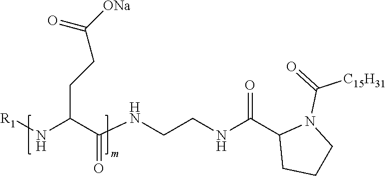 |
| | i = 0.04, DP (m) = 25 |
| | R₁ = H or pyroglutamate |
| AB15 | 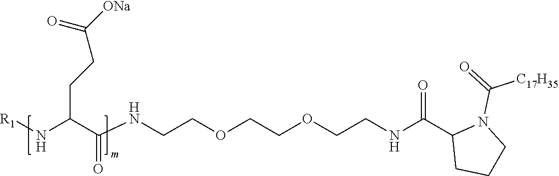 |
| | i = 0.033, DP (m) = 30 |
| | R₁ = H or pyroglutamate |
| AB16 | 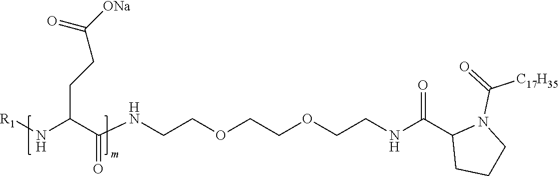 |
| | i = 0.021, DP (m) = 48 |
| | R₁ = H or pyroglutamate |
| AB17 | 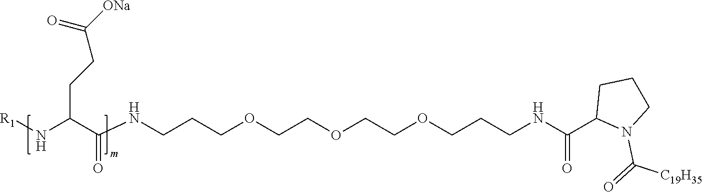 |
| | i = 0.038, DP (m) = 26 |
| | R₁ = H or pyroglutamate |
| AB18 | 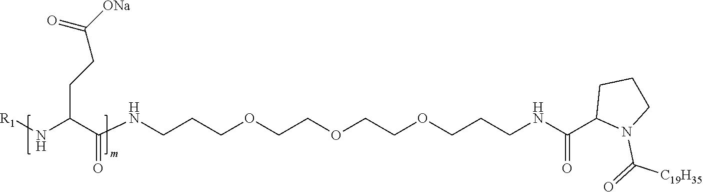 |
| | i = 0.045, DP (m) = 22 |
| | R₁ = H or pyroglutamate |
| AB19 | 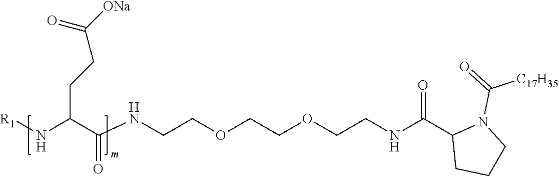 |
| | i = 0.015, DP (m) = 65 |
| | R₁ = H or pyroglutamate |
| AB20 | 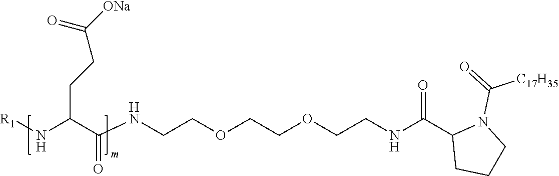 |
| | i = 0.017, DP (m) = 60 |
| | R₁ = CH₃—CO—, H or pyroglutamate |
| AB21 | 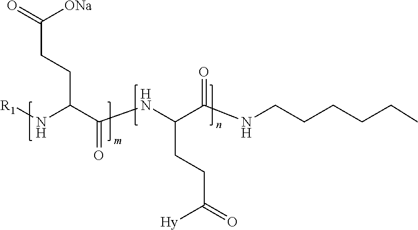 |
| | i = 0.056, DP (m + n) = 22 |
| | Hy = |
| | 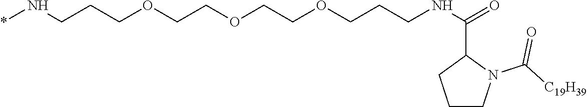 |
| | R₁ = H or pyroglutamate |
| AB21′ | 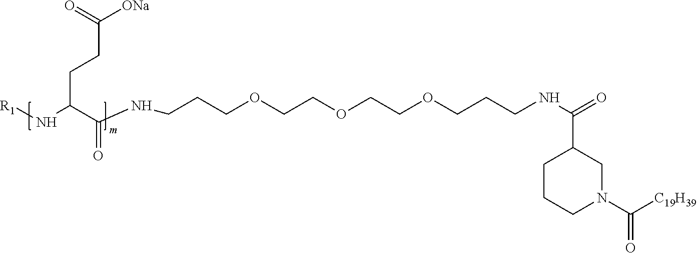 |
| | i = 0.04, DP (m) = 25 |
| | R₁ = H or pyroglutamate |
| AB22′ | 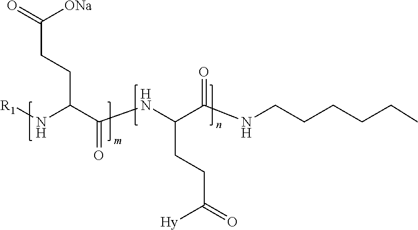 |
| | i = 0.16, DP (m + n) = 38 |
| | Hy = |
| | 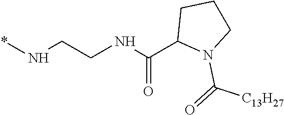 |
| | R₁ = CH₃CO or pyroglutamate |
| AB23 | 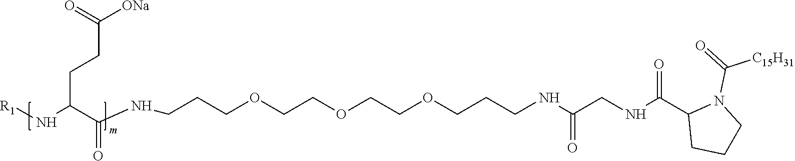 |
| | i = 0.045, DP (m) = 22 |
| | R₁ = H or pyroglutamate |
| AB23′ | 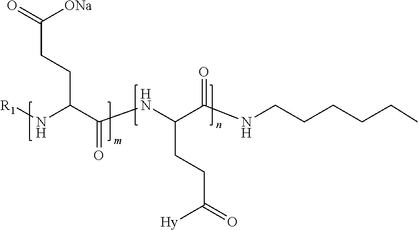 |
| | i = 0.10, DP (m + n) = 60 |
| | Hy = |
| | 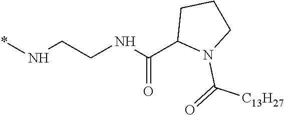 |
| | R₁ = H or pyroglutamate |
| AB24 | 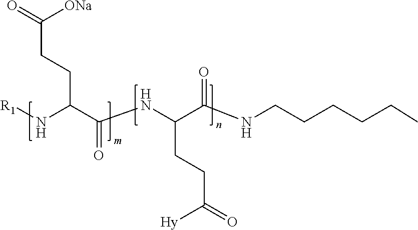 |
| | i = 0.089, DP (m + n) = 38 |
| | Hy = |
| | 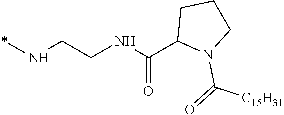 |
| | R₁ = CH₃—CO—, H or pyroglutamate |
| AB25 | 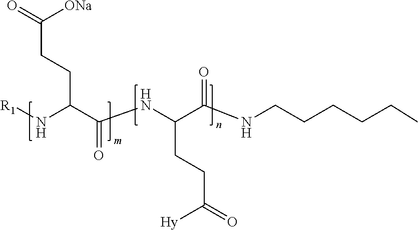 |
| | i = 0.16, DP (m + n) = 38 |
| | Hy = |
| | 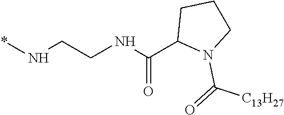 |
| | R₁ = CH₃—CO—, H or pyroglutamate |
| AB26′ | 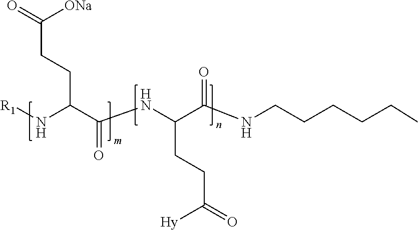 |
| | i = 0.20, DP (m + n) = 22 |
| | Hy = |
| | 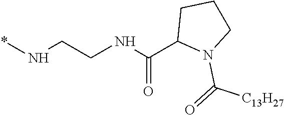 |
| | R₁ = H or pyroglutamate |
| AB27′ | 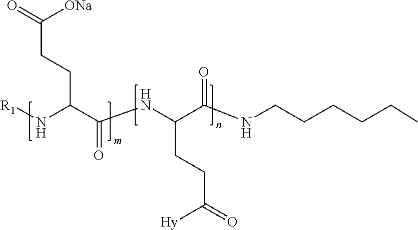 |
| | i = 0.15, DP (m + n) = 39 |
| | Hy = |
| | 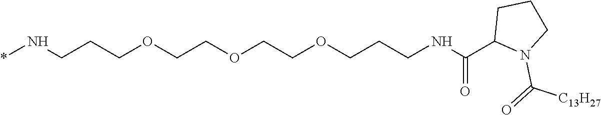 |
| | R₁ = H or pyroglutamate |
| AB28′ | 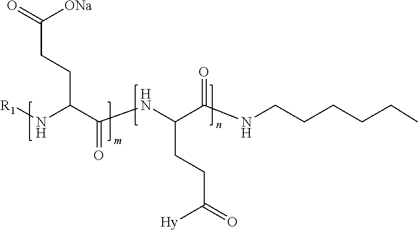 |
| | i = 0.15, DP (m + n) = 39 |
| | Hy = |
| | 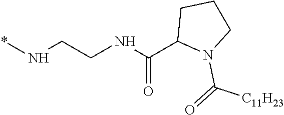 |
| | R₁ = H or pyroglutamate |
| AB28 | 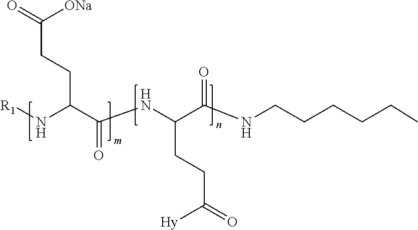 |
| | i = 0.1, DP (m + n) = 38 |
| | Hy = |
| | 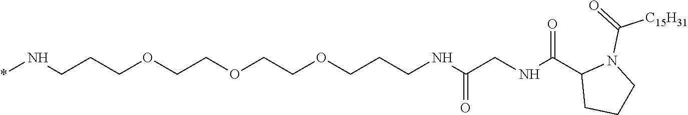 |
| | R₁ = CH₃—CO—, H or pyroglutamate |
| AB29 | 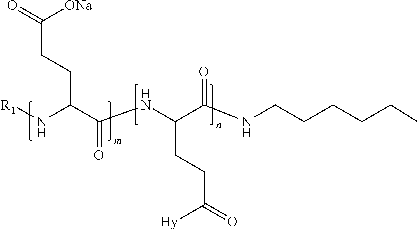 |
| | i = 0.15, DP (m + n) = 39 |
| | Hy = |
| | 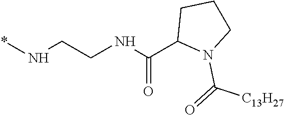 |
| | R₁ = H or pyroglutamate |
| AB29′ | 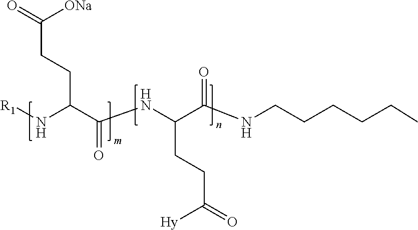 |
| | i = 0.15, DP (m + n) = 40 |
| | Hy = |
| | 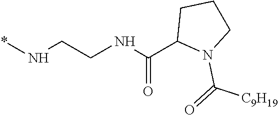 |
| | R₁ = pyroglutamate |
| AB30 | 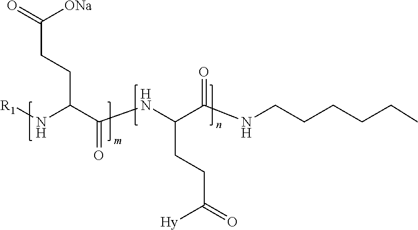 |
| | i = 0.20, DP (m + n) = 39 |
| | Hy = |
| | 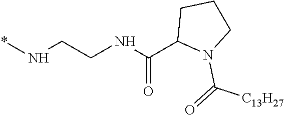 |
| | R₁ = H or pyroglutamate |
| AB32′ | 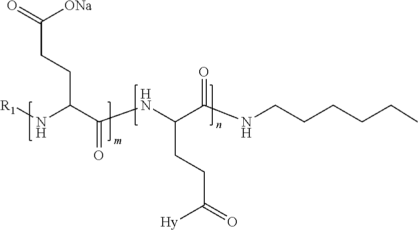 |
| | i = 0.109 DP (m + n) = 40 |
| | Hy = |
| | 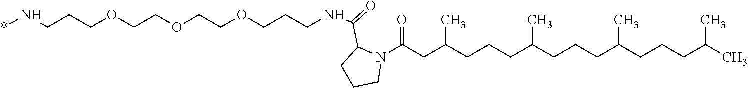 [SMILES: *NCCCOCCOCCOCCCNC(=O)C1CCCN1C(=O)CC(C)CCCC(C)CCCC(C)CCCC(C)C] |
| | R₁ = pyroglutamate |
| AB33 | 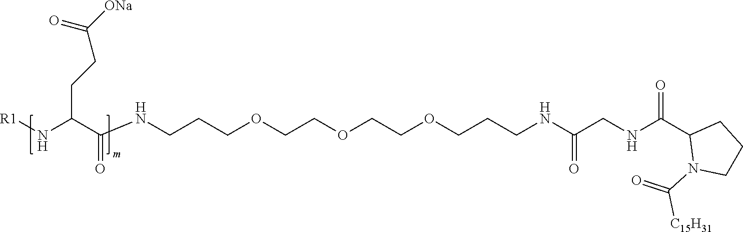 |
| | i = 0.045, DP (m) = 22 |
| | R₁ = H or pyroglutamate |
| AB34 | 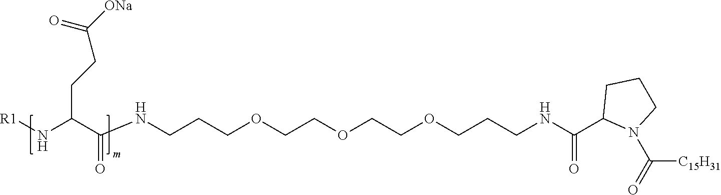 |
| | i = 0.043, DP (m) = 23 |
| | R₁ = H or pyroglutamate |
| AB35 | 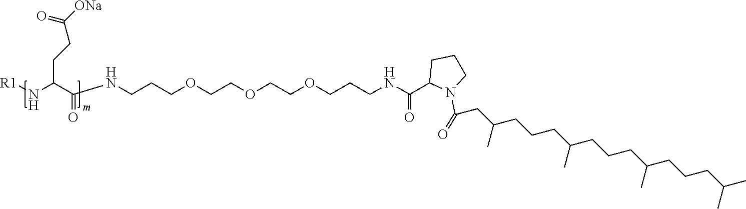 |
| | i = 0.045, DP (m) = 22 |
| | R₁ = H or pyroglutamate |
| AB36 | 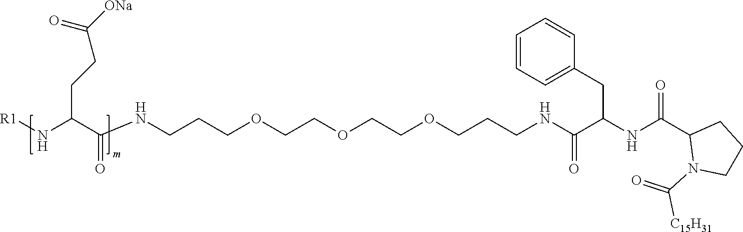 |
| | i = 0.045, DP (m) = 22 |
| | R₁ = H or pyroglutamate |
| AB37 | 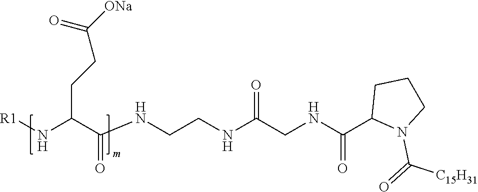 |
| | i = 0.042, DP (m) = 24 |
| | R₁ = H or pyroglutamate |

### Part AB: Synthesis of Co-Polyamino Acids

### EXAMPLE AB1: CO-POLYAMINO ACID AB1—SODIUM POLY-L-GLUTAMATE MODIFIED BY MOLECULE AA1 AND HAVING A NUMBER-AVERAGE MOLECULAR WEIGHT (MN) OF 2900 G/MOL

### Co-Polyamino Acid AB1-1: Poly-L-Glutamic Acid of Number-Average Molecular Weight (Mn) 3861 g/Mol from the Polymerization of γ-Benzyl-L-Glutamate N-Carboxyanhydride Initiated by Hexylamine

In a previously oven-dried flask, γ-benzyl-L-glutamate N-carboxyanhydride (89.9 g, 341 mmol) is placed under vacuum for 30 minutes, then anhydrous DMF (200 mL) is added. The mixture is then stirred under argon until complete dissolution, cooled to 4° C., then hexylamine (2.05 ml, 15.5 mmol) is quickly introduced. The mixture is stirred at 4° C. and room temperature for 2 days. The reaction medium is then heated at 65° C. for 2 hours, cooled to room temperature, then poured dropwise into diisopropyl ether (3 L) with stirring. The white precipitate is collected by filtration, washed with diisopropyl ether (2×200 mL), then dried under vacuum at 30° C. to give a poly (gamma-benzyl-L-glutamic acid) (PBLG).

A solution of hydrobromic acid (HBr) at 33% in acetic acid (240 mL, 1.37 mol) is added dropwise to a solution of PBLG (74.8 g) in trifluoroacetic acid (TFA, 340 mL) at 4° C. The mixture is stirred at room temperature for 2 hours, then poured dropwise onto a 1:1 (v/v) mixture of diisopropyl ether and water with stirring (4 L). After stirring for 2 hours, the heterogeneous mixture is left to stand overnight. The white precipitate is collected by filtration, washed with a 1:1 (v/v) mixture of diisopropyl ether and water (340 mL), then with water (340 mL).

The solid obtained is solubilized in water (1.5 mL) by adjusting the pH to 7 by adding 10 N aqueous sodium hydroxide solution, then 1N aqueous sodium hydroxide solution. After solubilization, the theoretical concentration is adjusted to 20 g/L theoretical by addition of water to obtain a final volume of 2.1 mL.

The solution is filtered through a 0.45 μm filter, then purified by ultrafiltration against a solution of NaCl 0.9%, then water until the conductimetry of the permeate is less than 50 μS/cm. The co-polyamino acid solution is then concentrated until a final volume of 1.8 L is obtained.

The aqueous solution is then acidified by adding a 37% hydrochloric acid solution until a pH of 2 is reached. After stirring for 4 hours, the precipitate obtained is filtered, washed with water (2×340 mL), then dried under vacuum at 30° C. to give a poly-L-glutamic acid of number-average molecular weight (Mn) 3861 g/mol with respect to a standard of polyoxyethylene (PEG).

### Co-Polyamino Acid AB1

Co-polyamino acid AB1-1 (10.0 g) is solubilized in DMF (700 mL) at 30° C.-40° C., then cooled to 0° C. molecule AA1 in the form of hydrochloride salt (1.64 g, 3.8 mmol) is suspended in DMF (23 mL) and triethylamine (0.39 g, 3.8 mmol) is then added and the mixture is slightly heated while stirring until complete dissolution. N-methylmorpholine (NMM, 7.6 g, 75 mmol) in DMF (14 mL) and ethyl chloroformate (ECF, 8.2 g, 75 mmol) are added to a co-polyamino acid solution at 0° C. After 10 minutes at 0° C., the solution containing the molecule AA1 is added and the medium maintained at 30° C. for 2 hours. The reaction mixture is poured dropwise over 5.5 L of water containing 15% by weight sodium chloride and HCl (pH 2), then left to stand overnight. The precipitate is collected by filtration and dried under vacuum for about 30 minutes. The white solid obtained is taken up in water (500 ml) and the pH is adjusted to 7 by slow addition of a 1N aqueous solution of NaOH. After filtration on a 0.45 m filter, the clear solution obtained is purified by ultrafiltration against 0.9% NaCl solution, then with water, until the conductimetry of the permeate is less than 50 μS/cm. After removal, the solution is filtered through a 0.2 μm filter and stored at 2-8° C.

Dry Extract: 24.9 mg/g

An average degree of polymerization (DP) of 23 is estimated by ¹H NMR in D₂O comparing the integration of the signals from the grafted hydrophobe to that of the signals from the main chain.

Based on ¹H NMR: i=0.05

The calculated average molecular weight of co-polyamino acid AB1 is calculated on the basis of the molar masses of radicals R₁ and R₂, aspartate and/or glutamate residues (including an amide linkage), hydrophobic radical, DS and DP.

The calculated average molecular weight of co-polyamino acid AB1 is 3945 g/mol. Aqueous HPLC-SEC (PEG calibrant): Mn=2900 g/mol.

### EXAMPLE AB2: CO-POLYAMINO ACID AB2—SODIUM POLY-L-GLUTAMATE MODIFIED BY MOLECULE AA1 AND HAVING A NUMBER-AVERAGE MOLECULAR WEIGHT (MN) OF 3700 G/MOL

A sodium poly-L-glutamate modified with molecule AA1 is obtained by a process similar to that used for the preparation of the co-polyamino acid AB1 applied to the hydrochloride salt of molecule AA1 (1.64 g, 3.8 mmol) and a poly-L-glutamic acid of Mn relative 5200 g/mol (10.0 g) obtained by a process similar to that used for the preparation of the co-polyamino acid AB1-1.

Dry extract: 14.1 mg/g

DP (estimated based on ¹H NMR): 35

Based on ¹H NMR: i=0.05

The calculated average molecular weight of co-polyamino acid AB2 is 5972 g/mol.

Aqueous HPLC-SEC (PEG calibrant): Mn=3700 g/mol.

### EXAMPLE AB3: CO-POLYAMINO ACID AB3—SODIUM POLY-L-GLUTAMATE MODIFIED BY MOLECULE AA1 AND HAVING A NUMBER-AVERAGE MOLECULAR WEIGHT (MN) OF 4900 G/MOL

A sodium poly-L-glutamate modified with molecule AA1 is obtained by a process similar to that used in the preparation of the co-polyamino acid AB1 applied to the hydrochloride salt of molecule AA1 (3.30 g, 7.6 mmol) and to a poly-L-glutamic acid of relative number-average molecular weight (Mn)/mol 5200 g/mol (10.0 g) obtained by a process similar to that used in the preparation of co-polyamino acid AB1-1.

Dry extract: 23.4 mg/g

DP (estimated based on ¹H NMR): 35

The calculated average molecular weight of co-polyamino acid AB3 is 6594 g/mol.

Based on ¹H NMR: i=0.10

Aqueous HPLC-SEC (PEG calibrant): Mn=4900 g/mol.

### EXAMPLE AB4: CO-POLYAMINO ACID AB4—SODIUM POLY-L-GLUTAMATE MODIFIED BY MOLECULE AA2 AND HAVING A NUMBER-AVERAGE MOLECULAR WEIGHT (MN) OF 1800 G/MOL

By a process similar to that used in the preparation of co-polyamino acid AB1 applied to the hydrochloride salt of molecule AA2 (1.09 g, 2.4 mmol) and a poly-L-glutamic acid of average mass Mn=5600 g/mol (6.3 g) obtained by a process similar to that used in the preparation of co-polyamino acid AB1-1 but with a benzyl ester deprotection step using trimethylsilane iodide according to the protocol described in publication J. Am. Chem. Soc. 2000, 122, 26-34 (Subramanian G., et al.), A sodium poly-L-glutamate modified with AA2 molecule is obtained.

Dry extract: 21.5 mg/g

DP (estimated based on ¹H NMR): 35

Based on ¹H NMR: i=0.052

The calculated average molecular weight of co-polyamino acid AB4 is 6022 g/mol.

Aqueous HPLC-SEC (PEG calibrant): Mn=1800 g/mol.

### EXAMPLE AB5: CO-POLYAMINO ACID AB5—SODIUM POLY-L-GLUTAMATE MODIFIED BY MOLECULE AA6 AND HAVING A NUMBER-AVERAGE MOLECULAR WEIGHT (MN) OF 2600 G/MOL

A sodium poly-L-glutamate modified with molecule AA6 is obtained by a process similar to that used in the preparation of co-polyamino acid AB1 applied to the hydrochloride salt of molecule AA6 (2.06 g, 3.8 mmol) and to a poly-L-glutamic acid (9.8 g) obtained by a process similar to that used in the preparation of polyamino acid AB1-1.

Dry extract: 20.9 mg/g

DP (estimated based on ¹H NMR): 23

Based on ¹H NMR: i=0.05

The calculated average molecular weight of co-polyamino acid AB5 is 4079 g/mol.

Aqueous HPLC-SEC (PEG calibrant): Mn=2600 g/mol.

### EXAMPLE AB6: CO-POLYAMINO ACID AB6—SODIUM POLY-L-GLUTAMATE MODIFIED BY MOLECULE AA7 AND HAVING A NUMBER-AVERAGE MOLECULAR WEIGHT (MN) OF 4000 G/MOL

A poly-L-glutamic acid of average mass Mn=3500 g/mol and a polymerization degree of 22 (10.0 g) obtained by a process similar to that used in the preparation of co-polyamino acid AB1-1 is solubilized in DMF (420 mL) at 30°-40° C. and maintained at this temperature. In parallel, the hydrochloride salt of the molecule AA7 (1.47 g, 2.3 mmol) is suspended in DMF (12 mL) and triethylamine (0.23 g, 2.3 mmol) is added then the mixture is gently heated with stirring until complete dissolution. NMM (7.6 g, 75 mmol), solution AA7, then 2-hydroxypyridine N-oxide (HOPO, 0.84 g, 7.5 mmol) are successively added to the co-polyamino acid solution in DMF. The reaction medium is then cooled to 0° C., then EDC (1.44 g, 7.5 mmol) is added and the medium is raised to room temperature for 2 hours. The reaction medium is filtered through a 0.2 mm woven filter and poured drop by drop onto 3.5 L of water containing NaCl 15% by weight and HCl (pH 2) with stirring. At the end of the addition, the pH is readjusted to 2 with a 37% HCl solution, and the suspension is allowed to stand overnight. The precipitate is collected by filtration, then rinsed with 100 mL of water. The white solid obtained is solubilized in 500 mL of water by slowly adding a 1N aqueous NaOH solution to pH 7 with stirring, then the solution is filtered through a 0.45 m filter. The clear solution obtained is purified by ultrafiltration against 0.9% NaCl solution, then with water, until the conductimetry of the permeate is less than 50 μS/cm. The solution is filtered through a 0.2 m filter and stored at 2-8° C.

Dry extract: 21.6 mg/g

DP (estimated based on ¹H NMR): 20

Based on ¹H NMR: i=0.025

The calculated average molecular weight of co-polyamino acid AB6 is 3369 g/mol.

Aqueous HPLC-SEC (PEG calibrant): Mn=4000 g/mol.

### EXAMPLE AB7: CO-POLYAMINO ACID AB7—SODIUM POLY-L-GLUTAMATE CAPPED AT ONE OF ITS ENDS BY AN ACETYL GROUP AND MODIFIED BY MOLECULE AA7 AND HAVING A NUMBER-AVERAGE MOLECULAR WEIGHT (MN) OF 3300 G/MOL

Co-polyamino acid AB7-1: poly-L-glutamic acid of number-average molecular weight (Mn) relative to 3600 g/mol and of DP 21 resulting from the polymerization of γ-benzyl-L-glutamate N-carboxyanhydride initiated by the hexylamine and capped at one of its ends by an acetyl group

In a previously oven-dried flask, γ-benzyl-L-glutamate N-carboxyanhydride (Glu (OBn)-NCA, 100.0 g, 380 mmol) is placed under vacuum for 30 minutes, then anhydrous DMF (225 mL) is added. The mixture is then stirred under argon until complete dissolution, cooled to 4° C., then hexylamine (1.78 g, 17 mmol) is quickly introduced. The mixture is stirred between 4° C. and room temperature for 2 days, then precipitated in diisopropyl ether (3.4 L). The precipitate is collected by filtration, washed twice with diisopropyl ether (225 mL), then dried to give a white solid which is dissolved in 450 mL of THF. DIPEA (31 mL, 176 mmol), then acetic anhydride (17 mL, 176 mmol) are successively added to this solution. After stirring overnight at room temperature, the solution is slowly poured into diisopropyl ether (3 L) under stirring. After stirring for 1 hour, the precipitate is filtered off, washed twice with diisopropyl ether (250 mL), then dried under vacuum at 30° C. to give a poly (gamma-benzyl-L-glutamic acid) capped at one of its ends by an acetyl group.

A solution of the above hydrobromic acid (HBr) at 33% in acetic acid (235 mL) is added dropwise to a solution of the above co-polyamino acid (72 g) in trifluoroacetic acid (TFA, 335 mL) at 4° C. The mixture is stirred at room temperature for 3 h30, then poured dropwise onto a 1:1 (v/v) mixture of diisopropyl ether and water with stirring (4 L). After stirring for 2 hours, the heterogeneous mixture is allowed to stand overnight. The white precipitate is collected by filtration, washed with a 1:1 (v/v) mixture of diisopropyl ether and water (340 mL), then with water (340 mL).

The solid obtained is then solubilized in water (1.5 L) by adjusting the pH to 7 by adding a 10 N aqueous solution of sodium hydroxide, then a 1N aqueous sodium hydroxide solution. After solubilization, the solution is diluted by adding water to obtain a final volume of 2.1 L. The solution is filtered through a 0.45 m filter, then purified by ultrafiltration against a solution of NaCl 0.9%, then water until the conductimetry of the permeate is less than 50 μS/cm. The co-polyamino acid solution is then concentrated until a final volume of 1.8 L is obtained.

The aqueous solution is then acidified by adding 37% hydrochloric acid solution until a pH of 2 is reached. After stirring for 4 hours, the precipitate obtained is filtered, washed with water (330 mL) and then dried under vacuum at 30° C. to produce a poly-L-glutamic acid of number-average molecular weight (Mn) 3600 g/mol with respect to a standard of polyoxyethylene (PEG), and average polymerization degree of 21.

### Co-Polyamino Acid AB7

A sodium poly-L-glutamate acid modified with the molecule AA7 is obtained by a process similar to that used in the preparation of the co-polyamino acid AB6 applied to the hydrochloride salt of molecule AA7 (1.43 g, 2.2 mmol) and the co-polyamino acid AB7-1 (10.0 g).

Dry extract: 24.3 mg/g

DP (estimated based on ¹H NMR): 21

Based on ¹H NMR: i=0.03

The calculated average molecular weight of co-polyamino acid AB7 is 3677 g/mol.

Aqueous HPLC-SEC (PEG calibrant): Mn=3300 g/mol.

### EXAMPLE AB8: CO-POLYAMINO ACID AB8—SODIUM POLY-L-GLUTAMATE MODIFIED BY MOLECULE AA7 AND HAVING A NUMBER-AVERAGE MOLECULAR WEIGHT (MN) OF 3600 G/MOL

Co-polyamino acid AB8-1: poly-L-glutamic acid of number-average molecular weight (Mn) 3800 g/mol and degree of polymerization of 24 resulting from the polymerization of γ-methyl-L-glutamate N-carboxyanhydride initiated by ammonia

A poly-L-glutamic acid is obtained by a process similar to that described in patent application FR-A-2 801 226 applied to γ-methyl-L-glutamic acid N-carboxyanhydride (25.0 g, 133.6 mmol) and 0.5 N ammonia solution in dioxane (12.1 mL, 6.05 mmol).

### Co-Polyamino AB8

A sodium poly-L-glutamate modified with the molecule AA7 is obtained by a process similar to that used in the preparation of the co-polyamino acid AB6 applied to the hydrochloride salt of molecule AA7 (2.1 g, 3.24 mmol) and co-polyamino acid AB8-1 (14.3 g).

Dry extract: 25.2 mg/g

DP (estimated based on ¹H NMR): 24

Based on ¹H NMR: i=0.03

The calculated average molecular weight of co-polyamino acid AB8 is 4099 g/mol.

Aqueous HPLC-SEC (PEG calibrant): Mn=3600 g/mol.

### EXAMPLE AB9: CO-POLYAMINO ACID AB9—SODIUM POLY-L-GLUTAMATE MODIFIED BY MOLECULE AA3 AND HAVING A NUMBER-AVERAGE MOLECULAR WEIGHT (MN) OF 3200 G/MOL

A sodium poly-L-glutamate modified by molecule AA3 is obtained by a process similar to that used in the preparation of co-polyamino acid AB1 applied to the hydrochloride salt of molecule AA3 and to a poly-L-glutamic acid obtained by a process similar to that used in the preparation of co-polyamino acid AB1-1.

Dry extract: 14.7 mg/g

DP (estimated based on ¹H NMR): 30

Based on ¹H NMR: i=0.12

The calculated average molecular weight of co-polyamino acid AB9 is 6192 g/mol.

Aqueous HPLC-SEC (PEG calibrant): Mn=3200 g/mol.

### EXAMPLE AB10: CO-POLYAMINO ACID AB10—SODIUM POLY-L-GLUTAMATE MODIFIED BY MOLECULE AA4 AND HAVING A NUMBER-AVERAGE MOLECULAR WEIGHT (MN) OF 2600 G/MOL

A sodium poly-L-glutamate modified by molecule AA4 is obtained by a process similar to that used in the preparation of co-polyamino acid AB7 applied to the hydrochloride salt of molecule AA4 and to a poly-L-glutamic acid obtained by a process similar to that used in the preparation of co-polyamino acid AB1-1.

Dry extract: 18.3 mg/g

DP (estimated based on ¹H NMR): 25

Based on ¹H NMR: i=0.08

The calculated average molecular weight of co-polyamino acid AB10 is 4870 g/mol.

Aqueous HPLC-SEC (PEG calibrant): Mn=2600 g/mol.

### EXAMPLE AB11: CO-POLYAMINO ACID AB11—SODIUM POLY-L-GLUTAMATE MODIFIED BY MOLECULE AA5 AND HAVING A NUMBER-AVERAGE MOLECULAR WEIGHT (MN) OF 2700 G/MOL

A sodium poly-L-glutamate modified by molecule AA5 is obtained by a process similar to that used in the preparation of co-polyamino acid AB6 applied to the hydrochloride salt of molecule AA5 and to a poly-L-glutamic acid obtained by a process similar to that used in the preparation of co-polyamino acid AB1-1.

Dry extract: 20.2 mg/g

DP (estimated based on ¹H NMR): 23

Based on ¹H NMR: i=0.05

The calculated average molecular weight of co-polyamino acid AB11 is 4072 g/mol.

Aqueous HPLC-SEC (PEG calibrant): Mn=2700 g/mol.

### EXAMPLE AB12: CO-POLYAMINO ACID AB12—SODIUM POLY-L-GLUTAMATE MODIFIED BY MOLECULE AA8 AND HAVING A NUMBER-AVERAGE MOLECULAR WEIGHT (MN) OF 3000 G/MOL

A sodium poly-L-glutamate modified by molecule AA8 is obtained by a process similar to that used in the preparation of co-polyamino acid AB1 applied to the hydrochloride salt of molecule AA8 and to a poly-L-glutamic acid obtained by a process similar to that used in the preparation of co-polyamino acid AB1-1.

Dry extract: 19.5 mg/g

DP (estimated based on ¹H NMR): 26

Based on ¹H NMR: i=0.04

The calculated average molecular weight of co-polyamino acid AB12 is 4477 g/mol.

Aqueous HPLC-SEC (PEG calibrant): Mn=3000 g/mol.

### EXAMPLE AB13: CO-POLYAMINO ACID AB13—SODIUM POLY-L-GLUTAMATE MODIFIED BY MOLECULE AA9 AND HAVING A NUMBER-AVERAGE MOLECULAR WEIGHT (MN) OF 3300 G/MOL

By a process similar to that used in the preparation of co-polyamino acid AB6 applied to the hydrochloride salt of molecule AA9 and a poly-L-glutamic acid obtained by a process similar to that used in the preparation of co-polyamino acid AB-1 Using isoamylamine as the initiator in place of hexylamine, a sodium poly-L-glutamate modified with molecule AA9 is obtained.

Dry extract: 22.3 mg/g

DP (estimated based on ¹H NMR): 35

Based on ¹H NMR: i=0.12

The calculated average molecular weight of co-polyamino acid AB13 is 7226 g/mol.

Aqueous HPLC-SEC (PEG calibrant): Mn=3300 g/mol.

### EXAMPLE AB14: CO-POLYAMINO ACID AB14—SODIUM POLY-L-GLUTAMATE MODIFIED AT ONE OF ITS ENDS BY MOLECULE AA1 AND HAVING A NUMBER-AVERAGE MOLECULAR WEIGHT (MN) OF 3400 G/MOL

Hydrochloride salt of molecule AA1 (2.03 g, 4.70 mmol), chloroform (5 mL), molecular sieve 4 Å (1.3 g), as well as Amberlite IRN 150 ion exchange resin (1.3 g) are successively added to a suitable container. After stirring for 1 hour on rollers, the medium is filtered and the resin is rinsed with chloroform. The mixture is evaporated, then co-evaporated with toluene. The residue is solubilized in anhydrous DMF (30 mL) for direct use in the polymerization reaction.

In a previously oven-dried flask, γ-benzyl-L-glutamate N-carboxyanhydride (25.59 g, 97.2 mmol) is placed under vacuum for 30 minutes then anhydrous DMF (140 mL) is added. The mixture is stirred under argon until complete solubilization, cooled to 4° C., then the solution of molecule AA1 prepared as described above is quickly introduced. The mixture is stirred at 4° C. and room temperature for 2 days, then heated at 65° C. for 2 hours. The reaction mixture is then cooled to room temperature, then poured dropwise into diisopropyl ether (1.7 L) with stirring. The white precipitate is collected by filtration, washed twice with diisopropyl ether (140 mL), then dried under vacuum at 30° C. to obtain a white solid. The solid is diluted in TFA (160 mL), and a solution of 33% hydrobromic acid (HBr) in acetic acid (62 mL, 354 mmol) is then added dropwise at 0° C. The solution is stirred for 2 hours at room temperature and is then poured dropwise on a 1:1 (v/v) mixture of diisopropyl ether/water and with stirring (1.9 L). After stirring for 2 hours, the heterogeneous mixture is allowed to stand overnight. The white precipitate is collected by filtration, washed successively with a 1:1 (v/v) mixture of diisopropyl ether and water (280 mL) then with water (140 mL). The obtained solid is solubilized in water (530 mL) by adjusting the pH to 7 by adding 10 N aqueous sodium hydroxide solution, then 1N aqueous sodium hydroxide solution. After solubilization, the theoretical concentration is adjusted to 20 g/L theoretical by addition of water to obtain a final volume of 800 mL. The mixture is filtered on a 0.45 μm filter, then purified by ultrafiltration against a 0.9% NaCl solution, then water until the conductimetry of the permeate is less than 50 μS/cm. The co-polyamino acid solution is then concentrated to about 30 g/L theoretical and the pH is adjusted to 7.0. The aqueous solution is filtered through a 0.2 μm filter and stored at 4° C.

Dry extract: 24.1 mg/g

DP (estimated by ¹H NMR)=25 where i=0.04

The calculated average molecular weight of co-polyamino acid AB14 is 3378 g/mol.

Aqueous HPLC-SEC (PEG calibrant): Mn=3400 g/mol.

### EXAMPLE AB15: CO-POLYAMINO ACID AB15—SODIUM POLY-L-GLUTAMATE MODIFIED AT ONE OF ITS ENDS BY THE MOLECULE AA6 AND HAVING A NUMBER-AVERAGE MOLECULAR WEIGHT (MN) 4100 G/MOL

By a process similar to that used in the preparation of the co-polyamino acid AB14 applied to the hydrochloride salt of molecule AA6 (2.16 g, 3.94 mmol) and 25.58 g (97.2 mmol) of γ-benzyl-L-glutamate N-carboxyanhydride, a poly-L-glutamate sodium modified at one of its ends by the molecule AA6 is obtained.

Dry extract: 45.5 mg/g

DP (estimated by ¹H NMR)=30 where i=0.033

The calculated average molecular weight of co-polyamino acid AB15 is 5005 g/mol.

Aqueous HPLC-SEC (PEG calibrant): Mn=4100 g/mol.

### EXAMPLE AB16: CO-POLYAMINO ACID AB16—SODIUM POLY-L-GLUTAMATE MODIFIED AT ONE END BY THE MOLECULE AA6 AND HAVING A NUMBER-AVERAGE MOLECULAR WEIGHT (MN) OF 6500 G/MOL

By a process similar to that used in the preparation of the co-polyamino acid AB14 applied to the hydrochloride salt of molecule AA6 (2.39 g, 4.36 mmol) and 50.0 g (189.9 mmol) of γ-benzyl-L-glutamate N-carboxyanhydride, a poly-L-glutamate sodium modified at one of its ends by molecule AA6 is obtained.

Dry extract: 28.5 mg/g

DP (estimated by ¹H NMR)=48 where i=0.021

The calculated average molecular weight of co-polyamino acid AB16 is 7725 g/mol.

Aqueous HPLC-SEC (PEG calibrant): Mn=6500 g/mol.

### EXAMPLE AB17: CO-POLYAMINO ACID AB17—SODIUM POLY-L-GLUTAMATE MODIFIED AT ONE END BY THE MOLECULE AA7 AND HAVING A NUMBER-AVERAGE MOLECULAR WEIGHT (MN) OF 3500 G/MOL

By a process similar to that used in the preparation of the co-polyamino acid AB14 applied to the hydrochloride salt of molecule AA7 (2.80 g, 4.32 mmol) and 25.0 g (94.9 mmol) of γ-benzyl-L-glutamate N-carboxyanhydride, a poly-L-glutamate sodium modified at one of its ends by molecule AA7 is obtained.

Dry extract: 25.2 mg/g

DP (estimated by ¹H NMR)=26 where i=0.038

The calculated average molecular weight of co-polyamino acid AB17 is 4500 g/mol.

Aqueous HPLC-SEC (PEG calibrant): Mn=3500 g/mol.

### EXAMPLE AB18: CO-POLYAMINO ACID AB18—SODIUM POLY-L-GLUTAMATE MODIFIED AT ONE END BY THE MOLECULE AA7 AND HAVING A NUMBER-AVERAGE MOLECULAR WEIGHT (MN) OF 3700 G/MOL

A sodium poly-L-glutamate modified at one of its end by the molecule AA7 is obtained by polymerization of glutamic acid γ-methyl N-carboxyanhydride (25.0 g, 133.6 mmol) using the hydrochloride salt of the molecule AA7 (2.80 g, 4.32 mmol) as initiator and by deprotecting methyl esters by using a 37% hydrochloric acid solution according to the process described in patent application FR-A-2 801 226.

Dry extract: 44.3 mg/g

DP (estimated by ¹H NMR)=22 where i=0.045

The calculated average molecular weight of the co-polyamino acid AB18 is 3896 g/mol.

Aqueous HPLC-SEC (PEG calibrant): Mn=3700 g/mol.

### EXAMPLE AB19: CO-POLYAMINO ACID AB19—SODIUM POLY-L-GLUTAMATE MODIFIED AT ONE OF ITS ENDS BY THE MOLECULE AA6 AND HAVING A NUMBER-AVERAGE MOLECULAR WEIGHT (MN) OF 10500 G/MOL

A sodium poly-L-glutamate modified at one of its ends by the molecule AA6 is obtained by a process similar to that used in the preparation of the co-polyamino acid AB14 applied to the hydrochloride salt of the molecule AA6 (1.64 g, 2.99 mmol) and to γ-benzyl-L-glutamate N-carboxyanhydride (49.3 g, 187 mmol).

Dry extract: 23.4 mg/g

DP (estimated by ¹H NMR)=65 where i=0.015

The calculated average molecular weight of the co-polyamino acid AB19 is 10293 g/mol.

Aqueous HPLC-SEC (PEG calibrant): Mn=10500 g/mol.

### EXAMPLE AB20: CO-POLYAMINO ACID AB20—SODIUM POLY-L-GLUTAMATE CAPPED AT ONE END BY AN ACETYL GROUP AND MODIFIED AT ONE END BY THE MOLECULE AA6 AND HAVING A NUMBER-AVERAGE MOLECULAR WEIGHT (MN) OF 10,400 G/MOL

The hydrochloride salt of the molecule AA6 (0.545 g, 1.00 mmol), chloroform (10 mL), molecular sieve 4 Å (3 g), as well as Amberlite IRN 150 ion exchange resin (3 g) are successively added to a suitable container. After stirring for 1 hour on rollers, the medium is filtered and the resin is rinsed with chloroform. The mixture is evaporated, then co-evaporated with toluene. The residue is solubilized in anhydrous DMF (10 mL) for direct use in the polymerization reaction.

In a previously oven-dried flask, γ-benzyl-L-glutamate N-carboxyanhydride (17.0 g, 64.6 mmol) is placed under vacuum for 30 minutes then anhydrous DMF (30 mL) is added. The mixture is stirred under argon until complete solubilization, cooled to 4° C., then the solution of the molecule AA6 prepared as described above is quickly introduced. The mixture is stirred between 4° C. and room temperature for 2 days, then precipitated in diisopropyl ether (0.6 L). The precipitate is collected by filtration, washed twice with diisopropyl ether (40 mL), then dried to give a white solid which is dissolved in 80 mL of THF. DIPEA (1.7 mL, 9.8 mmol), then acetic anhydride (0.9 mL, 9.5 mmol) are successively added to this solution. After stirring overnight at room temperature, the solution is slowly poured into diisopropyl ether (480 mL) over a period of 30 minutes with stirring. After 1 hour of stirring, the precipitate is filtered, washed twice with diisopropyl ether (80 ml), then dried under vacuum at 30° C. to obtain a poly (gamma-benzyl-L-glutamic acid) capped at one end by an acetyl group and modified at the other end by the molecule AA6 in the form of a white solid.

The solid is diluted in TFA (65 mL), and a solution of 33% hydrobromic acid (HBr) in acetic acid (45 mL, 257.0 mmol) is then added dropwise at 4° C. The solution is stirred for 2 hours at room temperature and is then poured dropwise on a 1:1 (v/v) mixture of diisopropyl ether/water and with stirring (780 mL). After stirring for 2 hours, the heterogeneous mixture is allowed to stand overnight. The white precipitate is collected by filtration, washed successively with a mixture of 1:1 (v/v) diisopropyl ether and water (70 mL) then with water (70 mL). The obtained solid is solubilized in water (300 mL) by adjusting the pH to 7 by adding 10 N aqueous sodium hydroxide solution, then 1N aqueous sodium hydroxide solution. After solubilization, the theoretical concentration is adjusted to 20 g/L theoretical by addition of water to obtain a final volume of 440 mL. The mixture is filtered on a 0.45 m filter, then purified by ultrafiltration against a 0.9% NaCl solution, then water until the conductimetry of the permeate is less than 50 μS/cm. The co-polyamino acid solution is then concentrated to about 30 g/L theoretical and the pH is adjusted to 7. The aqueous solution is filtered through a 0.2 m filter and stored at 4° C.

Dry extract: 21.5 mg/g

DP (estimated by ¹H NMR)=60 where i=0.017

The calculated average molecular weight of co-polyamino acid AB20 is 9619 g/mol.

Aqueous HPLC-SEC (PEG calibrant): Mn=10400 g/mol.

### EXAMPLE AB21: CO-POLYAMINO ACID AB21—SODIUM POLY-L-GLUTAMATE MODIFIED BY MOLECULE AA7 AND HAVING A NUMBER-AVERAGE MOLECULAR WEIGHT (MN) OF 3400 G/MOL

A sodium poly-L-glutamate modified with molecule AA7 is obtained by a process similar to that used in the preparation of co-polyamino acid AB6 applied to the hydrochloride salt of molecule AA7 (2.44 g, 2.4 mmol) and to a poly-L-glutamic acid (10 g) obtained by a process similar to that used in the preparation of polyamino acid AB1-1.

Dry extract: 22.7 mg/g

DP (estimated based on ¹H NMR): 22

Based on ¹H NMR: i=0.056

The calculated average molecular weight of co-polyamino acid AB21 is 4090 g/mol.

Aqueous HPLC-SEC (PEG calibrant): Mn=3400 g/mol.

### EXAMPLE AB21′

### Co-Polyamino Acid AB21′—Sodium Poly-L-Glutamate Modified at One of its Ends by the Molecule AA10 and Having a Number-Average Molecular Weight (Mn) of 3478 g/Mol

A sodium poly-L-glutamate modified at one of its ends by molecule AA10 is obtained by a process similar to that used in the preparation of co-polyamino acid AB14 applied to the hydrochloride salt of molecule AA10 (0.916 g, 1.38 mmol) and to γ-benzyl-L-glutamate N-carboxyanhydride (7.19 g, 27.3 mmol).

Dry extract: 14.8 mg/g

DP (estimated by NMR ¹H)=25 so i=0.04

The calculated average molecular weight of the co-polyamino acid AB21′ is 4364 g/mol.

Aqueous HPLC-SEC (PEG calibrant): Mn=3478 g/mol.

### EXAMPLE AB22′: CO-POLYAMINO ACID AB22′—SODIUM POLY-L-GLUTAMATE CAPPED AT ONE OF ITS ENDS BY AN ACETYL GROUP AND MODIFIED BY THE MOLECULE AA12 AND HAVING A NUMBER-AVERAGE MOLECULAR WEIGHT (MN) OF 4000 G/MOL

The hydrochloride salt of molecule AA12 (4.56 g, 11.29 mmol) is dissolved in chloroform (60 mL) and triethylamine (1.14 g, 11.29 mmol) is added. NMM (7.6 g, 75.26 mmol), then HOPO (2.51 g, 22.58 mmol) are successively added to a co-polyamino acid (10.0 g, 75.3 mmol) solution obtained according to a process similar to that used in the preparation of co-polyamino acid B7-1 in DMF (420 mL). The reaction medium is then cooled to 0° C., then EDC (4.33 g, 22.58 mmol) is added, the medium is stirred for 1 h at 0° C., then the solution of the molecule AA10 is added. The reaction mixture is stirred for 2 hours at between 0° C. and room temperature. The reaction medium is filtered through a 0.2 mm woven filter and poured drop by drop onto 3.95 L of water containing NaCl 15% by weight and HCl (pH 2) with stirring. At the end of the addition, the pH is readjusted to 2 with 37% HCl solution, and the suspension is allowed to stand overnight. The precipitate is collected by filtration, then solubilized in 780 mL of water by slow addition of a 1N aqueous NaOH solution to pH 7 with stirring. After filtration through a 0.45 μm filter, the solution is diluted by adding water, then acetone is added to obtain a solution containing 30% by weight of acetone. This solution is filtered through an activated charcoal filter, then the acetone is distilled (40° C., 100 mbar). After filtration through a 0.45 μm filter, the product is purified by ultrafiltration against a 0.9% NaCl aqueous solution, a carbonate buffer solution (150 mM), a 0.9% NaCl aqueous solution, a phosphate buffer (150 mM) solution, a 0.9% NaCl aqueous solution, then water until the conductimetry of the permeate is less than 50 μS/cm. The solution is then concentrated, filtered through a 0.2 am filter and stored at 2-8° C.

Dry extract: 19.7 mg/g

DP (estimated based on ¹H NMR): 38

Based on ¹H NMR: i=0.16

The calculated average molecular weight of the co-polyamino acid AB22′ is 7877 g/mol.

Organic HPLC-SEC (PEG Calibrator): Mn=4000 g/mol.

### EXAMPLE AB23

### Co-Polyamino Acid AB23—Sodium Poly-L-Glutamate Modified at One of its Ends by the Molecule AA14 and Having a Number-Average Molecular Weight (Mn) of 3600 g/Mol

A sodium poly-L-glutamate modified at one of its ends by molecule AA14 is obtained by a process similar to that used in the preparation of the co-polyamino acid AB14 applied to molecule AA14 in the form of free amine (0.820 g, 1.34 mmol) and γ-benzyl-L-glutamate N-carboxyanhydride (7.75 g, 29.4 mmol).

Dry extract: 16.8 mg/g

DP (estimated by NMR ¹H)=22 thus i=0.045

The calculated average molecular weight of co-polyamino acid AB23 is 3897 g/mol.

Aqueous HPLC-SEC (PEG calibrant): Mn=3600 g/mol.

### EXAMPLE AB23′: CO-POLYAMINO ACID AB23′—SODIUM POLY-L-GLUTAMATE MODIFIED BY MOLECULE AA12 AND HAVING A NUMBER-AVERAGE MOLECULAR WEIGHT (MN) OF 7600 G/MOL

Co-polyamino acid AB23′-1: poly-L-glutamic acid from the polymerization of γ-benzyl-L-glutamate N-carboxyanhydride initiated by hexylamine and capped at one of its ends by a pyroglutamate group

A poly-L-glutamic acid (20.0 g) obtained by a process similar to that used in the preparation of the co-polyamino acid AB1-1 is solubilized in DMF at 80° C., then maintained at this temperature. After 24 hours, the reaction medium is poured into a solution of NaCl at 15% and at pH 2. After 4 hours, the white solid is collected by filtration, rinsed with water, then dried under vacuum at 30° C.

### Co-Polyaminoamide AB23′

A sodium poly-L-glutamate acid modified with the molecule AA12 is obtained by a process similar to that used in the preparation of the co-polyamino acid AB22′ applied to the hydrochloride salt of molecule AA12 (2.742 g, 6.79 mmol) and the co-polyamino acid AB23′-1 (9.0 g).

Dry extract: 21.9 mg/g

DP (estimated based on ¹H NMR): 60

Based on ¹H NMR: i=0.1

The calculated average molecular weight of the co-polyamino acid AB23′ is 11034 g/mol.

Organic HPLC-SEC (PEG Calibrator): Mn=7600 g/mol.

### EXAMPLE AB24: CO-POLYAMINO ACID AB24—SODIUM POLY-L-GLUTAMATE CAPPED AT ONE OF ITS ENDS BY AN ACETYL GROUP AND MODIFIED BY MOLECULE AA1 AND HAVING A NUMBER-AVERAGE MOLECULAR WEIGHT (MN) OF 3900 G/MOL

A sodium poly-L-glutamate capped at one of its ends by an acetyl group and modified with molecule AA1 is obtained by a process similar to that used in the preparation of the co-polyamino acid AB6 applied to the hydrochloride salt of molecule AA1 (1.330 g, 3.08 mmol) and a poly-L-glutamic acid of Mn relative 5400 g/mol (4.0 g) obtained by a process similar to that used for the preparation of co-polyamino acid AB7-1.

Dry extract: 18.7 mg/g

DP (estimated based on ¹H NMR): 38

Based on ¹H NMR: i=0.089

The calculated average molecular weight of co-polyamino acid AB24 is 7088 g/mol.

Aqueous HPLC-SEC (PEG calibrant): Mn=3900 g/mol.

### EXAMPLE AB25: CO-POLYAMINO ACID AB25—SODIUM POLY-L-GLUTAMATE CAPPED AT ONE OF ITS ENDS BY AN ACETYL GROUP AND MODIFIED BY MOLECULE AA12 AND HAVING A NUMBER-AVERAGE MOLECULAR WEIGHT (MN) OF 3700 G/MOL

A poly-L-glutamic acid of average relative mass Mn=5400 g/mol and a polymerization degree of 38 (10.0 g) obtained by a process similar to that used in the preparation of co-polyamino acid AB7-1 is solubilized in DMF (420 mL) at 30° C. and maintained at this temperature. The hydrochloride salt of molecule AA12 (4.56 g, 11.29 mmol) is dissolved in chloroform (60 mL) and triethylamine (1.14 g, 11.29 mmol) is added. NMM (7.6 g, 75.26 mmol), then HOPO (2.51 g, 22.58 mmol) are successively added to the co-polyamino acid solution in DMF. The reaction medium is then cooled to 0° C., then EDC (4.33 g, 22.58 mmol) is added, the medium is stirred for 1 h at 0° C., then the solution of the molecule AA12 is added. The reaction mixture is stirred for 2 hours at between 0° C. and room temperature. The reaction medium is filtered through a 0.2 mm woven filter and poured drop by drop onto 3.95 L of water containing NaCl 15% by weight and HCl (pH 2) with stirring. At the end of the addition, the pH is readjusted to 2 with 37% HCl solution, and the suspension is allowed to stand overnight. The precipitate is collected by filtration, then solubilized in 780 mL of water by slow addition of a 1N aqueous NaOH solution to pH 7 with stirring. After filtering on a 0.45 m filter, the solution is diluted by adding water to obtain a volume of 900 mL, then acetone (485 mL) is added to obtain a solution containing 30% by weight of acetone. This solution is filtered through activated carbon filter (3M R53SLP) and the acetone is distilled (40° C., 100 mbar). After filtration through a 0.45 μm filter, the product is purified by ultrafiltration against a 0.9% NaCl aqueous solution, a carbonate buffer solution (150 mM), a 0.9% NaCl aqueous solution, a phosphate buffer (150 mM) solution, a 0.9% NaCl aqueous solution, then water until the conductimetry of the permeate is less than 50 μS/cm. The solution is then concentrated to a final volume of 600 mL. The solution is filtered through a 0.2 μm filter and stored at 2-8° C.

Dry extract: 19.7 mg/g

DP (estimated based on ¹H NMR): 38

Based on ¹H NMR: i=0.16

The calculated average molecular weight of co-polyamino acid AB25 is 7877 g/mol.

Aqueous HPLC-SEC (PEG calibrant): Mn=3700 g/mol.

### EXAMPLE AB26′: CO-POLYAMINO ACID AB26′—SODIUM POLY-L-GLUTAMATE MODIFIED BY MOLECULE AA12 AND HAVING A NUMBER-AVERAGE MOLECULAR WEIGHT (MN) OF 2700 G/MOL

A sodium poly-L-glutamate modified with molecule AA12 is obtained by a process similar to that used in the preparation of co-polyamino acid AB23′ applied to the hydrochloride salt of molecule AA12 and to a poly-L-glutamic acid obtained by a process similar to that used in the preparation of polyamino acid AB23′-1.

Dry extract: 23.9 mg/g

DP (estimated based on ¹H NMR): 22

Based on ¹H NMR: i=0.21

The calculated average molecular weight of the co-polyamino acid AB26′ is 4899 g/mol.

Organic HPLC-SEC (PEG Calibrator): Mn=2700 g/mol.

### EXAMPLE AB27′: CO-POLYAMINO ACID AB27′—SODIUM POLY-L-GLUTAMATE MODIFIED BY MOLECULE AA11-1 AND HAVING A NUMBER-AVERAGE MOLECULAR WEIGHT (MN) OF 4500 G/MOL

A sodium poly-L-glutamate modified with molecule AA11-1 is obtained by a process similar to that used in the preparation of co-polyamino acid AB23′ applied to the hydrochloride salt of molecule AA11-1 and to a poly-L-glutamic acid obtained by a process similar to that used in the preparation of polyamino acid AB23′-1.

Dry extract: 26.8 mg/g

DP (estimated based on ¹H NMR): 39

Based on ¹H NMR: i=0.15

The calculated average molecular weight of the co-polyamino acid AB27′ is 8808 g/mol.

Organic HPLC-SEC (PEG Calibrator): Mn=4500 g/mol.

### EXAMPLE AB28: CO-POLYAMINO ACID AB28—SODIUM POLY-L-GLUTAMATE CAPPED AT ONE OF ITS ENDS BY AN ACETYL GROUP AND MODIFIED BY MOLECULE AA14 AND HAVING A NUMBER-AVERAGE MOLECULAR WEIGHT (MN) OF 4700 G/MOL

By a process similar to that used in the preparation of co-polyamino acid AB6 applied to molecule AA14 (1.51 g, 2.46 mmol) and a poly-L-glutamic acid of Mn relative 5400 g/mol (3.27 g) obtained by a process similar to that used in the preparation of co-polyamino acid AB7-1, a sodium poly-L-glutamate capped at one of its ends by an acetyl group and modified by molecule AA14 is obtained after purification by ultrafiltration against a 0.9% NaCl aqueous solution, a carbonate buffer solution (150 mM), a 0.9% NaCl aqueous solution, a phosphate buffer solution (150 mM), an aqueous solution of NaCl at 0.9%, then water until the conductimetry of the permeate is less than 50 μS/cm. The co-polyamino acid solution is then concentrated to about 20 g/L theoretical and the pH is adjusted to 7. The aqueous solution is filtered through a 0.2 μm filter and stored at 4° C.

Dry extract: 6.1 mg/g

DP (estimated based on ¹H NMR): 38

Based on ¹H NMR: i=0.1

The calculated average molecular weight of co-polyamino acid AB28 is 8062 g/mol.

Aqueous HPLC-SEC (PEG calibrant): Mn=4700 g/mol.

### EXAMPLE AB28′: CO-POLYAMINO ACID AB28′—SODIUM POLY-L-GLUTAMATE MODIFIED BY MOLECULE AA12-1 AND HAVING A NUMBER-AVERAGE MOLECULAR WEIGHT (MN) OF 4000 G/MOL

A sodium poly-L-glutamate modified with molecule AA12-1 is obtained by a process similar to that used in the preparation of co-polyamino acid AB23′ applied to the hydrochloride salt of molecule AA12-1 and to a poly-L-glutamic acid obtained by a process similar to that used in the preparation of polyamino acid AB23′-1.

Dry extract: 22.9 mg/g

DP (estimated based on ¹H NMR): 39

Based on ¹H NMR: i=0.15

The calculated average molecular weight of the co-polyamino acid AB28′ is 7706 g/mol.

Organic HPLC-SEC (PEG Calibrator): Mn=4000 g/mol.

### EXAMPLE AB29: CO-POLYAMINO ACID AB29—SODIUM POLY-L-GLUTAMATE MODIFIED BY MOLECULE AA12 AND HAVING A NUMBER-AVERAGE MOLECULAR WEIGHT (MN) OF 4300 G/MOL

Co-polyamino acid AB29-1: poly-L-glutamic acid from the polymerization of γ-benzyl-L-glutamate N-carboxyanhydride initiated by hexylamine and capped at one of its ends by a pyroglutamate group.

A poly-L-glutamic acid obtained by a process similar to that used for the preparation of the co-polyamino acid AB1-1 is solubilized in DMF at 80° C., then maintained at this temperature. After 24 hours, the reaction medium is poured into a solution of NaCl at 15% and at pH 2. After 4 hours, the white solid is collected by filtration, rinsed with water, then dried under vacuum at 30° C.

### Co-Polyaminoamide AB29

A sodium poly-L-glutamate modified by molecule AA12 is obtained by a process similar to that used in the preparation of co-polyamino acid AB25 applied to the hydrochloride salt of molecule AA12 and co-polyamino acid AB29-1.

Dry extract: 22.9 mg/g

DP (estimated based on ¹H NMR): 39

Based on ¹H NMR: i=0.15

The calculated average molecular weight of co-polyamino acid AB29 is 7870 g/mol.

Organic HPLC-SEC (PEG Calibrator): Mn=4300 g/mol.

### EXAMPLE AB29′: CO-POLYAMINO ACID AB29′—SODIUM POLY-L-GLUTAMATE MODIFIED BY MOLECULE AA13-1 AND HAVING A NUMBER-AVERAGE MOLECULAR WEIGHT (MN) OF 4000 G/MOL

### Co-Polyamino Acid AB29′-1: Poly-L-Glutamic Acid from the Polymerization of γ-Benzyl-L-Glutamate N-Carboxyanhydride Initiated by Hexylamine

In a double-jacket reactor, γ-benzyl-L-glutamate N-carboxyanhydride (500 g, 1.90 mol) is solubilized in anhydrous DMF (1100 mL). The mixture is then stirred until complete dissolution, cooled to 0° C., then hexylamine (6.27 mL, 47.5 mmol) is introduced rapidly. The mixture is stirred at 0° C. for 5 hours, between 0° C. and 20° C. for 7 hours, then at 20° C. for 7 hours. The reaction medium is then heated at 65° C. for 2 hours, cooled to 55° C. and methanol (3300 mL) is introduced after 1 h30. The reaction mixture is then cooled to 0° C. and left under stirring for 18 hours. The white precipitate is collected by filtration, washed with diisopropyl ether (2×800 mL), then dried under vacuum at 30° C. to give a poly (gamma-benzyl-L-glutamic) acid (PBLG).

Pd/Al₂O₃ (36 g) is added to a PBLG (180 g) solution in N,N-dimethylacetamide (DMAc, 450 mL) under an argon atmosphere. The mixture is placed in a hydrogen atmosphere (10 bar) and stirred at 60° C. for 24 hours. After cooling at room temperature and filtration of the catalyst on P4 sinter and PTFE Omnipore hydrophilic membrane 0.2 μm, a water solution at pH 2 (2700 mL) is poured dropwise on the DMAc solution, on a 45 minute period with stirring. After stirring for 18 hours, the white precipitate is collected by filtration, washed with water, then dried under reduced pressure at 30° C.

### Co-Polyamino Acid AB29′

A sodium poly-L-glutamate modified by molecule AA13-1 is obtained by a process similar to that used in the preparation of co-polyamino acid AB23 applied to the hydrochloride salt of molecule AA13-1 and co-polyamino acid AB29′-1.

Dry extract: 16.1 mg/g

DP (estimated based on ¹H NMR): 40

Based on ¹H NMR: i=0.15

The calculated average molecular weight of the co-polyamino acid AB29′ is 7734 g/mol.

Organic HPLC-SEC (PEG Calibrator): Mn=4000 g/mol.

### EXAMPLE AB30: CO-POLYAMINO ACID AB30—SODIUM POLY-L-GLUTAMATE MODIFIED BY MOLECULE AA12 AND HAVING A NUMBER-AVERAGE MOLECULAR WEIGHT (MN) OF 4200 G/MOL

A sodium poly-L-glutamate modified by molecule AA12 is obtained by a process similar to that used in the preparation of co-polyamino acid AB29 applied to the hydrochloride salt of molecule AA12 and to a poly-L-glutamic acid obtained by a process similar to that used in the preparation of co-polyamino acid AB29-1.

Dry extract: 25.9 mg/g

DP (estimated based on ¹H NMR): 39

Based on ¹H NMR: i=0.2

The calculated average molecular weight of co-polyamino acid AB30 is 8509 g/mol.

Organic HPLC-SEC (PEG Calibrator): Mn=4200 g/mol.

### EXAMPLE AB32′: CO-POLYAMINO ACID AB32′—SODIUM POLY-L-GLUTAMATE MODIFIED BY MOLECULE AA14-1 AND HAVING A NUMBER-AVERAGE MOLECULAR WEIGHT (MN) OF 4700 G/MOL

A sodium poly-L-glutamate modified by molecule AA14-1 is obtained by a process similar to that used in the preparation of co-polyamino acid AB29′ applied to molecule AA14-1 and poly-L-glutamic acid AB29′-1.

Dry extract: 13.5 mg/g

DP (estimated based on ¹H NMR): 40

Based on ¹H NMR: i=0.109

The calculated average molecular weight of the co-polyamino acid AB32′ is 8599 g/mol.

Organic HPLC-SEC (PEG Calibrator): Mn=4700 g/mol.

### EXAMPLE AB33: CO-POLYAMINO ACID AB33—SODIUM POLY-L-GLUTAMATE MODIFIED AT ONE END BY THE MOLECULE AA15 AND HAVING A NUMBER-AVERAGE MOLECULAR WEIGHT (MN) OF 1800 G/MOL

By a process similar to that used in the preparation of co-polyamino acid AB14 applied to molecule AA15 (0.82 g, 1.34 mmol) and 7.75 g (29.4 mmol) of γ-benzyl-L-glutamate N-carboxyanhydride, a solution of sodium poly-L-glutamate modified at one of its ends by molecule AA15 is obtained.

Dry extract: 16.8 mg/g

DP (estimated by ¹H NMR)=22 where i=0.045

The calculated average molecular weight of co-polyamino acid AB33 is 3897 g/mol.

Aqueous HPLC-SEC (PEG calibrant): Mn=1800 g/mol.

### EXAMPLE AB34: CO-POLYAMINO ACID AB34—SODIUM POLY-L-GLUTAMATE MODIFIED AT ONE END BY THE MOLECULE AA4 AND HAVING A NUMBER-AVERAGE MOLECULAR WEIGHT (MN) OF 2600 G/MOL

In a previously oven-dried flask, γ-benzyl-L-glutamate N-carboxyanhydride (70.9 g, 269.3 mmol) is solubilized in anhydrous DMF (125 mL). The mixture is cooled to 4° C., then a solution of molecule AA4 in the form of neutral amine (6.80 g, 12.23 mmol) in DMF (35 mL) is introduced rapidly. The mixture is stirred between 4° C. and room temperature for 18 h, then heated at 65° C. for 2 hours. The reaction mixture is then cooled to room temperature, then poured dropwise into diisopropyl ether (2.4 L) with stirring. The white precipitate is collected by filtration, washed with diisopropyl ether (2×125 mL), then dried under reduced pressure at 30° C. to give a white solid. The solid is solubilized in N,N-dimethylacetamide (DMAc, 150 mL), then Pd/Al₂O₃ (6 g) is added under an argon atmosphere. The mixture is placed in a hydrogen atmosphere (10 bar) and stirred at 60° C. for 24 hours. After cooling to room temperature and filtration of the catalyst on a P4 sintered filter and PTFE Omnipore hydrophilic membrane 0.2 m, a water solution at pH 2 (900 mL) is poured dropwise on the DMAc solution, on a 45 minute period with stirring. After 18 hours of, the white precipitate is collected by filtration, washed with water, then dried under reduced pressure at 30° C. The obtained solid is solubilized in water (1.25 L) by adjusting the pH to 7 by addition of a 1N aqueous sodium hydroxide solution. The pH is then adjusted to pH 12 and the solution is kept under stirring for 1 h. After neutralization at pH 7, the solution is filtered through a 0.2 μm filter, diluted with ethanol to obtain a solution containing 30% mass of ethanol, then filtered through an activated carbon filter (3M R53SLP). The solution obtained is filtered through a 0.45 μm filter and purified by ultrafiltration against a 0.9% NaCl solution, then water until the conductimetry of the permeate is less than 50 μS/cm. The co-polyamino acid solution is then concentrated to approximately 30 g/L theoretical and the pH is adjusted to 7. The aqueous solution is filtered through a 0.2 μm filter and stored at 4° C.

Dry extract: 38.1 mg/g

DP (estimated by ¹H NMR)=23 where i=0.043

The calculated average molecular weight of co-polyamino acid AB34 is 3991 g/mol.

Organic HPLC-SEC (PEG Calibrator): Mn=2600 g/mol.

### EXAMPLE AB35: CO-POLYAMINO ACID AB35—SODIUM POLY-L-GLUTAMATE MODIFIED AT ONE END BY THE MOLECULE AA14-1 AND HAVING A NUMBER-AVERAGE MOLECULAR WEIGHT (MN) OF 2600 G/MOL

A solution of sodium poly-L-glutamate modified at one of its ends by molecule AA14-1 is obtained by a process similar to that used in the preparation of AB34 co-polyamino acid applied to molecule AA14-1 (0.4 g, 0.65 mmol) solution in chloroform (6.5 mL) and 3.79 g (14.4 mmol) of γ-benzyl-L-glutamate N-carboxyanhydride dissolved in DMF (6.5 mL), and by omitting the activated carbon filtration step.

Dry extract: 21.0 mg/g

DP (estimated by ¹H NMR)=22 where i=0.045

The calculated average molecular weight of co-polyamino acid AB35 is 3896 g/mol.

Organic HPLC-SEC (PEG Calibrator): Mn=2600 g/mol.

### EXAMPLE AB36: CO-POLYAMINO ACID AB36—SODIUM POLY-L-GLUTAMATE MODIFIED AT ONE END BY THE MOLECULE AA16 AND HAVING A NUMBER-AVERAGE MOLECULAR WEIGHT (MN) OF 2800 G/MOL

By a process similar to that used in the preparation of co-polyamino acid AB34 applied to molecule AA16 (3.28 g, 4.67 mmol) and 27.02 g (102.6 mmol) of γ-benzyl-L-glutamate N-carboxyanhydride, a solution of sodium poly-L-glutamate modified at one of its ends by molecule AA16 is obtained.

Dry extract: 23.9 mg/g

DP (estimated by ¹H NMR)=22 where i=0.045

The calculated average molecular weight of co-polyamino acid AB36 is 3987 g/mol.

Organic HPLC-SEC (PEG Calibrator): Mn=2800 g/mol.

### EXAMPLE AB37: CO-POLYAMINO ACID AB37—SODIUM POLY-L-GLUTAMATE MODIFIED AT ONE OF ITS ENDS BY THE MOLECULE AA17 AND HAVING A NUMBER-AVERAGE MOLECULAR WEIGHT (MN) OF 2800 G/MOL

By a process similar to that used in the preparation of co-polyamino acid AB34 applied to molecule AA17 (4.50 g, 9.73 mmol) and 56.33 g (214.0 mmol) of γ-benzyl-L-glutamate N-carboxyanhydride, a solution of sodium poly-L-glutamate modified at one of its ends by molecule AA17 is obtained.

Dry extract: 26.8 mg/g

DP (estimated by ¹H NMR)=24 where i=0.042

The calculated average molecular weight of co-polyamino acid AB37 is 4049 g/mol.

Organic HPLC-SEC (PEG Calibrator): Mn=2800 g/mol.

### Part B

### BB: Synthesis of Intermediate Hydrophobic Compounds Hy to Obtain the Radicals -Hy Wherein p=2

The hydrophobic intermediate compounds are represented in the following table by the corresponding hydrophobic molecule before co-polyamino acid grafting.

TABLE 1d
List of hydrophobic intermediates synthesized according to the invention.

| No | HYDROPHOBIC INTERMEDIATE COMPOUNDS |
|---|---|
| BA1 | 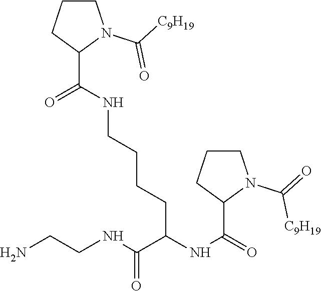 |
| BA2 | 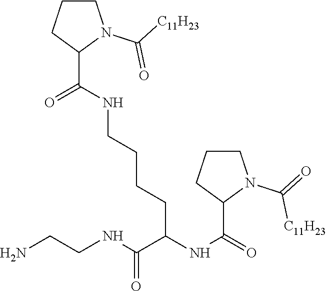 |
| BA3 | 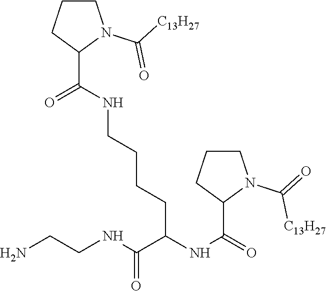 |
| BA4 | 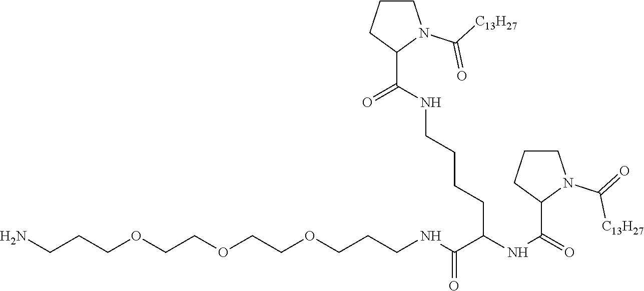 |
| BA5 | 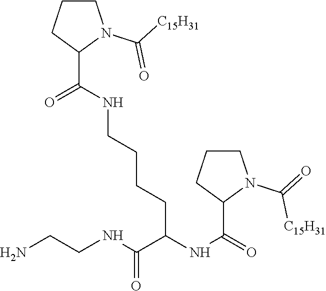 |
| BA6 | 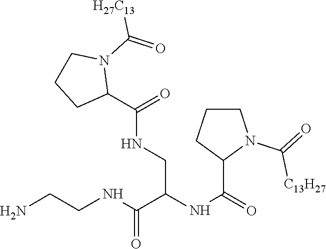 |
| BA7 | 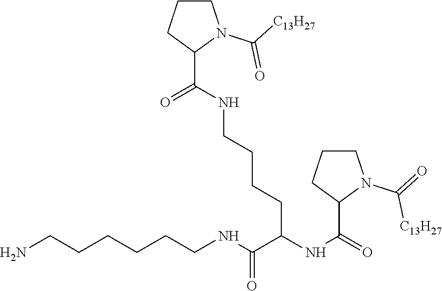 |

### Part BA: Synthesis of Hydrophobic Intermediates Wherein p=2

### EXAMPLE BA1: MOLECULE BA1

### Molecule B1: Product Obtained by the Reaction Between Decanoic Acid and L-Proline

Dicyclohexyl carbodiimide (DCC) (16.29 g, 78.96 mmol) and N-hydroxysuccinimide (NHS) (9.09 g, 78.96 mmol) are successively added to a solution of decanoic acid (14.28 g, 82.91 mmol) in THF (520 mL) at 0° C. After stirring for 60 hours at room temperature, the medium is cooled to 0° C. for 20 minutes, filtered through a sintered filter. L-proline (10 g, 86.86 mmol), diisopropylethylamine (DIPEA) (68.8 mL) and water (60 mL) are added to the filtrate. After stirring for 24 hours at room temperature, the medium is diluted with water (300 mL). The aqueous phase is washed with ethyl acetate (2×250 ml), acidified to pH ˜1 with a 1N HCl aqueous solution, then extracted with dichloromethane (3×150 ml). The combined organic phases are dried over Na₂SO₄, filtered, concentrated under vacuum, and the residue is purified by chromatography on silica gel (cyclohexane, ethyl acetate).

Yield: 14.6 g (69%)

NMR ¹H (CDCl₃, ppm): 0.87 (3H); 1.26 (12H); 1.65 (2H); 2.02 (3H); 2.34 (2H); 2.41 (1H); 3.48 (1H); 3.56 (1H); 4.58 (1H).

LC/MS (ESI): 270.2; (calculated ([M+H]+): 270.4).

### Molecule B2: Product Obtained by the Reaction Between Molecule B1 and L-Lysine

By a process similar to that used in the preparation of molecule B1 applied to molecule B1 (14.57 g, 54.07 mmol) and to L-lysine (4.15 g, 28.39 mmol), a yellow oil is obtained.

Yield: 16.4 g (93%)

NMR ¹H (CDCl₃, ppm): 0.88 (6H); 1.26 (24H); 1.35-1.65 (8H); 1.85-2.35 (12H); 2.53 (0.2H); 2.90 (0.8H); 3.45-3.75 (5H); 4.50-4.70 (3H); 7.82 (1H).

LC/MS (ESI): 649.6; (calculated ([M+H]+): 649.9).

### Molecule B3: Product Obtained by Reaction Between the Molecule B2 and Boc-Ethylenediamine

DIPEA (8.80 mL) and 2-(1H-benzotriazol-1-yl)-1-1,3,3-tetramethyluronium tetrafluoroborate (TBTU, 8.52 g, 26.54 mmol) are added at room temperature to a solution of molecule B2 (16.4 g, 25.27 mmol) in THF (170 mL). After 30 minutes of stirring, Boc-ethylenediamine (4.45 g, 27.8 mmol) is added. After stirring at room temperature for 2 hours, the solvent is evaporated under reduced pressure and the residue is diluted with ethyl acetate (400 mL). The organic phase is washed with water (250 mL), saturated aqueous solution of NaHCO₃ (250 ml), an aqueous solution of 1 N HCl (250 ml), a saturated aqueous solution of NaCl (250 ml) and is dried over Na₂SO₄. After filtration and concentration under vacuum, the residue obtained is purified by chromatography on silica gel (ethyl acetate, methanol) to produce a colorless oil.

Yield: 12.8 g (64%)

NMR ¹H (CDCl₃, ppm): 0.87 (6H); 1.25-1.60 (42H); 1.80-2.05 (4H); 2.15-2.45 (9H); 3.10-3.75 (10H); 4.30 (1H); 4.50 (2H); 5.50 (0.6H); 5.89 (0.2H); 6.15 (0.2H); 7.03 (1H); 7.47 (1H).

LC/MS (ESI): 791.8; (calculated ([M+H]+): 792.1).

### Molecule BA1

A 4 N HCl solution in dioxane (20.2 mL) is added to a solution of molecule B3 (12.78 g, 16.15 mmol) in dichloromethane (110 mL) at 5° C. After 20 hours of stirring at 5° C., the medium is concentrated under vacuum. The residue obtained is dissolved in methanol and evaporated under vacuum, this process being repeated 4 times to give a white solid of molecule BA1 in the form of hydrochloride salt.

Yield: 11.4 g (97%)

NMR ¹H (DMSO-d₆, ppm): 0.85 (6H); 1.25-1.50 (33H); 1.57 (1H); 1.70-2.40 (12H); 2.82 (2H); 3.00 (2H); 3.25-3.70 (6H); 4.05-4.50 (3H); 7.75-8.45 (6H).

LC/MS (ESI): 691.6; (calculated ([M+H]+): 692.0).

### EXAMPLE BA2: MOLECULE BA2

### Molecule B4: Product Obtained by the Reaction Between Lauric Acid and L-Proline

By a process similar to that used in the preparation of molecule B1, applied to lauric acid (31.83 g, 157.9 mmol) and to L-proline (20 g, 173.7 mmol), a yellow oil is obtained.

Yield: 34.3 g (73%)

NMR ¹H (CDCl₃, ppm): 0.87 (3H); 1.26 (16H); 1.70 (2H); 1.90-2.10 (3H); 2.35 (2H); 2.49 (1H); 3.48 (1H); 3.56 (1H); 4.60 (1H).

LC/MS (ESI): 298.2; (calculated ([M+H]+): 298.4).

### Molecule B5: Product Obtained by the Reaction Between Molecule B4 and L-Lysine

A white solid is obtained by a process similar to that used in the preparation of molecule B1 applied to molecule B4 (33.72 g, 113.36 mmol) and to L-lysine (8.70 g, 59.51 mmol).

Yield: 26.2 g (66%)

NMR ¹H (CDCl₃, ppm): 0.88 (6H); 1.26 (32H); 1.35-1.65 (8H); 1.85-2.35 (15H); 2.87 (1H); 3.40-3.75 (5H); 4.50-4.75 (3H); 7.87 (1H).

LC/MS (ESI): 705.6; (calculated ([M+H]+): 706.0).

### Molecule B6: Product Obtained by Reaction Between Boc-Ethylenediamine and Molecule B5

A colorless oil colorless is obtained by a process similar to that used in the preparation of molecule B3 applied to molecule B5 (25.74 g, 36.51 mmol) and Boc-ethylenediamine (6.43 g, 40.16 mmol).

Yield: 30.9 g (quantitative)

NMR ¹H (CDCl₃, ppm): 0.88 (6H); 1.35-1.65 (50H); 1.85-2.35 (13H); 3.05-3.75 (10H); 4.25-4.65 (3H); 5.50 (0.4H); 5.88 (0.2H); 6.16 (0.2H); 7.08 (1H); 7.26 (1H); 7.49 (0.2 hours)

LC/MS (ESI): 847.8; (calculated ([M+H]+): 848.2).

### Molecule BA2

Following a process similar to that used in the preparation of molecule BA1 applied to molecule B6 (30.9 g, 36.47 mmol), the residue obtained after concentration under vacuum is dissolved in methanol and evaporated under vacuum, this process being repeated 4 times to yield a white solid of molecule BA2 in the form of a hydrochloride salt after drying under reduced pressure.

Yield: 27.65 g (97%)

NMR ¹H (DMSO-d₆, ppm): 0.85 (6H); 1.10-2.40 (54H); 2.75-3.15 (4H); 3.25-3.60 (6H); 4.05-4.50 (3H); 7.50-8.50 (6H).

LC/MS (ESI): 747.6; (calculated ([M+H]+): 748.1).

### EXAMPLE BA3: MOLECULE BA3

### Molecule B7: Product Obtained by the Reaction Between Myristic Acid and L-Proline

A yellow oil is obtained by a process similar to that used in the preparation of molecule B1, applied to lauric acid (18.93 g, 82.91 mmol) and to L-proline (10 g, 86.86 mmol).

Yield: 20 g (78%)

NMR ¹H (CDCl₃, ppm): 0.88 (3H); 1.28 (20H); 1.70 (2H); 1.90-2.10 (3H); 2.36 (2H); 2.51 (1H); 3.47 (1H); 3.56 (1H); 4.61 (1H).

LC/MS (ESI): 326.2; (calculated ([M+H]+): 326.6).

### Molecule B8: Product Obtained by the Reaction Between Molecule B7 and L-Lysine

A white solid is obtained by a process similar to that used in the preparation of molecule B1 applied to molecule B7 (20.02 g, 61.5 mmol) and to L-lysine (4.72 g, 32.29 mmol).

Yield: 12.3 g (53%)

NMR ¹H (DMSO-d₆, ppm): 0.85 (6H); 1.26 (40H); 1.35-1.50 (6H); 1.50-2.10 (10H); 2.10-2.25 (4H); 3.01 (2H); 3.31-3.55 (4H); 4.10-4.40 (3H); 7.68 (0.6H); 7.97 (1H); 8.27 (0.4H); 12.50 (1H).

LC/MS (ESI): 761.8; (calculated ([M+H]+): 762.1).

### Molecule B9: Product Obtained by Reaction Between Boc-Ethylenediamine and Molecule B8

By a process similar to that used in the preparation of molecule B3 applied to molecule B8 (12 g, 15.77 mmol) and N-Boc-ethylenediamine (3.03 g, 18.92 mmol), a colorless oil is obtained after purification by chromatography column on silica gel (ethyl acetate, methanol).

Yield: 12.5 g (88%)

NMR ¹H (DMSO-d₆, ppm): 0.85 (6H); 1.20-1.55 (55H); 1.50-2.25 (14H); 2.95-3.10 (6H); 3.31-3.55 (4H); 4.10-4.40 (3H); 6.74 (1H); 7.60-8.25 (3H).

LC/MS (ESI): 904.1; (calculated ([M+H]+): 904.3).

### Molecule BA3

Following a process similar to that used in the preparation of molecule BA1 applied to molecule B9 (12.5 g, 13.84 mmol), the residue obtained after concentration under vacuum is dissolved in methanol and evaporated under vacuum, this process being repeated 4 times to yield a white solid of molecule BA3 in the form of a hydrochloride salt after drying under reduced pressure.

Yield: 9.2 g (79%)

NMR ¹H (DMSO-d₆, ppm): 0.85 (6H); 1.10-1.65 (48H); 1.70-2.35 (12H); 2.85 (2H); 3.01 (2H); 3.25-3.65 (6H); 4.10-4.50 (3H); 7.70-8.40 (6H).

LC/MS (ESI): 803.9; (calculated ([M+H]+): 804.2).

### EXAMPLE BA4: MOLECULE BA4

### Molecule B10: Product Obtained by Reaction Between Molecule B8 and Boc-1-Amino-4,7,10-Trioxa-13-Tridecane

By a process similar to that used in the preparation of molecule B3 applied to molecule B8 (29.80 g, 39.15 mmol) and Boc-1-amino-4.7,10-trioxa-13-tridecane amine (15.05 g, 46.96 mmol), a thick colorless oil is obtained.

Yield: 25.3 g (61%)

NMR ¹H (DMSO-d₆, ppm): 0.85 (6H); 1.25-2.35 (75H); 2.85-3.20 (6H); 3.25-3.65 (16H); 4.10-4.45 (3H); 6.38 (0.1H); 6.72 (0.9H); 7.50-8.25 (3H).

LC/MS (ESI): 1064.2; (calculated ([M+H]+): 1064.5).

### Molecule BA4

Following a process similar to that used in the preparation of molecule BA1 applied to molecule B10 (25.3 g, 23.8 mmol), the residue obtained after concentration under vacuum is dissolved in methanol and evaporated under vacuum, this process being repeated 4 times to yield a white solid of molecule BA4 in the form of a hydrochloride salt after drying under reduced pressure.

Yield: 20.02 g (84%)

NMR ¹H (DMSO-d₆, ppm): 0.85 (6H); 1.15-2.35 (66H); 2.80-3.20 (6H); 3.30-3.65 (16H); 4.10-4.45 (3H); 7.55-8.60 (6H).

LC/MS (ESI): 964.9; (calculated ([M+H]+): 964.6).

### EXAMPLE BA5: MOLECULE BA5

### Molecule B11: Product Obtained by Reaction Between Palmitoyl Chloride and L-Proline

A white solid of molecule B11 is obtained by a process similar to that used in the preparation of A26 molecule applied to palmitoyl chloride (15.39 g, 55.99 mmol) and L-proline (12.89 g, 111.98 mmol).

Yield: 19.10 g (96%)

NMR ¹H (CDCl₃, ppm): 0.88 (3H); 1.19-1.45 (24H); 1.58-1.74 (2H); 1.88-2.14 (3H); 2.15-2.54 (3H); 3.47 (1H); 3.58 (1H); 4.41 (0.1H); 4.61 (0.9H) 6.60-8.60 (1H).

LC/MS (ESI): 354.4; 707.8; (calculated ([M+H]+): 354.3; ([2M+H]+): 707.6).

### Molecule B12: Product Obtained by Reaction Between Molecule B11 and L-Lysine

By a process similar to that used in the preparation of molecule B1 applied to molecule B11 (19.10 g, 54.02 mmol) and L-lysine (4.15 g, 28.36 mmol), an oily residue is obtained after concentration of the reaction medium under reduced pressure. This residue is diluted in water (150 mL), washed with ethyl acetate (2×75 mL), then the aqueous phase is acidified to pH 1 by slow addition of 6N HCl. The product is extracted 3 times with dichloromethane, the organic phase is dried over Na₂SO₄ then filtered and concentrated under reduced pressure to give 11.2 g of yellow oily residue. Simultaneously, the organic phase of the above ethyl acetate is washed with an aqueous solution of 2N HCl (2×75 ml), a saturated aqueous solution of NaCl (75 ml), dried over Na₂SO₄ filtered and concentrated to give 10.2 g of yellow oily residue. A white solid is obtained after recrystallization of each of these residues in acetone.

Yield: 11.83 g (54%)

¹H RMN (CDCl₃, ppm): 0.87 (6H); 1.06-2.44 (70H); 2.78-2.96 (1H); 3.35-3.75 (5H); 4.28-4.43 (0.1H); 4.43-4.52 (0.2H); 4.52-4.61 (1.8H); 4.61-4.75 (0.9H); 7.74-8.02 (2H).

LC/MS (ESI): 818.0; (calculated ([M+H]+): 818.7).

### Molecule B13: Product Obtained by Coupling Between Molecule B12 and Boc-Ethylenediamine

By a process similar to that used in the preparation of molecule A27 applied to molecule B12 (18.00 g, 22.02 mmol) solution in THF and Boc-ethylenediamine (4.23 g, 26.43 mmol), a white solid is obtained after double recrystallization in acetonitrile

Yield: 17.5 g (83%)

1H NMR (DMSO-d6, ppm): 0.85 (6H); 1.15-2.29 (79H); 2.92-3.12 (6H); 3.30-3.59 (4H); 4.06-4.13 (0.65H); 4.16-4.29 (2H); 4.38-4.42 (0.35H); 6.71-6.76 (1H); 7.60-7.69 (1.3H); 7.76-7.81 (0.65H); 7.93-7.97 (0.35H); 8.00-8.04 (0.35H); 8.10-8.17 (0.35H).

LC/MS (ESI): 960.4 (calculated ([M+H]+): 960.8).

### Molecule BA5

By a process similar to that used in the preparation of molecule BA1 applied to molecule B13 (24.4 g, 25.43 mmol), the residue obtained after concentration under vacuum is solubilized in dichloromethane (150 mL), the organic phase is washed 2 twice with 2M aqueous sodium hydroxide solution (90 mL). Acetonitrile (120 mL) is added and dichloromethane is removed by concentration under reduced pressure. The medium is then left standing for 72 hours and a white solid is obtained after filtration and rinsing with acetonitrile, then drying under reduced pressure. This process is repeated 4 times.

Yield: 14.28 g (65%) 1H NMR (DMSO-d6, ppm): 0.85 (6H); 1.06-2.32 (70H); 2.53-2.63 (2H); 2.89-3.61 (10H); 4.04-4.43 (3H); 7.55-7.62 (0.65H); 7.65-7.72 (0.65H); 7.80 (0.65H); 7.91 (0.35H); 8.03 (0.35H); 8.14-8.23 (0.35H).

LC/MS (ESI): 860.0 (calculated ([M+H]+): 860.8).

### EXAMPLE BA6: MOLECULE BA6

### Molecule B14: Product Obtained by Coupling Between Molecule A26 and 2,3-Diaminopropionic Acid

By a process similar to that used in the preparation of molecule B1 applied to molecule A26 (80.00 g, 245.78 mmol) and to the dihydrochloride of 2,3-diaminopropionic acid (22.84 g, 129.04 mmol), a white solid is obtained after recrystallization in acetonitrile.

Yield: 69 g (78%)

1H NMR (DMSO-d6, ppm): 0.86 (6H); 1.08-1.38 (40H); 1.40-1.55 (4H); 1.68-2.30 (12H); 3.16-3.66 (6H); 4.20-4.39 (3H); 7.67-8.31 (2H); 12.70 (1H).

LC/MS (ESI): 719.4; 741.5; (calculated ([M+H]+): 719.6; ([M+Na]⁺): 741.6).

### Molecule B15: Product Obtained by Coupling Between Molecule B14 and Boc-Ethylenediamine

By a process similar to that used in the preparation of molecule A27 applied to molecule B14 (32.00 g, 44.50 mmol) and N-Boc-ethylenediamine (8.56 g, 53.40 mmol), a colorless oil is obtained after purification by chromatography column on silica gel (ethyl acetate, methanol).

Yield: 24.5 g (64%)

¹H NMR (DMSO-d6, ppm): 0.85 (6H); 1.16-2.42 (65H); 2.89-3.14 (4H); 3.17-3.66 (6H); 4.11-4.43 (3H); 6.77 (1H); 7.38-8.23 (3H).

LC/MS (ESI): 861.7 (calculated ([M+H]+): 861.7).

### Molecule BA6

By a process similar to that used in the preparation of molecule BA5 applied to molecule B15 (24.50 g, 28.45 mmol), a white solid is obtained after recrystallization in acetonitrile.

Yield: 19.7 g (91%)

1H NMR (DMSO-d6, ppm): 0.85 (6H); 1.10-2.40 (58H); 2.51-2.62 (2H); 2.90-3.16 (2H); 3.16-3.67 (6H); 4.04-4.47 (3H); 7.33-8.27 (3H).

LC/MS (ESI): 761.5 (calculated ([M+H]+): 761.6).

### EXAMPLE BA7: MOLECULE BA7

### Molecule B16: Product Obtained by the Reaction Between N-(Tert-Butoxycarbonyl)-1,6-Diaminohexane and Molecule B8

By a process similar to that used in the preparation of molecule A27 applied to molecule B8 (10 g, 13.14 mmol) and to N-(tert-butoxycarbonyl)-1.6-diaminohexane (3.41 g, 15.77 mmol) in dichloromethane, a white solid is obtained after recrystallization in acetonitrile.

Yield: 10.7 g (85%)

NMR ¹H (CDCl₃, ppm): 0.88 (6H); 1.17-2.40 (79H); 3.00-3.71 (10H); 4.26-4.58 (3H); 4.67 (1H); 6.74 (1H); 7.34-7.49 (2H).

LC/MS (ESI): 959.9; (calculated ([M+H]+): 959.8).

### Molecule BA7

Following a process similar to that used in the preparation of molecule BA1 applied to molecule B16 (10.5 g, 10.94 mmol), an aqueous solution of 2N NaOH is added dropwise to the reaction medium cooled to 0° C. The aqueous phase is extracted with dichloromethane and the organic phase is washed 3 times with 5% aqueous NaCl solution. After drying over Na₂SO₄, the organic phase is filtered, concentrated under vacuum, and the residue is recrystallized in acetonitrile.

Yield: 5.4 g (58%)

NMR ¹H (CDCl₃, ppm): 0.88 (6H); 1.19-2.40 (72H); 2.67 (2H); 3.03-3.70 (8H); 4.26-4.57 (3H); 6.71 (1H); 7.39-7.49 (2H).

LC/MS (ESI): 859.8; (calculated ([M+H]+): 859.7).

### BB: Synthesis of Co-Polyamino Acids

### Co-Polyamino Acids According to Formula VII or VIIa

TABLE 1
List of Co-Polyamino Acids according to Formula VII to VIIa

| | CO-POLYAMINO ACIDS BEARING CARBOXYLATE CHARGES AND |
|---|---|
| No | HYDROPHOBIC RADICALS |
| BB1 | 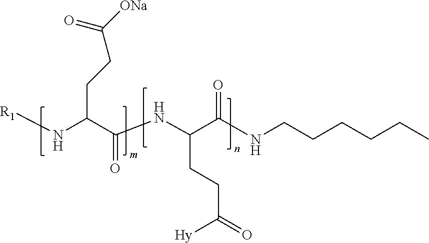 |
| | i = 0.05, DP (m + n) = 23 |
| | Hy = 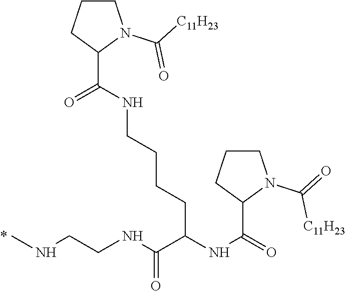 |
| | R₁ = H or pyroglutamate |
| BB2 | 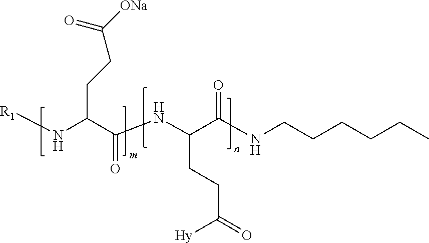 |
| | i = 0.047, DP (m + n) = 21 |
| | Hy = 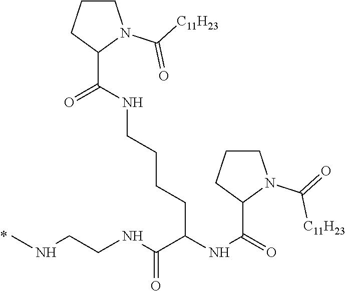 |
| | R₁ = H or pyroglutamate |
| BB3 | 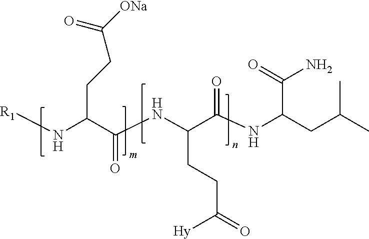 |
| | i = 0.049, DP (m + n) = 34 |
| | Hy = 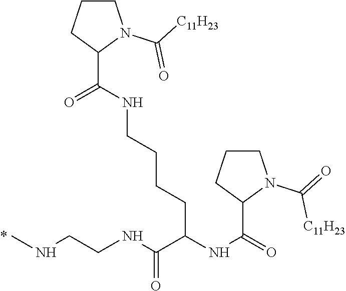 |
| | R₁ = H or pyroglutamate |
| BB4 | 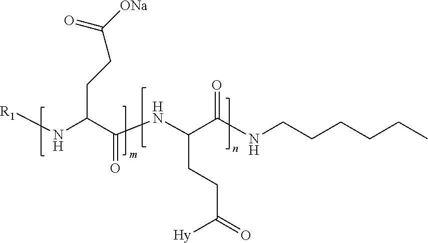 |
| | i = 0.04, DP (m + n) = 65 |
| | Hy = 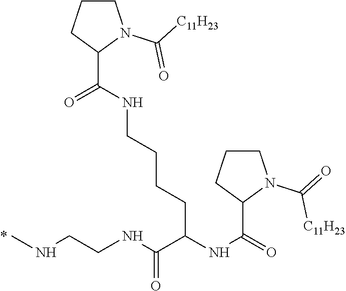 |
| | R₁ = H or pyroglutamate |
| BB5 | 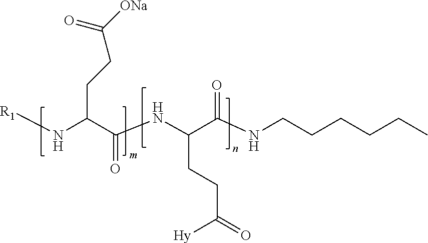 |
| | i = 0.042, DP (m + n) = 23 |
| | Hy = 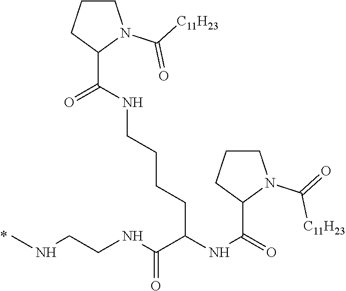 |
| | R1 = CH₃—CO—, H or pyroglutamate |
| BB6 | 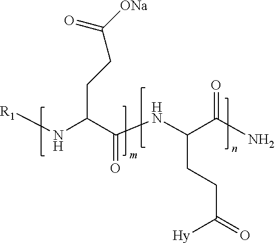 |
| | i = 0.04, DP (m + n) = 24 |
| | Hy = 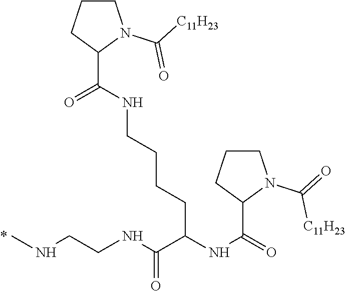 |
| | R1 = CH₃—CO—, H or pyroglutamate |
| BB7 | 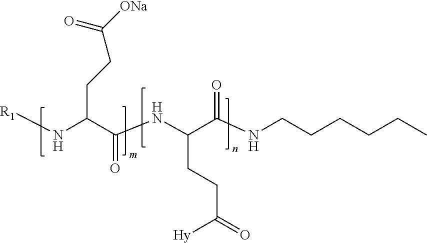 |
| | i = 0.042, DP (m + n) = 22 |
| | Hy = 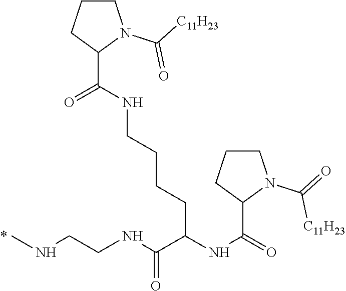 |
| | R₁ = H or pyroglutamate |
| BB8 | 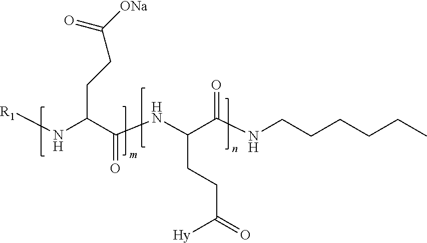 |
| | i = 0.026, DP (m + n) = 21 |
| | Hy = 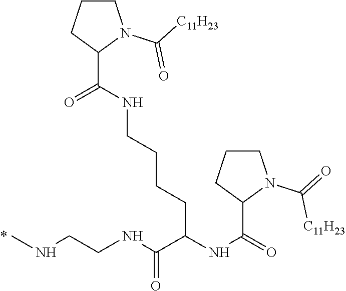 |
| | R₁ = H or pyroglutamate |
| BB9 | 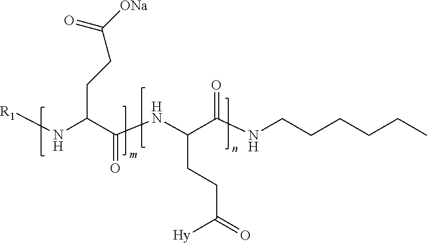 |
| | i = 0.05, DP (m + n) = 26 |
| | Hy = 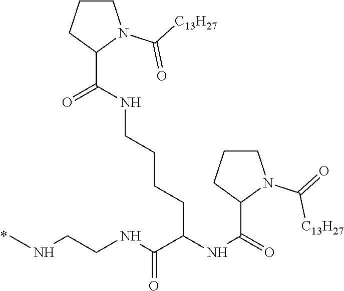 |
| | R₁ = H or pyroglutamate |
| BB10 | 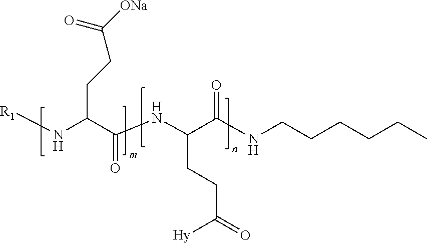 |
| | i = 0.029, DP (m + n) = 22 |
| | Hy = 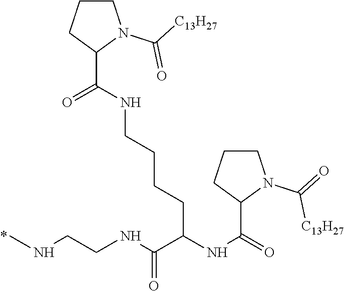 |
| | R₁ = H or pyroglutamate |
| BB11 | 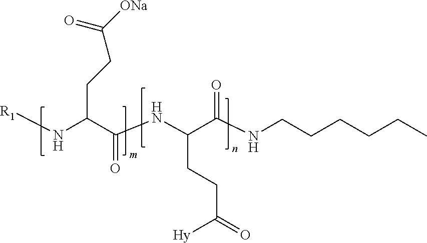 |
| | i = 0.032, DP (m + n) = 22 |
| | Hy = 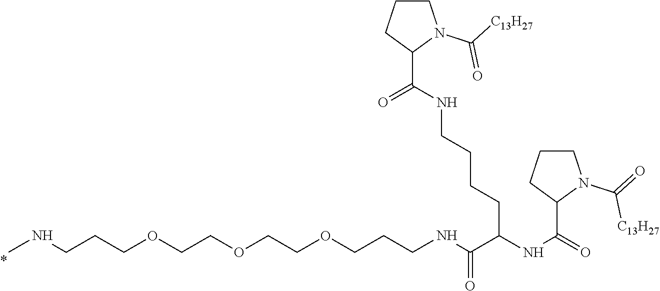 |
| | R1 = CH₃—CO—, H or pyroglutamate |
| BB12 | 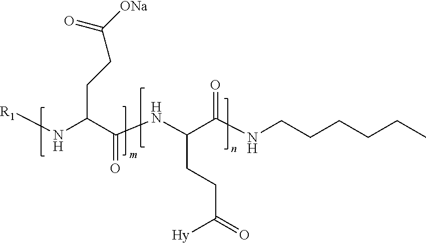 |
| | i = 0.03, DP (m + n) = 23 |
| | Hy = 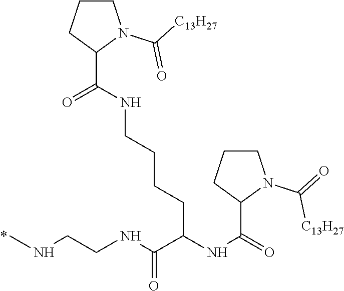 |
| | R1 = CH₃—CO—, H or pyroglutamate |
| BB13 | 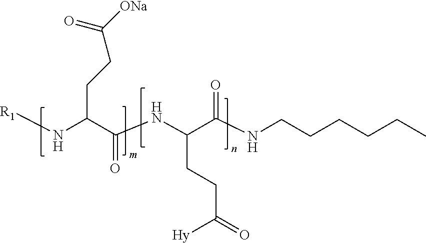 |
| | i = 0.08, DP = 25 |
| | Hy = 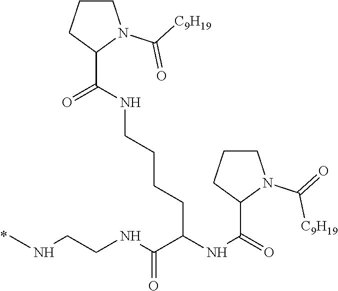 |
| | R₁ = H or pyroglutamate |
| BB19′ | 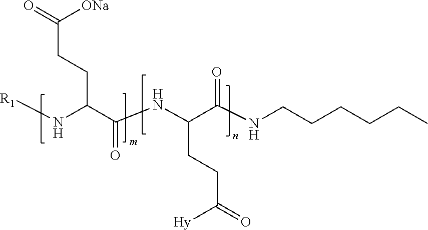 |
| | i = 0.045, DP (m + n) = 60 |
| | Hy = 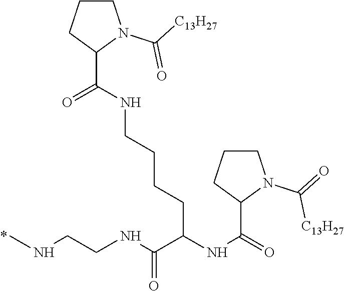 |
| | R₁ = pyroglutamate |

### Co-Polyamino Acids Defined by Formula VII or VIIb

TABLE 1e
list of co-polyamino acids synthesized according to the invention.

| | CO-POLYAMINOACIDES BEARING CARBOXYLATE LOADS AND |
|---|---|
| N° | HYDROPHOBIC RADICALS |
| BB14 | 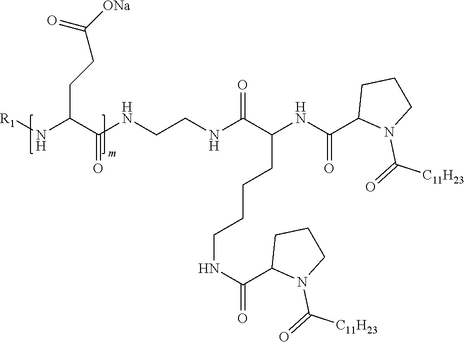 |
| | i = 0.034, DP (m) = 29 |
| | R₁ = H or pyroglutamate |
| BB15 | 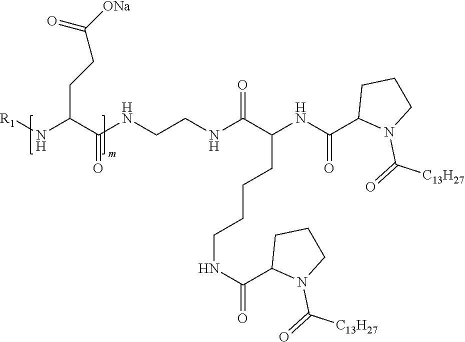 |
| | i = 0.042, DP (m) = 24 |
| | R₁ = H or pyroglutamate |
| BB16 | 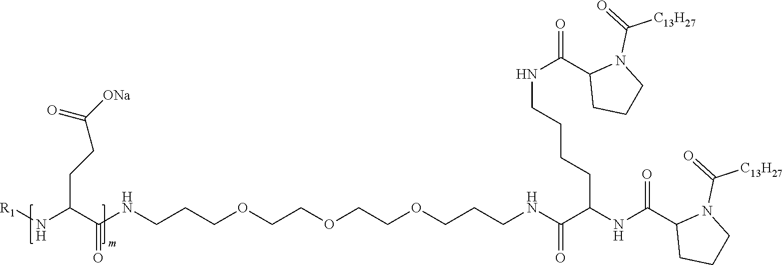 |
| | i = 0.043, DP (m) = 23 |
| | R1 = H or pyroglutamate |
| BB17 | 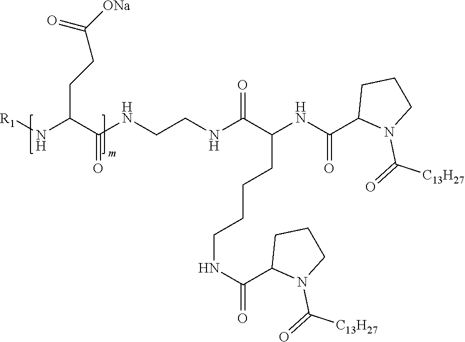 |
| | i = 0.015, DP (m) = 65 |
| | R₁ = H or pyroglutamate |
| BB18 | 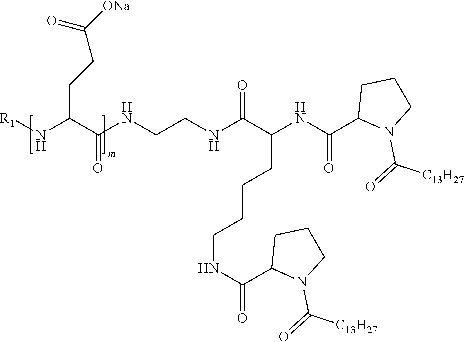 |
| | i = 0.025, DP (m) = 40 |
| | R₁ = H or pyroglutamate |
| BB19 | 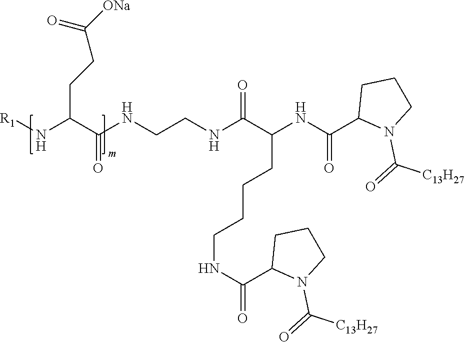 |
| | i = 0.04, DP (m) = 25 |
| | R₁ = H or pyroglutamate |
| BB20 | 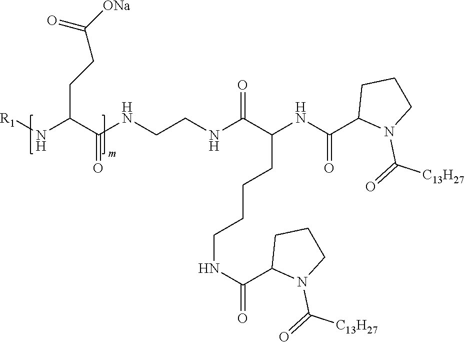 |
| | i = 0.059, DP (m) = 17 |
| | R₁ = H or pyroglutamate |
| BB21 | 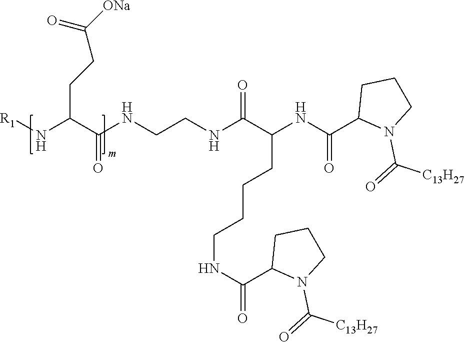 |
| | i = 0.11, DP (m) = 9 |
| | R₁ = H or pyroglutamate |
| BB22 | 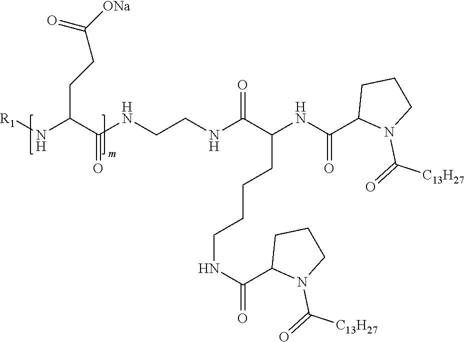 |
| | i = 0.048, DP (m) = 21 |
| | R₁ = H or pyroglutamate |
| BB23 | 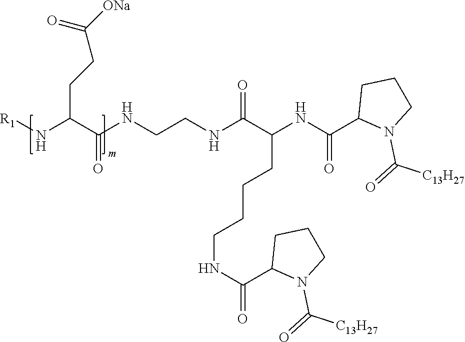 |
| | i = 0.048, DP (m) = 21 |
| | R₁ = H or pyroglutamate |
| BB24 | 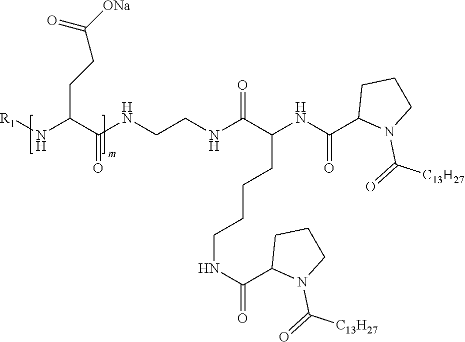 |
| | i = 0.040, DP (m) = 25 |
| | R₁ = H or pyroglutamate |
| BB25 | 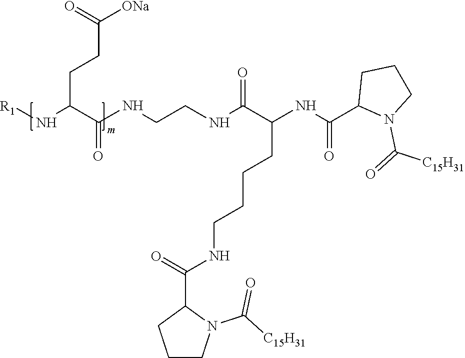 |
| | i = 0.043, DP (m) = 23 |
| | R₁ = H or pyroglutamate |
| BB26 | 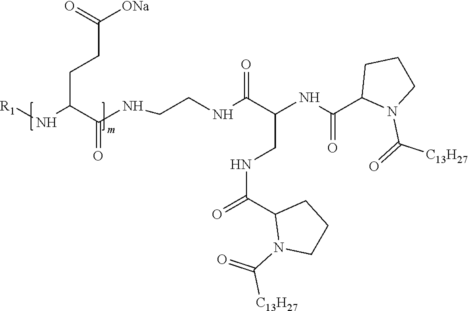 |
| | i = 0.048, DP (m) = 21 |
| | R₁ = H or pyroglutamate |
| BB27 | 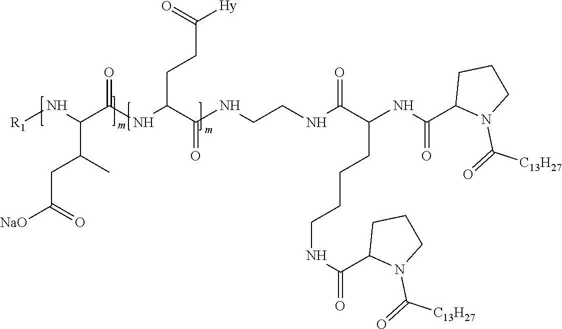 |
| | i = 0.089, DP (m + n) = 22 |
| | 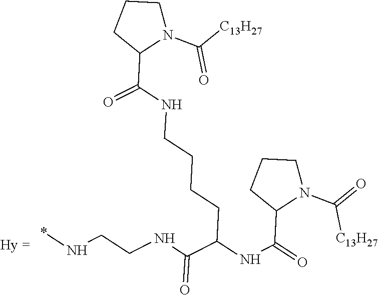 |
| | R₁ = H or pyroglutamate |
| BB42 | 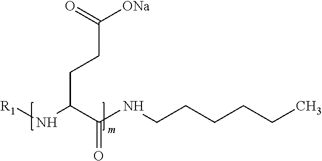 |
| | i = 0.045, DP (m) = 22 |
| | 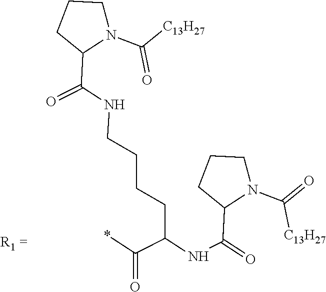 |
| BB25′ | 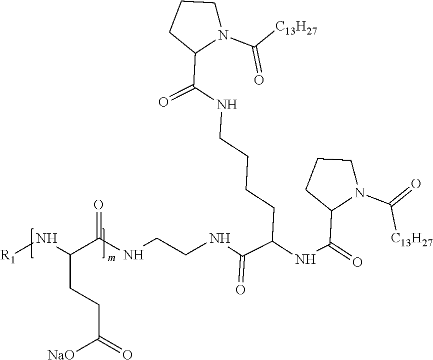 |
| | i = 0.09, DP (m) = 22 |
| | 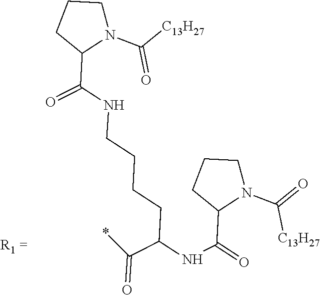 |
| BB43 | 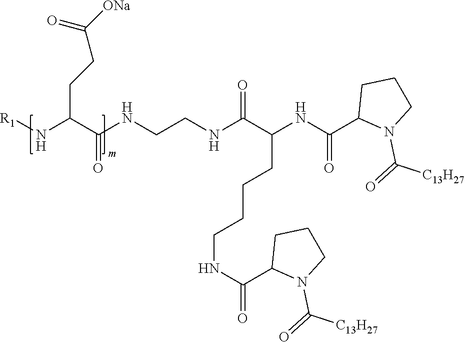 |
| | i = 0.09, DP (m) = 22 |
| | 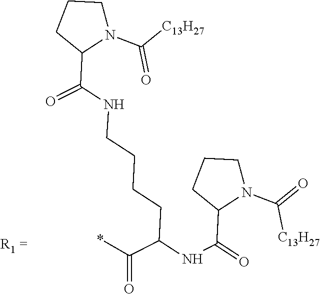 |
| BB44 | 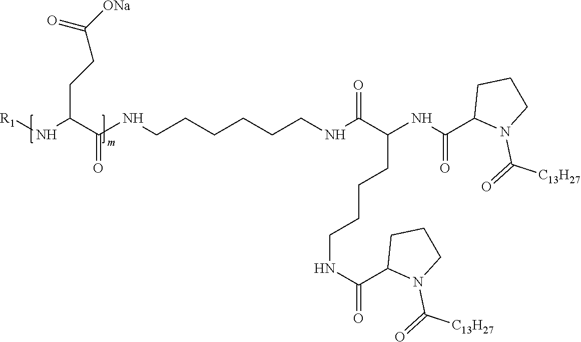 |
| | i = 0.04, DP (m) = 25 |
| | R₁ = H or pyroglutamate |

### Part BB: synthesis of co-polyamino acids

### EXAMPLE BB1: CO-POLYAMINO ACID BB1—SODIUM POLY-L-GLUTAMATE MODIFIED BY MOLECULE BA2 AND HAVING A NUMBER-AVERAGE MOLECULAR WEIGHT (MN) OF 2400 G/MOL

### Co-Polyamino Acid BB1-1: Poly-L-Glutamic Acid of Number-Average Molecular Weight (Mn) 3860 g/Mol from the Polymerization of γ-Benzyl-L-Glutamate N-Carboxyanhydride Initiated by Hexylamine

In a previously oven-dried flask, γ-benzyl-L-glutamate N-carboxyanhydride (90.0 g, 342 mmol) is placed under vacuum for 30 minutes, then anhydrous DMF (465 mL) is added. The mixture is then stirred under argon until complete dissolution, cooled to 4° C., then hexylamine (1.8 ml, 14 mmol) is quickly introduced. The mixture is stirred at 4° C. and room temperature for 2 days. The reaction medium is then heated at 65° C. for 4 hours, cooled to room temperature, then poured dropwise into diisopropyl ether (6 L) with stirring. The white precipitate is collected by filtration, washed with diisopropyl ether (500×250 mL), then dried under vacuum at 30° C. to give a poly (gamma-benzyl-L-glutamic acid) (PBLG).

A solution of hydrobromic acid (HBr) at 33% in acetic acid (135 mL, 0.77 mol) is added dropwise to a solution of PBLG (42.1 g) in trifluoroacetic acid (TFA, 325 mL) at 4° C. The mixture is stirred at room temperature for 2 hours, then poured dropwise onto a 1:1 (v/v) mixture of diisopropyl ether and water with stirring (1.6 L). After stirring for 1 hour 30 minutes, the heterogeneous mixture is allowed to stand overnight. The white precipitate is collected by filtration, washed with a 1:1 (v/v) mixture of diisopropyl ether and water (200 mL).

The obtained solid is solubilized in water (1 mL) by adjusting the pH to 7 by adding 10 N aqueous sodium hydroxide solution, then 1N aqueous sodium hydroxide solution. After solubilization, the theoretical concentration is adjusted to 25 g/L theoretical by addition of water to obtain a final volume of 1.5 mL.

The solution is filtered through a 0.45 μm filter, then purified by ultrafiltration against a solution of NaCl 0.9%, then water until the conductimetry of the permeate is less than 50 μS/cm.

The aqueous solution is then acidified by adding a 37% hydrochloric acid solution until a pH of 2 is reached. After stirring for 4 hours, the precipitate obtained is filtered, then dried under vacuum at 30° C. to give a poly-L-glutamic acid with a number-average molecular weight (Mn) of 3860 g/mol relative to a polyoxyethylene standard (PEG).

### Co-Polyamino Acid BB1

Co-polyamino acid BB1-1 (10.0 g) is solubilized in DMF (700 mL) at 30-40° C., then cooled to 0° C. The hydrochloride salt of molecule BA2 (2.95 g, 3.8 mmol) is suspended in DMF (45 mL) and triethylamine (0.39 g, 3.8 mmol) is then added to this suspension and the mixture is slightly heated with stirring until complete dissolution. N-methylmorpholine (NMM, 7.6 g, 75 mmol) in DMF (14 mL) and ethyl chloroformate (ECF, 8.1 g, 75 mmol) are added to a solution of co-polyamino acid at 0° C. After 10 minutes at 0° C., the solution of molecule BA2 is added and the medium maintained at 30° C. for 1 h. The reaction mixture is poured dropwise over 6 L of water containing NaCl (15% by weight) and HCl (pH 2), then left to stand overnight. The precipitate is collected by filtration, washed with sodium chloride solution at pH 2 (1 L) and dried under vacuum for about 1 hour. The white solid obtained is taken up in water (600 ml) and the pH is adjusted to 7 by slowly adding a 1N aqueous solution of NaOH. The volume is adjusted to 700 ml by addition of water. After filtering on a 0.45 μm filter, the clear solution obtained is purified by ultrafiltration against a solution of NaCl 0.9%, then water, until the conductimetry of the permeate is less than 50 μS/cm. After removal, the solution is filtered through a 0.2 μm filter and stored at 2-8° C.

Dry extract: 19.7 mg/g

DP (estimated based on ¹H NMR): 23

Based on ¹H NMR: i=0.05

The calculated average molecular weight of co-polyamino acid BB1 is 4350 g/mol.

Aqueous HPLC-SEC (PEG calibrant): Mn=2400 g/mol.

### EXAMPLE BB2: CO-POLYAMINO ACID BB2—SODIUM POLY-L-GLUTAMATE MODIFIED BY MOLECULE BA2 AND HAVING A NUMBER-AVERAGE MOLECULAR WEIGHT (MN) OF 4900 G/MOL

A poly-L-glutamic acid of number-average molecular weight (Mn) 4100 g/mol (5.0 g) obtained by a process similar to that used for the preparation of the co-polyamino acid BB1-1 is solubilized in DMF (205 mL) at 30-40° C. and maintained at this temperature. In parallel, the hydrochloride salt of the BA2 molecule (1.44 g, 1.84 mmol) is suspended in DMF (10 mL) and triethylamine (0.19 g, 1.84 mmol) is added then the mixture is gently heated with stirring until completely dissolved. The solution of molecule BA2 then 2-hydroxypyridine N-oxide (HOPO, 0.31 g, 2.76 mmol) are successively added to the co-polyamino acid solution in DMF, NMM (3.7 g, 36.7 mmol). The reaction medium is then cooled to 0° C., then EDC (0.53 g, 2.76 mmol) is added and the medium is raised to room temperature for 3 hours. The reaction mixture is poured dropwise over 1.55 L of water containing NaCl 15% by weight and HCl (pH 2) with stirring. At the end of the addition, the pH is readjusted to 2 with a 1 N HCl solution, and the suspension is allowed to stand overnight. The precipitate is collected by filtration, then rinsed with 100 mL of water. The white solid obtained is solubilized in 200 mL of water by slowly adding a 1N aqueous NaOH solution to pH 7 with stirring, then the solution is filtered through a 0.45 μm filter. The clear solution obtained is purified by ultrafiltration against 0.9% NaCl solution, then with water, until the conductimetry of the permeate is less than 50 μS/cm. The obtained solution is filtered through a 0.2 μm filter and stored at 2-8° C.

Dry extract: 16.3 mg/g

DP (estimated based on ¹H NMR): 21

Based on ¹H NMR: i=0.047

The calculated average molecular weight of co-polyamino acid BB2 is 3932 g/mol.

Aqueous HPLC-SEC (PEG calibrant): Mn=4900 g/mol.

### EXAMPLE BB3: CO-POLYAMINO ACID BB3—SODIUM POLY-L-GLUTAMATE MODIFIED BY MOLECULE BA2 AND HAVING A NUMBER-AVERAGE MOLECULAR WEIGHT (MN) OF 6400 G/MOL

### Co-Polyamino Acid BB3-1: Poly-L-Glutamic Acid of Number-Average Molecular Weight (Mn) 17500 g/Mol from the Polymerization of γ-Methyl-L-Glutamate N-Carboxyanhydride Triggered by L-Leucinamide

A poly-L-glutamic acid of number average mass (Mn) 17500 g/mol relative to a standard polymethyl methacrylate (PMMA) is obtained by polymerization of γ-methyl-N-carboxyanhydride of glutamic acid using L-leucinamide as an initiator and by deprotecting the methyl esters using a 37% hydrochloric acid solution according to the process described in patent application FR-A-2 801 226.

By a process similar to that used for the preparation of co-polyamino acid BB2 applied to the hydrochloride salt of molecule BA2 (3.23 g, 4.1 mmol) and to co-polyamino acid BB3-1 (11 g), a sodium poly-L-glutamate modified by molecule BA2 is obtained.

Dry extract: 27.5 mg/g

DP (estimated based on ¹H NMR): 34

Based on NMR ¹H: i=0.049

The calculated average molecular weight of co-polyamino acid BB3 is 6405 g/mol.

Aqueous HPLC-SEC (PEG calibrant): Mn=6400 g/mol.

### EXAMPLE BB4: CO-POLYAMINO ACID BB4—SODIUM POLY-L-GLUTAMATE MODIFIED BY MOLECULE BA2 AND HAVING A NUMBER-AVERAGE MOLECULAR WEIGHT (MN) OF 10500 G/MOL

By a process similar to that used for the preparation of co-polyamino acid BB2 applied to the hydrochloride salt of molecule BA2 (5 g, 6.35 mmol) and to a poly-L-glutamic acid with a number-average molecular weight Mn=10800 g/mol (21.7 g) obtained by a process similar to that used for the preparation of co-polyamino acid BB1-1, a sodium poly-L-glutamate modified by molecule BA2 is obtained.

Dry extract: 28.2 mg/g

DP (estimated based on ¹H NMR): 65

Based on ¹H NMR: i=0.04

The calculated average molecular weight of co-polyamino acid BB4 is 11721 g/mol.

Aqueous HPLC-SEC (PEG calibrant): Mn=10500 g/mol.

### EXAMPLE BB5: CO-POLYAMINO ACID BB5—SODIUM POLY-L-GLUTAMATE CAPPED AT ONE OF ITS ENDS BY AN ACETYL GROUP AND MODIFIED BY MOLECULE BA2 AND HAVING A NUMBER-AVERAGE MOLECULAR WEIGHT (MN) OF 3600 G/MOL

### Co-Polyamino Acid BB5-1: Poly-L-Glutamic Acid of Mn 3700 g/Mol from the Polymerization of γ-Benzyl-L-Glutamate N-Carboxyanhydride Initiated by Hexylamine and Capped at One End by an Acetyl Group

In a previously oven-dried flask, γ-benzyl-L-glutamate N-carboxyanhydride (100.0 g, 380 mmol) is placed under vacuum for 30 minutes, then anhydrous DMF (250 mL) is added. The mixture is then stirred under argon until complete dissolution, cooled to 4° C., then hexylamine (2.3 ml, 17 mmol) is quickly introduced. The mixture is stirred between 4° C. and room temperature for 2 days, then precipitated in diisopropyl ether (3.4 L). The precipitate is collected by filtration, washed twice with diisopropyl ether (225 mL), then dried to give a white solid which is dissolved in 450 mL of THF. N,N-diisopropylethylamine (DIPEA, 31 mL, 176 mmol), then acetic anhydride (17 mL, 176 mmol) are successively added to this solution. After stirring overnight at room temperature, the solution is slowly poured into diisopropyl ether (3 mL) over a period of 30 minutes with stirring. After stirring for 1 hour, the precipitate is filtered off, washed twice with diisopropyl ether (200 mL) and then dried under vacuum at 30° C. to give a poly (γ-benzyl-L-glutamic acid) capped at one end by an acetyl group.

A solution of hydrobromic acid (HBr) at 33% in acetic acid (235 mL, 1.34 mol) is added dropwise to a solution of co-polyamino acid capped (72 g) in trifluoroacetic acid (TFA, 335 mL) at 4° C. The mixture is stirred at room temperature for 3 h30, then poured dropwise onto a 1:1 (v/v) mixture of diisopropyl ether and water with stirring (4 L). After stirring for 2 hours, the heterogeneous mixture is allowed to stand overnight. The white precipitate is collected by filtration, washed with a 1:1 (v/v) mixture of diisopropyl ether and water (340 mL), then with water (340 mL). The obtained solid is then solubilized in water (1.5 L) by adjusting the pH to 7 by adding a 10N aqueous solution of sodium hydroxide, then a 1N aqueous sodium hydroxide solution. After solubilization, the solution is diluted to 20 g/L by adding water to obtain a final volume of 2.1 L. The solution is filtered through a 0.45 m filter, then purified by ultrafiltration against a solution of NaCl 0.9%, then water until the conductimetry of the permeate is less than 50 μS/cm. The co-polyamino acid solution is then concentrated until a final volume of 1.8 L. The aqueous solution is then acidified by adding 37% hydrochloric acid solution until a pH of 2 is reached. After stirring for 4 hours, the precipitate obtained is filtered, washed with water (330 mL), then dried under vacuum at 30° C. to give a poly-L-glutamic acid of number-average molar weight (Mn) 3700 g/mol relative to a standard of polyoxyethylene (PEG).

### Co-Polyamino Acid BB5

By a process similar to that used in the preparation of co-polyamino acid BB2 applied to the hydrochloride salt of molecule BA2 (6.92 g, 8.8 mmol) and co-polyamino acid BB5-1 (30.0 g), a sodium poly-L-glutamate capped at one end by an acetyl group and modified by molecule BA2 is obtained.

Dry extract: 29.4 mg/g

DP (estimated based on ¹H NMR): 23

Based on ¹H NMR: i=0.042

The calculated average molecular weight of co-polyamino acid BB5 is 4302 g/mol.

Aqueous HPLC-SEC (PEG calibrant): Mn=3600 g/mol.

### EXAMPLE BB6: CO-POLYAMINO ACID BB6—SODIUM POLY-L-GLUTAMATE CAPPED AT ONE OF ITS ENDS BY AN ACETYL GROUP AND MODIFIED BY MOLECULE BA2 AND HAVING A NUMBER-AVERAGE MOLECULAR WEIGHT (MN) OF 4100 G/MOL

A sodium poly-L-glutamate capped at one end by an acetyl group and modified by molecule BA2 is obtained by a process similar to that used in the preparation of the co-polyamino acid BB2 applied to the hydrochloride salt of molecule BA2 (5.8 g, 7.4 mmol) and to a poly-L-glutamic acid of number-average molecular mass Mn=3800 g/mol (25 g) obtained by a process similar to that used in the preparation of co-polyamino acid BB5-1 using ammonia instead of hexylamine.

Dry extract: 27.6 mg/g

DP (estimated based on ¹H NMR): 24

Based on ¹H NMR: i=0.04

The calculated average molecular weight of co-polyamino acid BB6 is 4387 g/mol.

Aqueous HPLC-SEC (PEG calibrant): Mn=4100 g/mol.

### EXAMPLE BB7: CO-POLYAMINO ACID BB7—SODIUM POLY-L-GLUTAMATE MODIFIED BY MOLECULE BA2 AND HAVING A NUMBER-AVERAGE MOLECULAR WEIGHT (MN) OF 4200 G/MOL

A sodium poly-L-glutamate modified with molecule BA2 is obtained by a process similar to that used in the preparation of the co-polyamino acid BB2 applied to the hydrochloride salt of molecule BA2 (7.07 g, 9.0 mmol) and to a poly-L-glutamic acid of number-average molecular mass Mn=3600 g/mol (30.0 g) obtained by a process similar to that used in the preparation of co-polyamino acid BB1-1.

Dry extract: 28.3 mg/g

DP (estimated based on ¹H NMR): 22

Based on ¹H NMR: i=0.042

The calculated average molecular weight of co-polyamino acid BB7 is 4039 g/mol.

Aqueous HPLC-SEC (PEG calibrant): Mn=4200 g/mol.

### EXAMPLE BB8: CO-POLYAMINO ACID BB8—SODIUM POLY-L-GLUTAMATE MODIFIED BY MOLECULE BA2 AND HAVING A NUMBER-AVERAGE MOLECULAR WEIGHT (MN) OF 5200 G/MOL

A sodium poly-L-glutamate modified with molecule BA2 is obtained by a process similar to that used in the preparation of the co-polyamino acid BB2 applied to the hydrochloride salt of molecule BA2 (0.85 g, 1.1 mmol) and to a poly-L-glutamic acid of number-average molecular mass Mn=4100 g/mol (5.0 g) obtained by a process similar to that used in the preparation of co-polyamino acid BB1-1.

Dry extract: 28.6 mg/g

DP (estimated based on ¹H NMR): 21

Based on ¹H NMR: i=0.026

The calculated average molecular weight of co-polyamino acid BB8 is 3620 g/mol.

Aqueous HPLC-SEC (PEG calibrant): Mn=5200 g/mol.

### EXAMPLE BB9: CO-POLYAMINO ACID BB9—SODIUM POLY-L-GLUTAMATE MODIFIED BY MOLECULE BA3 AND HAVING A NUMBER-AVERAGE MOLECULAR WEIGHT (MN) OF 4700 G/MOL

A sodium poly-L-glutamate modified with molecule BA3 is obtained by a process similar to that used in the preparation of the co-polyamino acid BB2 applied to the hydrochloride salt of molecule BA3 (3.05 g, 3.6 mmol) and to a poly-L-glutamic acid of number-average molecular mass Mn=4100 g/mol (10.0 g) obtained by a process similar to that used in the preparation of co-polyamino acid BB1-1.

Dry extract: 28.6 mg/g

DP (estimated based on ¹H NMR): 26

Based on ¹H NMR: i=0.05

The calculated average molecular weight of co-polyamino acid BB9 is 4982 g/mol.

Aqueous HPLC-SEC (PEG calibrant): Mn=4700 g/mol.

### EXAMPLE BB10: CO-POLYAMINO ACID BB10—SODIUM POLY-L-GLUTAMATE MODIFIED BY MOLECULE BA3 AND HAVING A NUMBER-AVERAGE MOLECULAR WEIGHT (MN) OF 4200 G/MOL

A sodium poly-L-glutamate modified with molecule BA3 is obtained by a process similar to that used in the preparation of the co-polyamino acid BB2 applied to the hydrochloride salt of molecule BA3 (1.90 g, 2.3 mmol) and to a poly-L-glutamic acid of number-average molecular mass Mn=3500 g/mol (10.0 g) obtained by a process similar to that used in the preparation of co-polyamino acid BB1-1.

Dry extract: 25.9 mg/g

DP (estimated based on ¹H NMR): 22

Based on ¹H NMR: i=0.029

The calculated average molecular weight of co-polyamino acid BB10 is 3872 g/mol.

Aqueous HPLC-SEC (PEG calibrant): Mn=4200 g/mol.

### EXAMPLE BB11: CO-POLYAMINO ACID BB11—SODIUM POLY-L-GLUTAMATE CAPPED AT ONE OF ITS ENDS BY AN ACETYL GROUP AND MODIFIED BY MOLECULE BA4 AND HAVING A NUMBER-AVERAGE MOLECULAR WEIGHT (MN) OF 3900 G/MOL

By a process similar to that used for the preparation of co-polyamino acid BB2 applied to the hydrochloride salt of molecule BA4 (2.21 g, 2.2 mmol) and to a poly-L-glutamic acid having a number-average molecular weight Mn=3700 g/mol (10 g) obtained by a process similar to that used for the preparation of co-polyamino acid BB5-1, a sodium poly-L-glutamate capped at one of its ends by an acetyl group and modified by molecule BA4 is obtained.

Dry extract: 28.1 mg/g

DP (estimated based on ¹H NMR): 22

Based on ¹H NMR: i=0.032

The calculated average molecular weight of co-polyamino acid BB11 is 4118 g/mol.

Aqueous HPLC-SEC (PEG calibrant): Mn=3900 g/mol.

### EXAMPLE BB12: CO-POLYAMINO ACID BB12—SODIUM POLY-L-GLUTAMATE CAPPED AT ONE OF ITS ENDS BY AN ACETYL GROUP AND MODIFIED BY MOLECULE BA3 AND HAVING A NUMBER-AVERAGE MOLECULAR WEIGHT (MN) OF 3900 G/MOL

By a process similar to that used for the preparation of co-polyamino acid BB2 applied to the hydrochloride salt of molecule BA3 (1.9 g, 2.3 mmol) and a poly-L-glutamic acid having a number-average molecular weight Mn=3600 g/mol (10 g) obtained by a process similar to that used for the preparation of co-polyamino acid BB5-1, a sodium poly-L-glutamate capped at one of its ends by an acetyl group and modified by molecule BA3 is obtained.

Dry extract: 26.7 mg/g

DP (estimated based on ¹H NMR): 23

Based on ¹H NMR: i=0.03

The calculated average molecular weight of co-polyamino acid BB12 is 4145 g/mol.

Aqueous HPLC-SEC (PEG calibrant): Mn=3900 g/mol.

### EXAMPLE BB13: CO-POLYAMINO ACID BB13—SODIUM POLY-L-GLUTAMATE MODIFIED BY MOLECULE BA1 AND HAVING A NUMBER-AVERAGE MOLECULAR WEIGHT (MN) OF 2800 G/MOL

By a process similar to that used for the preparation of co-polyamino acid BB1 applied to the hydrochloride salt of molecule BA1 (3.65 g, 5 mmol) and to a poly-L-glutamic acid with a number-average molecular weight Mn=3600 g/mol (10 g) obtained by a process similar to that used for the preparation of co-polyamino acid BB1-1, a sodium poly-L-glutamate modified by molecule BA1 is obtained.

Dry extract: 25.6 mg/g

DP (estimated based on ¹H NMR): 25

Based on ¹H NMR: i=0.08

The calculated average molecular weight of co-polyamino acid BB13 is 5253 g/mol.

Aqueous HPLC-SEC (PEG calibrant): Mn=2800 g/mol.

### EXAMPLE BB19′: CO-POLYAMINO ACID BB19′—SODIUM POLY-L-GLUTAMATE MODIFIED BY MOLECULE BA3 AND HAVING A NUMBER-AVERAGE MOLECULAR WEIGHT (MN) OF 7700 G/MOL

A sodium poly-L-glutamate modified by molecule BA3 is obtained by a process similar to that used in the preparation of co-polyamino acid AB23 applied to the hydrochloride salt of molecule BA3 and co-polyamino acid AB23′-1.

Dry extract: 25.3 mg/g

DP (estimated based on ¹H NMR): 60

Based on ¹H NMR: i=0.045

The calculated average molecular weight of the co-polyamino acid BB19′ is 11188 g/mol.

Organic HPLC-SEC (PEG Calibrator): Mn=7700 g/mol.

### EXAMPLE BB14: CO-POLYAMINO ACID BB14—SODIUM POLY-L-GLUTAMATE MODIFIED AT ONE OF ITS ENDS BY MOLECULE BA2 AND HAVING A NUMBER-AVERAGE MOLECULAR WEIGHT (MN) OF 4020 G/MOL

Hydrochloride salt of molecule BA2 (2.12 g, 2.70 mmol), chloroform (40 mL), molecular sieve 4 Å (1.5 g), as well as Amberlite IRN 150 ion exchange resin (1.5 g) are successively added to a suitable container. After stirring for 1 hour on rollers, the medium is filtered and the resin is rinsed with chloroform. The mixture is evaporated, then co-evaporated with toluene. The residue is solubilized in anhydrous DMF (20 mL) for direct use in the polymerization reaction.

In a previously oven-dried flask, γ-benzyl-L-glutamate N-carboxyanhydride (18 g, 68.42 mmol) is placed under vacuum for 30 minutes then anhydrous DMF (100 mL) is added. The mixture is stirred under argon until complete solubilization, cooled to 4° C., then the solution of molecule BA2 prepared as described above is quickly introduced. The mixture is stirred at 4° C. and room temperature for 2 days, then heated at 65° C. for 2 hours. The reaction mixture is then cooled to room temperature, then poured dropwise into diisopropyl ether (1.2 L) with stirring. The white precipitate is collected by filtration, washed twice with diisopropyl ether (100 mL), then dried under vacuum at 30° C. to obtain a white solid. The solid is diluted in TFA (105 mL), and a solution of 33% hydrobromic acid (HBr) in acetic acid (38 mL, 220 mmol) is then added dropwise at 0° C. The solution is stirred for 2 hours at room temperature and is then poured dropwise on a 1:1 (v/v) mixture of diisopropyl ether/water and with stirring (600 mL). After stirring for 2 hours, the heterogeneous mixture is allowed to stand overnight. The white precipitate is collected by filtration, washed successively with a 1:1 (v/v) mixture of diisopropyl ether and water (200 mL) then with water (100 mL). The obtained solid is solubilized in water (450 mL) by adjusting the pH to 7 by adding 10 N aqueous sodium hydroxide solution, then 1 N aqueous sodium hydroxide solution. The mixture is filtered through a 0.45 m filter, then is purified by ultrafiltration against 0.9% NaCl solution, then water until the conductimetry of the permeate is less than 50 μS/cm. The co-polyamino acid solution is then concentrated to approximately 30 g/L theoretical and the pH is adjusted to 7. The aqueous solution is filtered through a 0.2 m filter and stored at 4° C.

Dry extract: 22.3 mg/g

DP (estimated by ¹H NMR)=29 where i=0.034

The calculated average molecular weight of co-polyamino acid BB14 is 5089 g/mol.

Aqueous HPLC-SEC (PEG calibrant): Mn=4020 g/mol.

### EXAMPLE BB15: CO-POLYAMINO ACID BB15—SODIUM POLY-L-GLUTAMATE MODIFIED AT ONE OF ITS ENDS BY MOLECULE BA3 AND HAVING A NUMBER-AVERAGE MOLECULAR WEIGHT (MN) OF 3610 G/MOL

By a process similar to that used for the preparation of co-polyamino acid BB14 applied to the hydrochloride salt of molecule BA3 (3.62 g, 4.32 mmol) and to 25.0 g (94.97 mmol) of γ-benzyl-L-glutamate N-carboxyanhydride, a sodium poly-L-glutamate modified at one of its ends by molecule BA3 is obtained.

Dry extract: 26.5 mg/g

DP (estimated by ¹H NMR)=24 where i=0.042

The calculated average molecular weight of co-polyamino acid BB15 is 4390 g/mol.

Aqueous HPLC-SEC (PEG calibrant): Mn=3610 g/mol.

### EXAMPLE BB16: CO-POLYAMINO ACID BB16—SODIUM POLY-L-GLUTAMATE MODIFIED AT ONE OF ITS ENDS BY MOLECULE BA4 AND HAVING A NUMBER-AVERAGE MOLECULAR WEIGHT (MN) OF 3300 G/MOL

By a process similar to that used for the preparation of co-polyamino acid BB14 applied to the hydrochloride salt of molecule BA4 (5.70 g, 5.70 mmol) and to 29.99 g (113.9 mmol) of γ-benzyl-L-glutamate N-carboxyanhydride, a sodium poly-L-glutamate modified at one of its ends by molecule BA4 is obtained.

Dry extract: 32.3 mg/g

DP (estimated by ¹H NMR)=23 where i=0.043

The calculated average molecular weight of co-polyamino acid BB16 is 4399 g/mol.

Aqueous HPLC-SEC (PEG calibrant): Mn=3300 g/mol.

### EXAMPLE BB17: CO-POLYAMINO ACID BB17—SODIUM POLY-L-GLUTAMATE MODIFIED AT ONE OF ITS ENDS BY MOLECULE BA3 AND HAVING A NUMBER-AVERAGE MOLECULAR WEIGHT OF 10700 G/MOL

By a process similar to that used for the preparation of co-polyamino acid BB14 applied to the hydrochloride salt of molecule BA3 (2.51 g, 3 mmol) and to 52.7 g (200 mmol) of γ-benzyl-L-glutamate N-carboxyanhydride, a sodium poly-L-glutamate modified at one of its ends by molecule BA3 is obtained.

Dry extract: 24.5 mg/g

DP (estimated by ¹H NMR)=65 where i=0.015

The calculated average molecular weight of co-polyamino acid BB17 is 10585 g/mol.

Aqueous HPLC-SEC (PEG calibrant): Mn=10700 g/mol.

### EXAMPLE BB18: CO-POLYAMINO ACID BB18—SODIUM POLY-L-GLUTAMATE MODIFIED AT ONE OF ITS ENDS BY MOLECULE BA3 AND HAVING A NUMBER-AVERAGE MOLECULAR WEIGHT OF 6600 G/MOL

By a process similar to that used for the preparation of co-polyamino acid BB14 applied to the hydrochloride salt of molecule BA3 (2.51 g, 3 mmol) and to 31.6 g (120 mmol) of γ-benzyl-L-glutamate N-carboxyanhydride, a sodium poly-L-glutamate modified at one of its ends by molecule BA3 is obtained.

Dry extract: 27.3 mg/g

DP (estimated by ¹H NMR)=40 where i=0.025

The calculated average molecular weight of co-polyamino acid BB18 is 6889 g/mol.

Aqueous HPLC-SEC (PEG calibrant): Mn=6600 g/mol.

### EXAMPLE BB19: CO-POLYAMINO ACID BB19—SODIUM POLY-L-GLUTAMATE MODIFIED AT ONE OF ITS ENDS BY MOLECULE BA3 AND HAVING A NUMBER-AVERAGE MOLECULAR WEIGHT (MN) OF 3400 G/MOL

A sodium poly-L-glutamate modified at one end by molecule BA3 is obtained by a process similar to that used in the preparation of co-polyamino acid BB14 applied to the hydrochloride salt of molecule BA3 (36.26 g, 43.2 mmol) and to au γ-benzyl-L-glutamate N-carboxyanhydride (250.0 g, 949.7 mmol).

Dry extract: 22.4 mg/g

DP (estimated by ¹H NMR)=25 where i=0.04

The calculated average molecular weight of co-polyamino acid BB19 is 4540 g/mol.

Aqueous HPLC-SEC (PEG calibrant): Mn=3400 g/mol.

### EXAMPLE BB20: CO-POLYAMINO ACID BB20—SODIUM POLY-L-GLUTAMATE MODIFIED AT ONE OF ITS ENDS BY THE MOLECULE BA3 AND HAVING A NUMBER-AVERAGE MOLECULAR WEIGHT (MN) OF 2500 G/MOL

By a process similar to that used for the preparation of co-polyamino acid BB14 applied to molecule BA3 in the form of a free amine (1.017 g, 12.7 mmol) and to γ-benzyl-L-glutamate N-carboxyanhydride (5.0 g, 19.0 mmol), a sodium poly-L-glutamate modified at one of its ends by molecule BA3 is obtained.

Dry extract: 11.2 mg/g

DP (estimated by ¹H NMR)=17 where i=0.059

The calculated average molecular weight of co-polyamino acid BB20 is 3332 g/mol.

Aqueous HPLC-SEC (PEG calibrant): Mn=2500 g/mol.

### EXAMPLE BB21

### Co-Polyamino Acid BB21—Sodium Poly-L-Glutamate Modified at One of its Ends by Molecule BA3 and Having a Number-Average Molecular Weight (Mn) of 1100 g/Mol

By a process similar to that used for the preparation of co-polyamino acid BB14 applied to molecule BA3 in the form of a free amine (3.814 g, 4.75 mmol) and to γ-benzyl-L-glutamate N-carboxyanhydride (10.0 g, 38.0 mmol), a sodium poly-L-glutamate modified at one of its ends by molecule BA3 is obtained.

Dry extract: 16.1 mg/g

DP (estimated by ¹H NMR)=9 where i=0.11

The calculated average molecular weight of co-polyamino acid BB21 is 2123 g/mol.

Aqueous HPLC-SEC (PEG calibrant): Mn=1100 g/mol.

### EXAMPLE BB22

### Co-Polyamino Acid BB22—Sodium Poly-D-Glutamate Modified at One of its Ends by Molecule BA3 and Having a Number-Average Molecular Weight (Mn) of 2900 g/Mol

A sodium poly-D-glutamate modified at one of its ends by molecule BA3 is obtained by a process similar to that used in the preparation of the co-polyamino acid BB14 applied to molecule BA3 in the form of free amine (2.77 g, 3.45 mmol) and γ-benzyl-D-glutamate N-carboxyanhydride (20.0 g, 76.0 mmol).

Dry extract: 15.2 mg/g

DP (estimated by ¹H NMR)=21 where i=0.048

The calculated average molecular weight of co-polyamino acid BB22 is 3936 g/mol.

Aqueous HPLC-SEC (PEG calibrant): Mn=2900 g/mol.

### EXAMPLE BB23

### Co-Polyamino Acid BB23—Random Copolymer of Unit D- or Sodium L-Glutamate Modified at One of its Ends by the Molecule BA3 and Having a Number-Average Molecular Weight (Mn) of 2800 g/Mol

In a previously oven-dried flask, γ-benzyl-L-glutamate N-carboxyanhydride (20.0 g, 76.00 mmol) and γ-benzyl-D-glutamate N-carboxyanhydride (20.0 g, 76.00 mmol) are placed under vacuum for 30 minutes, then anhydrous DMF (75 mL) is added. The mixture is stirred under argon until complete solubilization, cooled to 4° C., then a solution of molecule BA3 in the form of free amine (5.55 g, 6.91 mmol) in chloroform (14.5 ml) is introduced rapidly. The mixture is stirred between 4° C. and room temperature for 18 h, then heated at 65° C. for 2 hours. The reaction mixture is then cooled to room temperature, then poured dropwise into diisopropyl ether (1.2 L) with stirring. The white precipitate is collected by filtration, washed twice with diisopropyl ether (80 mL), then dried under vacuum at 30° C. to obtain a white solid. The solid is diluted in TFA (152 mL), and a solution of 33% hydrobromic acid (HBr) in acetic acid (106 mL, 220 mmol) is then added dropwise at 0° C. The solution is stirred for 3 hours at room temperature and is then poured dropwise on a 1:1 (v/v) mixture of diisopropyl ether/water and with stirring (1.84 L). The aqueous phase is separated in a dropping funnel and the pH is adjusted to 7.2 by adding a 10N aqueous solution of NaOH. After adding water (250 ml), the mixture is filtered by a 0.45 filter. μm, then purified by ultrafiltration against 0.9% NaCl solution, then water until the conductimetry of the permeate is less than 50 μS/cm. The co-polyamino acid solution is then concentrated to about 25 g/L, filtered through 0.2 μm and stored at 4° C.

Dry extract: 28.2 mg/g

DP (estimated by ¹H NMR)=21 where i=0.048

The calculated average molecular weight of co-polyamino acid BB23 is 3936 g/mol.

Aqueous HPLC-SEC (PEG calibrant): Mn=2800 g/mol.

### EXAMPLE BB24: CO-POLYAMINO ACID BB24—BLOCK COPOLYMER OF POLY-D-GLUTAMATE AND SODIUM POLY-L-GLUTAMATE MODIFIED AT ONE OF ITS ENDS BY MOLECULE BA3 AND HAVING A NUMBER-AVERAGE MOLECULAR WEIGHT (MN) OF 2800 G/MOL

in a previously oven-dried flask, γ-benzyl-L-glutamate N-carboxyanhydride (13.5 g, 51.3 mmol) is placed under vacuum for 30 minutes, then anhydrous DMF (52 mL) is added. The mixture is stirred under argon until complete solubilization, cooled to 0° C., then a solution of molecule BA3 in the form of free amine (3.43 g, 4.27 mmol) in chloroform (8.6 ml) is quickly introduced. The mixture is stirred at 0° C. for 24 hours, then a solution of γ-tert-butyl-L-glutamate N-carboxyanhydride (13.5 g, 58.9 mmol) in DMF (15 mL) is added. The mixture is stirred at 0° C. and room temperature for 21 hours, then heated at 65° C. for 2 hours. The reaction mixture is then cooled to room temperature, then poured dropwise into diisopropyl ether (0.8 L) with stirring. The white precipitate is collected by filtration, washed twice with diisopropyl ether (52 mL), then dried under vacuum at 30° C. to obtain a white solid. The solid is diluted in TFA (96 mL), and a solution of 33% hydrobromic acid (HBr) in acetic acid (68 mL, 388 mmol) is then added dropwise at 0° C. The solution is stirred for 2 hours at room temperature and is then poured dropwise on a 1:1 (v/v) mixture of diisopropyl ether/water and with stirring (1.2 L). After stirring for 2 hours, the heterogeneous mixture is allowed to stand overnight. The white precipitate is collected by filtration, washed successively with a 1:1 (v/v) mixture of diisopropyl ether and water (100 mL) then with water (100 mL). The obtained solid is solubilized in water (900 mL) by adjusting the pH to 7 by adding 10 N aqueous sodium hydroxide solution, then 1 N aqueous sodium hydroxide solution. The mixture is filtered through a 0.45 μm filter, then is purified by ultrafiltration against 0.9% NaCl solution, then water until the conductimetry of the permeate is less than 50 μS/cm. The co-polyamino acid solution is then concentrated to about theoretical 20 g/L and the pH is adjusted to 7. The aqueous solution is filtered through a 0.2 μm filter and stored at 4° C.

Dry extract: 23.9 mg/g

DP (estimated by ¹H NMR)=25 where i=0.04

The calculated average molecular weight of co-polyamino acid BB24 is 4541 g/mol.

Aqueous HPLC-SEC (PEG calibrant): Mn=2800 g/mol.

### EXAMPLE BB25

### Co-Polyamino Acid BB25—Sodium Poly-L-Glutamate Modified at One of its Ends by Molecule BA5 and Having a Number-Average Molecular Weight (Mn) of 2800 g/Mol

By a process similar to that used for the preparation of co-polyamino acid BB14 applied to molecule BA5 in the form of a free amine (1.70 g, 1.98 mmol) and to γ-benzyl-L-glutamate N-carboxyanhydride (11.46 g, 43.5 mmol), a sodium poly-L-glutamate modified at one of its ends by molecule BA5 is obtained.

Dry extract: 19.8 mg/g

DP (estimated by ¹H NMR)=23 where i=0.043

The calculated average molecular weight of co-polyamino acid BB25 is 4295 g/mol.

Aqueous HPLC-SEC (PEG calibrant): Mn=2800 g/mol.

### EXAMPLE BB26

### Co-Polyamino Acid BB26—Sodium Poly-L-Glutamate Modified at One of its End by Molecule BA6 and Having a Number-Average Molecular Weight (Mn) of 2900 g/Mol

By a process similar to that used for the preparation of co-polyamino acid BB14 applied to molecule BA6 in the form of a free amine (3.05 g, 4.01 mmol) and to γ-benzyl-L-glutamate N-carboxyanhydride (22.78 g, 86.5 mmol), a sodium poly-L-glutamate modified at one of its ends by molecule BA6 is obtained.

Dry extract: 16.9 mg/g

DP (estimated by ¹H NMR)=21 where i=0.048

The calculated average molecular weight of co-polyamino acid BB26 is 3894 g/mol.

Aqueous HPLC-SEC (PEG calibrant): Mn=2900 g/mol.

### EXAMPLE BB27

### Co-Polyamino Acid BB27—Sodium Poly-L-Glutamate Modified at One of its Ends by Molecule BA3 and Modified by Molecule BA3 and Having a Number-Average Molecular Weight (Mn) of 2300 g/Mol

Co-polyamino acid BB27-1: poly-L-glutamic acid with a number-average molecular weight (Mn) of 3600 g/mol modified at one of its ends by molecule BA3 and capped at the other end by pidolic acid.

In a previously oven-dried flask, γ-benzyl-L-glutamate N-carboxyanhydride (122.58 g, 466 mmol) is placed under vacuum for 30 minutes then anhydrous DMF (220 mL) is added. The mixture is stirred under argon until complete solubilization, cooled to 10° C., then a solution of molecule BA3 in the form of free amine (17.08 g, 21.3 mmol) in chloroform (40 ml) is quickly introduced. The mixture is stirred at 0° C. and room temperature for 2 days, then heated at 65° C. for 4 hours. The reaction mixture is then cooled to 25° C., then pidolic acid (13.66 g, 105.8 mmol) is added, HOBt (2.35 g, 15.3 mmol) and EDC (20.28 g, 105.8 mmol) are added. After stirring for 24 hours at 25° C., the solution is concentrated under vacuum to eliminate chloroform and 50% of DMF. The reaction mixture is then heated to 55° C. and 1150 mL of methanol is added after 1 h. The reaction mixture is then cooled to 0° C. After 18 hours, the white precipitate is collected by filtration, washed three times with 270 mL of diisopropyl ether, then dried under vacuum at 30° C. to obtain a white solid. The solid is diluted in TFA (390 mL), and a solution of 33% hydrobromic acid (HBr) in acetic acid (271 mL, 1547 mmol) is then added dropwise at 0° C. The solution is stirred for 2 hours at room temperature and is then poured dropwise on a 1:1 (v/v) mixture of diisopropyl ether/water and with stirring (970 mL). After stirring for 2 hours, the heterogeneous mixture is allowed to stand overnight. The white precipitate is collected by filtration, washed successively with diisopropyl ether (380 mL), then twice with water (380 mL). The obtained solid is solubilized in water (3.6 L) by adjusting the pH to 7 by adding a 10N aqueous solution of sodium hydroxide, then a 1N aqueous sodium hydroxide solution. The mixture is filtered through a 0.45 μm filter, then purified by ultrafiltration against 0.9% NaCl solution, 0.1N NaOH solution, 0.9% NaCl solution, phosphate buffer solution (150 mM), a solution of NaCl 0.9% then water until the conductimetry of the permeate is below 50 μS/cm. The co-polyamino acid solution is then concentrated to about 30 g/L theoretical, filtered through 0.2 microns and acidified to pH 2 with stirring by addition of a solution of HCl 37%. The precipitate is then collected by filtration, washed twice with water, then dried under vacuum at 30° C. to obtain a white solid.

### Co-Polyamino Acid BB27

By a process similar to that used for the preparation of co-polyamino acid BB2 applied to molecule BA3 in the form of a free amine (1.206 g, 1.50 mmol) and to co-polyamino acid BB27-1 (5.5 g, 33.4 mmol), a sodium poly-L-glutamate modified at one of its ends by molecule BA3 and modified by molecule BA3 is obtained.

Dry extract: 19.0 mg/g

DP (estimated based on ¹H NMR): 22

Based on ¹H NMR: i=0.089

The calculated average molecular weight of co-polyamino acid BB27 is 4826 g/mol.

Aqueous HPLC-SEC (PEG calibrant): Mn=2300 g/mol.

### EXAMPLE BB42

### Co-Polyamino Acid BB42—Sodium Poly-L-Glutamate Modified at One of its Ends by Molecule B8 and Having a Number-Average Molecular Weight (Mn) of 3200 g/Mol

DCC (0.659 g, 3.19 mmol) and NHS (0.365 g, 3.17 mmol) are introduced to a solution of molecule B8 (2.366 g, 3.11 mmol) in DMF (19.5 mL). After stirring for 16 hours at room temperature, the solution is filtered to be used directly in the next reaction.

In a previously oven-dried flask, γ-benzyl-L-glutamate N-carboxyanhydride (18.0 g, 68.4 mmol) is placed under vacuum for 30 minutes then anhydrous DMF (40 mL) is added. The mixture is then stirred under argon until complete dissolution, cooled to 0° C., then hexylamine (0.411 ml, 3.11 mmol) is quickly introduced. After 30 hours of stirring at 0° C., the previously prepared solution of molecule B8 is added. The solution is stirred at between 0° C. and room temperature for 72 hours, then poured dropwise into diisopropylether (0.9 L) with stirring. The white precipitate is collected by filtration, washed with diisopropyl ether (5×100 mL), then dried under reduced pressure at 30° C. to give a white solid. The solid is diluted in TFA (69 mL), then the solution is cooled to 4° C. A solution of HBr at 33% in acetic acid (48 mL, 0.274 mol) is then added dropwise. The mixture is stirred at room temperature for 2 hours, then poured dropwise onto a 1:1 (v/v) mixture of diisopropyl ether and water with stirring (0.8 L). After stirring for 2 hours, the heterogeneous mixture is allowed to stand overnight. The white precipitate is collected by filtration, washed with a 1:1 (v/v) mixture of diisopropyl ether and water (70 mL), then with water (70 mL). The obtained solid is then solubilized in water (0.42 L) by adjusting 10 the pH to 7 by adding a 10N aqueous solution of sodium hydroxide, then a 1N aqueous sodium hydroxide solution. After solubilization, the solution is diluted to 20 g/L by adding water to obtain a final volume of 0.63 L. The solution is filtered through a 0.45 μm filter, then purified by ultrafiltration against a solution of NaCl 0.9%, then water until the conductimetry of the permeate is less than 50 μS/cm. The obtained solution is filtered through a 0.2 μm filter and stored at 2-8° C.

Dry extract: 22.2 mg/g

DP (estimated based on ¹H NMR): 22

Based on ¹H NMR: i=0.045

The calculated average molecular weight of co-polyamino acid BB42 is 4160 g/mol.

Aqueous HPLC-SEC (PEG calibrant): Mn=3200 g/mol.

### EXAMPLE BB25′: SODIUM CO-POLYAMINO ACID BB25′-POLY-L-GLUTAMATE MODIFIED AT ONE OF ITS ENDS BY MOLECULE BA3 AND ON THE OTHER END BY MOLECULE B8 AND HAVING A NUMBER-AVERAGE MOLECULAR WEIGHT (MN) OF 2000 G/MOL

DCC (0.257 g, 1.24 mmol) and NHS (0.143 g, 1.24 mmol) are added to a solution of molecule B8 (0.946 g, 1.24 mmol) in DMF (8 mL). After stirring for 16 hours at room temperature, the solution is filtered to be used directly in the next reaction.

In a previously oven-dried flask, γ-benzyl-L-glutamate N-carboxyanhydride e (6.0 g, 22.8 mmol) is placed under vacuum for 30 minutes, then anhydrous DMF (14 mL) is added. The mixture is then stirred under argon until complete dissolution, cooled to 0° C., then a solution of molecule BA3 in the form of a free amine (0.832 g, 1.04 mmol) in chloroform (2.0 mL) is quickly introduced. After 18 hours of stirring at 0° C., the previously prepared solution of molecule B8 is added. The solution is stirred at between 0° C. and room temperature for 22 hours, then poured dropwise into diisopropylether (0.34 L) with stirring. The white precipitate is collected by filtration, washed with diisopropyl ether (7×15 mL), then dried under reduced pressure at 30° C. to give a white solid. The solid is diluted in TFA (23 mL), then the solution is cooled to 4° C. A solution of HBr at 33% in acetic acid (15 mL, 85.7 mmol) is then added dropwise. The mixture is stirred at room temperature for 2 hours, then poured dropwise onto a 1:1 (v/v) mixture of diisopropyl ether and water with stirring (0.28 L). After stirring for 2 hours, the heterogeneous mixture is allowed to stand overnight. The white precipitate is collected by filtration, washed twice with a 1:1 (v/v) mixture of diisopropyl ether and water (24 mL), then twice with water (24 mL). The obtained solid is then solubilized in water (0.16 L) by adjusting the pH to 12 by adding a 10N aqueous sodium hydroxide solution, then a 1N aqueous sodium hydroxide solution. After 30 minutes the pH is adjusted to 7 by slowly adding an aqueous solution of 1N HCl. The solution is filtered through a 0.45 μm filter, then purified by ultrafiltration against a solution of NaCl 0.9%, then water until the Permeate conductimetry is less than 50 μS/cm. The obtained solution is filtered through a 0.2 μm filter and stored at 2-8° C.

Dry extract: 18.9 mg/g

DP (estimated based on ¹H NMR): 22

Based on ¹H NMR: i₁=0.09

The calculated average molecular weight of the co-polyamino acid BB25′ is 4871 g/mol.

Aqueous HPLC-SEC (PEG calibrant): Mn=2000 g/mol.

### EXAMPLE BB44: CO-POLYAMINO ACID BB44—SODIUM POLY-L-GLUTAMATE MODIFIED AT ONE OF ITS ENDS BY MOLECULE BA7 AND HAVING A NUMBER-AVERAGE MOLECULAR WEIGHT (MN) OF 3300 G/MOL

A sodium poly-L-glutamate modified at one end by molecule BA7 is obtained by a process similar to that used in the preparation of co-polyamino acid BB14 applied to molecule BA7 as free amine (4.45 g, 5.18 mmol) and 30.0 g (113.96 mmol) of γ-benzyl-L-glutamate N-carboxyanhydride.

Dry extract: 29.0 mg/g

DP (estimated by ¹H NMR)=25 where i=0.04

The calculated average molecular weight of co-polyamino acid BB44 is 4597 g/mol.

Aqueous HPLC-SEC (PEG calibrant): Mn=3300 g/mol.

### Part C: Compositions

The glucagon used is human glucagon from a peptide synthesis process. It is manufactured by Bachem (reference 4074733).

The nicotinamide used, tested according to the recommendations of the European Pharmacopoeia, comes from Sigma-Aldrich (reference 72347).

The Treprostinil used is manufactured by Ferrer Internacional (reference 1100269).

### EXAMPLE C1-I: 2 MG/ML GLUCAGON SOLUTION IN HYDROCHLORIC ACID CONTAINING 2 MG/ML L-METHIONINE

94.7 mg of glucagon DS powder is added to a 50 ml Falcon tube, followed by 45 ml of a 0.003 N hydrochloric acid solution containing 2 mg/ml of L-methionine. Glucagon powder is mixed by repeated inversions of the tube until complete dissolution of the glucagon. At 2 mg/ml, the glucagon solution is then filtered through a membrane (0.22 m).

### EXAMPLE C2-I: GLUCAGON SOLUTION AT 4 MG/ML IN HYDROCHLORIC ACID CONTAINING 2 MG/ML OF L-METHIONINE

Glucagon (160 mg) powder is introduced into a 45 ml Falcon tube, then 40 ml of 0.006 N aqueous hydrochloric acid solution containing 2 mg/ml of L-methionine is added. Glucagon powder is mixed by repeated inversions of the tube until complete dissolution of the glucagon. The glucagon solution at 4 mg/ml is then filtered through a membrane (0.22 m).

### EXAMPLE C3-I: 2 MG/ML GLUCAGON SOLUTION IN HYDROCHLORIC ACID

In a 20 mL Falcon tube is introduced 23.4 mg of powdered glucagon DS followed by 11.3 mL of 0.003N hydrochloric acid solution. The glucagon powder is mixed by repeated inversions of the tube until complete dissolution of glucagon. At 2 mg/ml, the glucagon solution is then filtered through a membrane (0.22 m).

### EXAMPLE C4-I: GLUCAGON SOLUTION AT 4 MG/ML IN HYDROCHLORIC ACID

Glucagon (160 mg) powder is introduced into a 45 ml Falcon tube. An aqueous solution of 0.003 N hydrochloric acid (40 ml) is added. Glucagon powder is mixed by repeated inversions of the tube until complete dissolution of the glucagon. The glucagon solution at 4 mg/ml is then filtered through a membrane (0.22 m).

### EXAMPLE CR0-I: PREPARATION OF CO-POLYAMINO ACID SOLUTIONS BB15 AT 7.6 MG/ML AND GLUCAGON AT 2 MG/ML CONTAINING 1 MG/ML OF L-METHIONINE

A 15.22 mg/mL co-polyamino acid BB15 solution is added to concentrated solutions of glycerol (to obtain 300 mOsmol/kg in the final formulation), of phosphate buffer (4 mM) and m-cresol (54 mM). At this stage, additives, such as citrate, nicotinamide, Treprostinil and magnesium, can be added. The composition is briefly stirred until dissolution of the co-polyamino acid, then the solution is filtered through a membrane (0.22 μm).

6.5 ml of a freshly prepared glucagon solution, according to the protocol described in example C2-I, are mixed with 6.5 ml of the co-polyamino acid solution BB15, as prepared above, to obtain the final compositions CR0-1-I to CR0-11-I (Table 2). The pH of the solution is adjusted to pH 7.2±0.1 by adding 1 N NaOH/HCl, then filtered through a membrane (0.22 m).

A visual inspection is performed to determine whether or not a clear solution is obtained. A visual inspection of the samples is performed to detect visible particles, or turbidity. This inspection is borne out according to the recommendations of the European pharmacopoeia (EP 2.9.20): the samples are subjected to a lighting of at least 2000 lux and are observed on a white background and a black background. The composition is deemed limpid when the particles are visible in half of the samples the composition.

### EXAMPLE CS0-I: PREPARATION OF CO-POLYAMINO ACID SOLUTIONS BB15 AT 6.3 MG/ML AND GLUCAGON AT 2 MG/ML CONTAINING 1 MG/ML OF L-METHIONINE

A 12.6 mg/L co-polyamino acid BB15 solution is added to solutions concentrated in glycerol (to obtain 300 mOsmol/kg in the final formulation), in phosphate buffer (4 mM) and m-cresol (54 mM). At this stage, additives, such as citrate, nicotinamide, Treprostinil and magnesium, can be added. The composition is briefly stirred until dissolution of the co-polyamino acid, then the solution is filtered through a membrane (0.22 μm).

6.5 ml of a freshly prepared glucagon solution, according to the protocol described in example C2-I, are mixed with 6.5 ml of the co-polyamino acid solution, as prepared above, to obtain the final compositions CS0-1 to CS0-4 (Table 2). The pH of the solution is adjusted to pH 7.2±0.1 by adding 1N NaOH/HCl, then filtered through a membrane (0.22

A visual inspection is performed to determine whether a clear solution is obtained or not. In comparison, the glucagon solution at neutral pH is not soluble above 0.2 mg/mL.

### EXAMPLE CT0-I: PREPARATION OF SOLUTIONS OF CO-POLYAMINO ACID BB15 AT 7.6 MG/ML AND GLUCAGON AT 2 MG/ML

A 15.22 mg/mL co-polyamino acid BB15 solution is added to concentrated solutions in glycerol (to obtain 300 mOsmol/kg in the final formulation), in phosphate buffer (4 mM) and m-cresol (54 mM). At this stage, additives, such as citrate, nicotinamide, Treprostinil and magnesium, can be added. The composition is briefly stirred until dissolution of the co-polyamino acid, then the solution is filtered through a membrane (0.22 μm).

6.5 ml of a freshly prepared glucagon solution, according to the protocol described in example C4-I, are mixed with 6.5 ml of the co-polyamino acid BB15 solution, as prepared above, to obtain the final compositions CT0-1-I to CT0-3-I (Table 3). The pH of the solution is adjusted to pH 7.2±0.1 by adding 1 N NaOH/HCl, then filtered through a membrane (0.22 m).

A visual inspection is performed to determine whether a clear solution is obtained or not.

TABLE 2
Compositions and visual appearance of 2 mg/mL glucagon
solutions at pH 7.2 at different concentrations of
co-polyamino acid BB15 containing m-cresol (27 mM),
1 mg/mL of L-methionine and phosphate buffer (2 mM).

| | Co- | Co- | | | |
|---|---|---|---|---|---|
| | polyamino | polyamino | Glyc- | | Visual |
| Composi- | acid BB15 | acid ratio/ | erol | | appear- |
| tion | [symbol] | Glucagon | (MM) | Additive | ance of |
| CR0-1-I | 7.6 | 3 | 260 | | clear |
| CR0-2-I | 7.6 | 3 | 220 | 10 mM | clear |
| | | | | citrate | |
| CR0-3-I | 7.6 | 3 | 249 | 20 mM | clear |
| | | | | nicotinamide | |
| CR0-4-I | 7.6 | 3 | 228 | 40 mM | clear |
| | | | | nicotinamide | |
| CR0-5-I_{—} | 7.6 | 3 | 188 | 80 mM | clear |
| | | | | nicotinamide | |
| CR0-6-I | 7.6 | 3 | 263 | 2 mM Mg | clear |
| CR0-7-I | 7.6 | 3 | 255 | 5 mM Mg | clear |
| CR0-8-I | 7.6 | 3 | 242 | 10 mM Mg | clear |
| CR0-9-I | 7.6 | 3 | 268 | 13 ng/mL | clear |
| | | | | Treprostinil | |
| CR0-10-I | 7.6 | 3 | 268 | 128 ng/mL | clear |
| | | | | Treprostinil | |
| CR0-11-I | 7.6 | 3 | 268 | 1000 ng/mL | clear |
| | | | | Treprostinil | |
| CS0-1-I | 6.3 | 2.5 | 257 | — | clear |
| CS0-2-I | 6.3 | 2.5 | 248 | 5 mM citrate | clear |
| CS0-3-I | 6.3 | 2.5 | 168.7 | 5 mM | clear |
| | | | | citrate + | |
| | | | | 80 mM | |
| | | | | nicotinamide | |
| CS0-4-I | 6.3 | 2.5 | 188 | 80 mM | clear |
| | | | | nicotinamide | |

[symbol]

indicates data missing or illegible when filed

TABLE 3
Compositions and visual appearance of glucagon solutions at 2 mg/mL at pH 7.2 at 7.6 mg/mL
in co-polyamino acid BB15 containing m-cresol (27 mM) and phosphate buffer (2 mM).

| | Co-polyamino acid | Co-polyamino acid | Glycerol | | Visual appearance |
|---|---|---|---|---|---|
| Composition | BB15 concentration | ratio/Glucagon | (MM) | Additive | [symbol] |
| CT0-1-I | 7.6 | 3 | 260 | | clear |
| CT0-2-I | 7.6 | 3 | 220 | 10 mM | clear |
| | | | | citrate | |
| CT0-3-I | 7.6 | 3 | 175 | 80 mM | clear |
| | | | | nicotinamide | |

[symbol]

indicates data missing or illegible when filed

### EXAMPLE CV0-I: PREPARATION OF A GLUCAGON SOLUTION AT 1 MG/ML CONTAINING CO-Polyamino ACID BB15 AT VARIOUS CONCENTRATIONS, A PHOSPHATE BUFFER (2 MM) AND GLYCEROL AT PH 7.2

A co-polyamino acid BB15 solution is added to a bottle containing concentrated solutions of excipients (phosphate, glycerol) and optionally additives (m-cresol, citrate, nicotinamide). The composition is briefly stirred until dissolution of the co-polyamino acid, then the solution is filtered through a membrane (0.22 μm).

The equivolumic mixture of this solution with the freshly prepared glucagon solution, as described in example C1-I, leads to the final compositions CV0-1-I to CV0-15-I containing 1 mg/mL of glucagon. The pH of the solution is adjusted to pH 7.2±0.1 by adding 1 N NaOH/HCl, then filtered through a membrane (0.22 μm). The details of the compositions are summarized in the table below.

A visual inspection is performed to determine whether a clear solution is obtained or not. In comparison, the glucagon solution at neutral pH is not soluble above 0.2 mg/mL.

TABLE 3b
Compositions and visual appearance of 1 mg/mL glucagon solutions at pH 7.2 at different concentrations
of co-polyamino acid BB15 containing 1 mg/mL of L-methionine and phosphate buffer (2 mM).

| | Co-polyamino acid | | | | |
|---|---|---|---|---|---|
| | BB15 concentration | Co-polyamino acid | Glycerol | | Visual appearance |
| Composition | (mg/ml) | ratio/Glucagon | (MM) | Additive | of the solution |
| CV0-1-I | 3.8 | 3 | 255 | 10 mM citrate | clear |
| CV0-2-I | 3.8 | 3 | 235 | 15 mM citrate | clear |
| CV0-3-I | 3.8 | 3 | 215 | 20 mM citrate | clear |
| CV0-4-I | 4.5 | 3 | 255 | 10 mM citrate | clear |
| CV0-5-I | 4.5 | 3 | 235 | 15 mM citrate | clear |
| CV0-6-I | 4.5 | 3 | 215 | 20 mM citrate | clear |
| CV0-7-I | 3.8 | 3 | 235 | 10 mM citrate + | clear |
| | | | | 20 mM nicotinamide | |
| CV0-8-I | 3.8 | 3 | 215 | 10 mM citrate + | clear |
| | | | | 40 mM nicotinamide | |
| CV0-9-I | 3.8 | 3 | 175 | 10 mM citrate + | clear |
| | | | | 80 mM nicotinamide | |
| CV0-10-I | 3.8 | 3 | 85 | 10 mM citrate + | clear |
| | | | | 170 mM nicotinamide | |
| CV0-11-I | 3.8 | 3 | 255 | 40 mM nicotinamide | clear |
| CV0-12-I | 3.8 | 3 | 215 | 80 mM nicotinamide | clear |
| CV0-13-I | 3.8 | 3 | 125 | 170 mM nicotinamide | clear |
| CV0-14-I | 4.00 | 3.13 | 247 | 10 mM citrate | clear |
| CV0-15-I | 5.11 | 4 | 225 | 10 mM citrate + | clear |
| | | | | 20 mM nicotinamide | |

### Monitoring of the Physical Stability of the Compositions

The previously prepared compositions were transferred into cartridges (3 ml OMPI easy-to-fill—Ref P40B4100.3250) at a rate of 1 mL per cartridge and placed under static conditions at 37° C. and 4° C.

In order to detect the occurrence of visible particles, fibrils or turbidity, visual inspection of samples placed under static conditions is performed after 0, 1, 2, 3, 4, 5, 6 weeks at 37° C. and once a month for 4° C. conditions. The present inspection is carried out according to the recommendations of the European Pharmacopoeia (EP 2.9.20): the samples are subjected to a lighting of at least 2000 Lux and are observed facing a white background and a black background to comply with the recommendations of the European Pharmacopoeia. The composition is deemed unstable when the particles are visible in half of the samples. Stable means that at the time of inspection, at least half of the samples were devoid of particles, fibrils or turbidity.

Study of the physical stabilities of the previously prepared compositions described in the table below was carried out on volumes of 1 ml of composition in cartridges containing 3 ml (WIPO—ref: P40B4100.3250). In comparison, the glucagon solution at acidic pH of 2 mg/mL is stable for only 2 days at 37° C.

| | | Concentration of | | | |
|---|---|---|---|---|---|
| | Glucagon | co-polyamino | | Stability | Stability |
| | concentration | acids BB15 | | 37° C. | 4° C. |
| Examples | (mg/mL) | (mg/mL) | Additives | (weeks) | (month) |
| CR0-2-I | 2 | 7.6 | 10 mM | >3 | >8 |
| | | | citrate | | |
| CR0-3-I | 2 | 7.6 | 20 mM | >6 | >10 |
| | | | Nicotinamide | | |
| CR0-4-I | 2 | 7.6 | 40 mM | >6 | >10 |
| | | | Nicotinamide | | |
| CR0-5-I | 2 | 7.6 | 80 mM | >6 | >10 |
| | | | Nicotinamide | | |
| CR0-6-I | 2 | 7.6 | 2 mM Mg | >6 | >10 |
| CR0-8-I | 2 | 7.6 | 13 ng/mL | >6 | >10 |
| | | | Treprostinil | | |
| CR0-9-I | 2 | 7.6 | 128 ng/mL | >6 | >10 |
| | | | Treprostinil | | |
| CR0-10-I | 2 | 7.6 | 1000 ng/mL | >6 | >10 |
| | | | Treprostinil | | |

The compositions according to the invention containing nicotinamide and Treprostinil exhibit physical stability at 37° C. under static conditions in cartridge greater than three weeks at 37° C. and eight months at 4° C. The addition of co-polyamino acids thus enables the solubilization and stabilization of the glucagon at neutral pH.

| | | Concen- | | |
|---|---|---|---|---|
| | Glucagon | tration of | | |
| | concen- | co-polyamino | | Stability |
| | tration | acids BB15 | | 37° C. |
| Examples | (mg/mL) | (mg/mL) | Additives | (weeks) |
| CV0-1-I | 1 | 3.8 | 10 mM citrate | >3 |
| CV0-2-I | 1 | 3.8 | 15 mM citrate | >3 |
| CV0-3-I | 1 | 3.8 | 20 mM citrate | >3 |
| CV0-4-I | 1 | 4.5 | 10 mM citrate | >3 |
| CV0-5-I | 1 | 4.5 | 15 mM citrate | >3 |
| CV0-6-I | 1 | 4.5 | 20 mM citrate | >3 |
| CV0-7-I | 1 | 3.8 | 10 mM citrate + | >3 |
| | | | 20 mM | |
| | | | nicotinamide | |
| CV0-8-I | 1 | 3.8 | 10 mM citrate + | >3 |
| | | | 40 mM | |
| | | | Nicotinamide | |
| CV0-9-I | 1 | 3.8 | 10 mM citrate + | >3 |
| | | | 80 mM | |
| | | | Nicotinamide | |
| CV0-10-I | 1 | 3.8 | 10 mM citrate + | 3 |
| | | | 170 mM | |
| | | | nicotinamide | |
| CV0-11-I | 1 | 3.8 | 40 mM | 3 |
| | | | Nicotinamide | |

The compositions according to the invention containing citrate, nicotinamide or a combination of both exhibit physical stability at 37° C. under static conditions in cartridge of at least two weeks at 37° C. The addition of co-polyamino acids makes it possible to solubilize and stabilize glucagon at neutral pH.

### Results of Visual Observations on the Mixture and ThT Fibrillation Measurements

The previously prepared compositions were aliquoted into a triplicate 96-well plate (3*150 μL) and placed under static conditions at 37° C. and 4° C.

### Principle

The poor stability of a peptide can lead to the formation of amyloid fibrils, defined as ordered macromolecular structures. These may result in the formation of a gel within the sample.

The thioflavin fluorescence monitoring test T (ThT) is used to analyze the physical stability of the solutions. Thioflavin is a small probe molecule with a characteristic fluorescence signature when it binds to amyloid-type fibrils (Naiki et al. (1989) Anal. Bio Chem. 177, 244-249; LeVine (1999) Methods. Enzymol. 309, 274-284).

This method makes it possible to monitor fibril formation at low concentrations of ThT in undiluted solutions. This monitoring is performed under accelerated stability conditions: with stirring and at 37° C.

### Experimental Conditions

Samples were prepared just before the start of the measurement. The preparation of each composition is described in the corresponding example. Thioflavin T was added to the composition from a concentrated stock solution in order to induce a negligible dilution of the composition. The concentration of Thioflavin T in the composition is 40 μM.

A volume of 150 μL of the composition was introduced into a well of a 96-well plate after which 2.7 μL of concentrated solution of ThT was introduced. Each composition was analyzed in three tests (triplicate) within the same plate. The plate was sealed with a transparent film in order to prevent evaporation of the composition.

This plate was then placed in the enclosure of a plate reader (Xenius XC, SAFAS). The temperature is adjusted to 37° C., and lateral stirring at 960 rpm with an amplitude of 1 mm is applied.

A reading of the fluorescence intensity in each well is made with an excitation wavelength of 442 nm, and an emission wavelength of 482 nm over time.

The fibrillation process is manifested by a strong increase in fluorescence after a delay called latency time.

Latency time is determined graphically, by calculating the time when the tangent on the linear growth phase intersects the x-axis.

The reported latency time value corresponds to the average of latency time measurements made on three wells.

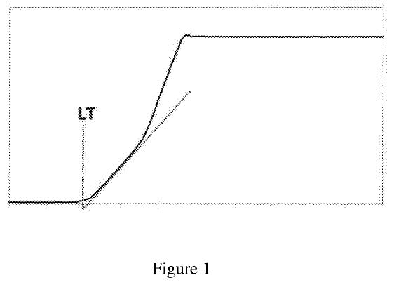

An example of a graphic determination is shown in FIG. 1.

This figure is a graphic representation of latency time determination (LT) by monitoring the fluorescence of Thioflavin T, on a curve showing the fluorescence value (in a.u. arbitrary units) and the time in minutes on the horizontal axis.

The latency time results obtained are shown in the table below.

In comparison, the glucagon solution at acidic pH at 2 mg/mL shows a fibrillation time of about 0.6 hours.

| | | Concen- | | | |
|---|---|---|---|---|---|
| | Glucagon | tration of | | | Fibril- |
| | concen- | co-polyamino | | | lation |
| Compo- | tration | acids BB15 | | | latency |
| sitions | (mg/mL) | (mg/mL) | Ratio | Additives | time |
| CS0-2-I | 2 | 6.3 | 2.5 | 5 mM citrate | >15 h |
| CS0-3-I | 2 | 6.3 | 2.5 | 5 mM citrate + | >15 h |
| | | | | 80 mM | |
| | | | | nicotinamide | |
| CS0-4-I | 2 | 6.3 | 2.5 | 80 mM | >15 h |
| | | | | Nicotinamide | |

The composition CS0-4 containing nicotinamide produce latency times greater than 15 hours with a BB15/glucagon molar ratio of 2.5 compared to a few minutes for the glucagon solution alone at acidic pH.

### Part D: Pharmacodynamics and Pharmacokinetics

### D1: Protocol for Measuring the Pharmacodynamics and Pharmacokinetics of Glucagon Solutions

Domestic pigs weighing 50 to 100 kg, previously catheterized in the jugular vein, are fasted 5.5 hours before the start of the experiment. The 44 μg/kg octreotide dose is administered subcutaneously at the flank, outside the area identified for injection of the test item, 40 minutes prior to administration of the glucagon formulations. In the hour before the injection of glucagon, 3 blood samples are collected to determine the basal level of glucose and glucagon.

The injection of glucagon formulations at a dose of 1 μg/kg is carried out subcutaneously in the flank of the animal, in an identified area, using an insulin pen (Sanofi, JuniorStar®) equipped with a 31 G needle.

Blood samples are then collected every 5 minutes for 20 minutes, then two samples at 30 and 45 minutes, and finally every 30 minutes up to 2.5 hours. After each sample collection, the catheter is rinsed with a diluted heparin solution. A drop of blood is obtained to determine blood glucose using a glucometer.

Glucose pharmacodynamics curves expressed in adjusted basal blood glucose concentration are then plotted. The following pharmacodynamic parameters were then determined by non-compartmental analysis with Phoenix WinNonlin software:
AUC-BG0-30 min corresponding to the area under the blood glucose curve as over time and the basal blood glucose value between 0 and 30 minutes post-administration
Tonset_20 mg/dL corresponding to the time needed to increase blood glucose by 20 mg/dL compared to the basal value

Tonset_20 mg/dL is commonly used to evaluate the onset of the hyperglycemic effect of glucagon formulations. AUC-BG0-30 min is commonly used to evaluate the early hyperglycemic effect of glucagon formulations.

The remaining blood is collected in a P800 tube and centrifuged to isolate plasma. Glucagon levels in the plasma samples are measured by the ELISA enzyme immunoassay method for each pig.

The pharmacokinetic curves expressed in basal level delta are then plotted.

The following pharmacokinetic parameters were then determined by non-compartmental analysis with Phoenix WinNonlin software:
T max glucagon corresponding to the time required to reach the maximum concentration of glucagon in the plasma
Early T50% C max glucagon corresponding to the time required to reach 50% of the maximum concentration of glucagon in plasma before T max
AUC-gluc0-15 min corresponding to the area under the curve of glucagon concentrations as a function of time between 0 and 15 minutes post-administration
AUC-gluc0-30 min corresponding to the area under the curve of glucagon concentrations as a function of time between 0 and 30 minutes post-administration Early T50% C max insulin and T max glucose are commonly used to evaluate the onset of insulin absorption. AUC-gluc0-15 min and AUC-gluc0-30 min are commonly used to evaluate early exposure to insulin in plasma. Early T50% C max insulin parameter is estimated by linear interpolation.

### D2: Results of Pharmacodynamics and Pharmacokinetics of Insulin Solutions of Examples CR0-1-I and CR0-5-I

| | | co-polyamino | Nicotinamide | Number |
|---|---|---|---|---|
| Example | Glucagon | acid | (mM) | of pigs |
| CR0-1-I | rh-glucagon | BB15 | — | 10 |
| CR0-5-I | rh-glucagon | BB15 | 80 | 9 |

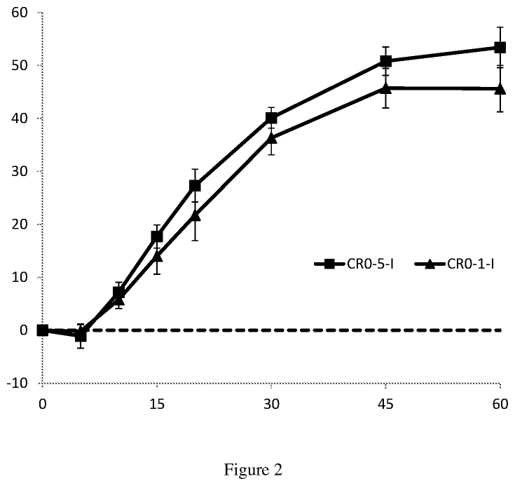

The pharmacodynamic results obtained with the compositions described in Examples CR0-1-I and CR0-5-I are illustrated in the FIG. 2. The analysis of these profiles indicates that the composition of example CR0-5-I comprising co-polyamino acid 13BB1315 and 80 mM nicotinamide (curve plotted with the squares corresponding to example CR0-5) makes it possible to obtain a faster action than that of the composition of example CR0-1-I comprising only co-polyamino acid B B15 (curve drawn with the triangles corresponding to example CR0-1-I). Pharmacodynamic parameters are reported in the following table:

| | Tonset_20 mg/dL (min) | AUC-BG0-30 min (min*mg/dL) |
|---|---|---|
| Example | Mean ± SD | Mean ± SD |
| CR0-1-I | 20 ± 6 | 442 ± 234 |
| CR0-5-I | 17 ± 4 | 524 ± 188 |

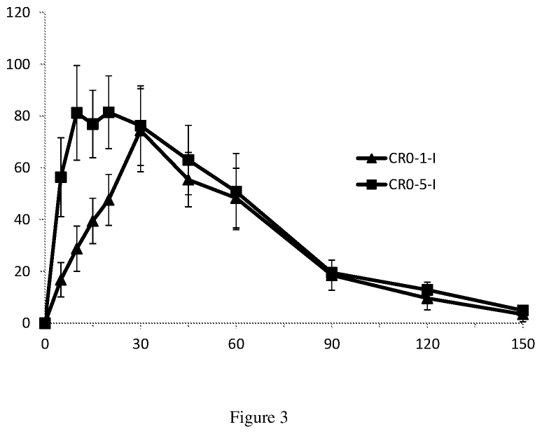

The pharmacokinetic results obtained with the compositions described in examples CR0-1-I and CR0-5-I are shown in the FIG. 3. The analysis of these profiles indicates that the composition of example CR0-5-I comprising co-polyamino acid BB15 and 80 mM nicotinamide (curve plotted with the squares corresponding to example CR0-5-I) makes it possible to obtain a faster action than that of the composition of example CR0-1-I comprising only co-polyamino acid BB15 (curve drawn with the triangles corresponding to example CR0-1-I). Pharmacokinetic parameters are reported in the following table:

| | Tmax | | T50% Cmax | AUC-Gluc0-15 min | AUC-Gluc0-30 min |
|---|---|---|---|---|---|
| | glucagon (min) | | glucagon (min) | (min*pmol/L) | (min*pmol/L) |
| Example | Median [min-max] | | Mean ± SD | Mean ± SD | Mean ± SD |
| CR0-1-I | 30 | [10-60] | 16 ± 11 | 326 ± 287 | 1,154 ± 550 |
| CR0-5-I | 20 | [5-30] | 6 ± 3 | 893 ± 508 | 2,071 ± 967 |

The addition of nicotinamide therefore accelerates the absorption and effect of glucagon in the presence of co-polyamino acid BB15.

### D3: Results of Pharmacodynamics and Pharmacokinetics of Glucagon Solutions of Examples CR0-1-I and CV0-14-I

| | | co-polyamino | Citrate | Number |
|---|---|---|---|---|
| Example | Glucagon | acid | (mM) | of pigs |
| CR0-1-I | rh-glucagon | BB15 | — | 10 |
| CV0-14-I | rh-glucagon | BB15 | 10 | 10 |

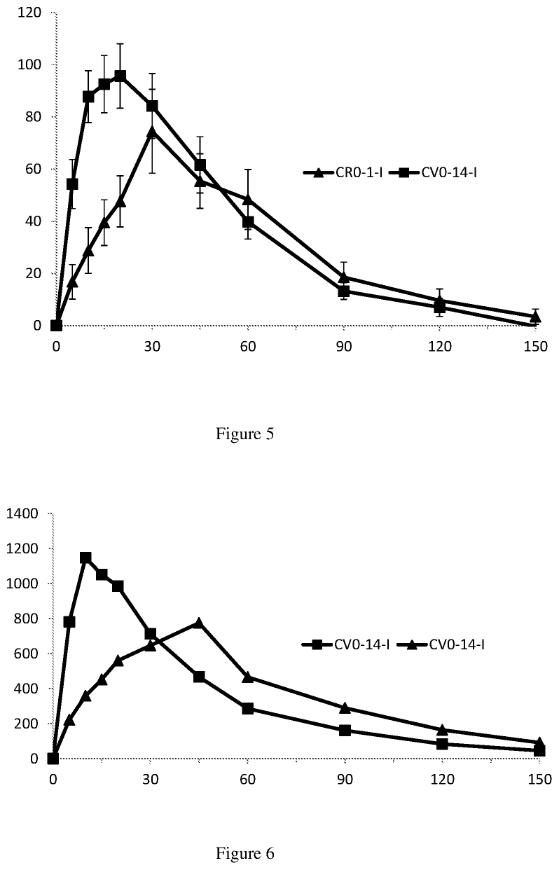

The pharmacodynamic results obtained with the compositions described in Examples CR0-1-I and CV0-14-1 are illustrated in the FIG. 5. The analysis of these profiles indicates that the composition of example CV0-14-I comprising co-polyamino acid BB315 and 10 mM citrate (curve plotted with the squares corresponding to example CV0-14-I) facilitate the obtention of a faster action than that of the composition of example CR0-1-I comprising only co-polyamino acid BB15 (curve drawn with the triangles corresponding to example CR0-1-I). Pharmacodynamic parameters are reported in the following table:

| | Tonset_20 mg/dL (min) | AUC-BG0-30 min (min*mg/dL) |
|---|---|---|
| Example | Mean ± SD | Mean ± SD |
| CR0-1-I | 19.6 ± 6.3 | 442 ± 234 |
| CV0-14-I | 13.3 ± 3.9 | 718 ± 238 |

The pharmacokinetic results obtained with the compositions described in examples CR0-1-I and CV0-14-I are shown in the FIG. 6. The analysis of these profiles indicates that the composition of the example CV0-14-I comprising the co-polyamino acid BB15 and 10 mM citrate (curve plotted with the squares corresponding to the example CV0-14-I) enable a glucagon absorption faster than that of the composition of example CR0-1-I comprising only co-polyamino acid BB15 (curve drawn with the triangles corresponding to example CR0-1-I). Pharmacokinetic parameters are reported in the following table:

| | Tmax | T50% Cmax | AUC-Gluc0-15 min | AUC-Gluc0-30 min |
|---|---|---|---|---|
| | glucagon (min) | glucagon (min) | (min*pmol/L) | (min*pmol/L) |
| Example | Median [min-max] | Mean ± SD | Mean ± SD | Mean ± SD |
| CR0-1-I | 30 [10-60] | 16 ± 11 | 326 ± 287 | 1,154 ± 550 |
| CV0-14-I | 20 [10-45] | 5 ± 2 | 941 ± 347 | 2298 ± 826 |

The addition of citrate therefore accelerates the absorption and effect of glucagon in the presence of co-polyamino acid BB15.

### D4: Pharmacokinetic Results of Glucagon Solutions of Examples CV0-14-I and CV0-15-I

| | | co- | | | |
|---|---|---|---|---|---|
| | | polyamino | Citrate | Nicotinamide | Number |
| Example | Glucagon | acid | (mM) | (mM) | of pigs |
| CV0-14-I | rh- [symbol] | BB15 | 10 | — | 13 |
| CV0-15-I | rh- [symbol] | BB15 | 10 | 20 | 12 |

[symbol]

indicates data missing or illegible when filed

The pharmacokinetic results obtained with the compositions described in examples CV0-14-I and CV0-15-I are shown in the FIG. 6. The analysis of these profiles indicates that the composition of example CV0-15-I comprising the co-polyamino acid BB15, 10 mM citrate and 20 mM of nicotinamide, and injected intra-muscularly (curve drawn with squares corresponding to the example CV0-15-I enable a glucagon absorption faster than that of the composition of example CV0-14-I comprising co-polyamino acid BB15 and 10 mM citrate, and injected subcutaneously (curve drawn with the triangles corresponding to the example CV0-14-I). Pharmacokinetic parameters are reported in the following table:

| | Tmax | | T50% Cmax | AUC-Gluc0-15 min | AUC-Gluc0-30 min |
|---|---|---|---|---|---|
| | glucagon (min) | | glucagon (min) | (min*pmol/L) | (min*pmol/L) |
| Example | Median [min-max] | | Mean ± SD | Mean ± SD | Mean ± SD |
| CV0-14-I | 30 | [10-60] | 14.6 ± 8.2 | 4,032 ± 2,337 | 12,595 ± 5,415 |
| CV0-15-I | 17.5 | [5-45] | 6.0 ± 2.9 | 12,270 ± 11,773 | 25,849 ± 19,272 |

In the presence of co-polyamino acid BB15 and citrate, the addition of nicotinamide and the intramuscular injection thus accelerates the absorption of glucagon compared to subcutaneous administration.

### DESCRIPTION OF FIGURES

FIG. 1:

This figure is a graphic representation of latency time determination (LT) by monitoring the fluorescence of Thioflavin T, on a curve showing the fluorescence value (in ua arbitrary units) on the y-axis and the time in minutes on the horizontal axis.

FIG. 2:

The median pharmacodynamic glucose curves expressed by the difference in glucose versus baseline level are shown in FIG. 2. This figure represents on the x-axis, the post-injection time and on the y-axis the percentage of glucose level.

This figure illustrates the pharmacodynamic results of glucagon obtained with the compositions described in examples CR0-1-I (solid triangles) and CR0-5—(solid squares).

FIG. 3:

The pharmacokinetic curves are illustrated in FIG. 3. This figure represents on the x-axis, post-injection time and on the y-axis, the glucose level (pmol/L) versus baseline.

This figure illustrates the pharmacokinetic results of glucagon obtained with the compositions described in examples CR0-1-I (solid triangles) and CR0-5-1 (solid squares).

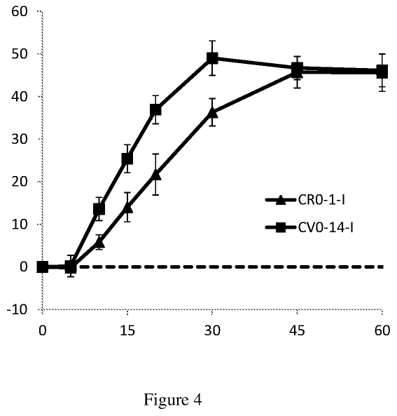

FIG. 4:

The median pharmacodynamic glucose curves expressed by the difference in glucose versus baseline level are shown in FIG. 4. This figure represents on the x-axis, the post-injection time and on the y-axis the percentage of glucose level.

This figure illustrates the pharmacodynamic results of glucagon obtained with the compositions described in examples CR0-1-I (solid triangles) and CV0-14-1 (solid squares).

FIG. 5:

The pharmacokinetic curves are illustrated in FIG. 5. This figure represents on the x-axis, post-injection time and on the y-axis, the glucose level (pmol/L) versus baseline.

This figure illustrates the pharmacokinetic results of glucagon obtained with the compositions described in examples CR0-1-I (solid triangles) and CV0-14-I (solid squares).

FIG. 6:

The median pharmacodynamic glucose curves expressed by the difference in glucose versus baseline level are shown in FIG. 6. This figure represents on the x-axis, the post-injection time and on the y-axis the percentage of glucose level.

This figure shows the pharmacodynamic results of glucagon obtained with the compositions described in examples CV0-14-I (full triangles) and CV0-15-1 (full squares).

### EXAMPLE CR0-II: PREPARATION OF A GLUCAGON SOLUTION AT 2 MG/ML CONTAINING DIFFERENT CO-POLYAMINO ACIDS OF THE INVENTION, A PHOSPHATE BUFFER (2 MM) AND GLYCEROL AT PH 7.2

A co-polyamino acid solution is added to concentrated solutions in glycerol (to obtain 300 mOsmol/kg in the final formulation), in phosphate buffer (4 mM) and m-cresol (54 mM). At this stage additives (citrate, nicotinamide, Treprostinil) can be added. The composition is briefly stirred until dissolution of the co-polyamino acid, then the solution is filtered through a membrane (0.22 μm).

The equivolumic mixture of this solution with a freshly prepared solution of glucagon, according to the protocol described in example C2-I, leads to the final compositions CR0-1-II to CR0-4-II (Table 2a). The pH of the solution is adjusted to pH 7.2±0.1 by adding 1 N NaOH/HCl, then filtered through a membrane (0.22 m).

A visual inspection is performed to determine whether or not a clear solution is obtained. A visual inspection of the samples is performed to detect visible particles, or turbidity. This inspection is carried out according to the recommendations of the European pharmacopoeia (EP 2.9.20): the samples are subjected to a lighting of at least 2000 lux and are observed on a white background and a black background. The composition is deemed limpid when the particles are visible in half of the samples the composition.

TABLE 2a
Compositions and visual appearance of 2 mg/mL glucagon solutions at
pH 7.2 at different concentrations of co-polyamino acid containing
m-cresol (27 mM), 1 mg/mL of L-methionine and phosphate buffer (2 mM).

| | Co-polyamino acid | | | | | |
|---|---|---|---|---|---|---|
| | concentration | | BC/Glu | Glycerol | | Visual appearance |
| Composition | (mg/ml) | BC | ratio | (mM) | Additive | of the solution |
| CR0-1-II | 7.2 | AB29 | 1.6 | 255 | 10 mM citrate | clear |
| CR0-2-II | 7.2 | AB29 | 1.6 | 175 | 10 mM citrate + | clear |
| | | | | | 80 mM | |
| | | | | | nicotinamide | |
| CR0-3-II | 7.8 | AB30 | 1.6 | 255 | 10 mM citrate | clear |
| CR0-4-II | 7.8 | AB30 | 1.6 | 175 | 10 mM citrate + | clear |
| | | | | | 80 mM | |
| | | | | | nicotinamide | |

### CS0 EXAMPLE-II: PREPARATION OF A GLUCAGON SOLUTION AT 1 MG/ML CONTAINING DIFFERENT CO-POLYAMINO ACIDS OF THE INVENTION, A PHOSPHATE BUFFER (2 MM) AND GLYCEROL AT PH 7.2

A co-polyamino acid solution is added to a bottle containing concentrated solutions of excipients (phosphate, glycerol) and optionally additives (m-cresol, citrate). The composition is briefly stirred until dissolution of the co-polyamino acid, then the solution is filtered through a membrane (0.22 μm).

The equivolumic mixture of this solution with the freshly prepared glucagon solution, as described in example C1-I, leads to the final compositions CS0-1-II a CS0-4-II containing 1 mg/mL of glucagon. The pH of the solution is adjusted to pH 7.2±0.1 by adding 1 N NaOH/HCl, then filtered through a membrane (0.22 μm). The details of the compositions are summarized in the table below.

A visual inspection is performed to determine whether or not a clear solution is obtained. In comparison, the glucagon solution at neutral pH is not soluble above 0.2 mg/mL.

TABLE 3a
Compositions and visual appearance of 1 mg/mL glucagon solutions at pH 7.2 at different concentrations
of co-polyamino containing 1 mg/mL of L-methionine and phosphate buffer (2 mM).

| | [symbol] acid | | | | | |
|---|---|---|---|---|---|---|
| | concentration | co-polyamino | Co-polyamino acid | Glycerol | | appearance of |
| Composition | [symbol] | acid | ratio/Glucagon | (mM) | Additive | the solution |
| CS0-1-II | 3.5 | AB29 | 1.6 | 255 | 10 mM citrate | clear |
| CS0-2-II | 3.5 | AB29 | 1.6 | 175 | 10 mM citrate + | clear |
| | | | | | 80 mM | |
| | | | | | nicotinamide | |
| CS0-3-II | 3.8 | AB30 | 1.6 | 255 | 10 mM citrate | clear |
| CS0-4-II | 3.8 | AB30 | 1.6 | 175 | 10 mM citrate + | clear |
| | | | | | 80 mM | |
| | | | | | nicotinamide | |

[symbol]

indicates data missing or illegible when filed

### EXAMPLE C5-III: TRIS BUFFER SOLUTION AT 500 MM AT PH 8.3

3.0 g of Trizma® base (Sigma-Aldrich 04577) are weighed in a 50 ml flask. Water is added without making up to the mark. Once the powder is solubilized, the pH of the solution is adjusted to pH 7.2±0.1 by adding 1 N NaOH/HCl and the solution is filtered through a membrane (0.22 μm). The water volume is adjusted to obtain the concentrated solution of Tris.

### EXAMPLES CA: PREPARATION OF 1 MG/ML GLUCAGON SOLUTIONS CONTAINING VARIOUS CO-POLYAMINO ACIDS, TRIS BUFFER (2 MM), GLYCERIN AND VARIOUS CONCENTRATIONS OF SODIUM CHLORIDE AND ZINC CHLORIDE AT PH 7.2

A concentrated glycerol solution (in order to obtain 300 mOsmol/kg in the final formulation), a concentrated solution of Tris buffer (example C5), additives (NaCl, zinc chloride) and co-polyamino acid in solution or in lyophilizate form are successively added to a bottle. The composition is stirred until dissolution of the co-polyamino acid. The pH of the solution is adjusted to pH 7.2±0.1 by adding 1 N NaOH/HCl, the solution is then filtered through a membrane (0.22 μm).

The equivolumic mixture of this solution is added to the freshly prepared glucagon solution, as described in Example C1-I, and gives the final compositions CA1-III to CA6-III and CA7-III to CA32-III containing 1 mg/mL of glucagon. The pH of the solution is adjusted to pH 7.2±0.1 by adding 1 N NaOH/HCl, then filtered through a membrane (0.22 μm). Details of the compositions are summarized in tables 1 and 2.

A visual inspection is performed to determine whether or not a clear solution is obtained (by comparison, the glucagon solution at neutral pH is not soluble above 0.2 mg/mL). A visual inspection of the samples is performed to detect visible particles, or turbidity. This inspection is carried out according to the recommendations of the European pharmacopoeia (EP 2.9.20): the samples are subjected to a lighting of at least 2000 lux and are observed on a white background and a black background. The composition is deemed limpid when the particles are visible in half of the samples the composition.

TABLE 4
Compositions and visual appearance of glucagon solutions at 1 mg/mL containing
Tris buffer (2 mM), 1 mg/mL L-methionine in the presence of different concentrations
of co-polyamino acid, sodium chloride and zinc chloride pH 7.2.

| | | Concentration of | Copolyamino | | | | Visual |
|---|---|---|---|---|---|---|---|
| | Co-polyamino | co-polyamino acid | acid/glucagon | NaCl | Zn | Glycerol | appearance of |
| Solution | acid | (mg/mL) | ratio | (mM) | (mM) | (mM) | the solution |
| CA1-III | AB14 | 10.7 | 9.0 | 0 | 0 | 289 | clear |
| CA2-III | | 10.7 | 9.0 | 25 | 0 | 239 | clear |
| CA3-III | | 10.7 | 9.0 | 50 | 0 | 189 | clear |
| CA4-III | | 10.7 | 9.0 | 0 | 1:29 AM | 285 | clear |
| CA5-III | | 10.7 | 9.0 | 25 | 1:29 AM | 235 | clear |
| CA6-III | | 10.7 | 9.0 | 50 | 1:29 AM | 185 | clear |

TABLE 5
Compositions and visual appearance of 1 mg/mL glucagon solutions at pH 7.2 containing Tris buffer (2
mM), 1 mg/mL L-methionine and variable concentrations of co-polyamino acid, sodium chloride and zinc.

| | | Concentration of | Copolyamino | | | | Visual |
|---|---|---|---|---|---|---|---|
| | Copolyamino | co-polyamino acid | acid/glucagon | NaCl | ZnCl₂ | Glycerin | appearance of |
| Composition | acid | (mg/mL) | ratio | (mM) | (mM) | (mM) | the solution |
| CA7-III | AB14 | 5.9 | 5 | 0 | 0 | 274 | Clear |
| CA8-III | | 5.9 | 5 | 50 | 0 | 174 | Clear |
| CA9-III | | 5.9 | 5 | 100 | 0 | 74 | Clear |
| CA10-III | | 5.9 | 5 | 200 | 0 | 0 | Clear |
| CA11-III | | 5.9 | 5 | 300 | 0 | 0 | Clear |
| CA12-III | | 5.9 | 5 | 0 | 2.87 | 266 | Clear |
| CA13-III | | 5.9 | 5 | 50 | 2.87 | 166 | Clear |
| CA14-III | | 5.9 | 5 | 100 | 2.87 | 66 | Clear |
| CA15-III | | 5.9 | 5 | 200 | 2.87 | 0 | Clear |
| CA16-III | | 5.9 | 5 | 300 | 2.87 | 0 | Clear |
| CA17-III | | 5.6 | 5 | 0 | 0 | 282 | Clear |
| CA18-III | | 5.6 | 5 | 25 | 0 | 232 | Clear |
| CA19-III | AB33 | 5.6 | 5 | 50 | 0 | 182 | Clear |
| CA20-III | | 5.6 | 5 | 100 | 0 | 82 | Clear |
| CA21-III | | 5.6 | 5 | 200 | 0 | 0 | Clear |
| CA22-III | | 5.6 | 5 | 300 | 0 | 0 | Clear |
| CA23-III | | 5.6 | 5 | 0 | 2.87 | 274 | Clear |
| CA24-III | | 5.6 | 5 | 25 | 2.87 | 224 | Clear |
| CA25-III | | 5.6 | 5 | 50 | 2.87 | 174 | Clear |
| CA26-III | | 5.6 | 5 | 100 | 2.87 | 74 | Clear |
| CA27-III | | 5.6 | 5 | 200 | 2.87 | 0 | Clear |
| CA28-III | | 5.6 | 5 | 300 | 2.87 | 0 | Clear |
| CA29-III | AB37 | 5.4 | 5 | 0 | 2.87 | 283 | Clear |
| CA30-III | | 5.4 | 5 | 50 | 2.87 | 183 | Clear |
| CA31-III | | 5.4 | 5 | 100 | 2.87 | 83 | Clear |
| CA32-III | | 5.4 | 5 | 200 | 2.87 | 0 | Clear |

### EXAMPLE DA1-III: PHYSICAL STABILITY IN CARTRIDGE AT 37° C. OF GLUCAGON SOLUTIONS AT 1 MG/ML IN THE PRESENCE OF CO-POLYAMINO ACID B3, TRIS BUFFER (2 MM), SODIUM CHLORIDE AND ZINC CHLORIDE AT PH 7.2

The study of the physical stabilities of the compositions of examples CA1-III and CA6-III described in the table above was carried out on volumes of 1 ml of composition in cartridges containing 3 ml (OMPI—ref: P40B4100.3250). In comparison, the glucagon solution at acidic pH of 1 mg/mL is stable for only 2 days at 37° C.

Results of the visual inspections are reported in the following table.

TABLE 8
Results of the physical stabilities of glucagon solutions
at 1 mg/mL at pH 7.2 containing Tris buffer (2 mM), 1
mg/mL of L-methionine and variable concentrations of
co-polyamino acid, sodium chloride and chloride zinc.

| | | | | | Stability |
|---|---|---|---|---|---|
| | | Concentration of | | | time at |
| | Co-polyamino | co-polyamino acid | NaCl | Zn | 37° C. |
| Solution | acid | (mg/mL) | (mM) | (mM) | (week) |
| CA1-III | AB14 | 10.7 | 0 | 0 | <2 |
| CA6-III | | 10.7 | 50 | 1.29 | >4 |

The addition of BioChaperone enables the solubilization and stabilization of glucagon at neutral pH while the glucagon in solution at acidic pH is stable for only a few days at 37° C. (2 days). The combination of salt and zinc chloride improves latency times of co-polyamino acid AB14/glucagon compositions.

### EXAMPLE DB1-III: STABILITY OF 1 MG/ML SOLUTIONS OF GLUCAGON CONTAINING CO-POLYAMINO ACID, TRIS BUFFER (2 MM), GLYCEROL AND VARYING CONCENTRATIONS OF SODIUM CHLORIDE AND ZINC CHLORIDE AT PH 7.2

The latency time results obtained are shown in the table below. In comparison, under these conditions, glucagon alone is insoluble in solution at physiological pH and glucagon solution at 1 mg/mL atacidic pH shows a fibrillation time of approximately 0.5 hours.

TABLE 10
Latency time measurement of CA7-III, CA13-III, CA14-
III, CA16-III and CA30-III to CA32-III solutions.

| | Co- | | | | Fibrillation |
|---|---|---|---|---|---|
| | polyamino | Copolyamino acid/ | NaCl | Zn | time |
| Solution | acid | glucagon ratio | (mM) | (mM) | (h) |
| CA7-III | AB14 | 5 | 0 | 0 | <2 |
| CA13-III | | 5 | 50 | 2.87 | >4 |
| CA14-III | | 5 | 100 | 2.87 | >7 |
| CA16-III | | 5 | 300 | 2.87 | >18 |
| CA30-III | AB37 | 5 | 50 | 2.87 | >3 |
| CA31-III | | 5 | 100 | 2.87 | >4 |
| CA32-III | | 5 | 200 | 2.87 | >6 |

The addition of salt or zinc alone or as a mixture in the co-polyamino acid/glucagon compositions makes it possible to obtain longer latency times.

1. A composition in the form of an injectable aqueous solution, the pH of which is comprised from 6.0 to 8.0, comprising at least:
a) human glucagon;
b) a co-polyamino acid bearing carboxylate charges and hydrophobic Hy radicals, the said co-polyamino acid consisting of glutamic or aspartic units and said hydrophobic Hy radicals from according to formula I below: 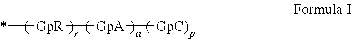
wherein
GpR is a radical according to formulas II, II′ or II″: 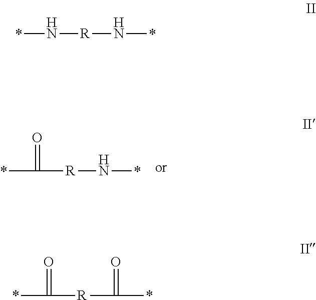
GpA is a radical according to formulas III or III′: 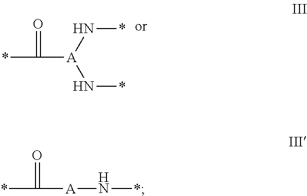
GpC is a radical according to formula IV: 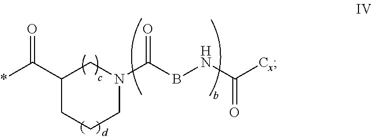
* indicate the attachment sites of the various groups;
a is an integer equal to 0 or 1;
b is an integer equal to 0 or 1;
p is an integer equal to 1 or 2 and
if p is equal to 1 then a is equal to 0 or 1 and GpA is a radical according to formula III′ and,
if p is 2 then a is 1, and GpA is a radical according to formula III;
c is an integer equal to 0 or 1, and if c is 0 then d is 1 or 2;
d is an integer of 0, 1 or 2;
r is an integer equal to 0, 1 or 2, and
if r is equal to 0, then the hydrophobic radical according to formula I is bound to the co-polyamino acid through a covalent bond between a carbonyl of the hydrophobic radical and a nitrogen atom in the N-terminal position of the co-polyamino acid, thereby forming an amide function from the reaction of an amine function at the N-terminal position of the precursor of the co-polyamino acid and an acid function borne by the precursor of the hydrophobic radical, and
if r is equal to 1 or 2, then the hydrophobic radical according to formula I is bound to the co-polyamino acid:
through a covalent bond between a nitrogen atom of the hydrophobic radial and a carbonyl of the copolyamino acid, thus forming an amide function from the reaction of an amine function of the precursor of the hydrophobic radical and an acid function borne by the precursor of the co-polyamino acid or
through a covalent bond between a carbonyl of the hydrophobic radical and a nitrogen atom in N-terminal position of the co-polyamino acid, thus forming an amide function from the reaction of an acid function of the precursor of the hydrophobic radical and an amine function in N-terminal position borne by the precursor of the co-polyamino acid;
R is a radical chosen from the group consisting of a linear or branched divalent alkyl radical comprising from 1 to 12 carbon atoms, a divalent linear or branched alkyl radical comprising from 1 to 12 carbon atoms bearing one or more —CONH₂ functions or an unsubstituted ether or polyether radical comprising from 4 to 14 carbon atoms and from 1 to 5 oxygen atoms, a divalent linear or branched alkyl radical comprising from 1 to 12 carbon atoms bearing one or more unsaturated rings or a unsubstituted ether or polyether radical comprising from 4 to 14 carbon atoms and from 1 to 5 oxygen atoms;
more precisely, R is a radical chosen from the group consisting of:
a divalent alkyl radical, linear or branched, comprising from 2 to 12 carbon atoms if GpR is a radical according to formula II, from 1 to 11 carbon atoms if GpR is a radical according to formula II′ or from 1 to 10 carbon atoms if GpR is a radical according to formula II″;
a divalent, linear or branched alkyl radical, comprising from 2 to 11 carbon atoms if GpR is a radical according to formula II, from 1 to 11 carbon atoms if GpR is a radical according to formula II′ or from 1 to 10 carbon atoms if GpR is a radical according to formula II″, said alkyl radical bearing one or more functions —CONH₂, and
A is a linear or branched alkyl radical comprising from 1 to 8 carbon atoms and and/or substituted by a radical resulting from a saturated, unsaturated or aromatic ring;
B is a radical chosen from the group consisting of an unsubstituted ether or polyether radical comprising from 4 to 14 carbon atoms and from 1 to 5 oxygen atoms or a linear or branched alkyl radical, and/or comprising an aromatic ring, comprising from 1 to 9 carbon atoms;
Cₓ is a linear or branched monovalent alkyl radical, and/or comprising a cyclic part, wherein x indicates the number of carbon atoms and:
if p is equal to 1, x is comprised from 11 to 25 (11≤x≤25)
if p is equal to 2, x is comprised from 9 to 15 (9≤x≤15),
the ratio i between the number of hydrophobic radicals and the number of glutamic or aspartic units being comprised between 0<i≤0.5;
when several hydrophobic radicals are borne by a co-polyamino acid they are therefore identical or different,
the degree of polymerization DP of glutamic or aspartic units is comprised from 5 to 250;
the free acid functions being in the form of an alkaline cation salt chosen from the group consisting of Na+ and K+.

2. The composition according to claim 1, wherein said hydrophobic radicals are chosen from the hydrophobic radicals according to formula I wherein p=1, represented by formula V below: 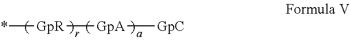
GpR, GpA, GpC, r and a as defined in claim 1.

3. The composition according to claim 1, wherein the said hydrophobic radicals are chosen from the hydrophobic radicals according to formula I wherein a=1 and p=2, represented by formula VI below: 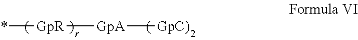
wherein
GpR, GpA, GpC, r and a as defined in claim 1.

4. The composition according to claim 1, wherein the co-polyamino acid bearing carboxylate charges and hydrophobic radicals is chosen from the co-polyamino acids according to formula VII below: 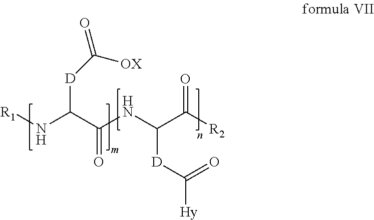
wherein,
D represents, independently, either a —CH₂— group (aspartic unit) or a —CH₂—CH₂-group (glutamic unit),
Hy is a hydrophobic radical chosen from hydrophobic radicals according to formula I, V or VI,
R₁ is a hydrophobic radical chosen from the hydrophobic radicals of according to formula I, V or VI, or a radical chosen from the group consisting of a H, a C₂ to C₁₀ linear acyl group, a C₃ to C₁₀ branched acyl group, a benzyl, a terminal “amino acid” unit and a pyroglutamate,
R2 is a hydrophobic radical chosen from hydrophobic radicals according to formula I, V or VI, or a radical —NR′R″, R′ and R″ identical or different chosen from the group consisting of H, linear or branched alkyls or cyclical in C2 to C10, benzyl and said R′ and R″ alkyls may form together one or more saturated carbon rings, unsaturated and/or aromatic and/or may contain heteroatoms, chosen from the group consisting of O, N and S,
X represents a H or a cationic entity chosen from the group consisting of metal cations;
n+m represents the degree of polymerization DP of the co-polyamino acid, namely the average number of monomeric units per co-polyamino acid chain and 5≤n+m≤250.

5. The composition according to claim 4 wherein the co-polyamino acid bearing carboxylate charges and hydrophobic radicals is chosen from the co-polyamino acids according to formulas VII wherein R₁=R′₁ and R₂=R′₂, according to formula VIIa below: 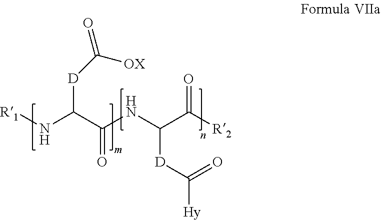
wherein,
m, n, X, D and Hy have the definitions provided above,
R′1 is a radical chosen from the group consisting of H, linear C₂ to C₁₀ acyl group, branched C₄ to C₁₀ acyl group, benzyl, terminal amino acid unit and pyroglutamate,
R′2 is an identical or different radical —NR′R″, R′ and R″ chosen from the group consisting of H, linear or branched or cyclic C₂ to Co₁₀ alkyls, benzyl and said R′ and R″ alkyls which can form together one or more saturated, unsaturated and/or aromatic carbon rings and/or which may contain heteroatoms chosen from the group consisting of O, N and S.

6. The composition according to claim 4, wherein the co-polyamino acid bearing carboxylate charges and hydrophobic radicals is chosen from
the co-polyamino acids according to formula VII wherein n=0 according to formula VIIb below: 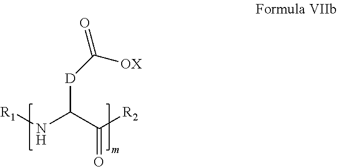
wherein m, X, D, R1 and R2 as defined in claim 4 and at least R1 or R2 is a hydrophobic radical according to formula I, V or VI.

7. The composition according to claim 6, wherein the co-polyamino acid bearing carboxylate charges and hydrophobic radicals is chosen from the co-polyamino acids according to formula VIIb wherein R₂ is a hydrophobic radical according to formula I, V or VI wherein r=1 or 2 and GpR is according to Formula II′.

8. The composition according to claim 1, wherein the co-polyamino acid bearing carboxylate charges and hydrophobic radicals is chosen from the co-polyamino acids according to formulas VII, VIIa and VIIb: 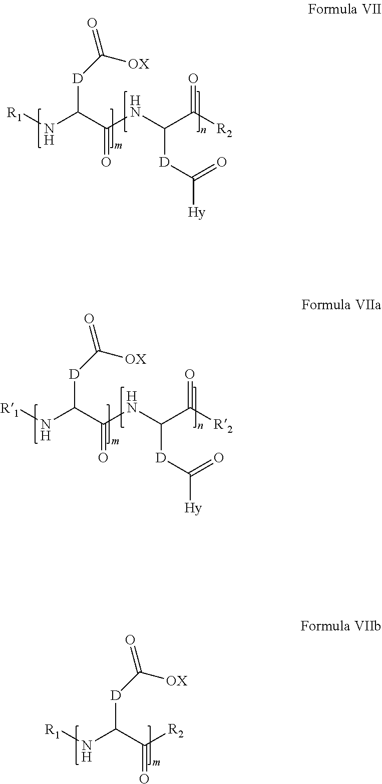
wherein the at least one co-polyamino acid polyamino acid is chosen from co-polyamino acids wherein group D is a —CH₂— (aspartic unit) group.

9. A composition according to claim 1, wherein the co-polyamino acid bearing carboxylate charges and hydrophobic radicals is chosen from the co-polyamino acids according to formulas VII, VIIa and VIIb: 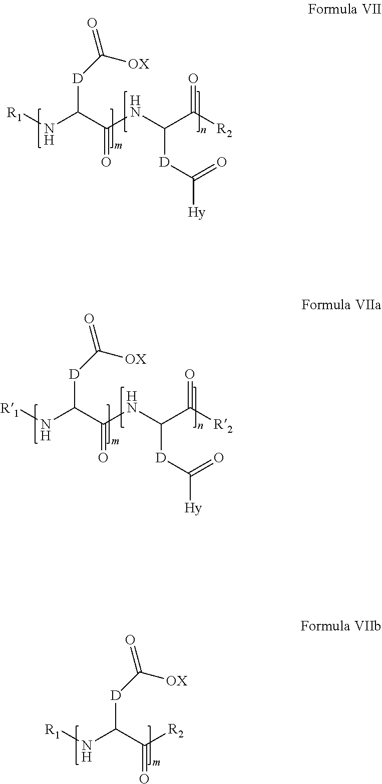
wherein the at least one co-polyamino acid polyamino acid is chosen from co-polyamino acids wherein group D is a —CH₂—CH₂— (glutamic unit) group.

10. The composition according to claim 1, wherein the concentration of co-polyamino acid bearing carboxylate charges and hydrophobic radicals is at most 40 mg/ml.

11. The composition according to claim 1, wherein the concentration of human glucagon is comprised from 0.25 to 5 mg/ml.

12. The composition according to claim 1, wherein the molar ratio [hydrophobic radical]/[human glucagon] is less than 15.

13. The composition according to claim 1, wherein it further comprises a polyanionic compound.

14. The composition according to claim 13, wherein the polyanionic compound is chosen from the group consisting of polycarboxylic acids and their Na⁺, K⁺, Ca²⁺ or Mg²⁺ salts.

15. The composition according to claim 13, wherein the polycarboxylic acid is chosen from the group consisting of citric acid, tartaric acid and their N⁺, K⁺, Ca²⁺ or Mg²⁺ salts.

16. The composition according to claim 15, wherein the polycarboxylic acid is citric acid and its N⁺, K⁺, Ca²⁺ or Mg²⁺ salts.

17. The composition according to claim 1, wherein it further comprises an absorption promoter chosen from absorption promoters, diffusion promoters or vasodilator agents, alone or as a mixture.

18. The composition according to claim 1, wherein it further comprises a zinc salt.

19. The composition according to claim 1, wherein it further comprises a gastrointestinal hormone.

20. The composition according to claim 18, wherein the gastrointestinal hormone is chosen from the group consisting of exenatide, liraglutide, lixisenatide, albiglutide and dulaglutide, their analogues or derivatives and their pharmaceutically acceptable salts.

21. The composition according to claim 19, wherein the concentration of gastrointestinal hormone is in a range of 0.01 to 10 mg/mL.
